(19) United States
(12) Patent Application Publication
(10) Pub. No.: US 2023/0183689 A1
(43) Pub. Date: Jun. 15, 2023

(54) Compositions and Methods for Genome Editing

(71) Applicant: Shape Therapeutics Inc., Seattle, WA (US)

(72) Inventor: Debojit BOSE, Seattle, WA (US)

(21) Appl. No.: 17/925,870

(22) PCT Filed: May 26, 2021
(86) PCT No.: PCT/US2021/034272

§ 371 Date: Nov. 17, 2022

Related U.S. Application Data
(60) Provisional application No. 63/030,121, filed on May 26, 2020.
(60) Provisional application No. 63/112,328, filed on Nov. 11, 2020.
(60) Provisional application No. 63/119,881, filed on Dec. 1, 2020.
(60) Provisional application No. 63/178,221, filed on Apr. 22, 2021.

(51) Int. Cl. C12N 15/11

(52) U.S. Cl. CPC C12N 15/111; C12N 2310/11; C12N 2310/315; C12N 2310/3231; C12N 2310/344; C12N 2310/346; C12N 2320/34; C12N 2320/53

(57) ABSTRACT

Described herein are compositions for targeting and editing genomes. Also described herein are methods for targeting and editing genomes utilizing the compositions in the instant disclosure.

### CROSS REFERENCE

This application claims priority under 35 U.S.C. § 119 to Provisional Application Ser. No. 63/030,121, filed May 26, 2020, Provisional Application Ser. No. 63/112,328, filed Nov. 11, 2020, Provisional Application Ser. No. 63/119,881, filed Dec. 1, 2020, and Provisional Application Ser. No. 63/178,221, filed Apr. 22, 2021, the disclosures of which are incorporated herein by reference in their entirety.

### SUMMARY

Disclosed herein are engineered guide RNAs that comprise: (a) at least one chemical modification, and (b) a targeting domain, wherein the targeting domain hybridizes to a target RNA when administered to a subject, thereby forming a complex that recruits an RNA editing entity present in a cell of the subject; wherein the RNA editing entity, when associated with the engineered guide RNA and the target RNA, performs a targeted editing of a base of a nucleotide of the target RNA. In some embodiments, the engineered guide RNA comprises a mismatch relative to the target RNA. In some embodiments, the mismatch comprises a base in the engineered guide RNA opposite to and unpaired with a base in the target RNA molecule. In some embodiments, the mismatch comprises an A/C mismatch and wherein the A is in the target RNA molecule and the C is in the engineered guide RNA. In some embodiments, the A in the A/C mismatch comprises the base of the nucleotide in the target RNA molecule chemically modified by the RNA editing entity. In some embodiments, the engineered guide RNA comprises a C opposite the base of the nucleotide in the target RNA chemically modified by the RNA editing entity. In some embodiments, the target RNA molecule comprises a G adjacent to and 5′ of the base of the nucleotide in the target RNA chemically modified by the RNA editing entity. In some embodiments, the engineered guide further comprises a G adjacent to and 5′ of the C opposite to and unpaired with the A in the target RNA molecule chemically modified by the RNA editing entity. In some embodiments, the engineered guide RNA comprises an unmodified nucleotide on either side of the mismatch. In some embodiments, the engineered guide RNA does not comprise a second mismatch within 2 nucleotides of the mismatch. In some embodiments, the chemical modification is positioned: (a) proximal to a 5′ end of the engineered guide RNA; (b) proximal to a 5′ end of a region of the engineered guide RNA; or (c) both (a) and (b). In some embodiments, the chemical modification is positioned: (a) proximal to a 3′ end of the engineered guide RNA; (b) proximal to a 3′ end of a region of the engineered guide RNA; or (c) both (a) and (b). In some embodiments, the chemical modification is positioned proximal to a 5′ end of the engineered guide RNA, proximal to a 5′ end of a region of the engineered guide RNA, proximal to a 3′ end of the engineered guide RNA, proximal to a 3′ end of a region of the engineered guide RNA, or any combination thereof. In some embodiments, the engineered guide RNA, when present in an aqueous solution and not bound to the target RNA, does not bind to the RNA editing entity with a dissociation constant less than about 100 nM. In some embodiments, the chemical modification comprises a substitution of one or both of non-linking phosphate oxygen atoms in a phosphodiester backbone linkage of the engineered guide RNA as provided in Table 2. In some embodiments, the chemical modification comprises a substitution of one or more of linking phosphate oxygen atoms in a phosphodiester backbone linkage of the engineered guide RNA as provided in Table 2. In some embodiments, the chemical modification comprises a modification to a sugar of a nucleotide of the engineered guide RNA as provided in Table 2. In some embodiments, the modification to the sugar of the nucleotide of the engineered guide RNA comprises at least one locked nucleic acid (LNA). In some embodiments, the modification to the sugar of the nucleotide of the engineered guide RNA comprises at least one unlocked nucleic acid (UNA). In some embodiments, the modification to the sugar comprises a modification of a constituent of the sugar, wherein the sugar comprises a ribose sugar. In some embodiments, the modification to the constituent of the ribose sugar of the nucleotide of the engineered guide RNA comprises a 2′-O-methyl group. In some embodiments, the chemical modification comprises replacement of a phosphate moiety of the engineered guide RNA with a dephospho linker as provided in Table 2. In some embodiments, the chemical modification comprises a modification of a phosphate backbone of the engineered guide RNA as provided in Table 2. In some embodiments, the engineered guide RNA comprises a phosphothioate group. In some embodiments, the chemical modification comprises a modification to a base of a nucleotide of the engineered guide RNA. In some embodiments, the chemical modification comprises an unnatural base of a nucleotide as provided in Table 2. In some embodiments, the chemical modification comprises a morpholino group, a cyclobutyl group, pyrrolidine group, or peptide nucleic acid (PNA) nucleoside surrogate. In some embodiments, the chemical modification comprises at least one stereopure nucleic acid as provided in Table 2. In some embodiments, the engineered guide RNA comprises from 1 to 100 chemical modifications, each of which can be independently the same or different. In some embodiments, the chemical modification does not comprise a naturally occurring chemical modification to a nucleic acid in a eukaryotic cell. In some embodiments, the chemical modification increases specificity of the engineered guide RNA binding to the target RNA compared to a specificity of an otherwise identical reference polynucleotide without the at least one chemical modification. In some embodiments, the chemical modification increases resistance to nuclease digestion of the engineered guide RNA compared to resistance to nuclease digestion of an otherwise identical reference polynucleotide without the at least one chemical modification as measured in an in vitro assay. In some embodiments, the chemical modification decreases immunogenicity of the engineered guide RNA compared to immunogenicity of an otherwise identical reference polynucleotide without the at least one chemical modification as measured in an in vitro assay. In some embodiments, the target RNA comprises RAB7A, ABCA4, SERPINA1, SERPINA1 E342K, HEXA, LRRK2, SNCA, APP, Tau, CFTR, ALAS1, ATP7B, ATP7B G1226R, HFE C282Y, LIPA c.894 G>A, PCSK9 start site, or SCNN1A start site, a fragment any of these, or any combination thereof. In some embodiments, the target RNA comprises SERPINA1 E342K. In some embodiments, the engineered guide RNA has at least 70%, at least 80%, at least 85%, at least 90%, at least 92%, at least 95%, at least 97%, or at least 99% sequence identity with any one of SEQ ID NOs: 1-2. In some embodiments, the target RNA encodes ABCA4. In some embodiments, the RNA editing entity is: (a) ADAR or APOBEC; (b) a catalytically active fragment of (a); (c) fusion polypeptide comprising (a) or (b); or (d) any combination of (a)-(c). In some embodiments, the RNA editing entity comprises ADAR, and wherein the ADAR comprises ADAR1, ADAR2, ADAR3, or a combination thereof. In some embodiments, the RNA editing entity is endogenous to the cell of the subject. In some embodiments, the RNA editing entity is exogenously provided. In some embodiments, the engineered polynucleotide further comprises a structural loop stabilized scaffold. In some embodiments, the structural loop stabilized scaffold comprises a stem loop, a junction, a T junction, a clover leaf, a pseudoknot, or any combination thereof. In some embodiments, the structural loop stabilized scaffold comprises at least 1, least 2, at least 3, at least 4, at least 5, at least 6, at least 7, at least 8, at least 9, or at least 10 stem loop structures. In some embodiments, the structural loop stabilized scaffold comprises a tRNA scaffold. In some embodiments, the engineered polynucleotide further comprises an RNA editing entity recruiting domain. In some embodiments, the engineered guide RNA is conjugated to a targeting moiety. In some embodiments, the targeting moiety targets a neuronal cell. In some embodiments, the targeting moiety targets a liver cell. In some embodiments, the targeting moiety targets a macular cell. In some embodiments, the engineered guide RNA is encapsulated in particles. In some embodiments, the particles comprise nanoparticles. In some embodiments, the particles comprise liposomes.

Also disclosed herein are pharmaceutical compositions in unit dose form comprising: (a) an engineered guide RNA as described herein; and (b) a pharmaceutically acceptable: excipient, carrier, or diluent. In some embodiments, an engineered polynucleotide comprises (a) at least one chemical modification, and (b) a targeting domain, wherein the targeting domain hybridizes to a target RNA when administered to a subject, thereby forming a complex that recruits an RNA editing entity present in a cell of the subject; wherein the RNA editing entity, when associated with the engineered guide RNA and the target RNA, performs a targeted editing of a base of a nucleotide of the target RNA. In some embodiments, the engineered guide RNA comprises a mismatch relative to the target RNA. In some embodiments, the mismatch comprises a base in the engineered guide RNA opposite to and unpaired with a base in the target RNA molecule. In some embodiments, the mismatch comprises an A/C mismatch and wherein the A is in the target RNA molecule and the C is in the engineered guide RNA. In some embodiments, the A in the A/C mismatch comprises the base of the nucleotide in the target RNA molecule chemically modified by the RNA editing entity. In some embodiments, the engineered guide RNA comprises a C opposite the base of the nucleotide in the target RNA chemically modified by the RNA editing entity. In some embodiments, the target RNA molecule comprises a G adjacent to and 5′ of the base of the nucleotide in the target RNA chemically modified by the RNA editing entity. In some embodiments, the engineered guide further comprises a G adjacent to and 5′ of the C opposite to and unpaired with the A in the target RNA molecule chemically modified by the RNA editing entity. In some embodiments, the engineered guide RNA comprises an unmodified nucleotide on either side of the mismatch. In some embodiments, the engineered guide RNA does not comprise a second mismatch within 2 nucleotides of the mismatch. In some embodiments, the chemical modification is positioned: (a) proximal to a 5′ end of the engineered guide RNA; (b) proximal to a 5′ end of a region of the engineered guide RNA; or (c) both (a) and (b). In some embodiments, the chemical modification is positioned: (a) proximal to a 3′ end of the engineered guide RNA; (b) proximal to a 3′ end of a region of the engineered guide RNA; or (c) both (a) and (b). In some embodiments, the chemical modification is positioned proximal to a 5′ end of the engineered guide RNA, proximal to a 5′ end of a region of the engineered guide RNA, proximal to a 3′ end of the engineered guide RNA, proximal to a 3′ end of a region of the engineered guide RNA, or any combination thereof. In some embodiments, the engineered guide RNA, when present in an aqueous solution and not bound to the target RNA, does not bind to the RNA editing entity with a dissociation constant less than about 100 nM. In some embodiments, the chemical modification comprises a substitution of one or both of non-linking phosphate oxygen atoms in a phosphodiester backbone linkage of the engineered guide RNA as provided in Table 2. In some embodiments, the chemical modification comprises a substitution of one or more of linking phosphate oxygen atoms in a phosphodiester backbone linkage of the engineered guide RNA as provided in Table 2. In some embodiments, the chemical modification comprises a modification to a sugar of a nucleotide of the engineered guide RNA as provided in Table 2. In some embodiments, the modification to the sugar of the nucleotide of the engineered guide RNA comprises at least one locked nucleic acid (LNA). In some embodiments, the modification to the sugar of the nucleotide of the engineered guide RNA comprises at least one unlocked nucleic acid (UNA). In some embodiments, the modification to the sugar comprises a modification of a constituent of the sugar, wherein the sugar comprises a ribose sugar. In some embodiments, the modification to the constituent of the ribose sugar of the nucleotide of the engineered guide RNA comprises a 2′-O-methyl group. In some embodiments, the chemical modification comprises replacement of a phosphate moiety of the engineered guide RNA with a dephospho linker as provided in Table 2. In some embodiments, the chemical modification comprises a modification of a phosphate backbone of the engineered guide RNA as provided in Table 2. In some embodiments, the engineered guide RNA comprises a phosphothioate group. In some embodiments, the chemical modification comprises a modification to a base of a nucleotide of the engineered guide RNA. In some embodiments, the chemical modification comprises an unnatural base of a nucleotide as provided in Table 2. In some embodiments, the chemical modification comprises a morpholino group, a cyclobutyl group, pyrrolidine group, or peptide nucleic acid (PNA) nucleoside surrogate. In some embodiments, the chemical modification comprises at least one stereopure nucleic acid as provided in Table 2. In some embodiments, the engineered guide RNA comprises from 1 to 100 chemical modifications, each of which can be independently the same or different. In some embodiments, the chemical modification does not comprise a naturally occurring chemical modification to a nucleic acid in a eukaryotic cell. In some embodiments, the chemical modification increases specificity of the engineered guide RNA binding to the target RNA compared to a specificity of an otherwise identical reference polynucleotide without the at least one chemical modification. In some embodiments, the chemical modification increases resistance to nuclease digestion of the engineered guide RNA compared to resistance to nuclease digestion of an otherwise identical reference polynucleotide without the at least one chemical modification as measured in an in vitro assay. In some embodiments, the chemical modification decreases immunogenicity of the engineered guide RNA compared to immunogenicity of an otherwise identical reference polynucleotide without the at least one chemical modification as measured in an in vitro assay. In some embodiments, the target RNA comprises RAB7A, ABCA4, SERPINA1, SERPINA1 E342K, HEXA, LRRK2, SNCA, APP, Tau, CFTR, ALAS1, ATP7B, ATP7B G1226R, HFE C282Y, LIPA c.894 G>A, PCSK9 start site, or SCNN1A start site, a fragment any of these, or any combination thereof. In some embodiments, the target RNA comprises SERPINA1 E342K. In some embodiments, the engineered guide RNA has at least 70%, at least 80%, at least 85%, at least 90%, at least 92%, at least 95%, at least 97%, or at least 99% sequence identity with any one of SEQ ID NOs: 1-2. In some embodiments, the target RNA encodes ABCA4. In some embodiments, the RNA editing entity is: (a) ADAR or APOBEC; (b) a catalytically active fragment of (a); (c) fusion polypeptide comprising (a) or (b); or (d) any combination of (a)-(c). In some embodiments, the RNA editing entity comprises ADAR, and wherein the ADAR comprises ADAR1, ADAR2, ADAR3, or a combination thereof. In some embodiments, the RNA editing entity is endogenous to the cell of the subject. In some embodiments, the RNA editing entity is exogenously provided. In some embodiments, the engineered polynucleotide further comprises a structural loop stabilized scaffold. In some embodiments, the structural loop stabilized scaffold comprises a stem loop, a junction, a T junction, a clover leaf, a pseudoknot, or any combination thereof. In some embodiments, the structural loop stabilized scaffold comprises at least 1, least 2, at least 3, at least 4, at least 5, at least 6, at least 7, at least 8, at least 9, or at least 10 stem loop structures. In some embodiments, the structural loop stabilized scaffold comprises a tRNA scaffold. In some embodiments, the engineered polynucleotide further comprises an RNA editing entity recruiting domain. In some embodiments, the engineered guide RNA is conjugated to a targeting moiety. In some embodiments, the targeting moiety targets a neuronal cell. In some embodiments, the targeting moiety targets a liver cell. In some embodiments, the targeting moiety targets a macular cell. In some embodiments, the engineered guide RNA is encapsulated in particles. In some embodiments, the particles comprise nanoparticles. In some embodiments, the particles comprise liposomes.

Also disclosed herein are methods of treating or preventing a disease or a condition in a subject in need thereof, the method comprising: administering to the subject an engineered guide RNA as described herein, or a pharmaceutical composition as described herein. In some embodiments, the engineered polynucleotide or the pharmaceutical composition is administered to the subject intrathecally, intraocularly, intravitreally, retinally, intravenously, intramuscularly, intraventricularly, intracerebrally, intracerebellarly, intracerebroventricularly, intraperenchymally, subcutaneously, or a combination thereof. In some embodiments, the disease or the condition comprises a neurological disease or condition. In some embodiments, the neurological or neurodevelopmental disease or condition comprises Parkinson's disease, Alzheimer's disease, or dementia. In some embodiments, the disease or the condition comprises a liver disease or condition. In some embodiments, the liver disease or condition comprises liver cirrhosis. In some embodiments, the liver disease or condition comprises alpha-1 antitrypsin deficiency (AAT deficiency). In some embodiments, the disease or the condition comprises macular degeneration. In some embodiments, the macular degeneration comprises Stargardt's disease. In some embodiments, an engineered polynucleotide comprises (a) at least one chemical modification, and (b) a targeting domain, wherein the targeting domain hybridizes to a target RNA when administered to a subject, thereby forming a complex that recruits an RNA editing entity present in a cell of the subject; wherein the RNA editing entity, when associated with the engineered guide RNA and the target RNA, performs a targeted editing of a base of a nucleotide of the target RNA. In some embodiments, the engineered guide RNA comprises a mismatch relative to the target RNA. In some embodiments, the mismatch comprises a base in the engineered guide RNA opposite to and unpaired with a base in the target RNA molecule. In some embodiments, the mismatch comprises an A/C mismatch and wherein the A is in the target RNA molecule and the C is in the engineered guide RNA. In some embodiments, the A in the A/C mismatch comprises the base of the nucleotide in the target RNA molecule chemically modified by the RNA editing entity. In some embodiments, the engineered guide RNA comprises a C opposite the base of the nucleotide in the target RNA chemically modified by the RNA editing entity. In some embodiments, the target RNA molecule comprises a G adjacent to and 5′ of the base of the nucleotide in the target RNA chemically modified by the RNA editing entity. In some embodiments, the engineered guide further comprises a G adjacent to and 5′ of the C opposite to and unpaired with the A in the target RNA molecule chemically modified by the RNA editing entity. In some embodiments, the engineered guide RNA comprises an unmodified nucleotide on either side of the mismatch. In some embodiments, the engineered guide RNA does not comprise a second mismatch within 2 nucleotides of the mismatch. In some embodiments, the chemical modification is positioned: (a) proximal to a 5′ end of the engineered guide RNA; (b) proximal to a 5′ end of a region of the engineered guide RNA; or (c) both (a) and (b). In some embodiments, the chemical modification is positioned: (a) proximal to a 3′ end of the engineered guide RNA; (b) proximal to a 3′ end of a region of the engineered guide RNA; or (c) both (a) and (b). In some embodiments, the chemical modification is positioned proximal to a 5′ end of the engineered guide RNA, proximal to a 5′ end of a region of the engineered guide RNA, proximal to a 3′ end of the engineered guide RNA, proximal to a 3′ end of a region of the engineered guide RNA, or any combination thereof. In some embodiments, the engineered guide RNA, when present in an aqueous solution and not bound to the target RNA, does not bind to the RNA editing entity with a dissociation constant less than about 100 nM. In some embodiments, the chemical modification comprises a substitution of one or both of non-linking phosphate oxygen atoms in a phosphodiester backbone linkage of the engineered guide RNA as provided in Table 2. In some embodiments, the chemical modification comprises a substitution of one or more of linking phosphate oxygen atoms in a phosphodiester backbone linkage of the engineered guide RNA as provided in Table 2. In some embodiments, the chemical modification comprises a modification to a sugar of a nucleotide of the engineered guide RNA as provided in Table 2. In some embodiments, the modification to the sugar of the nucleotide of the engineered guide RNA comprises at least one locked nucleic acid (LNA). In some embodiments, the modification to the sugar of the nucleotide of the engineered guide RNA comprises at least one unlocked nucleic acid (UNA). In some embodiments, the modification to the sugar comprises a modification of a constituent of the sugar, wherein the sugar comprises a ribose sugar. In some embodiments, the modification to the constituent of the ribose sugar of the nucleotide of the engineered guide RNA comprises a 2′-O-methyl group. In some embodiments, the chemical modification comprises replacement of a phosphate moiety of the engineered guide RNA with a dephospho linker as provided in Table 2. In some embodiments, the chemical modification comprises a modification of a phosphate backbone of the engineered guide RNA as provided in Table 2. In some embodiments, the engineered guide RNA comprises a phosphothioate group. In some embodiments, the chemical modification comprises a modification to a base of a nucleotide of the engineered guide RNA. In some embodiments, the chemical modification comprises an unnatural base of a nucleotide as provided in Table 2. In some embodiments, the chemical modification comprises a morpholino group, a cyclobutyl group, pyrrolidine group, or peptide nucleic acid (PNA) nucleoside surrogate. In some embodiments, the chemical modification comprises at least one stereopure nucleic acid as provided in Table 2. In some embodiments, the engineered guide RNA comprises from 1 to 100 chemical modifications, each of which can be independently the same or different. In some embodiments, the chemical modification does not comprise a naturally occurring chemical modification to a nucleic acid in a eukaryotic cell. In some embodiments, the chemical modification increases specificity of the engineered guide RNA binding to the target RNA compared to a specificity of an otherwise identical reference polynucleotide without the at least one chemical modification. In some embodiments, the chemical modification increases resistance to nuclease digestion of the engineered guide RNA compared to resistance to nuclease digestion of an otherwise identical reference polynucleotide without the at least one chemical modification as measured in an in vitro assay. In some embodiments, the chemical modification decreases immunogenicity of the engineered guide RNA compared to immunogenicity of an otherwise identical reference polynucleotide without the at least one chemical modification as measured in an in vitro assay. In some embodiments, the target RNA comprises RAB7A, ABCA4, SERPINA1, SERPINA1 E342K, HEXA, LRRK2, SNCA, APP, Tau, CFTR, ALAS1, ATP7B, ATP7B G1226R, HFE C282Y, LIPA c.894 G>A, PCSK9 start site, or SCNN1A start site, a fragment any of these, or any combination thereof. In some embodiments, the target RNA comprises SERPINA1 E342K. In some embodiments, the engineered guide RNA has at least 70%, at least 80%, at least 85%, at least 90%, at least 92%, at least 95%, at least 97%, or at least 99% sequence identity with any one of SEQ ID NOs: 1-2. In some embodiments, the target RNA encodes ABCA4. In some embodiments, the RNA editing entity is: (a) ADAR or APOBEC; (b) a catalytically active fragment of (a); (c) fusion polypeptide comprising (a) or (b); or (d) any combination of (a)-(c). In some embodiments, the RNA editing entity comprises ADAR, and wherein the ADAR comprises ADAR1, ADAR2, ADAR3, or a combination thereof. In some embodiments, the RNA editing entity is endogenous to the cell of the subject. In some embodiments, the RNA editing entity is exogenously provided. In some embodiments, the engineered polynucleotide further comprises a structural loop stabilized scaffold. In some embodiments, the structural loop stabilized scaffold comprises a stem loop, a junction, a T junction, a clover leaf, a pseudoknot, or any combination thereof. In some embodiments, the structural loop stabilized scaffold comprises at least 1, least 2, at least 3, at least 4, at least 5, at least 6, at least 7, at least 8, at least 9, or at least 10 stem loop structures. In some embodiments, the structural loop stabilized scaffold comprises a tRNA scaffold. In some embodiments, the engineered polynucleotide further comprises an RNA editing entity recruiting domain. In some embodiments, the engineered guide RNA is conjugated to a targeting moiety. In some embodiments, the targeting moiety targets a neuronal cell. In some embodiments, the targeting moiety targets a liver cell. In some embodiments, the targeting moiety targets a macular cell. In some embodiments, the engineered guide RNA is encapsulated in particles. In some embodiments, the particles comprise nanoparticles. In some embodiments, the particles comprise liposomes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

This patent application contains at least one drawing executed in color. Copies of this patent or patent application with color drawing(s) will be provided by the Office upon request and payment of the necessary fee.

FIGS. 1A-1D illustrate increased nucleic acid editing by three chemically modified guide RNAs. FIG. 1A illustrates exemplary chemically modified gRNAs. FIG. 1B illustrates increased percentage of RAB7A edited by the gRNAs 2-3 of FIG. 1A in HEPG2 cells as determined by droplet digital PCR (ddPCR) or by Sanger sequencing. FIG. 1C illustrates increased percentage of RAB7A edited by the gRNAs 2-4 of FIG. 1A in K562 cells. FIG. 1D illustrates Sanger sequencing traces showing successful editing of RAB7A from A to G (indicated by an arrow).

FIGS. 2A-2D illustrate increased nucleic acid editing by chemically modified gRNAs. FIG. 2A illustrates four exemplary chemically modified gRNAs. FIG. 2B illustrates increased percentage of RAB7A edited by gRNA 2 and 3 of FIG. 2A as determined by ddPCR. FIG. 2C illustrates increased on target percent RAb7A editing by gRNA 2 and 3 of FIG. 2A as determined by Sanger sequencing. FIG. 2D shows Sanger sequencing traces showing the editing of the RAB7A from A to G (target) and off target editing (arrows pointing to +9nt and −9nt).

FIG. 3 illustrates temporal RAB7A editing with a chemically modified gRNA (gRNA2 of FIG. 2A) over 48 hours.

FIG. 4 shows Sanger sequencing traces demonstrating RAB7A editing in retinal epithelial cells using chemically modified guide RNAs of the present disclosure (gRNA3 of FIG. 1A as compared to the unmodified gRNA1 of FIG. 1A).

FIG. 5 shows SNCA 3′UTR editing using chemically modified guide RNAs of the present disclosure.

FIG. 6 shows RAB7A editing in K562 cells using chemically modified guide RNAs of the present disclosure of varying lengths.

FIG. 7 shows schematics of the various gRNAs tested (top left), results of RNA editing (top right), and gRNA stability (bottom).

FIG. 8 shows an RNA agarose gel and a graph of RNA editing for guides of a length of 100 and 50.

FIG. 9 shows that chemically modified gRNAs successfully mediated editing of all 3 targets at higher levels than the IVT guides without modifications.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

Use of polynucleotides for recruiting enzymes a target nucleic acid for genetic editing may be utilized for therapeutic purposes. For example, nucleic acid guided Cas proteins can edit genetic mutations to treat diseases or conditions. Genetic editing of DNA remains risky. The possibility of introducing permanent genetic mutation via off-target effects can be significant. The efficiency and specificity of editing genes at RNA level also remain limited. Notability, the polynucleotides for guiding the nucleic acid editing entities to edit nucleic acid can: induce off-target editing; be degraded due to hydrolysis or nuclease digestion; and be ineffective for recruiting of the nucleic acid editing entities to the target nucleic acid. Accordingly, there remains a need for compositions and methods to efficiently and specifically recruit nucleic acid editing entities to edit a target nucleic acid for treating diseases or conditions. There also remains a need for polynucleotides that can target nucleic acid and recruit nucleic acid editing entity with increased efficiency and specificity. There also remains a need for polynucleotides, upon administering to a subject in need thereof, exhibiting: increased resistance to degradation; and decreased immunogenicity. Some genetic editing methods include using polynucleotides for guiding nucleic acid editing entities such as enzymes for editing at an RNA level, where the nucleic acid editing enzymes can edit RNA such as pre-mRNA or mRNA.

### Overview

Described herein are chemically modified guide RNAs engineered to facilitate editing of a target RNA polynucleotide implicated in a disease or condition. Such chemically modified guide RNAs comprise a targeting domain antisense to the target RNA polynucleotide. As disclosed herein, hybridization of the targeting domain of the chemically modified guide RNA to the target RNA polynucleotide in a mammalian cell provides a duplex substrate that is recognized by RNA editing enzymes (e.g. deaminases) in the mammalian cell that perform a targeted chemical modification of a base of a nucleotide of the target RNA polynucleotide, resulting in conversion of the base to a different base site specifically. Accordingly, the chemically modified guide RNAs described herein can be deployed to treat diseases or conditions through site directed editing of the target RNA polynucleotide.

Described herein are compositions and methods for editing genes or transcripts for treating a disease or a condition. The compositions and methods described herein present an improvement over the currently available modalities for editing genes for treating a disease or a condition. In some embodiments, the compositions and methods described herein edit a gene or a transcript of the gene that causes a disease or a condition. In some embodiments, the compositions and methods described herein comprise contacting a cell with an engineered guide RNA described herein. In some embodiments, the compositions and methods described herein comprise administering the engineered guide RNA described herein to a subject in need thereof. In some cases, the compositions and methods described herein comprise utilizing the engineered guide RNA to recruit a nucleic acid editing entity to edit a gene or transcript of the gene that causes the disease or the condition, thereby treating the disease or the condition. In some embodiments, the engineered guide RNA recruits a nucleic acid editing entity to edit and restore the mutation causing the disease or the condition back to a wild type, thereby treating the disease or the condition. In some embodiments, the nucleic acid editing entity is a nucleic acid editing enzyme. In some embodiments, the nucleic acid editing entity is an RNA editing enzyme.

In some embodiments, the engineered guide RNA comprises at least one chemical modification. In some embodiments, the at least one chemical modification increases the efficiency of the engineered guide RNA in recruiting the nucleic acid editing entity to edit the disease or condition causing gene or transcript. In some embodiments, the at least one chemical modification increases the specificity of the engineered guide RNA to recruit the nucleic acid editing entity to edit the disease or condition causing gene or transcript. In some cases, the disease or condition is associated with a mutation in a DNA molecule or RNA molecule encoding ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. In some examples, a protein encoded for by a mutated DNA molecule or RNA molecule encoding ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. contributes to, at least in part, the pathogenesis or progression of a disease. In some examples, the mutation in the DNA or RNA molecule is relative to an otherwise identical reference DNA or RNA molecule.

In some cases, the at least one chemical modification increases the resistance of the engineered guide RNA to nuclease degradation or hydrolysis. In some cases, the at least one chemical modification increases the half-life of the engineered guide RNA. In some cases, the at least one chemical modification increases the half-life of the engineered guide RNA in a cell. In some instances, the at least one chemical modification increases the half-life of the engineered guide RNA when the engineered guide RNA is administered to a subject in need thereof. In some instances, the at least one chemical modification decreases immunogenicity induced by the engineered guide RNA when the engineered guide RNA is administered to a subject in need thereof.

### Compositions

Described herein, in some cases, is a composition comprising a chemically modified engineered guide RNA. In some embodiments, prior to chemical modification, an engineered modified guide RNA for recruiting a nucleic acid editing entity to a target nucleic acid can be encoded by a vector polynucleotide. In some cases, an engineered modified guide RNA can be chemically synthesized directly. In some instances, the guide nucleic acid can be a guide RNA (gRNA). In some cases, the engineered guide RNA recruits the nucleic acid editing entity to the target nucleic acid. In some embodiments, the nucleic acid editing entity can be a DNA editing entity (e.g., Cas9 or Cas12). In some embodiments, the nucleic acid editing entity can be an RNA editing entity. In some embodiments, the nucleic acid editing entity is endogenous to the cell contacted by the engineered guide RNA. In some instances, the nucleic acid editing entity is encoded by a vector. In some instances, the nucleic acid editing entity is not encoded by a vector. In some cases, the nucleic acid editing entity is encoded by the same vector that also encodes a non-chemically modified engineered guide RNA. Nucleic acid editing entity can include cytidine deaminase (e.g. APOBEC1, APOBEC3A, APOBEC3B, APOBEC3C, APOBEC3D, APOBEC3F, APOBEC3G, or APOBEC3H) for converting cytosine C to U/T; adenosine deaminase (e.g. ADAR, ADAR1, ADAR2, or ADAR3) for converting adenosine A to I/G; methyl transferase (Dnmt1 transferase, Dnmt3a, or Dnmt3b) for converting cytosine to methyl cytosine; demethylase for converting methyl cytosine to cytosine; or Tet methylcytosine dioxygenase 1.

In some embodiments, a CRISPR-Cas editing enzyme can be mutated to be deactivated. Said “dead Cas” or “dCas” systems can be coupled to an RNA editing entity (e.g., ADAR) for RNA editing of a target RNA of the present disclosure.

In some embodiments, the engineered guide RNA described herein can be used to target a nucleic acid. The target nucleic acid may be an RNA such as an mRNA. In some cases, the engineered guide RNA described herein can be used to target RNA. Targeting an RNA can be a process by which RNA can be enzymatically modified post synthesis on specific nucleosides. Targeting of RNA can comprise any one of an insertion, deletion, or substitution of a nucleotide(s). Examples of RNA targeting include pseudouridylation (the isomerization of uridine residues) and deamination (removal of an amine group from cytidine to give rise to uridine, or C-to-U editing or from adenosine to inosine, or A-to-I editing).

Targeting of RNA can be a way to modulate expression of a polypeptide. For example, through modulation of polypeptide-encoding dsRNA substrates that enter the RNA interference (RNAi) pathway. This modulation may then act at the chromatin level to modulate expression of the polypeptide.

Targeting of RNA can also be a way to regulate gene translation. RNA editing can be a mechanism in which to regulate transcript recoding by regulating the triplet codon to introduce silent mutations and/or non-synonymous mutations.

Provided herein are compositions that comprise an RNA editing entity or a biologically active fragment thereof and methods of using the same. In an aspect, an RNA editing entity can comprise an adenosine Deaminase Acting on RNA (ADAR), Adenosine Deaminase Acting on tRNA (ADAT), and biologically active fragments thereof of either of these. ADARs and ADATs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR and ADAT enzymes share a common domain architecture comprising a variable number of amino-terminal dsRNA binding domains (dsRBDs) and a single carboxy-terminal catalytic deaminase domain. Human ADARs and ADATs possess two or three dsRBDs. Evidence suggests that ADARs and ADATs can form homodimer as well as heterodimer with other ADARs or and ADATs when bound to double-stranded RNA, however it may be currently inconclusive if dimerization is required for editing to occur.

Three human ADAR genes have been identified (ADARs 1-3) with ADAR1 (official symbol ADAR) and ADAR2 (ADARB1) proteins having well-characterized adenosine deamination activity. ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain. ADAT catalyzes the deamination on tRNAs. ADAT may also named tadA in E. coli. Three human ADAT genes have been identified (ADATs 1-3).

Specific RNA editing can lead to transcript recoding. Because inosine shares the base pairing properties of guanosine, the translational machinery interprets edited adenosines as guanosine, altering the triplet codon, which can result in amino acid substitutions in protein products. More than half the triplet codons in the genetic code could be reassigned through RNA editing. Due to the degeneracy of the genetic code, RNA editing can cause both silent and non-synonymous amino acid substitutions.

In some cases, targeting an RNA can affect splicing. Adenosines targeted for editing can be disproportionately localized near splice junctions in pre-mRNA. Therefore, during formation of a dsRNA ADAR substrate, intronic cis-acting sequences can form RNA duplexes encompassing splicing sites and potentially obscuring them from the splicing machinery. Furthermore, through modification of select adenosines, ADARs can create or eliminate splicing sites, broadly affecting later splicing of the transcript. Similar to the translational machinery, the spliceosome interprets inosine as guanosine, and therefore, a canonical GU 5′ splice site and AG 3′ acceptor site can be created via the deamination of AU (IU=GU) and AA (AI=AG), respectively. Correspondingly, RNA editing can destroy a canonical AG 3′ splice site (IG=GG).

In an aspect, an RNA editing entity comprises an ADAR. In some embodiments, an ADAR can comprise any one of: ADAR1, ADAR1p110, ADAR1p150, ADAR2, ADAR3, APOBEC protein, or any combination thereof. In some embodiments, the ADAR RNA editing entity is ADAR1. Additionally, or alternatively, the ADAR RNA editing entity may be ADAR2. Additionally, or alternatively, the ADAR RNA editing entity may be ADAR3. In an aspect, an RNA editing entity can be a non-ADAR In some cases, an RNA editing entity can comprise at least about 80% sequence homology to APOBEC1, APOBEC2, ADAR1, ADAR1p110, ADAR1p150, ADAR2, ADAR3, or any combination thereof. Alternate editing entities are also contemplated, such as those from a clustered regularly interspaced short palindromic repeats (CRISPR) system.

In some cases, an RNA editing entity can be a virus-encoded RNA-dependent RNA polymerase. In some cases, an RNA editing entity can be a virus-encoded RNA-dependent RNA polymerase from measles, mumps, or parainfluenza. In some instances, an RNA editing entity can be an enzyme from Trypanosoma brucei capable of adding or deleting a nucleotide or nucleotides in a target RNA. In some instances, an RNA editing entity can be an enzyme from Trypanosoma brucei capable of adding or deleting an Uracil or more than one Uracil in a target RNA. In some instances, an RNA editing entity can comprise a recombinant enzyme. In some cases, an RNA editing entity can comprise a fusion polypeptide.

In an aspect, an RNA editing entity can be recruiting by a subject engineered guide RNA. In some embodiments, an engineered guide RNA can recruit an RNA editing entity that, when associated with the engineered guide RNA and the target RNA or not associated with the target RNA, facilitates: an editing of a base of a nucleotide of a polynucleotide of the region of the target RNA, a modulation of the expression of a polypeptide encoded by a subject target RNA, such as RAB7A, ABCA4, SERPINA1, SERPINA1 E342K, HEXA, LRRK2, SNCA, APP, CFTR, ALAS1, ATP7B, ATP7B G1226R, HFE C282Y, LIPA c.894 G>A, PCSK9 start site, SCNN1A start site, GBA, PINK1, Tau; or a combination thereof. An engineered guide RNA can contain an RNA editing recruiting domain to be capable of recruiting an RNA editing entity.

### Engineered Polynucleotides

Described herein, in some embodiments, is an engineered polynucleotide. In some embodiments, the engineered polynucleotide is an engineered RNA. In some embodiments, the engineered polynucleotide is an engineered guide RNA. In some embodiments, the engineered guide RNA, upon binding to a target RNA, forms a structural feature in association with the target RNA, said structural feature at least in part recruits an RNA editing entity (e.g. enzyme) to the target RNA. In some embodiments, the engineered guide RNA can comprise a structural loop stabilized scaffold described herein. The recruited RNA editing entity can chemically modify at least one base of the nucleotide of the target RNA. In some embodiments, the engineered guide RNA is single-stranded. In some embodiments, the engineered guide RNA is at least partially single-stranded. In some embodiments, the engineered guide RNA is partially single-stranded. In some embodiments, the engineered guide RNA comprises at least one chemical modification. In some embodiments, the engineered guide RNA of, when present in an aqueous solution and not bound to the target RNA, does not bind to the RNA editing entity with a dissociation constant less than about 1 pM, 5 pM, 10 pM, 50 pM, 100 pM, 500 pM, 1 nM, 5 nM, 10 nM, 50 nM, 100 nM, 500 nM, 1 pM, 5 pM, 10 pM, 50 pM, 100 pM, 500 pM, or 1 mM. In some embodiments, the engineered guide RNA does not have an intramolecular structure feature. In some embodiments, the engineered guide RNA, when present in an aqueous solution and not bound to the target RNA, does not have an intramolecular structure feature such as a hairpin, a bulge, a polynucleotide loop, a structural domain, or any combination thereof. In some embodiments, the engineered guide RNA may be referred herein as an engineered polynucleotide.

In some embodiments, the engineered guide RNA, when upon binding to the target RNA, forms at least one structure feature to recruit the RNA editing entity, said structure feature can be any one or any combination of the structure feature described herein. In some embodiments, the structural feature comprises a bulge, an internal loop, a hairpin, or any combination thereof. In some embodiments, the structural feature can be a bulge, where the bulge can be an asymmetric bulge or a symmetric bulge. In some embodiments, the structural feature can be an internal loop, where the internal loop can be an asymmetric internal loop or a symmetric internal loop.

In some embodiments, the engineered guide RNA can comprise at least one chemical modification. In some embodiments, the engineered guide RNA can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or more chemical modifications. In some embodiments, the engineered guide RNA comprises no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 chemical modifications. In some embodiments, the engineered guide RNA can comprise at least 1, 2, 3, 4, 5, 6, 7, 8, 9, 10, 50, 100, or more chemically modified nucleotides at the 5′ end of the engineered guide RNA. In some embodiments, the engineered guide RNA can comprise at least 1, 2, 3, 4, 5, 6, 7, 8, 9, 10, 50, 100, or more chemically modified nucleotides at the 3′ end of the engineered guide RNA. In some embodiments, the engineered guide RNA can comprise at least 1, 2, 3, 4, 5, 6, 7, 8, 9, 10, 50, 100, or more chemically modified nucleotides at both the 5′ and the 3′ end of the engineered guide RNA. In some embodiments, the engineered guide RNA can comprise at least one chemical modification in the targeting domain of the engineered guide RNA. In some embodiments, the engineered guide RNA can comprise at least one chemical modification in the nucleotide bases adjacent the targeting domain. In some embodiments, the at least one chemical modification can be introduced within an intramolecular secondary structure.

In some embodiments, the chemical modifications of the engineered guide RNA can comprise at least one substitution of one or both of non-linking phosphate oxygen atoms in a phosphodiester backbone linkage of the engineered guide RNA. In some embodiments, the at least one chemical modification of the engineered guide RNA can comprise a substitution of one or more of linking phosphate oxygen atoms in a phosphodiester backbone linkage of the engineered guide RNA. A non-limiting example of a chemical modification of a phosphate oxygen atom is a sulfur atom. Additional non-limiting examples are included in Table 2. In some embodiments, the chemical modifications of the engineered guide RNA can comprise at least one chemical modification to a sugar of a nucleotide of the engineered guide RNA. In some embodiments, the chemical modifications of the engineered guide RNA can comprise at least one chemical modification to the sugar of the nucleotide, where the chemical modification comprises at least one locked nucleic acid (LNA). In some embodiments, the chemical modifications of the engineered guide RNA can comprise at least one chemical modification to the sugar of the nucleotide of the engineered guide RNA comprising at least one unlocked nucleic acid (UNA). In some embodiments, the chemical modifications of the engineered guide RNA can comprise at least one chemical modification to the sugar comprising a modification of a constituent of the sugar, where the sugar is a ribose sugar. In some embodiments, the chemical modifications of the engineered guide RNA can comprise at least one chemical modification to the constituent of the ribose sugar of the nucleotide of the engineered guide RNA comprising a 2′-O-Methyl group. In some embodiments, the chemical modifications of the engineered guide RNA can comprise at least one chemical modification comprising replacement of a phosphate moiety of the engineered guide RNA with a dephospho linker. In some embodiments, the chemical modifications of the engineered guide RNA can comprise at least one chemical modification of a phosphate backbone of the engineered guide RNA. In some embodiments, the engineered guide RNA can comprise a phosphothioate group. In some embodiments, the chemical modifications of the engineered guide RNA can comprise at least one chemical modification comprising a modification to a base of a nucleotide of the engineered guide RNA. In some embodiments, the chemical modifications of the engineered guide RNA can comprise at least one chemical modification comprising an unnatural base of a nucleotide. In some embodiments, the chemical modifications of the engineered guide RNA can comprise at least one chemical modification comprising a morpholino group, a cyclobutyl group, pyrrolidine group, or peptide nucleic acid (PNA) nucleoside surrogate. In some embodiments, the chemical modifications of the engineered guide RNA can comprise at least one chemical modification comprising at least one stereopure nucleic acid. In some embodiments, the at least one chemical modification can be positioned proximal to a 5′ end of the engineered guide RNA. In some embodiments, the at least one chemical modification can be positioned proximal to a 3′ end of the engineered guide RNA. In some embodiments, the at least one chemical modification can be positioned proximal to both 5′ and 3′ ends of the engineered guide RNA.

In some embodiments, an engineered guide RNA described herein can be an engineered guide polynucleotide. An engineered guide RNA described herein can be an engineered guide RNA (gRNA). An engineered guide RNA or an engineered guide RNA as described herein can include various domains. A “domain” can refer to a region of an engineered guide RNA. In some cases, a domain can be described in terms of a function of the domain. For instance, a “targeting domain” can refer to a region of the engineered guide RNA that can be at least partially complementary to a target RNA; an “RNA editing entity recruiting domain” can refer to a domain that can be capable of associating with or recruiting an RNA editing entity as described herein; and a “spacer domain” can refer to a domain that provides space between other domains. In some instances, recitation of a domain name does not limit the domain to a particular function. For example, a “targeting domain” that can be at least partially complementary to a target RNA can in some instances recruit an RNA editing entity.

In some embodiments, an engineered guide RNA does not comprise a sequence encoding a sequence configured for RNA interference (RNAi). In some embodiments, an engineered guide RNA may not comprise a sequence configured for RNAi. In some cases, an engineered guide RNA may not comprise a sequence encoding a short interfering RNA (siRNA), microRNA (miRNA), short hairpin RNA (shRNA), or Dicer substrate.

In some aspects, the engineered guide RNA can be produced from a precursor of the engineered guide RNA. In some cases, a precursor of the engineered guide RNA can be linear. For example, a precursor of the engineered guide RNA can be a linear mRNA transcribed from a plasmid. In another example, a precursor of the engineered guide RNA can be constructed to be a linear polynucleotide with domains such as a ribozyme domain and a ligation domain that allow for circularization of the engineered guide RNA in a cell. The linear engineered guide RNA with the ligation and ribozyme domains can be transfected into a cell where it can be circularized. In some cases, the engineered guide RNA can be circular. In some cases, the engineered guide RNA can comprise DNA, RNA or both. In some cases, a precursor of the engineered guide RNA can comprise a precursor of the engineered guide RNA. In some cases, a precursor of the engineered guide RNA can be used to produce an engineered guide RNA.

In some cases, the engineered guide RNA as described herein (i.e. chemically modified guide RNA) or a precursor of the engineered chemically modified guide RNA as described herein (e.g., a vector encoding the engineered guide RNA prior to chemical modification) can comprise a spacer domain. In some cases, an engineered guide RNA or a precursor of the engineered guide RNA as described herein does not comprise a spacer domain. In some embodiments, when a targeting domain at least partially binds to a target RNA, a spacer domain can be separated from the targeting domain by at least 1 nucleotide, and if the spacer domain binds to the target RNA, the binding of the spacer domain does not produce an edit of the target RNA at the portion of the target RNA that binds to the spacer domain. In some cases, when a spacer domain can be adjacent to a 5′ end or a 3′ end of the targeting domain, the spacer domain may not be complementary to a target RNA.

In some embodiments, an engineered guide RNA, or a precursor of the engineered guide RNA can be at least 10, 20, 30, 40, 50, 60, 70, 80, 90, 100, 200, 300, 400, 500, 600, 700, 800, 900, 1000, 2500 or 5000 nucleotides in length. In some embodiments, an engineered guide RNA, or a precursor of the engineered guide RNA is no greater than 10, 20, 30, 40, 50, 60, 70, 80, 90, 100, 200, 300, 400, 500, 600, 700, 800, 900, 1000, 2500 or 5000 nucleotides in length. In some cases, an engineered guide RNA (e.g. an engineered guide polynucleotide), or a precursor of the engineered guide RNA can comprise about: 20 nucleotides to about 5000 nucleotides, 20 nucleotides to about 50 nucleotides, 40 nucleotides to about 80 nucleotides, 70 nucleotides to about 140 nucleotides, 80 nucleotides to about 160 nucleotides, 90 nucleotides to about 200 nucleotides, 100 nucleotides to about 250 nucleotides, 150 nucleotides to about 350 nucleotides, 200 nucleotides to about 500 nucleotides, 450 nucleotides to about 800 nucleotides, 750 nucleotides to about 1250 nucleotides, 1000 nucleotides to about 2000 nucleotides, or about 2000 nucleotides to about 5000 nucleotides.

In some embodiments, a spacer domain can be separated from a targeting domain by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or more nucleotides.

In some embodiments, the engineered guide RNA does not comprise a 5′ reducing hydroxyl, a 3′ reducing hydroxyl, or both, capable of being exposed to a solvent. In some instances, an engineered guide RNA can comprise a secondary structure that can be less susceptible to hydrolytic degradation than an mRNA naturally present in a human cell.

In some embodiments, the engineered guide RNA can facilitate an edit of a target RNA, for example, via an RNA editing entity. In some instances, an engineered guide RNA can have an increased editing efficiency to a target RNA by at least about 90%, relative to an otherwise comparable polynucleotide that can comprise a 5′ reducing hydroxyl, a 3′ reducing hydroxyl, or both. In some embodiments, an editing efficiency can be determined by: transfecting a target RNA into a primary cell line; transfecting an engineered guide RNA and an otherwise comparable polynucleotide that can comprise a 5′ reducing hydroxyl, a 3′ reducing hydroxyl or both, into a primary cell line; and sequencing the target RNA. In some embodiments, an editing efficiency can be determined by transfecting a target RNA into a cell; transfecting an engineered guide RNA and an otherwise comparable polynucleotide that can comprise the 5′ reducing hydroxyl, the 3′ reducing hydroxyl or both, into a primary cell line; and mass spectroscopy of the target RNA. In some embodiments, an edit of a base of a nucleotide of a target RNA by an RNA editing entity can be determined in an in vitro assay comprising: directly or indirectly introducing (e.g., transfecting) the target RNA into a cell; directly or indirectly introducing (e.g., transfecting) the engineered guide RNA into a cell; and sequencing the target RNA. In some cases, transfecting the target RNA into a cell can comprise transfecting a plasmid encoding for the target RNA into a cell. In some instances, transfecting an engineered guide RNA into a cell can comprise transfecting a precursor of the engineered guide RNA, or an engineered guide RNA (e.g. plasmid) that encodes for the precursor of the engineered guide RNA into a cell. In some cases, sequencing can comprise Sanger sequencing of a target RNA after the target RNA has been converted to cDNA by reverse transcriptase. In some embodiments, the sequencing can be performed from ddPCR product.

In some instances, a targeting domain can have a sequence length of from about: 20 nucleotides to about 1,000 nucleotides, 10 nucleotides to about 100 nucleotides, 50 nucleotides to about 500 nucleotides or about 400 nucleotides to about 1000 nucleotides in length.

In some cases, the target RNA can comprise a nuclear RNA, a cytoplasmic RNA, or a mitochondrial RNA. In some embodiments, the target RNA can comprise an intergenic DNA (including, without limitation, heterochromatic DNA), a messenger RNA (mRNA), a pre-messenger RNA (pre-mRNA), a transfer RNA (tRNA), a ribosomal RNA (rRNA), a ribozyme, cDNA, a recombinant polynucleotide, a branched polynucleotide, a plasmid, a vector, an isolated DNA of a sequence, an isolated RNA of a sequence, a sgRNA, a guide RNA, a nucleic acid probe, a primer, an snRNA, a long non-coding RNA, a small RNA, a snoRNA, a siRNA, a miRNA, a tRNA-derived small RNA (tsRNA), an antisense RNA, an shRNA, or a small rDNA-derived RNA (srRNA).

Provided herein are polynucleotides and compositions that comprise the same. In an aspect, a polynucleotide can be an engineered polynucleotide. In some embodiments, the engineered polynucleotide can be an engineered guide RNA. In some embodiments, an engineered guide RNA of the disclosure can be utilized for RNA editing, for example to prevent or treat a disease or condition.

In some cases, the disease or condition is associated with a mutation in a DNA molecule or RNA molecule encoding ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. In some examples, a protein encoded for by a mutated DNA molecule or RNA molecule encoding ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. contributes to, at least in part, the pathogenesis or progression of a disease. In some examples, the mutation in the DNA or RNA molecule is relative to an otherwise identical reference DNA or RNA molecule.

In some cases, an engineered guide RNA can be used in association with a subject RNA editing entity to edit a target RNA or modulate expression of a polypeptide encoded by the target RNA. In an embodiment, compositions disclosed herein can include engineered guide RNAs capable of facilitating editing by subject RNA editing entities such as ADAR or ADAT polypeptides or biologically active fragments thereof.

In some embodiments, an engineered guide RNA, an engineered guide RNA, or a precursor engineered guide RNA can form a secondary structure comprising a stem-loop, a cruciform, a toe hold, a mismatch bulge, or any combination thereof. In some cases, a secondary structure can comprise a stem, a hairpin loop, a pseudoknot, a bulge, an internal loop, a multiloop, a G-quadruplex, or any combination thereof. In some embodiments, an engineered guide RNA, an engineered guide RNA, or a precursor engineered guide RNA can adopt an A-form, a B-form, a Z-form, or any combination thereof.

An engineered guide RNA, an engineered guide RNA, or a precursor if the engineered guide RNA comprising a secondary structure can significantly enhance affinity of binding to a target RNA, enhance specificity of binding to a target RNA, enhance efficiency of editing of a target RNA, reduce off-target editing, reduce editing of a non-target RNA, enhance efficiency of recruiting an RNA editing entity, or a combination thereof, as compared to a comparable guide RNA or a comparable polynucleotide that lacks the secondary structure. An engineered guide RNA, an engineered guide RNA, or a precursor of engineered guide RNA comprising a bulge can significantly enhance affinity of binding to a target RNA, enhance specificity of binding to a target RNA, enhance efficiency of editing of a target RNA, reduce off-target editing, reduce editing of a non-target RNA, enhance efficiency of recruiting an RNA editing entity, or a combination thereof, as compared to a comparable guide RNA or comparable polynucleotide that lacks the bulge. An engineered guide RNA, an engineered guide RNA, or a precursor of the engineered guide RNA comprising a stem-loop, a cruciform, a toe hold, a mismatch bulge, a stem, a hairpin loop, a pseudoknot, an internal loop, a multiloop, a G-quadruplex, or any combination thereof can significantly enhance affinity of binding to a target RNA, enhance specificity of binding to a target RNA, enhance efficiency of editing of a target RNA, reduce off-target editing, reduce editing of a non-target RNA, enhance efficiency of recruiting an RNA editing entity, or a combination thereof, as compared to a comparable guide RNA or comparable polynucleotide that lacks the stem-loop, the cruciform, the toe hold, the mismatch bulge, the stem, the hairpin loop, the pseudoknot, the internal loop, the multiloop, the G-quadruplex, or any combination thereof.

An RNA editing entity recruiting domain can comprise at least about: 80%, 85%, 90%, 95%, 99% or 100% sequence homology to at least about 20 nucleotides of: an Alu domain, an APOBEC recruiting domain, a GluR2 domain, a TALEN recruiting domain, a Zn-finger polypeptide recruiting domain, a mega-TAL recruiting domain, or a Cas13 recruiting domain. In some embodiments, an RNA editing entity recruiting domain can comprise at least about 80% sequence homology to at least about 20 nucleotides of an Alu domain. In some cases, an RNA editing entity recruiting domain can comprise at least about 80% sequence homology to an Alu-recruiting domain. In some embodiments, an RNA editing entity recruiting domain can comprise at least about: 80%, 85%, 90%, or 95% sequence homology to at least about: 15, 20, 25, 30, or 35 nucleic acids of an Alu domain. In some cases, at least a portion of an RNA editing entity recruiting domain can comprise at least about 80% sequence homology to an Alu domain encoding sequence. In some embodiments, at least a portion of an RNA editing entity recruiting domain can comprise at least about 85% sequence homology to an Alu domain encoding sequence. In some cases, at least a portion of an RNA editing entity recruiting domain can comprise at least about 90% sequence homology to an Alu domain encoding sequence. In some embodiments, at least a portion of an RNA editing entity recruiting domain can comprise at least about 95% sequence homology to an Alu domain encoding sequence. In some cases, an Alu-domain-encoding sequence can be a non-naturally occurring sequence. In some embodiments, an Alu-domain-encoding sequence can comprise a modified portion. In some cases, an Alu-domain-encoding sequence can comprise a portion of a naturally occurring Alu-domain-encoding-sequence.

In some embodiments, an RNA editing entity recruiting domain can comprise at least about 80% sequence homology to at least about 20 nucleotides of an APOBEC domain. In some cases, an RNA editing entity recruiting domain can comprise at least about 80% sequence homology to an APOBEC-recruiting domain. In some embodiments, an RNA editing entity recruiting domain can comprise at least about: 80%, 85%, 90%, or 95% sequence homology to at least about: 15, 20, 25, 30, or 35 nucleic acids of an APOBEC domain. In some cases, at least a portion of an RNA editing entity recruiting domain can comprise at least about 80% sequence homology to an APOBEC domain encoding sequence. In some embodiments, at least a portion of an RNA editing entity recruiting domain can comprise at least about 85% sequence homology to an APOBEC domain encoding sequence. In some cases, at least a portion of an RNA editing entity recruiting domain can comprise at least about 90% sequence homology to an APOBEC domain encoding sequence. In some embodiments, at least a portion of an RNA editing entity recruiting domain can comprise at least about 95% sequence homology to an APOBEC domain encoding sequence. In some cases, an APOBEC-domain-encoding sequence can be a non-naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence.

In some cases, an RNA editing entity recruiting domain can comprise at least about 80% sequence homology to at least about 20 nucleotides of a GluR2 domain. In some embodiments, an RNA editing entity recruiting domain can comprise at least about 80% sequence homology to a GluR2-recruiting domain. In some cases, an RNA editing entity recruiting domain can comprise at least about: 80%, 85%, 90%, or 95% sequence homology to at least about: 15, 20, 25, 30, or 35 nucleic acids of a GluR2 domain. In some embodiments, at least a portion of an RNA editing entity recruiting domain can comprise at least about 80% sequence homology to a GluR2 domain encoding sequence. In some cases, at least a portion of an RNA editing entity recruiting domain can comprise at least about 85% sequence homology to a GluR2 domain encoding sequence. In some embodiments, at least a portion of an RNA editing entity recruiting domain can comprise at least about 90% sequence homology to a GluR2 domain encoding sequence. In some cases, at least a portion of an RNA editing entity recruiting domain can comprise at least about 95% sequence homology to a GluR2 domain encoding sequence. In some embodiments, a GluR2-domain-encoding sequence can be a non-naturally occurring sequence. In some cases, a GluR2-domain-encoding sequence can comprise a modified portion. In some embodiments, a GluR2-domain-encoding sequence can comprise a portion of a naturally occurring GluR2-domain-encoding sequence. In some cases, at least a portion of a recruiting domain can comprise at least about 80% sequence identity to an encoding sequence that recruits an ADAR. In some instances, an RNA editing entity recruiting domain can comprise at least about 80% sequence homology to an MS2-bacteriophage-coat-protein-recruiting domain.

An RNA editing entity can comprise an endogenous enzyme. In some instances, an RNA editing entity can comprise a recombinant enzyme. In some cases, an RNA editing entity can comprise a fusion polypeptide. In some embodiments, an RNA editing entity can comprise APOBEC1, APOBEC2, APOBEC3A, APOBEC3B, APOBEC3C, APOBEC3D (“APOBEC3E” now can refer to this), APOBEC3F, APOBEC3G, APOBEC3H, APOBEC4, Activation-induced (cytidine) deaminase (AID), ADAR1, ADAR1p110, ADAR1p150, ADAR2, ADAR3, or any combination thereof. In some cases, an RNA editing entity can comprise at least about 80% sequence homology to APOBEC1, APOBEC2, APOBEC3A, APOBEC3B, APOBEC3C, APOBEC3D (“APOBEC3E” now can refer to this), APOBEC3F, APOBEC3G, APOBEC3H, APOBEC4, Activation-induced (cytidine) deaminase (AID), ADAR1, ADAR1p110, ADAR1p150, ADAR2, ADAR3, or any combination thereof. In some cases, an RNA editing entity can be a virus-encoded RNA-dependent RNA polymerase. In some cases, an RNA editing entity can be a virus-encoded RNA-dependent RNA polymerase from measles, mumps, or parainfluenza. In some instances, an RNA editing entity can be an enzyme from Trypanosoma brucei capable of adding or deleting a nucleotide or nucleotides in a target RNA. In some instances, an RNA editing entity can be an enzyme from Trypanosoma brucei capable of adding or deleting an Uracil or more than one Uracil in a target RNA.

Engineered guide RNAs can be engineered in any way suitable for RNA targeting. In an aspect, an engineered guide RNA generally comprises at least a targeting sequence that allows it to hybridize to a region of a target RNA. In some cases, a targeting sequence can also be referred to as a targeting domain or a targeting region. In some cases, a targeting sequence of an engineered guide RNA allows the engineered guide RNA to target an RNA sequence through base pairing, such as Watson Crick base pairing. In an embodiment, the targeting sequence can be located at either the N-terminus or C-terminus of the engineered guide RNA. In some cases, the targeting sequence is located at both termini. The targeting sequence can be of any length. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In an embodiment, an engineered guide RNA comprises a targeting sequence that is about 75-100, 80-110, 90-120, or 95-115 nucleotides in length. In an embodiment, an engineered polynucleotide is an engineered guide RNA.

In some cases, a subject targeting sequence comprises at least partial sequence complementarity to a region of a target RNA that at least partially encodes a subject polypeptide for example RAB7A, ABCA4, SERPINA1, SERPINA1 E342K, HEXA, LRRK2, SNCA, APP, Tau, CFTR, ALAS1, ATP7B, ATP7B G1226R, HFE C282Y, LIPA c.894 G>A, PCSK9 start site, SCNN1A start site, GBA, PINK1, or Tau. In some cases, a targeting sequence comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence may have a single base mismatch. In other cases, the targeting sequence of a subject engineered guide RNA comprises more than one mismatch. Without wishing to be bound by theory, a chemically modified guide polynucleotide as described herein may comprise a larger degree of mismatches relative to a comparable unmodified guide polynucleotide. For examples, a chemically modified guide can have at least about 1, 2, 3, 4, 5, 6, 7, 8, 9, 10, 11, 12, 13, 14, 15, 16, 20, 30, 40 or up to about 50 base mismatches. In some aspects, nucleotide mismatches can be associated with structural features provided herein. In some aspects, a targeting sequence comprises at least about 1, 2, 3, 4, 5, 6, 7, 8, 9, 10, 11, 12, 13, 14, or up to about 15 nucleotides that differ in complementarity from a wildtype RNA of a subject target RNA. In some cases, a targeting sequence comprises at least 50 nucleotides having complementarity to a target RNA. In some cases, a targeting sequence comprises from 50 to 150 nucleotides having complementarity to a target RNA. In some cases, a targeting sequence comprises from 50 to 200 nucleotides having complementarity to a target RNA. In some cases, a targeting sequence comprises from 50 to 250 nucleotides having complementarity to a target RNA. In some cases, a targeting sequence comprises from 50 to 300 nucleotides having complementarity to a target RNA. In some cases, a targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to a target RNA. In some cases, a targeting sequence comprises more than 50 nucleotides total and has at least 50 nucleotides having complementarity to a target RNA. In some cases, a targeting sequence comprises from 50 to 400 nucleotides total and has from 50 to 150 nucleotides having complementarity to a target RNA. In some cases, a targeting sequence comprises from 50 to 400 nucleotides total and has from 50 to 200 nucleotides having complementarity to a target RNA. In some cases, a targeting sequence comprises from 50 to 400 nucleotides total and has from 50 to 250 nucleotides having complementarity to a target RNA. In some cases, a targeting sequence comprises from 50 to 400 nucleotides total and has from 50 to 300 nucleotides having complementarity to a target RNA. In some cases, the at least 50 nucleotides having complementarity to a target RNA are separated by one or more mismatches, one or more bulges, or one or more loops, or any combination thereof. In some cases, the from 50 to 150 nucleotides having complementarity to a target RNA are separated by one or more mismatches, one or more bulges, or one or more loops, or any combination thereof. In some cases, the from 50 to 200 nucleotides having complementarity to a target RNA are separated by one or more mismatches, one or more bulges, or one or more loops, or any combination thereof. In some cases, the from 50 to 250 nucleotides having complementarity to a target RNA are separated by one or more mismatches, one or more bulges, or one or more loops, or any combination thereof. In some cases, the from 50 to 300 nucleotides having complementarity to a target RNA are separated by one or more mismatches, one or more bulges, or one or more loops, or any combination thereof. For example, a targeting sequence comprises a total of 54 nucleotides wherein, sequentially, 25 nucleotides are complementarity to a target RNA, 4 nucleotides form a bulge, and 25 nucleotides are complementarity to a target RNA. As another example, a targeting sequence comprises a total of 118 nucleotides wherein, sequentially, 25 nucleotides are complementarity to a target RNA, 4 nucleotides form a bulge, 25 nucleotides are complementarity to a target RNA, 14 nucleotides form a loop, and 50 nucleotides are complementary to a target RNA.

In some embodiments, the engineered guide RNA described herein comprises a targeting sequence or a targeting domain that is at least partially complementary to nucleic acid sequences as shown in Table 1. In some embodiments, the engineered guide RNA described herein comprises a targeting sequence or a targeting domain that is at least 70%, at least 80%, at least 85%, at least 90%, at least 92%, at least 95%, at least 97%, or at least 99% sequence complementary with SEQ ID NOs: 1-2. In some embodiments, the engineered guide RNA described herein comprises a targeting sequence or a targeting domain that is at least partially identical to nucleic acid sequences as shown in Table 1. In some embodiments, the engineered guide RNA described herein comprises a targeting sequence or a targeting domain that is at least 70%, at least 80%, at least 85%, at least 90%, at least 92%, at least 95%, at least 97%, or at least 99% sequence identical with SEQ ID NOs: 1-2.

TABLE 1
Nucleic acid sequences of SERPINA1 and ABCA4

SEQ ID NO: 1
SERPINA1 (ENST00000355814.8)
noneTGGGCAGGAACTGGGCACTGTGCCCAGGGCATGCACTGCCTCCACGCAGCAACC
CTCAGAGTCCTGAGCTGAACCAAGAAGGAGGAGGGGGTCGGGCCTCCGAGGAAGGC
CTAGCCGCTGCTGCTGCCAGGAATTCCAGGTTGGAGGGGCGGCAACCTCCTGCCAGC
CTTCAGGCCACTCTCCTGTGCCTGCCAGAAGAGACAGAGCTTGAGGAGAGCTTGAGG
AGAGCAGGAAAGGTGGGACATTGCTGCTGCTGCTCACTCAGTTCCACAGgtgggagggaca
gcagggcttagagtgggggtcattgtgcagatgggaaaacaaaggcccagagaggggaagaaatgcccaggagctaccgagggcaggc
gacctcaaccacagcccagtgctggagctgtgagtggatgtagagcagcggaatatccattcagccagctcaggggaaggacaggggccc
tgaagccaggggatggagctgcagggaagggagctcagagagaaggggaggggagtctgagctcagtttcccgctgcctgaaaggagg
gtggtacctactcccttcacagggtaactgaatgagagactgcctggaggaaagctcttcaagtgtggcccaccccaccccagtgacaccag
cccctgacacgggggagggagggcagcatcaggaggggctttctgggcacacccagtacccgtctctgagctttccttgaactgttgcatttt
aatcctcacagcagctcaacaaggtacataccgtcaccatccccattttacagatagggaaattgaggctcggagcggttaaacaactcacct
gaggcctcacagccagtaagtgggttccctggtctgaatgtgtgtgctggaggatcctgtgggtcactcgcctggtagagccccaaggtgga
ggcataaatgggactggtgaatgacagaaggggcaaaaatgcactcatccattcactctgcaagtatctacggcacgtacgccagctcccaa
gcaggtttgcgggttgcacagcgggcgatgcaatctgatttaggcttttaaagggattgcaatcaagtggggccccactagcctcaaccctgta
cctcccctcccctccacccccagcagtctccaaaggcctccaacaaccccagagtgggggccatgtatccaaagaaactccaagctgtatac
ggatcacactggttttccaggagcaaaaacagaaacaggcctgaggctggtcaaaattgaacctcctcctgctctgagcagcctggggggca
gactaagcagagggctgtgcagacccacataaagagcctactgtgtgccaggcacttcacccgaggcacttcacaagcatgcttgggaatg
aaacttccaactctttgggatgcaggtgaaacagttcctggttcagagaggtgaagcggcctgcctgaggcagcacagctcttctttacagatg
tgcttccccacctctaccctgtctcacggccccccatgccagcctgacggttgtgtctgcctcagtcatgctccatttttccatcgggaccatcaa
gagggtgtttgtgtctaaggctgactgggtaactttggatgagcggtctctccgctctgagcctgtttcctcatctgtcaaatgggctctaaccca
ctctgatctcccagggcggcagtaagtcttcagcatcaggcattttggggtgactcagtaaatggtagatcttgctaccagtggaacagccact
aaggattctgcagtgagagcagagggccagctaagtggtactctcccagagactgtctgactcacgccaccccctccaccttggacacagg
acgctgtggtttctgagccaggtacaatgactcctttcggtaagtgcagtggaagctgtacactgcccaggcaaagcgtccgggcagcgtag
gcgggcgactcagatcccagccagtggacttagcccctgtttgctcctccgataactggggtgaccttggttaatattcaccagcagcctcccc
cgttgcccctctggatccactgcttaaatacggacgaggacagggccctgtctcctcagcttcaggcaccaccactgacctgggacagtgaat
cgtaagtatgcctttcactgcgagaggttctggagaggcttctgagctccccatggcccaggcaggcagcaggtctggggcaggaggggg
gttgtggagtgggtatccgcctgctgaggtgcagggcagatggagaggctgcagctgagctcctattttcataataacagcagccatgagggt
tgtgtcctgtttcccagtcctgcccggtcccccctcggtacctcctggtggatacactggttcctgtaagcagaagtggatgagggtgtctaggt
ctgcagtcctggcaccccaggatgggggacaccagccaagatacagcaacagcaacaaagcgcagccatttctttctgtttgcacagctcct
ctgtctgtcgggggctcctgtctgttgtctcctataagcctcaccacctctcctactgcttgggcatgcatctttctccccttctatagatgaggagg
ttaaggtccagagaggggtggggaggaacgccggctcacattctccatcccctccagatatgaccaggaacagacctgtgccaggcctcag
ccttacatcaaaatgggcctccccatgcaccgtggacctctgggccctcctgtcccagtggaggacaggaagctgtgaggggcactgtcacc
cagggctcaagctggcattcctgaataatcgctctgcaccaggccacggctaagctcagtgcgtgattaagcctcataaccctccaaggcagt
tactagtgtgattcccattttacagatgaggaagatggggacagagaggtgaataactggccccaaatcacacaccatccataattcgggctca
ggcacctggctccagtccccaaactcttgaacctggccctagtgtcactgtttctcttgggtctcaggcgctggatggggaacaggaaacctg
ggctggacttgaggcctctctgatgctcggtgacttcagacagttgctcaacctctctgttctcttgggcaaaacatgataacctttgacttctgtc
ccctcccctcaccccacccgaccttgatctctgaagtgttggaaggatttaatttttcctgcactgagttttggagacaggtcaaaaagatgacca
aggccaaggtggccagtttcctatagaacgcctctaaaagacctgcagcaatagcagcaagaactggtattctcgagaacttgctgcgcagc
aggcacttcttggcattttatgtgtatttaatttcacaatagctctatgacaaagtccacctttctcatctccaggaaactgaggttcagagaggttaa
gtaacttgtccaaggtcacacagctaatagcaagttgacgtggagcaatctggcctcagagcctttaattttagccacagactgatgctcccctc
ttcatttagccaggctgcctctgaagttttctgattcaagacttctggcttcagctttgtacacagagatgattcaatgtcaggttttggagtgaaatc
tgtttaatcccagacaaaacatttaggattacatctcagttttgtaagcaagtagctctgtgatttttagtgagttatttaatgctctttggggctcaatt
tttctatctataaaatagggctaataatttgcaccttatagggtaagctttgaggacagattagatgatacggtgcctgtaaaacaccaggtgttag
taagtgtggcaatgatggtgacgctgaggctgatgtttgcttagcatagggttaggcagctggcaggcagtaaacagttggataatttaatgga
aaatttgccaaactcagatgctgttcactgctgagcaggagccccttcctgctgaaatggtcctggggagtgcagcaggctctccgggaagaa
atctaccatctctcgggcaggagctcaacctgtgtgcaggtacagggagggcttcctcacctggtgcccactcatgcattacgtcagttattcct
catccctgtccaaaggattcttttctccattgtacagctatgaagctagtgctcaaagaagtgaagtcatttaccccaggccccctgccagtaagt
gacagggcctggtcacacttgggtttatttattgcccagttcaacaggttgtttgaccataggcgagattctcttccctgcaccctgccgggttgct
cttggtcccttattttatgctcccgggtagaaatggtgtgagattaggcagggagtggctcgcttccctgtccctggccccgcaaagagtgctcc
cacctgccccgatcccagaaatgtcaccatgaagccttcattcttttggtttaaagcttggcctcagtgtccgtacaccatggggtacttggccag
atggcgactttctcctctccagtcgccctcccaggcactagcttttaggagtgcagggtgctgcctctgatagaagggccaggagagagcag
gttttggagtcctgatgttataaggaacagcttgggaggcataatgaacccaacatgatgcttgagaccaatgtcacagcccaattctgacattc
atcatctgagatctgaggacacagctgtctcagttcatgatctgagtgctgggaaagccaagacttgttccagctttgtcactgacttgctgtata
gcctcaacaaggccctgaccctctctgggcttcaaactcttcactgtgaaaggaggaaaccagagtaggtgatgtgacaccaggaaagatgg
atgggtgtgggggaatgtgctcctcccagctgtcaccccctcgccaccctccctgcaccagcctctccacctcctttgagcccagaattcccct
gtctaggagggcacctgtctcatgcctagccatgggaattctccatctgttttgctacattgaacccagatgccattctaaccaagaatcctggct
gggtgcaggggctctcgcctgtaaccccagcactttgggaggccaaggcaggcggatcaagaggtcaggagttcaagacctgcctggcca
acacggtgaaacctcagctctactaaaaatacaaaaattagccaggcgtggtggcacacgcctgtaatcccagctatttgggaagctgagaca
gaagaatttcttgaacccgggaggtggaggtttcagtgagccgagatcacgccactgcactccaccctggcagataaagcgagactctgtct
caaaaaaaacccaaaaacctatgttagtgtacagagggccccagtgaagtcttctcccagccccactttgcacaactggggagagtgaggcc
ccaggaccagaggattcttgctaaaggccaagtggatagtgatggccctgccagggctagaagccacaacctctggccctgaggccactca
gcatatttagtgtccccaccctgcagaggcccaactccctcctgaccactgagccctgtaatgatgggggaatttccataagccatgaaggact
gcacaaagttcagttgggaagtgaaagagaaattaaagggagatggaaatatacagcactaattttagcaccgtctttagttctaacaacacta
gctagctgaagaaaaatacaaacatgtattatgtaatgtgtggtctgttccatttggattacttagaggcacgagggccaggagaaaggtggtg
gagagaaaccagctttgcacttcatttgttgctttattggaaggaaacttttaaaagtccaagggggttgaagaatctcaatatttgttatttccagct
ttttttctccagtttttcatttcccaaattcaaggacacctttttctttgtattttgttaagatgatggttttggttttgtgactagtagttaacaatgtggctg
ccgggcatattctcctcagctaggacctcagttttcccatctgtgaagacggcaggttctacctagggggctgcaggctggtggtccgaagcct
gggcatatctggagtagaaggatcactgtggggcagggcaggttctgtgttgctgtggatgacgttgactttgaccattgctcggcagagcct
gctctcgctggttcagccacaggccccaccactccctattgtctcagccccgggtatgaaacatgtattcctcactggcctatcacctgaagcct
ttgaatttgcaacacctgccaacccctccctcaaaagagttgccctctcagatccttttgatgtaaggtttggtgttgagacttatttcactaaattct
catacataaacatcactttatgtatgaggcaaaatgaggaccagggagatgaatgacttgtcctggctcatacacctggaaagtgacagagtca
gattagatcccaggtctatctgaagttaaaagaggtgtcttttcacttcccacctcctccatctactttaaagcagcacaaacccctgctttcaagg
agagatgagcgtctctaaagcccctgacagcaagagcccagaactgggacaccattagtgacccagacggcaggtaagctgactgcagga
gcatcagcctattcttgtgtctgggaccacagagcattgtggggacagccccgtctcttgggaaaaaaaccctaagggctgaggatccttgtg
agtgttgggtgggaacagctcccaggaggtttaatcacagcccctccatgctctctagctgttgccattgtgcaagatgcatttcccttctgtgca
gcagtttccctggccactaaatagtgggattagatagaagccctccaagggcttccagcttgacatgattcttgattctgatctggcccgattcct
ggataatcgtgggcaggcccattcctcttcttgtgcctcattttcttcttttgtaaaacaatggctgtaccatttgcatcttagggtcattgcagatgta
agtgttgctgtccagagcctgggtgcaggacctagatgtaggattctggttctgctacttcctcagtgacattgaatagctgacctaatctctctg
gctttggtttcttcatctgtaaaagaaggatattagcattagcacctcacgggattgttacaagaaagcaatgaattaacacatgtgagcacgga
gaacagtgcttggcatatggtaagcactacgtacattttgctattcttctgattctttcagtgttactgatgtcggcaagtacttggcacaggctggtt
taataatccctaggcacttccacgtggtgtcaatccctgatcactgggagtcatcatgtgccttgactcggggcctggcccccccatctctgtctt
gcagGACAATGCCGTCTTCTGTCTCGTGGGGCATCCTCCTGCTGGCAGGCCTGTGCTGC
CTGGTCCCTGTCTCCCTGGCTGAGGATCCCCAGGGAGATGCTGCCCAGAAGACAGAT
ACATCCCACCATGATCAGGATCACCCAACCTTCAACAAGATCACCCCCAACCTGGCT
GAGTTCGCCTTCAGCCTATACCGCCAGCTGGCACACCAGTCCAACAGCACCAATATCT
TCTTCTCCCCAGTGAGCATCGCTACAGCCTTTGCAATGCTCTCCCTGGGGACCAAGGC
TGACACTCACGATGAAATCCTGGAGGGCCTGAATTTCAACCTCACGGAGATTCCGGA
GGCTCAGATCCATGAAGGCTTCCAGGAACTCCTCCGTACCCTCAACCAGCCAGACAG
CCAGCTCCAGCTGACCACCGGCAATGGCCTGTTCCTCAGCGAGGGCCTGAAGCTAGT
GGATAAGTTTTTGGAGGATGTTAAAAAGTTGTACCACTCAGAAGCCTTCACTGTCAAC
TTCGGGGACACCGAAGAGGCCAAGAAACAGATCAACGATTACGTGGAGAAGGGTAC
TCAAGGGAAAATTGTGGATTTGGTCAAGGAGCTTGACAGAGACACAGTTTTTGCTCT
GGTGAATTACATCTTCTTTAAAGgtaaggttgctcaaccagcctgagctgttcccatagaaacaagcaaaaatattctca
aaccatcagttcttgaactctccttggcaatgcattatgggccatagcaatgcttttcagcgtggattcttcagttttctacacacaaacactaaaat
gttttccatcattgagtaatttgaggaaataatagattaaactgtcaaaactactgacagctctgcagaacttttcagagcctttaatgtccttgtgta
tactgtatatgtagaatatataatgcttagaactatagaacaaattgtaatacactgcataaagggatagtttcatggaacatactttacacgactct
agtgtcccagaatcagtatcagttttgcaatctgaaagacctgggttcaaatcctgcctctaacacaattagcttttgacaaaaacaatgcattcta
cctctttgaggtgctaatttctcatcttagcatggacaaaataccattcttgctgtcaggtttttttaggattaaacaaatgacaaagactgtggggat
ggtgtgtggcatacagcaggtgatggactcttctgtatctcaggctgccttcctgcccctgaggggttaaaatgccagggtcctgggggcccc
agggcattctaagccagctcccactgtcccaggaaaacagcataggggaggggaggtgggaggcaaggccaggggctgcttcctccact
ctgaggctcccttgctcttgaggcaaaggagggcagtggagagcagccaggctgcagtcagcacagctaaagtcctggctctgctgtggcc
ttagtgggggcccaggtccctctccagccccagtctcctccttctgtccaatgagaaagctgggatcaggggtccctgaggcccctgtccact
ctgcatgcctcgatggtgaagctctgttggtatggcagaggggaggctgctcaggcatctgcatttcccctgccaatctagaggatgaggaaa
gctctcaggaatagtaagcagaatgtttgccctggatgaataactgagctgccaattaacaaggggcagggagccttagacagaaggtacca
aatatgcctgatgctccaacattttatttgtaatatccaagacaccctcaaataaacatatgattccaataaaaatgcacagccacgatggcatctc
ttagcctgacatcgccacgatgtagaaattctgcatcttcctctagttttgaattatccccacacaatctttttcggcagcttggatggtcagtttcag
caccttttacagatgatgaagctgagcctcgagggatgtgtgtcgtcaagggggctcagggcttctcagggaggggactcatggtttctttattc
tgctacactcttccaaaccttcactcacccctggtgatgcccaccttcccctctctccagGCAAATGGGAGAGACCCTTTG
AAGTCAAGGACACCGAGGAAGAGGACTTCCACGTGGACCAGGTGACCACCGTGAAG
GTGCCTATGATGAAGCGTTTAGGCATGTTTAACATCCAGCACTGTAAGAAGCTGTCCA
GCTGGGTGCTGCTGATGAAATACCTGGGCAATGCCACCGCCATCTTCTTCCTGCCTGA
TGAGGGGAAACTACAGCACCTGGAAAATGAACTCACCCACGATATCATCACCAAGTT
CCTGGAAAATGAAGACAGAAGgtgattccccaacctgagggtgaccaagaagctgcccacacctcttagccatgttggg
actgaggcccatcaggactggccagagggctgaggagggtgaaccccacatccctgggtcactgctactctgtataaacttggcttccagaa
tgaggccaccactgagttcaggcagcgccatccatgctccatgaggaggacagtacccaggggtgaggaggtaaaggtctcgtccctggg
gacttcccactccagtgtggacactgtcccttcccaatatccagtgcccagggcagggacagcagcaccaccacacgttctggcagaaccaa
aaaggaacagatgggcttcctggcaaaggcagcagtggagtgtggagttcaagggtagaatgtccctggggggacgggggaagagcctg
tgtggcaaggcccagaaaagcaaggttcggaattggaacagccaggccatgttcgcagaaggcttgcgtttctctgtcactttatcggtgctgt
tagattgggtgtcctgtagtaagtgatacttaaacatgagccacacattagtgtatgtgtgtgcattcgtgattatgcccatgccctgctgatctagt
tcgttttgtacactgtaaaaccaagatgaaaatacaaaaggtgtcgggttcataataggaatcgaggctggaatttctctgttccatgccagcacc
tcctgaggtctctgctccaggggttgagaaagaacaaagaggctgagagggtaacggatcagagagcccagagccaagctgcccgctcac
accagaccctgctcagggtggcattgtctccccatggaaaaccagagaggagcactcagcctggtgtggtcactcttctcttatccactaaacg
gttgtcactgggcactgccaccagccccgtgtttctctgggtgtagggccctggggatgttacaggctgggggccaggtgacccaacactac
agggcaagatgagacaggcttccaggacacctagaatatcagaggaggtggcatttcaagcttttgtgattcattcgatgttaacattctttgact
caatgtagaagagctaaaagtagaacaaaccaaagccgagttcccatcttagtgtgggtggaggacacaggagtaagtggcagaaataatc
agaaaagaaaacacttgcactgtggtgggtcccagaagaacaagaggaatgctgtgccatgccttgaatttcttttctgcacgacagGTCT
GCCAGCTTACATTTACCCAAACTGTCCATTACTGGAACCTATGATCTGAAGAGCGTCC
TGGGTCAACTGGGCATCACTAAGGTCTTCAGCAATGGGGCTGACCTCTCCGGGGTCA
CAGAGGAGGCACCCCTGAAGCTCTCCAAGgtgagatcaccctgacgaccttgttgcaccctggtatctgtaggga
agaatgtgtgggggctgcagctctgtcctgaggctgaggaaggggccgagggaaacaaatgaagacccaggctgagctcctgaagatgc
ccgtgattcactgacacgggacgtggtcaaacagcaaagccaggcaggggactgctgtgcagctggcactttcggggcctcccttgaggtt
gtgtcactgaccctgaatttcaactttgcccaagaccttctagacattgggccttgatttatccatactgacacagaaaggtttgggctaagttgttt
caaaggaatttctgactccttcgatctgtgagatttggtgtctgaattaatgaatgatttcagctaaagatgacacttattttggaaaactaaaggcg
accaatgaacaactgcagttccatgaatggctgcattatcttggggtctgggcactgtgaaggtcactgccagggtccgtgtcctcaaggagct
tcaagccgtgtactagaaaggagagagccctggaggcagacgtggagtgacgatgctcttccctgttctgagttgtgggtgcacctgagcag
ggggagaggcgcttgtcaggaagatggacagaggggagccagccccatcagccaaagccttgaggaggagcaaggcctatgtgacagg
gagggagaggatgtgcagggccagggccgtccagggggagtgagcgcttcctgggaggtgtccacgtgagccttgctcgaggcctggga
tcagccttacaacgtgtctctgcttctctcccctccagGCCGTGCATAAGGCTGTGCTGACCATCGACGAGAA
AGGGACTGAAGCTGCTGGGGCCATGTTTTTAGAGGCCATACCCATGTCTATCCCCCCC
GAGGTCAAGTTCAACAAACCCTTTGTCTTCTTAATGATTGAACAAAATACCAAGTCTC
CCCTCTTCATGGGAAAAGTGGTGAATCCCACCCAAAAATAACTGCCTCTCGCTCCTCA
ACCCCTCCCCTCCATCCCTGGCCCCCTCCCTGGATGACATTAAAGAAGGGTTGAGCTG
GTCCCTGCCTGCATGTGACTGTAAATCCCTCCCATGTTTTCTCTGAGTCTCCCTTTGCC
TGCTGAGGCTGTATGTGGGCTCCAGGTAACAGTGCTGTCTTCGGGCCCCCTGAACTGT
GTTCATGGAGCATCTGGCTGGGTAGGCACATGCTGGGCTTGAATCCAGGGGGGACTG
AATCCTCAGCTTACGGACCTGGGCCCATCTGTTTCTGGAGGGCTCCAGTCTTCCTTGT
CCTGTCTTGGAGTCCCCAAGAAGGAATCACAGGGGAGGAACCAGATACCAGCCATGA
CCCCAGGCTCCACCAAGCATCTTCATGTCCCCCTGCTCATCCCCCACTCCCCCCCACC
CAGAGTTGCTCATCCTGCCAGGGCTGGCTGTGCCCACCCCAAGGCTGCCCTCCTGGGG
GCCCCAGAACTGCCTGATCGTGCCGTGGCCCAGTTTTGTGGCATCTGCAGCAACACA
AGAGAGAGGACAATGTCCTCCTCTTGACCCGCTGTCACCTAACCAGACTCGGGCCCT
GCACCTCTCAGGCACTTCTGGAAAATGACTGAGGCAGATTCTTCCTGAAGCCCATTCT
CCATGGGGCAACAAGGACACCTATTCTGTCCTTGTCCTTCCATCGCTGCCCCAGAAAG
CCTCACATATCTCCGTTTAGAATCAGGTCCCTTCTCCCCAGATGAAGAGGAGGGTCTC
TGCTTTGTTTTCTCTATCTCCTCCTCAGACTTGACCAGGCCCAGCAGGCCCCAGAAGA
CCATTACCCTATATCCCTTCTCCTCCCTAGTCACATGGCCATAGGCCTGCTGATGGCTC
AGGAAGGCCATTGCAAGGACTCCTCAGCTATGGGAGAGGAAGCACATCACCCATTGA
CCCCCGCAACCCCTCCCTTTCCTCCTCTGAGTCCCGACTGGGGCCACATGCAGCCTGA
CTTCTTTGTGCCTGTTGCTGTCCCTGCAGTCTTCAGAGGGCCACCGCAGCTCCAGTGC
CACGGCAGGAGGCTGTTCCTGAATAGCCCCTGTGGTAAGGGCCAGGAGAGTCCTTCC
ATCCTCCAAGGCCCTGCTAAAGGACACAGCAGCCAGGAAGTCCCCTGGGCCCCTAGC
TGAAGGACAGCCTGCTCCCTCCGTCTCTACCAGGAATGGCCTTGTCCTATGGAAGGCA
CTGCCCCATCCCAAACTAATCTAGGAATCACTGTCTAACCACTCACTGTCATGAATGT
GTACTTAAAGGATGAGGTTGAGTCATACCAAATAGTGATTTCGATAGTTCAAAATGG
TGAAATTAGCAATTCTACATGATTCAGTCTAATCAATGGATACCGACTGTTTCCCACA
CAAGTCTCCTGTTCTCTTAAGCTTACTCACTGACAGCCTTTCACTCTCCACAAATACAT
TAAAGATATGGCCATCACCAAGCCCCCTAGGATGACACCAGACCTGAGAGTCTGAAG
ACCTGGATCCAAGTTCTGACTTTTCCCCCTGACAGCTGTGTGACCTTCGTGAAGTCGC
CAAACCTCTCTGAGCCCCAGTCATTGCTAGTAAGACCTGCCTTTGAGTTGGTATGATG
TTCAAGTTAGATAACAAAATGTTTATACCCATTAGAACAGAGAATAAATAGAACTAC
ATTTCTTGCA
SEQ ID NO: 2
ABCA4 (ENST00000370225.4)
GGACACAGCGTCCGGAGCCAGAGGCGCTCTTAACGGCGTTTATGTCCTTTGCTGTCTG
AGGGGCCTCAGCTCTGACCAATCTGGTCTTCGTGTGGTCATTAGCATGGGCTTCGTGA
GACAGATACAGCTTTTGCTCTGGAAGAACTGGACCCTGCGGAAAAGGCAAAAGgtaaca
gttactgtctgtggtttaaaaatgaggtgtggagcaaataaacaggttggaagtgtggggtggggtggtggggtagggtggtggggcagggt
ggggggttgtgagcagtcagtgggcttgtcgccgattagcactgaagcagtgtttagctggacggcctttctgtgggcccctctgacagtgcc
cttcccaggaagatgtgtttctctgtcctcagccacatgaaaatcttttgcctaccgtgcctgtcaatccattgcctgcccgcccctcccccaccc
cccgttttacacctgcctgtccagtctaccgctctctagggcatccacgctgagcagtgggaagaactttaagccctgaagagcaggccaaag
gcaagcaagaaccccctcgaacagcttcccagcttagtgaggccttatttcattgattctctgaggcacattgttttttcacatgttagcatttctga
aattgggatgcagctcacgatcaagtcacagtttaactggacacattatttttctttcttagtggtgcagaaaagtaacagtgtgtcttacaattgac
tgcgtcctagattctgtgagatgcaatacgttattaaccatcacgcacatttcctgaactctttcaatgagcagacaccagcctgggttagactgg
agccctaaaagcacgacacagattccaccctggactggcttctgttctgcctgggaaaacccaaagtacgtttggagaccaagagcaacata
aagtagcataggtggaatagtccatgagaagtgcgagcaaaaggtgccggagatcagagaacaccaagactgtacttgtaaatgacaactg
gctttgtgcaattttttctgggaaaggataaggagtgactatagaactgtaaagaaagaatgcactttgctacagccttgcagagttgtgcaaatg
ccgatgactaaaggagctgaaagaggaaggaggggataagggatgggggctgggtaggggtgagattaggaccctgggagctgcaagc
cactggagagatcaggaggaaagggagggagacctgctttaggcgagaagagaacagtatttgttccaaatctcggttcagaataagttcat
gtaggtgatggggccaactggaacaggtgaaggcctatgaatgagtgtctcagttagggtctccttagagtttaatatgaaaaggtgttagcta
agtacagagctggtacctgagagagtaaaaggaaactctaaggtatcatggaggtagcaattgcaggacacagctcccacccctagggctg
agagaaccaagggaagagacaggaattattaagacttggagcatagatgagaggtctgtggagctgacattaggacttgggaggaaggcgt
gcatggaggctgctgctggatctctgaacctgacctcgggtctggacccctgaggagaaagccctggcaggttggtgcatgtggggccgag
ggacaatagcttaacaaccagcataaaagagagcagcatgggacacgcttcaaccatgcgcatggatggctccaaaacctgtgtgtggctg
gcccaggacgcagggaggctgcagggggaagagacaagttaaacctgacttgtctgggaagcaccattgtcctcaggtcactttcctctgtc
aagcctggtgctgaagttatctgttgtctccaggggccaagtattaagagtaatcagaaactcagtcctttcttctaggagcttcccttcttgcatg
aaaatcctgataaaactggaaaaaaaaacctcatgattaaattttttcatgtattcattctttccttctatcaaaaaataatctccaggcaccgtgcta
ggttcattggtatacaatggcaacaagacctcccagcccctgcctatgtgaggcatctgtggactgcggaggaaaatccaatatgccattgttct
ctctttcccataagaaattacaattctcagttcattttattctcactgtgctctttgtgaccctcaaagggggtcacatgataacaggactgtagctgc
tggcctaaaatgagcccattcctgtggcgctcatgtcgctgtgacagagaataaccctgttttcagaatgctctggtgccctccctctcaatctgg
cctttcgctggcatgggtgggcgactcctgctcagggactctgccttctccacagtgtgctcccagggagatggagccactcgggctgaggg
ccttggccagggcacctcccagggctgggcctggtctgggctggcgttcactggatgccatcctgatggcctggaaattgagatttctgtctg
gcacgcctcccgatggctccccacctgctaccacattccaggagcttccaggatgtctgggtaagacagaggcacccccaacagattcagta
gctctgagagggatctgtggctccttcctaagcttgcggttcttctggaaacttctgcctctagaagatggtccctctaagaaaagtacaaccac
ccagcccataattcagctcccaggttttccctcaaacctccatgtctcctgtaagcagagcaagagtaaaatcagataccaaatttcctcattcct
cagctcccaatccctaagggcataagatgaaaatcttcagatctctgctttcctccctctttttttcttcctctgttaacatttgtcaagtgttactaagt
gtctggcactgtactaagtgcatcacctccctgaactctccgaacagttccacgagagaggcctctctgtgatccccccggtactgatgaggtc
actgaggctccagagaaggattagtaactggtggggttggacctgggattcacacccatgctgcgtgacccaggacaggcaggcatggcc
gttacaccacactgacccccgtggatcgagatctatccaatagtctggtcactgatatcactaagatagagtggccatataatttatcatccaatc
agggcagttttgcaagtgaaagggagcactattaataattgcactgggacaataaatgtaaaccaacactggacctggaaaactgggacgtgt
gtttgccctataccaaggtaagctagacacagccactgccttcatggagttcagaaccaggcaggggcggctcccacgtataattactgtgca
gcacaacgtggagaccgtggagtagaaggaaacacggatgggaggtgaggaggaggtctgtgagctcagaggaggcaccggggctgg
agagggtgagagaagacttcccaaggagttcatcctgataacgtgcattcccaatgacgagcgctctctccactgcacaagacaagtatacat
ctgcccgtgttggctgtggacctggcgctgtgtcagggagggtttatgaagatcactaggtgggtctcttggtgtcatcccttcatcccagcttct
gggttaggatggatatctgtgggggggcctgaggactcatgaaagtggggcgctaatcatgttttggacaccacaccctggagcacctggga
cagctgtggcctttgtcctgggttcagcatcaagccgaggatgtggcaagtaaagagaggctgggcaccaactccagtgtacccaggctccg
ggtcatgtttgtccaggctaagaattctgtcctggttctcagtgcagaaggaagaatcatggggctcattttaggccttggctgccttctgttaaatt
gaaaacagagcaggaaggaagaaaatttaacaggctcagttctaaaacaacaagcacaactgtgcccttgccagaaacccctcctccccatg
ttgattgaatggtaaagagaggaggggaggtgagagggagagagagagagaggaagagagagagaaaggaaagaaaggaaagaaga
agaaagaaagaaaaggaaagaaagaaagaaagaaagaaagaaagaaagaaagaaagaaagaaagaaagaaagagaaagaaagaaag
gagggagggagggaaggggaaaagaaaagaaaagaaaaagaaaaaaagaaggaaataccagtttgggaaaaaagaattttccaccagc
ccttctgagccttggctgggcttaattaaagttacagacatgtgtaaagggcagggtagggggagtctgagctgctgagaaaacatgtttttaat
tatactgtggaatttctccctggggtatgcctgtacgcagttaagcgtcaaggacagggatgccgctctggggaggggaagctgagcatgatt
ttggaagccggcagaagaggctattgtgaaaaccagacctgtcaggctaggaaaagaatggctggtggtctttgaccagggagtgacgcgt
gaaatgcagcaaccgcccccgccccccgccaaaaacaaacacactctcacagagttagaacaacagtgacctctcaacaaatatttttcaaa
gattaccaaccaaccattacctagagcagcggttctcaaccttggctgcacggtggaactacctgagacgtgttaaaaagaagaaccctgatg
tcccatgccccaagattctgatgtagttgatctggggtatgatctgagaccccggcatgttttcagcctgcagccacatgagaagtgctgaccta
atcaacaggggtgatgatttgaggggcggggactataggcaaaaaaaaacagcctaattcaaggatgagaagagggcacaggtgaggtgg
gaacagtcctagggccagacaaagaaggaagggagaaaggaggtgctgatccctcccctactcctgagaggaggcctttaagtcaccgtg
ccttgtggagaccagattcttcaaaaatacaagaatgagtgagtgagggagtgggtggatgccaggagagtgcgtgacaagccttgcaagg
gaggatgacaatgcactagcttggtttggaaattttacccctggaacaggcaggccaagctggctggtcccctccctgatacacagccctccc
tctttatatatggagcaggggacggtgtgtggctggtttcttagcaagcaccatggttccaagttggcaactggggagttctgaatccaaaaag
gagggagatgaacgtaagtggagggcaggcctacaaggttgcagataagcttaattctgtctccttactcttctgcctttgcaacaaccctgtga
tcttgcgacaaccctgtaaggcaataacaaatggctcatgtttattgagtgttacctcatgccatattgtgctttcgtgtttaacacaattgtctcattt
caccctcacgactgctctgggaggtaggtcctggtatcacatccatttcacagatgagaccatttggcacggaagagttgagtgggctgccca
aggtcacatagctaagatggaacaggctggataggaaccccagtaacttgacctcagagtaaccttctcttaaccctgagtgtacactgtagga
aaaatgagcagtcccatttcagagaggacaaaactgagactcagaggttaagcaagccccaaagtggttgttaacccagatcttcccactaac
tcccaaatcagcatcagtgtttaacgtaccagacctctcccagatagatgttgccgcatggaagacagccgatctacgtgatagaaagccaata
ttgcaagcagtcgtctaaaggagtcaaatgtgttggatttgaactggatgtctcatttctttggtgaagacactggaaacaacttccaggtttcatc
aattgctcctatcactcaacgttgctatcttactgaacttgttccccagccttacccactgatggaatgatccagaatggaagacaagacaccaat
gtacatgaccctgggggaggctgtttcttaaatctacagactgttggtgacctgagccccatgtcaccaaaggctttcctggagaagcctccta
gaccagtcttgacaaaggctcactcattccgtggatatttattgggcacctattatgagttctgccccatgtggggtgctggaatcacagtagtga
caacgacagatgaggttcctgtcctcaggaagcttactgcccttgagggcttcacttacttggaggagtgatgaacctgaagtgcggtgtgtgtt
aagaagcggaagtccagggccaggcgcggtggctcacgcctgtaatcccagcactttgggaggctgaggcaggcggatcaccaggtcag
gagatcgagaccatcctggctaacatggtgaaaccccgtctctactaaaaatacaaaaaaattagccgggcatggtggtgggcacctgcagt
cccagctactcaggaggctgaggcaggagagtggcgtgaacctgggaggcagagcttgcagtgagccaagatcgtgccactgcactcca
gcctgggcaacagagtgagactccgtctcaaaaagaaaaaaaaaagtgcctcacggagagtctattcttttcttcccatattgtgtgtgtgtgtg
cgcgcttcctccaacacatcctccctatatatattttgagtaaaacatcttgtaaaaagttacagctacataatcaccacctgtccctaaatagttttt
gctttttctttcttcaatgcacgatcattttcccccatcaatttattttttagtttcttataatcttgttgccagtaggctgttttttaaaaagcagaacatgg
tttgttcttactagcaggaaaggagcatttattgagcctctgctatggtgtcttttattttgctgagagcctatttacatttctttgagaggaaaacaac
aaagggttacatgaaagaccatgtgaatagcccctagctgatctattaaacttgctattccccggccagctgcttcagatctccttcagatcttatg
tgtttccttcctaaggtccctggagtaagggttgcatagacctattctactctccaactcacatgtccctctccctcttcctctccataattccacatct
ccaacccccacccctatgtgcaatgccacagggtgtggactgccacagccactggatctgcttttggaatcaagagtccttaagctccaaatg
gaaccgaaatttaaataccaactttcaaccatatgttaacatcagcagcctcttccaatgtaaaaacccatggcagtgtgccctgctttgtttcttta
agcaatagaaacttgaaggaagcatgttggtaggccagatttttgttggctttgcaatggatcacagtcatttattcactcattcattcactgattcat
taaatgaccacatttgcaagggcaaggtaatggggagggccagaaaggacactggccccagaaacaggaggctggattttggttctgatgc
tgccactgctgatgtgacactgcacaggtcacctgcctcctctgagcctctttccttaactgcagagtgagtggctacagagaaatctttactacc
tgttagatcagcattacctgggagcttgttagaaatgcaagctctggtggggccatactgaacccaaatctgcattcatgtgcatagtgacagct
aaaatgcactgaagcagatgatcttgatgatcctttatgaaagtctcatgctaatgcagttttctaaaatagaggcagagtggaacccagatgga
cacaaaatctggttgatataataaaacaaggtagagggtgtatggtggggagggggtaaaggaaggaaactgtttaggtaaagataccacaa
ccaaagtcctactgcacacatgggatctgaggagggctgtgtctgctctggttacgttttctataatctcttagcaccactgaactttctctctttttg
ttttgtttttccagATTCGCTTTGTGGTGGAACTCGTGTGGCCTTTATCTTTATTTCTGGTCTTGA
TCTGGTTAAGGAATGCCAACCCACTCTACAGCCATCATGAATgtaagcatagcagggtagcttgggc
aagccctgaagagactttggtctgggccttttgtctagaaagatcttggggtgggagtgtggggatcagatctgcttatcatcatttcatgtctatg
atgcatgtaacagatttatcaatgttacacaaattataatttttaaaaagtctttagagacagggtctcactctgttgccgaggctggagtacagtgt
taggaccatggcacactgcagcttctatctcttgggctcaagtgatcctcctgcctgggcttccaaagtgctggaattataggcatgagccactg
ctcccagctaatttttttgttttttgtggagacagagtcactacattgcccgggctggtcttgaactcctggcctcaagtgatcctcccacctcagc
gttctaaagcactgggattacaagcatgagccaccttgtccagcccaaattttcatgttttaatcctacacattctaagcaaatacttgtgtgtagtt
actaagggactgtgcacttatttttgtttgctttgttgttgctagtttttatttttttatacctaaactctctcgttttaaagagaacagatttgtagatgagt
tctcgaaaatatttcaggaatcaatatagagaatatgttatacatggtgccagagaaaaatgaggacaagagatgctatacaatcgtactgaaga
aaaattttatttcttggacccctgaggtgtctgcagacctgaaaggaacctagtgagagcctcttttacactctgcccctgtgggaaagccttcac
ctggtttccggccctctatgtggtgaatgtggaagcctcaagcgttatgcaaatctgcccagtcctctattcttgatcttcaccttctcgttcatgagt
ttcaggccccagttctgaatcagcctcctgtccatcagactcttctttacctctccccgaggagcccataacctgcagccctactgcatgcttggg
gtaggtgctcagttcaccgtggttgaaggaatagacgagcgtctgctcaagcagcagcagcaactgcgtggagtcttcttgaactaacactcc
tatgcccctctcggcacaaaatgacgtgtccccccttgcttccccttcacatttccacccatgcctattacaacatccgtctgtctccccactacac
cgggagcttgagagaagaggccatgtctctagcacccagcacagggactggcacacatgagatgctcctgcttcttaaatgctgagaatgaa
ggaggacatcagaggggcccgggccccttcccaaaaaggccaactcctaggtctgcatcctgcttggtctccatgactaatcccgtcttgtcc
tcattttctgttttaaagGCCATTTCCCCAACAAGGCGATGCCCTCAGCAGGAATGCTGCCGTGGC
TCCAGGGGATCTTCTGCAATGTGAACAATCCCTGTTTTCAAAGCCCCACCCCAGGAGA
ATCTCCTGGAATTGTGTCAAACTATAACAACTCCATgtaagtgttgagatccctaccatgcaggggaggaa
gttgcacaccccttcacgtgctgaaatgcacacgtgcgtgcacggagcatggagcactgagtgttcttgtggctttgctgagcccctaacctctt
aggagcagagcaggtttcctctctggaacattctgttaactgtcagggcacttggggagaaatctccaagctaaggccacgtgcacaaaatttc
ttggtccttatatccccagaatgtgacctggagtctgatggcagcccgctgcagagatgtgtccactgccttctggtcattgacctgcttgggtg
gagtgaatcattgtaggagaaaaactcagttccctcaccctgatcaacctggacagatctctcttcctttaaaagctttcttggacatctaagggct
aggaaaaatgtcagggagcattgggaaggtaaatgaagtcaggtttacaaagtcaagtttacttcttgggagaaaaatacaatttccaaatcctc
tgttataattgccatcggccccctggagtggtgagatctcggaatatggctcgggtgcagtggctcttcactgtgggcctgcaggctattctgaa
aagctgatgaaaaccaatgacccctcttccaagaaaaatggccacataccaaacattacactgtacatctgatttcagggaattgtagatgcca
ggttagtagcctcaggtctagggtcaaaattcaagtcgaatcccacaggaagagggtctgccttcggaattccctttcagagcattgggagaa
catcatgggagcatattctagagacagaggcttagggtgtggacagggccatccctcacccactgtgctgaccttaagcagcaccttgtgcag
cccatacctgaaggccaccagcaaaggcctgttggggagcaggctttacccgacctgtataaacaccaggctaggtgaaaactgagatacct
ggttactttagttttttccttgggggagctcagtatgattcttccaggagaagcctgcttttagactaaaaagaaaaaaagtttgataggtcaaccta
atgattggaggtggccttccccactgtgaacaaactatggctgcatgtgccctacaatggcagagttgagtagttgtgatagagactgtatgatc
tgtaagcctgtaatttttatgtttgctgacccctggattaccagatgatagaagaggaaacatctgtcttcctagcaaagtcaaggaagtggcattt
agcaggactcatattgctgcaagcactgccttgcagttttagtttacaactgcactttcagcttaagaaacacctgcccatccagagagatcgtgt
ggggtcacatggtgggatcagggaggcctgaagacagctcagtggaggctgcatggagctttggtgggaacggccctggcagtgtctata
gatgttattgcggaaaactgaggggtgggagttggagaagggggctccagactctagctgtacttggcatttgaacccggaaagttgggtttc
atgttttgcactcacattatgagtgaaatattggcttattcaaggttcttttgcttgcaaggcacggaaacccattcaagcaatcttaaaccccagaa
ggaaatctatgatttggatactagacattctcacagagccaagggcagcaaggcggggctcaggagaggcaggccaagacctggagagct
gtcaggagctgcttcctcaactctcttccatctgggcctgccagccctggcctctgtatctactccattcacctctctccatggaccagtctcccct
gctcctcaatgcctgggctgccattgttcatgcaattcacaatacctcggcctgggcaatcagaagctcatctctgaacaccatccaaattcctg
ggaacaaatcgggttgacccagctttattctccctgtcccatcagccttggcagaggcgtgcatgtgcatgcgtgccaatgtgtgtgtgcaggg
aggtccttgtggatgaagcatggctgtcagagcctacctgcgtgaatgggtggaagggcaggtctcagagaattgggtaaaaactggataaa
ccctccagtgatatccaccaatgtcaccctgtttaaggcttctctgggcaagagacacacagagcatgggaccgagaggcgagcagaccct
gccaaaactgggagactgaatagatcgctcaccatccttgtcagttagcctatatgtacaaggaagtaaaattatctctttctcctgccttggcagt
attgtaaggatactcaatgtagtagctaggccagacacatagtatctttaaatatagcatgagatggccaagcacggtggctcatgcctgtaatc
ccagcactttgggaggctgaggcgggtggatcacgaggtcaggagatcgagaccatcctggctaacacgatgaagccccgtctctactaaa
aatataaaaaattagctgggtgtggtggcgggcgcctgtagtcccagctactcgggaggctgaggcaggagaatagcgtgaacccggggg
gcagagcttgcagtgagccgagatcacgccactgcactccagcctgggtgacagagcgagataaaaaaaaaaaatagcatgagatattatta
ctgttataaaaataacagctatttccttattaatgaggctttgtccttacagCTTGGCAAGGGTATATCGAGATTTTCAA
GAACTCCTCATGAATGCACCAGAGAGCCAGCACCTTGGCCGTATTTGGACAGAGCTA
CACATCTTGTCCCAATTCATGGACACCCTCCGGACTCACCCGGAGAGAATTGCAGgtaa
gcatgactgcagtgctctcaagcatcatttccctcacctatggagagactgaagatataggaaagaacagggagagttggtgaaaaatatact
agcggaggcaggaagggatggggtctggaggcggcttgaacatcaccttggtgaagatgcctcttcctccacagaagcctggaaggtagg
aagttgggaaggaaggcaggaaaggtctcatccacgttaagtctagagacagaaagaatgctaagagagatggcactatgggaagtatgag
gctaggtcaagggctagaagcaggggagacgagtttacagagtttcgtaaagatatagagcaactctcacagagttctagagcgagagctaa
ccaggaacatgaagcagcaaggccaactatcattaaggagccagggaggtcagagatcatgtattatcatgacataaatatgcataattgtact
atttctcccagtaatatttagcacccaggccccgaggcagagcaagtggagagtgggtgatgcagggctgggggtgtgtatggaggcacca
cagaaggtcaacaggcagcgggctgaaggcagggactggactacatgcatcaagtccaggctgcacgaggaaggatgagaaggcagat
gagcacggaaatggactgggggaaatgaagaggcaagggaatagaagtctcagtgggtgccatgaccctgtttaagtgattgagaaaatga
acaagatgaaaaggttaatggctgtggtcagaaagtgaaatatgtgaattcaggatttcgaaggtagggtgggtgatgactggcccccagatg
cggccatggtgaagtggggcaaaggtgcaggtgcatggtgaggggaaggaggaaatgggaggtgatgatgttggccccacacggacac
cacggttgtgcaggaagatggcaggagctgggcaccagggtgggagccacctggagtcaggaagagtgaagagaaaggatgaagagg
ctccctctcctgtgtctctcctccccaggagaagaacaagaaacaatccgaaagtaataacaccaatgtgcctttacaaagtgtgagtgggtgtt
gtgtgctgtcacgtgtgtagtaggctcctctgtggatggctagagggactggacatggccactggatcccacttgcaagagcagaggaaaag
agtggtcgtgaggaagtaaagccccccaaaatccaggggttgctgcagctttgggtgtggagcgtgccctctgaggaaaggctgctctggg
ggagattgcccaggaaacggggctcagaggccacgaaagcagctgttaggggcttctgggagatgtgtgctcctaggattagggagttgac
tctaaggatgaccttagaggttaacagggatgagaaaggggtcaccaaggggtctaccaggggaatgggagaggctgtattgatagaacag
cttctgctgcaggttccaaacaagaaatgtgggagaatggttgaaatcagccccgggggcaccttcccgtgcatgcgtgcagctccttcaaca
ttcagtcgaccttcagtgcctcctgtgagccaggcactgggctagtctctgggggtggagagatgagtcaggcaaatgccagccctcagagg
gctcacagggcagaaggtgagagatgagtgagcagaaaatgaccacagcgcgtgtggggcccagtggagggaaggaggggattcagg
agcacaggagagtcaacaggggaaacttctccgaggagaatctgatcctcctcccatctggccaccttctgaagccctctctccccatccaag
tgagaaaggacaggcgtatgaccagattggtgtatgaagatgctgaattacgttctcattgtttcaaactagtaaaccatagattttatgtagtaac
ttctacaaactgcattacaaacactccattctttgttgccctgggtagaagtttattttagtgagcccaagtttgaggaaccttatatggtatgagtac
aattaccattttaatagtaagaaatcccccttcccctgtgtaccaaccagaaggtgtttttttcctaatttaaacaaacagatgcagacgtgggctgt
ccagctcctggcgggatgacatacctcatgcatccagtgggtttgatgatgaggcagacatttcacttaagtgcctgatcatcagattgagtcct
gctgggaggaagtgtgaaggaagtaatttcaaaccacagtttctctgtggcttttacaatgtggatatgagaaccaaaatcactacttcttaaccc
cagagcaggactgattttgaattggtatgcaggcggttccttctgcaggcttcgggctgtgagaagtccctaacagagcaaatctggggacaa
gggctcaggaaaggttggccacggccccctaggaatgggggctctgcaagatccctggccttagaggctgtgagagggaacaggggtcc
atccccaagtaagggacacggtctttgaggaaatcccaggccagggcctgaagggcactgtcaggaacacaggctgtttcagtctgttgaga
ttcaccggggcgctgctcactgtgagcacggactcctcaggccaatgtggcagaagagcccacctttgaaagcgagcgggtgggggtggc
ggggctggtgctggtgcgtgcttctgcacagccacctgggaaggtatgccgctggttgacccaggcagaggttttctttcatggcaaacctgc
agtactgcattctcagcagggaggattaatggtaaaagaccaggcatggagcccccttccctctccctcgaagcaagctctgtggtctctcaat
catctttaaaacaccttcttcccgggagcctcctacattctcctggcttccctcccacccccaccctcagctcctggggcctcagcagccccacc
cccaagcctctaatcttcccagggaagggaacaagaagaaccacattttaaacgaaatttatttttctttcctcaggctcccagttcacatttctcc
ctcaggagtctagggaagcttctgtctggtatcggcctcctcttcacctgggcccccgccctcctcaggtgtaccagaagccagcacactccc
ccttcccccccagagccacagcagccctgtctcctgggtggtcttgtgtgccaagcctgggcaacatcactcccagcttttcttgttttgcccctt
ctccccagcaagatatttgtatgtaaggtcaggtgagtgagttaaagaataacgaagagataaacagtcaaatggagtcctgactgtcaggtca
agacaacagttatttactgaatgcctcatgtcattcaacagacatttattgagactctgattggatgtcagtctttaatgctgggtgtcagagagag
gtgacttcaagggcttgcatctgtgcacccagcattgctaggtacaatgaggagtataataaaagcaggagccatagcccccaactctcaaga
gatctcccatgtgtgtatgtctgcatatgcgtgcgtgtgcatgtgtgcgcatgtgtgcatgtgtgtgtgcatgtgtgtgcatgcgtgtgtgtgtgcg
tgtgttggggatggtgttggtggagtgagagtgtacaaggctgtgtatgaaggggtaattgggaaaagaacaatggagctggcacccaggg
acaggaggaaaagcaggagggctgggtttggaagacagccggatttatgtttttgaagagggaagactagaatataagggagcagcccttct
cagagccctcctcctcccttcgggccctgtgtccagctttccccaaagtccttggatctttcctatgcaaaggggagtgacagtgggcaccact
ctcagggaacccattactgtgagagaagccactgtgccactgtgtggtcgaacttcaagaccggcttcccctgccccagctgcatggacagg
cctgtggggttggcgcaagacccttccagaggaaactagctgcaacataaatccggatatggtgctgttcagggaaaggcacaacctgggg
atgagaagggtggctgtccagcacacaggggcaggcctcttggccactgggggaggggagaatttggagaggaagaggatgggatgcc
gtggaattgggaccaggaaagaatggggacatgtgatggttaaagctagttagagaagaactgggagataaacagtcacccatgcccctga
agcactcggggtgaagagattggcattttcacgcaccccagtgctttccctttgtgttgaagtcccttcgtagacatccaggcccataaggctctt
ctctggccagagcctcatgaactatagcactagcagggttgaggccaagcattggccctggaagccagccgaggaggagggtgcttgtgtg
aatctcccaggaggggtaagaattatattaattcgatcataataagcatttattgagtgctgttttgaggcctgggagctaagcacttcacattcctt
accccgcatcaacaatcctatgaggtagatgtggaaaatgcagacacggggacaggctcaatcacttgccccaaggtcaccttaactgttag
gtgttctttatgcctccttataaagaaaccctgcttcccacaggtgttgagaggagctggagggagcttgactagggctcatcaggcaagcccc
ggcatgtgcctggctctcctctttctacctggagcttttcctgcccttaatggccccaactcatttctcttagtccatgtcagtgccctgagcatctca
gcccaagctgagatgatagaaacacccagaggggtcctctaccctgtgacagctgcggtgtgggaagagcacgtgtctcctccaatcctaga
ccagagtttctcagcctcagcatcactgacacttggggctagataatcctttgtgtgggggagggaggagtgtcttgggccttgcaggatgttta
gcagcatctctggcctctacccaccagcacctccccagttgtgacacccagaaatgtctttagatcttgccaaatatttccaggaggatgaaatt
cccctgtttcagttccccagccccacctcaatgagaagcactgtcctagaccaaccccacaaagcatctgacacccccatccagccctggcta
actttttccaccttcttactaaattgggcccagctgcttcagcagtcaatgtgttgggggcagcccactggcaagagcctcacctctaggggctc
ccagagaccccaagaacagaaccttcctctgagagttgagttacaagtgtttccaatcgactctggctgttttcctttttttgacccatttccccttc
aacaccctgttctttctcttattcatatgtagGAAGAGGAATACGAATAAGGGATATCTTGAAAGATGAAG
AAACACTGACACTATTTCTCATTAAAAACATCGGCCTGTCTGACTCAGTGGTCTACCT
TCTGATCAACTCTCAAGTCCGTCCAGAGCAGgtagggggatgtcactggccagtggtccctggaggggaggg
aagcacccagcctgagaaaggcaagaaatatattggcttttttcttctttcttccttgtgttcacattcagaatccatcacttaatgccttgtatttaga
aaaaaaccgggggatcacttgagatcgtgatcattttcaacataggattcgaagctgtacacatcctggtgaccttaaaacatctcaggtttttata
actggaaggaaccttagagatcatggggcacaaccttctctttatagatgaggaaacagaaatctattcatttattactcaaatatttagggacagt
tgtaggtactagaacacagtgtgaaccagacaggcaaaaccccaggccagggagcttccattccagtggggccacaggcgatgctcaggt
aagcagagactccgctgtgtgacttctggctgtgatgggtgctgcaaggaaaatccggtagagtcgagggttagagagggacggaggggc
aggtttaagggggatgctcaggaaggccttcctgaggaggtggtatttgagcagagttgtctgtcagccacacagtaagtgagaggggagtt
ccgggcttggaagctgccagcacagtgctggcaagtgctggggtggcgtcccgaggctacagaacctgagatgctgcagaagagcccact
tctgctttcctggaccacttccttctcagcaccaggcaaactccttcttctatcccctggcacatttctgacctgtgtatacgcccccaatttatctaa
cccctttaaataatctcctctatttatgcagagcattcttaccactaactcacgacttgcacatcccttagctcccttactcctcacaacaatcctgag
atgggtcagagaaggaggcttgcgcgtctggtgatggggtgatttgtgcacagttacagggctagaaattgtcagagccagatggaatccag
gtcctctcaatcctaatccagtgtttcttacttcagtcctgtggctctcaaagcccagagaccagcagcatcagcgatgcctgggagcttgttag
gaatgcaaattatcagggcccactccaggtgaactgggtccaaagccctgggataaggcctagcaatctgtgcttcacaagccctccaggtg
attccgcaggctcaggtgtgagagctgcagctgtcctctgggccttctgggctccccgcccagcttcttcagtgtgatgaacacagcgagaat
gctagatctgcagcagctgatatcccagacaccctcccgactccctcctggctgggtctgatcctcctccagactccaggagagaacgagac
ataaacagaacttcagagcctgtgttaaccctgagatcaaggtctgcacagggtgctgtctgagtccagaggagtgagggaccccaccccac
ctggtcagcaccagctcctggaagcaggttctcacactggttccctgcacaatgaaggagctcatacctgcttttctggcttctcagaccctgag
gttttcaccgaaactagacaaggggaacctagggtcagcctggaggcagggtgagcttggcgcctgcagtgcccaggccctgggtggtgc
ggctccggccaggccctgtttagcttcctctcccacccccacagagggggtgctgtcggcaccgattgctcattttcccctttgctttctcttcag
ctcgtaaaactcaagtcctgacaatgccttgatgacttccagttggtaataaaagggagatgaagataaggacaggaatttcggggaaatttctt
tccagttccttactaatgtgacatttagatctctagtactgtgcttctggcatcagtgccaaggcctttcatgttggagaatggaggccggggtcac
caggttgtgcctttatttcatgttgctggctctgatgagctgatgctctgctgattagcaaacgctgagccatctgcgcttcgcagaggcacgttc
cagccaacccggccctccctgcccacttcccaggatgctttgccttgtgggctcacctgtcttctagctcctgatctgtatctccacctccatcca
gttccggggctccttatcagcactgttcccagaactgtccatcacgatggcaacgttctctctgggcgctgtccaacatgggagctcgcctctgt
gttgtcactcatgctcattgaacatggatttgtgtcctttaccatcaggactggatacccctcctggtcctttctgcctggggtcttagcacagctca
gaaggaacctcaccattccctctctccatctagggaattagaagatgacaggggcacagttctctggctcacccccagcccagtaaactcctg
gacatgcttcaaggcccagctcagatgttgcctcctcagtgaaataatttataaacccacccttctttgtcctgccttctccctcttccctactcact
ggagagttaacaggtgatggttaagctctgggttcaaatctcacaaggccacacacttagctatgtgacttcaggcaagttaattaaccactctg
tgcctctcgtttcctcatttgtaaaatggaaatagtaaaagtgcctaccagcatggcagttgaagttaaaagaaataatatatgtgaacacttggaa
gggcgcctgacacatagtaaactctcagtaaatactagctgcttttagtggctattcttaacacaccctcttcagtgctctggtttcactatgttttat
gggtccctgagatcgaaagtgtccacaccgactcatggtcagctgtaacctgtgcctcgtgtggggaccaggctgccatgtgtagtctggaca
gtgtaggaggtggcagagctcaggcctgttctgccctccagcccagagagccacgtcgttagatgtcatgggagactgtggtgccccggga
atctcacgaatttgcccacggtactcagtgtctgtccaatgctatgggagtccaggactctaggagccagttaaggtgctgggtggccacaggt
ccctggccaaggtccaggcctctcccctgccacctgatcctcgagaggccatcacgagggttgtacttcaagaaccactatccttgagctacc
taggagctgcagaatgtgcactctgcagggcttagggcctgcagacaagatagatgcagggtgtctagttaaattcgaacttcagataaacaa
caaataattttttcaaataattgtgttctattcggtccctatttgggacatatttgtactaaaaagtattcatttatctgaaattcagattcgactgggcat
ctggtgcttttgtttgctaaatccaagagcaaatttgttctagctacttctcaaccccaccttcagagaggaagccttgatggtactgtaacatcatg
ctgtaagaaggggatcccttgaattgtaaatggcactctgataagatgaggtatggggattgtattggtttcctgttgctgctgtcataaattacca
caaacttagtggcttcaaacaacacagatgcattatcttacagttctggaggtcacaagtctgaaagttagggcatcagcaggactgcattcctt
actgcggagttctagagaaaaatccattttcctgcctccttcagcctccagagacacgccacattctttggctagtggtctgcttccatctccaag
gccagtgggggcttatcaagtctttctcacatcacatgactctgtttcttctgcctccctcttctacatttaagggacccttgtgattacacaggggc
ccacctagaaaagccaaaataatctccttattttaaaatcagctaatcagtggctttaatcccatctgcgatcttaattcctgtcgccatgtaacaca
aggtattcccaggttctgtgggttaggacgtgggtgtctttcctaccacagggcagtttctagtgttgcctcttctccctgcagTTCGCTCA
TGGAGTCCCGGACCTGGCGCTGAAGGACATCGCCTGCAGCGAGGCCCTCCTGGAGCG
CTTCATCATCTTCAGCCAGAGACGCGGGGCAAAGACGGTGCGCTATGCCCTGTGCTC
CCTCTCCCAGGGCACCCTACAGTGGATAGAAGACACTCTGTATGCCAACGTGGACTT
CTTCAAGCTCTTCCGTGTGgtaagggaggggtttggctgctcgccaattgcaaggtgattcctggggtagcagagcctcac
gaattgaccttggggagggcgtgagcctggtgttctggacaatccttgcaaaagctccaggctcccagggctcaaaaaatcacaactgatagt
atttctagaacagtggcccagggacccagaagtcactatgaggttcaccattaggtatgtggctgtggcatgtttgtgtccactctaaatgtggg
gataatcccctttacctcctctaacagagtggtaaaggaaggaggaggcctggtttgactccctgacctgctatttcctagccaggtgatcatgg
taagatattgaaccttttctggtcccagtactcatctataaaacaaatataatactttacagagtggtaggaattatacaagaaaagtatacgcaaa
acatttcataaattttaataaatgatggccccatgcttcttcctctggaaatggtctcaacctcaatggttggtgtttctagagagaaaaaacgaca
gagaaagtttcatagtctcaaaaatttggaaagccctgatctagctcaaccctttgttctagaactgcatcccagacagactgcttgggacctga
aaatatctcctcctttgctagaaggataagatgagaaggaattagataaaggaggtgtagagcagaggttttcacactgcaaagtgcataaaaa
ccatcagagggccgggcgcagtggctcacgcctgtaatcccagcactttgggaggccgaggcgggcggatcatgaggtcaggagataga
gaccatcctggctaacacggtgaaaccccgtctctactaaaaaacacacacacacaaaaattagccaggtgtggtggcgggcgcctgtaatc
ccagctactgaggaggctgaggccggagaatggcgtgaacccgggaggcggagcttgcagtgagccgagattgcgccactgcactccag
cctgggtgacagagcaagactccgtctcaataaaaacaaacaaacaaacaaaccaaaaaaacccatcagagaagttggtaaaagatgcaag
tgctaaatccccacccccaatcactgtgattcagaagaaccaggccaggcccagaatctatcctgttaccttaggcgattctgatgaagaccatt
gtaggccacactttcagaaacactcaaaattagaatccttcagagaaggtggcatatataatatttctagcatggaattatgttttttttcttttgccta
cattttaatttctagaactgtgttgtagggaatgtcagtcactaagaacttgattgaggaactgtgttttgtctgtttcatgactgctctctcaagtccc
aggaaactcactttcagcttgtcttaaaaagcaagctgaaggcttttaaaaatgaagcaacatgaaataagacaccgcagtttctggcacggtc
cacgcttaatccccttcaatgtgtgactttccgtggaaagttactctacgattttcccagctcgtcagggtggggccccagagtgagtatgaagg
gtcagagcctagggatgccaccatcagtgagagcccaggaccccagaaaaggtctcttggctcaccacactgtaggaaaaataaaaagcaa
tgtagtccaaatgtctctatccaaagtttcaaaaagaacttgattttagacacgctccttgacttgttttcagaatcagacagaagagtgaggcaac
aaaggtcccttattccaggcagctgaataccagcacagccaggagtccagtgctggtgtttgcagagccaccagaggctccctctcaggtgt
ccagggcccgcatgctttgtagaatgggcagaatgagcaatgtctgtgcacctgggctttgcaggcagggcctgggtacccaggttcgtgca
atcctctcgtcaccatgaagggagcagcatcattcttcccttcttgaagcaccttggccaccagtataggtaaatttacctcccaggacatgacc
attgattctgggatgtcaatgccagagatagtagggtaaatcggcacctgggtaaaactttccattggagactagaaccaaaactcaggacact
ggcttccaaatgtttctttatcagacaagaaagaccaagtctttccttacgtcttcacatgctgccttggcaaatgctagcattcacaaaccctggg
ctaccttgacctgtcacccttgcagacctcagacgggtcctgggggcttgctttctcggtttctgtatgcaggcactcaaacctgcatcaggcac
ctgtgaagggccgggcactgtgctgaggccaaggctccaaatgtgaaccttccaccctcactgaactcacagccagaccagagacaagca
aacaggacatttcacagcagtgcagcctagaaagggccaacaccagcagcatttgtccccccgagcggtagcttttagaagcttccccagtg
attcaatgtgtcctacaaatgcctggcccccactcccagagattctgagtcagctggcctagggtgcagccttgacttcactgtgttaaaaagctt
cccagataagtccaatgtccggccaagattgagaatcactgacctagagtttaatttaccacctcagtctctatagaccacgcataataatagtac
cccacacacctctgagggtccaaagaactttcatttgatcacccatgagaccaccgtggtgtggagatgctttctctctcctgttctcttaacaaa
gctggtgagcgacagagcctgcagtggaccgggagatggcccagaggagaaagctctgccgtagtcggcctcagttaaccacggagcac
cacccctacctgctctcctctcactcctgcttccgtctcggtggagaaagatccaaccgaagcaggacacatctagtcttctggtgcctttaaaat
gtacttttccatttgacaaatggattacactaaaaacaaaaatttacaaaaaaaaaaaaaaaacctgaaagaaattgcaggcattaaaatgggac
tttgcctttattgctcctgggcccatcctatttgggtttttagaaaaacaagcctgaggcaggcccagaaaggctcagggcagaccctccgatcc
tctgaaaggagcatcaggcaggcaggggttgctccggggccagggaaggggccccgctgggacgcggctgttattgcagctggttggcg
cgcagccatgcttagctgcagtgcgggaatgctgggccttctgttctgggctgtttctcatacgcacgtaggccagtgtataaataaggttttatt
aaatgccaaatgagttctcattaacaaagaaagagggaaaatctcagtaaaccaccgtgacggcatctacccactttgagtcaggagctggg
ggtgtgagtgcaacctccgagacaagggaacctgtggagcccagagaatcggaggggggcgctggggttagcaccgactgagaccagct
gtgttttctctcggttccttggagatcagaagtgagtgttgtcatcttcaaacaatccaaaggcagtacccatggccttactacatccctcccacac
catcccacccatccccgcgcgtacactcacacgctcatttgcacactatcgcacacgctcacttgcgtgcgcacacacagattggtgacctag
gtggactgggagagaaataagagccaaatgactggattttctccaaggaaatttattaatagcccctcttggtttcacctgaaggagcttgtcttc
acctgcggcctttgcaggcttaacgcccccagcttgaaacccagaagctcagacttgggcccaaggtattattagtgccaacactacctgaaa
tgtttcgcacctcataaaaatggtgtgtcagtttcgggtgagaggttgggacgcttcccatctgatttggcccaaggcatgcatgcccctccttct
ccttcccctcctcctccccctcttccccctaccatccttcctgttttctctccaactctggtgcacagctttgaaatcttgctgagaagcaaatctgtc
ccttctgctttgaatgtttatttgtggaagttcggcaggggaaccgaggcgggtgccaagacctgccatgctgctgggaagtctgagtctccct
cccttccccctcctaaatgcttgttgatagagaaaagtcagcctcctcggcatttgggctcacggttttcctttgaaaatgcttccagtgtggcatg
attcagctttcttttctgtcccccaaccactgctctgttgtcatttttacttttctgattgcattttatccgtgtctctttgactacggggtggctggacgtt
gagttccaggaagaaaagggcccaatcttggggttctgactacatgcgcccatcaatgtcctgtttcattcttggctctggctccctgaattcctg
agtcactggggagaagcgtgggtggaccgccccctacccagtgagagttgccacagttgctgctctcctgggtcattggttgcagattgttaa
acttcacctatgcatttcaactttcgggtggatattgctacgtcaagtgtctgggaaagcccccacagctacaggattttacagtgaggtcccact
aatgacttgatgtcatgacttcctcattctttccaatttctcccacttctccataagggttttgggaaggggagaagagaaaggagtgattcctgag
tgccagtaccagggaacagcagggctgttgggaggaaacaaaactaaatcaggaaggtttttgttgttgtttttggggggttttatgaaaatattc
aagccacagcaaatatatttgatttatagcattagtattttttctgcctgcatctacaaaaatctttacctattaccatcaaaatatcctctgggtgaat
ggatttcaacaaagaagaaataaaaatgaaatagaagagaggccccttcgtgcacattgagcctactggctggattgtcacttgcctgccttga
tgtcttttcagctccaggcaggcagtaggccagggcttattttcatgacagatcagatgttcttttatggatttacaaagaaagaaatactgagaa
gtcaaaactgaagtcacttaagacaagagcaggcccctgggaaggctgccattgaggataatgagtcctggggtcctggcctttgttcagtaa
atacgcactaggcgcctacaatgtgtgcaccaatgtgtgaggcgtcaggttctctccagggtcagttggttttaagaaaggttttggcttctgata
tgttttatctctacagaacagtagctcttaacctttcttatgggttaggattaccttcgagaatctgactacagctctagacctgttccctaaagaaaa
ctaagttcacagggacacacaggatggggctcatggagcagctgaagccagaccccaggttaatagcctttacattaaaatgtttttctaccta
ccactaatatgcattctttagtaagcggtctcaatatacaccgattcttccttaactctgtttatgaagtattcagcatcctccctgcccccttcagcat
cctccctgcccctgagcacaggatccaatggcgtgaggaccacaggcctgggcagctgctggggcatacaggcatctcttagtggctgaga
gactgggccctggctctatgttggctcctaacttgctgccatttaaaggaaatcttagcctcccatccgtaaaatcgagaaaataagacttgtcct
acacagctcatgaaatagtaatgaaattcacattagagaagagatggaaaaacactttgaacaaaaagcattttgctcttataaaagcacagcct
cttttgagaggccctttgctccccatttctccttcttcagacccccccagactaggagaaggtctgtctcatggagtgaccttttggctgcctctag
attccaagctcagttttgctttcattaaccacagatactgggacggacagaaaaagacctagtttctgttgagccaaagagtctcataacttgtctg
ttcacatacccaagagcccaccctctagttgagacactcagttccctctcattctgggagactgcatgtctctgtgacctcctggtagagaccgtt
tgacatgtcccccaaccccccagtgattgagtctgaattctccactgatgacgcatttcctagcactcagggtgtcccctcctggttgccccctca
ccactgaagcccgcttcctcccttttcatttgatgcttaacaactgtcagtttgcaagaaacatgcttcaaatccacattctcccagttgcctagcaa
caacttccctcccggataaatgtgggtttcctgtagctcagcccaggactgaacacagcagcacacacttctgtccactgcttcaactgcttttca
cctctggtctgcatgccttcaagactgcagctcatccctcccttcagaaccttccatagcctgcagaggccatgtctgccccaaaaagacacatt
gaacctgaggctacttatttacccttgtgttaggtatatcctcaacttagaaattaatactgtttccagattgtcttctttgaatcacagaaagtaaaac
aacaaaacattcaatgcttaagacatttcatgtgcggttgggtgacatctgtttgatgaacacatttgatccaaagcatcagaaatactatgccaa
caagactttttaggaggtgataaacatgtctgttctaccttaagaaaaaaatattacacagtcccaagggagagacatggttttgatcccagaca
acccaagcagagacctcttagggccggaatcatcttggctgctgcctaggaccttatatcaatttcttaagcacaggatcaaggcctaaaggcc
ccttagactgacctcagttagtagaggcagatcccttcacagccttatcttccttagaggtctagtctgaccttgaacttcggctggcagtgctgtc
agttgtgatgtgtgacatggaagagttatttgttacttggaaaattaagagaacttatttggcataggaaattgtgtgtgtgtgtgtgtgtgtgtgtgt
gtgtgtgtgtgtgtgtgtgtgagatgatgtttgccattttgatctgtgacttttttttccagaaatagtttctcagttccattccaactaaacttacagtct
cttccggttctttgacagaaacaattcatgtgaatttgaacagataatagggaagggggaaccaaaagaagaggagagccctgggaaagttat
tttataatttatggcaacctcagtcaggcaactgtgaacaggtacatatggagggctccctcgggactaggcagtattcagagatgtaaggtgt
gaggaccggaccctcatcatttaccattcccactaaaaagagctgggaaggaaattgtagctgtagcaccaggcacgtaactggagcttagta
actatttggtgaaggaatattattaaattattaacaagatggaaaaaagggtattaaccacacaaaaatacatctcaagctattgtttctctgttccct
ttcccccaaattcctagtcttgctcttatctggctgtctctctagtcactctttcttgctgactctcttcacgttcctttctccacctggaattcctgggcc
ctccccttttactgacagacactgtcctcactctcacagtcatcagtttgtctctttacaaacctcagctcaagtgtcacttccccgtccccaggtga
aactgactgctccctccctgtaagtcaccatgatgactgctatatatagccctcatggaacctaaaacctcaacagacacagtctctttcctactct
gttatagtttatttactcattaattaccacaacacgtattattgagcacctactgtgtaccatgcccagaagataaaagacaaacaaaataaaacct
attcctatgcttaatgagtttacagtctagtggagagatagatacattaaaaaataacagcaaaccaaaataaaagtggtaaataaatgcactga
gaaagacaggaatagctaggaggggcacctaatccctagggaaggaaagctggaagagcatggtgatgggggaagaaggctttctggag
aaggtgaggtagtttgaaatgagttgactctggccagtaggggtagagtgagaatggggtgagacagggtgggttggtcattttgatccattag
tcctcaaagtgataggactagtggctaaggactgcaggctttacagaagcctacaaaactatttgagatttgaagtttttttttttttttaattggctcc
aaaagaaaatgaaaaaactttagaattataatgaatgaatattaaatgaatatttaaggaaggtaattttattcaacttcattgttaaatttagttaaaa
caagcccttgagtttcattcaacactgttttatcataccgttgatgagagaaaacaaaactgattcctggccagggccactgtcagcgtggggttt
gcacatctttcccatgtctgcttgggttagctccaggtactcctgtttcccccacatccccaagatgtgcccattagtggaaacggtgtgtctgcat
gattccaacgtgagtgagtgtgggtgtgggagtgagtgcccctgccatgggagggcatcctgtccaggttagattcctaccttgtgccctgag
ctgctgggatggaatccagccacccatgactctgaactgaaataattgggtgaataattatcttactttttaattaatctttgaaaatgtatgtatagtt
cacatgtatttcaatatttaatattagaagtattttagtctttattttgaagtttggtgatttattgtaaccagaaacaagctatagaaacttaattttgggc
caagtgcagtggctcacacctataatcccagcattttgggaggccgaggcagacgcatcacttgaggtccggagttcaagatcagcctggcc
aacatggtaaaaccctgtctctactaaaaaatacaaaaattagccagatgtggtgggcacctgtagtcccagctacttgggtggctgaagcag
gagaatcacttgaacccgggaggcggagcagtgagcagagatcgtgccactgcactcccacctaggcgacagtgtgacactccatctcaaa
aaaaaaaaaaaatagaaaagaaagaaacttaattctggtttatatcaattagcctgtggtaaaattggtttcattatagccatttcacttagttgaagt
ttccaataacctgtggatgaattaagtgaggatttactatattcataaaatcttaaattccaaagcctgtttgcagttcaggtttttccactttacaaac
acttctaagtattcacaatgattgcttaaaattcataccagataaatcattaaataagttgttcaaagtcaaataatttcataagtaaaaattaggagc
ttttagaaaactatacctacatagacctagacctatagatagacagagatctgaatagatatggacacagatgctttccaaagtgttcatgtgatgt
gtggtggagtttcaagaccagagtgtgcctggggcctgcagaagtaaaggagaggggatggagagaagattgtccacatggccatgggca
atctcccacccacactcaagtgaggaagacaggaaacaaattcagaaagaagagaaaataatcaaaactgatgggagcttgtgactgattta
cttatgcgcagcctccctggagacatgagtgtggctgttccttaggttgtgcctctgggctcctaccccctcttagatgccttcctattatctagga
cctggttgctttttgtctgcatagcttctttggattccagtctttgatgccagcttcctcctaaagtagcctttcagatgtcccttggttaccctctgcta
tctaagggctcatcctaccccacactcattcccagcaccaatttctggatctccaggctggagatttagacaatgggatgggaagaacccatga
tgggtcccagacagaaagtggtgccagccacagaaagggcacacaggcacagaagttggtttggggtaagacgatgtggtcagttcagaa
cacgctggatctaggcagatgcccagcagacagttggatatgtaagtctgaagctctggggagaggtctaggttggaggtacagatttagaa
gtcatcaacaaaaaggtagcagattaaatgataaaggaaatgagactatc

In some aspects, an engineered guide RNA comprises an RNA editing entity recruiting domain. An RNA editing entity can be recruited by an RNA editing entity recruiting domain on an engineered guide RNA. In some cases, a subject engineered guide RNA is configured to facilitate editing of a base of a nucleotide of a polynucleotide of a region of a subject target RNA, modulation expression of a polypeptide encoded by the subject target RNA, or both. In some cases, an engineered guide RNA can be configured to facilitate an editing of a base of a nucleotide or polynucleotide of a region of an RNA by a subject RNA editing entity. In order to facilitate editing, an engineered guide RNA of the disclosure may recruit an RNA editing entity. In certain embodiments, an engineered guide RNA lacks an RNA editing entity recruiting domain. Either way, a subject engineered guide RNA can be capable of binding an RNA editing entity, or be bound by it, and facilitate editing of a subject target RNA.

Various RNA editing entity recruiting domains can be utilized. In an embodiment, a recruiting domain comprises: Glutamate ionotropic receptor AMPA type subunit 2 (GluR2), APOBEC, MS2-bacteriophage-coat-protein-recruiting domain, Alu, a TALEN recruiting domain, a Zn-finger polypeptide recruiting domain, a mega-TAL recruiting domain, or a Cas13 recruiting domain, combinations thereof, or modified versions thereof. In certain embodiments, more than one recruiting domain can be included in an engineered guide RNA of the disclosure. In cases where a recruiting sequence is present, the recruiting sequence can be utilized to position the RNA editing entity to effectively react with a subject target RNA after the targeting sequence, for example an antisense sequence, hybridizes to a target RNA. In some cases, a recruiting sequence can allow for transient binding of the RNA editing entity to the engineered guide RNA. In other cases, the recruiting sequence allows for permanent binding of the RNA editing entity to the polynucleotide. A recruiting sequence can be of any length. In some cases, a recruiting sequence is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length. In some cases, a recruiting sequence is about 45 nucleotides in length. In some cases, at least a portion of a recruiting sequence comprises at least 1 to about 75 nucleotides. In some cases, at least a portion of a recruiting sequence comprises about 45 nucleotides to about 60 nucleotides. In some cases, at least a portion of a recruiting sequence comprises at least 1 to about 500 nucleotides.

In some embodiments, an RNA editing entity recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some embodiments, a recruiting domain comprises a GluR2 sequence, or a sequence having at least about 80%, 85%, 90%, 95%, 98%, 99%, or 100% identity to: GUGGAAUAGUAUAACAAUAUGCUAAAUGUUGUUAUAGUAUCCCAC (SEQ ID NO: 3). In some cases, a recruiting domain can comprise at least about 80% sequence homology to at least about 10, 15, 20, 25, or 30 nucleotides of SEQ ID NO: 3. In some embodiments, a recruiting domain can comprise at least about 90%, 95%, 96%, 97%, 98%, or 99% sequence homology to SEQ ID NO: 3.

Additional, RNA editing entity recruiting domains are described herein. In some instances, a recruiting domain comprises an apolipoprotein B mRNA editing entity, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an MS2-bacteriophage-coat-protein-recruiting domain. In another embodiment, a recruiting domain can be from an Alu domain. In some cases, a recruiting domain can comprise at least about: 80%, 85%, 90%, or 95% sequence homology to at least about: 15, 20, 25, 30, or 35 nucleotides of an APOBEC, MS2-bacteriophage-coat-protein-recruiting domain, or Alu domain.

Any number of recruiting sequences can be found in a polynucleotide of the present disclosure. In some cases, at least about 1, 2, 3, 4, 5, 6, 7, 8, 9, or up to about 10 recruiting sequences may be included in a polynucleotide. Recruiting sequences can be located at any position of subject polynucleotides. In some cases, a recruiting sequence is on an N-terminus, middle, or C-terminus of a polynucleotide. A recruiting sequence can be upstream or downstream of a targeting sequence. In some cases, a recruiting sequence flanks a targeting sequence of a subject polynucleotide. A recruiting sequence can comprise all ribonucleotides or deoxyribonucleotides, although a recruiting sequence comprising both ribo- and deoxyribonucleotides is in some cases not excluded.

In cases where a recruiting sequence is absent, an engineered polynucleotide may be capable of associating with a subject RNA editing entity (e.g., ADAR) to facilitate editing of a target RNA and/or modulate expression of a polypeptide encoded by a subject target RNA. This may be achieved through structural features. Structural features may comprise any one of a: mismatch, symmetrical bulge, asymmetrical bulge, symmetrical internal loop, asymmetrical internal loop, hairpins, wobble base pairs, a structured motif, circularized RNA, chemical modification, or any combination thereof. In an aspect, a double stranded RNA (dsRNA) substrate, for example hybridized polynucleotide strands, can be formed upon hybridization of an engineered polynucleotide of the present disclosure to a target RNA. Described herein is a feature, which corresponds to one of several structural features that may be present in a dsRNA substrate of the present disclosure. Examples of features include a mismatch, a bulge (symmetrical bulge or asymmetrical bulge), an internal loop (symmetrical internal loop or asymmetrical internal loop), or a hairpin (a hairpin comprising a non-targeting domain). Engineered polynucleotides of the present disclosure may have from 1 to 50 features. Engineered polynucleotides of the present disclosure may have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features.

As disclosed herein, a structured motif comprises two or more features in a dsRNA substrate.

A double stranded RNA (dsRNA) substrate may be formed upon hybridization of an engineered guide RNA of the present disclosure to a target RNA. As disclosed herein, a mismatch may refer to a nucleotide in a guide RNA that is unpaired to an opposing nucleotide in a target RNA within the dsRNA. A mismatch can comprise any two nucleotides that do not base pair, are not complementary, or both. In some embodiments, a mismatch is an A/C mismatch. An A/C mismatch may comprise a C in an engineered guide RNA of the present disclosure opposite an A in a target RNA. An A/C mismatch may comprise an A in an engineered guide RNA of the present disclosure opposite an C in a target RNA. In an embodiment, a G/G mismatch may comprise a G in an engineered guide RNA of the present disclosure opposite a G in a target RNA. In some embodiments, a mismatch positioned 5′ of the edit site may facilitate base-flipping of the target A to be edited. A mismatch may also help confer sequence specificity. In an embodiment, a mismatch comprises a G/G mismatch. In an embodiment, a mismatch comprises an A/C mismatch, wherein the A is in the target RNA and the C is in the targeting sequence of the engineered polynucleotide. In another embodiment, the A in the A/C mismatch is the base of the nucleotide in the target RNA edited by a subject RNA editing entity.

In some embodiments, a engineered guide RNA and a target RNA can have at least one mismatch. In some embodiments, a engineered guide RNA and a target RNA can have one mismatch. In some embodiments, an engineered guide RNA and a target RNA can have an A/C, A/G, U/C, U/G, C/A, C/U, G/A, G/U mismatch, or any combination thereof. In some embodiments, an engineered guide RNA and a target RNA can have a A/C mismatch. In some embodiments, an A in an A/C, A/G, C/A, or G/A can be modified by the RNA editing entity. In other cases, a C in an A/C, C/A, C/U. or U/C can be modified by the RNA editing entity. In other cases, a U in a U/C, C/U, G/U. or U/G can be modified by the RNA editing entity. In other cases, a G in a U/G, G/U, G/A. or A/G can be modified by the RNA editing entity. In some embodiments, an A in an A/C mismatch can be modified by the RNA editing entity. Such modifications can comprise any modification, for example chemical modifications induced by any RNA editing entity described herein and thereof. In an embodiment, a modification reverts a mismatch in a target RNA to a residue present in an otherwise comparable WT RNA.

Some embodiments relate to an engineered guide RNA comprising a mismatch. In some embodiments, the engineered guide RNA comprises an unmodified nucleotide on a first side (e.g. a side that is 5′ to the mismatch, or a side that is 3′ to the mismatch) of the nucleotide that forms the mismatch. In some embodiments, the engineered guide RNA comprises an unmodified nucleotide on either side of the nucleotide that forms the mismatch. In some embodiments, the engineered guide RNA comprises an unmodified nucleotide on a first side of a nucleotide opposite the nucleotide that forms the mismatch (e.g. a side that is 5′ to the mismatch and opposite the mismatch, or a side that is 3′ to the mismatch and opposite the mismatch). In some embodiments, the engineered guide RNA comprises an unmodified nucleotide on either side of a nucleotide opposite the nucleotide that forms the mismatch.

In some aspects, an engineered guide RNA can be designed utilizing methods that comprise tiling. For example, an engineered guide RNA can be selected from a plurality of candidate engineered guide RNAs that have been tiled against a nucleic acid or polypeptide of a subject target RNA. In an embodiment, an engineered guide RNA can be selected from a group of engineered guide RNAs that have been tiled against a nucleic acid or polypeptide of a subject target RNA, such as APP, SNCA, and/or Tau. In some cases, tiling can comprise tiling engineered guide RNAs across regulatory elements of subject targets. In some cases, tiling can comprise tiling engineered guide RNAs across any one of: a poly(A) tail, a microRNA response element (MRE), an AU-rich element (ARE), 5′UTR, 3′UTR, or any combination thereof of subject target sequence.

In some aspects, the engineered guide RNA that is tiled against a target RNA or nucleic acid can be pooled for use in a method described herein. In some cases, engineered guide RNAs can be pooled for detecting a target in a single assay. The pooling of engineered guide RNAs that are tiled against a single target can enhance the detection of a target RNA using the methods described herein. The tiling for example, can be sequential along the target nucleic acid or target polypeptide. Sometimes, the tiling can be overlapping along the target nucleic acid or target RNA. In some instances, the tiling comprises gaps between the tiled engineered guide RNA along the target nucleic acid or target RNA. In some instances, the tiling of an engineered guide RNA can be non-sequential. Often, a method for detecting a target nucleic acid and/or target RNA can comprise contacting a target nucleic acid or target RNA to a pool of engineered guide RNAs and an RNA editing entity and/or nuclease, wherein an engineered guide RNA of the pool of engineered guide RNAs comprises a targeting sequence to a sequence of a target; and assaying for editing.

In an aspect, a structural feature can form in an engineered polynucleotide independently. In other cases, a structural feature can form when an engineered polynucleotide binds to a target RNA. A structural feature can also form when an engineered polynucleotide associates with other molecules such as a peptide, a nucleotide, or a small molecule. In certain embodiments, a structural feature of an engineered polynucleotide can be formed independent of a target RNA, and its structure can change as a result of the engineered polypeptide hybridization with a target RNA region. In certain embodiments, a structural feature is present when an engineered polynucleotide is in association with a target RNA.

In some cases, a structural feature is a hairpin. In some cases, an engineered polynucleotide can lack a hairpin domain. In other cases, an engineered polynucleotide can contain a hairpin domain or more than one hairpin domain. A hairpin can be located anywhere in a polynucleotide. As disclosed herein, a hairpin may be an RNA duplex wherein a single RNA strand has folded in upon itself to form the RNA duplex. The single RNA strand folds upon itself due to having nucleotide sequences upstream and downstream of the folding region base pairs to each other. A hairpin may have from 10 to 500 nucleotides in length of the entire duplex structure. The stem-loop structure of a hairpin may be from 3 to 15 nucleotides long. A hairpin may be present in any of the engineered polynucleotides disclosed herein. The engineered polynucleotides disclosed herein may have from 1 to 10 hairpins. In some embodiments, the engineered polynucleotides disclosed herein have 1 hairpin. In some embodiments, the engineered polynucleotides disclosed herein have 2 hairpins. As disclosed herein, a hairpin may refer to a recruitment hairpin or a hairpin or a non-recruitment hairpin. A hairpin can be located anywhere within the engineered polynucleotides of the present disclosure. In some embodiments, one or more hairpins is present at the 3′ end of an engineered polynucleotide of the present disclosure, at the 5′ end of an engineered polynucleotide of the present disclosure or within the targeting sequence of an engineered polynucleotide of the present disclosure, or any combination thereof.

In aspect, a structural feature comprises a recruitment hairpin, as disclosed herein. A recruitment hairpin may recruit an RNA editing entity, such as ADAR. In some embodiments, a recruitment hairpin comprises a GluR2 domain. In some embodiments, a recruitment hairpin comprises an Alu domain.

In yet another aspect, a structural feature comprises a non-recruitment hairpin. A non-recruitment hairpin, as disclosed herein, may exhibit functionality that improves localization of the engineered polynucleotide to the target RNA. In some embodiments, the non-recruitment hairpin improves nuclear retention. In some embodiments, the non-recruitment hairpin comprises a hairpin from U7 snRNA.

In another aspect, a structural feature comprises a wobble base. A wobble base pair may refer to two bases that weakly pair. For example, a wobble base pair of the present disclosure may refer to a G paired with a U.

A hairpin of the present disclosure can be of any length. In an aspect, a hairpin can be from about 5-200 or more nucleotides. In some cases, a hairpin can comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 or more nucleotides. In other cases, a hairpin can also comprise 5 to 10, 5 to 20, 5 to 30, 5 to 40, 5 to 50, 5 to 60, 5 to 70, 5 to 80, 5 to 90, 5 to 100, 5 to 110, 5 to 120, 5 to 130, 5 to 140, 5 to 150, 5 to 160, 5 to 170, 5 to 180, 5 to 190, 5 to 200, 5 to 210, 5 to 220, 5 to 230, 5 to 240, 5 to 250, 5 to 260, 5 to 270, 5 to 280, 5 to 290, 5 to 300, 5 to 310, 5 to 320, 5 to 330, 5 to 340, 5 to 350, 5 to 360, 5 to 370, 5 to 380, 5 to 390, or 5 to 400 nucleotides. A hairpin may be a structural feature formed from a single strand of RNA with sufficient complementarity to itself to hybridize into a double stranded RNA motif/structure consisting of double-stranded hybridized RNA separated by a nucleotide loop.

In some cases, a structural feature may be a bulge. A bulge can comprise a single (intentional) nucleic acid mismatch between the target strand and an engineered polynucleotide strand. In other cases, more than one consecutive mismatch between strands constitutes a bulge as long as the bulge region, mismatched stretch of bases, may be flanked on both sides with hybridized, complementary dsRNA regions. A bulge can be located at any location of a polynucleotide. In some cases, a bulge may be located from about 30 to about 70 nucleotides from a 5′ hydroxyl or the 3′ hydroxyl.

In an embodiment, a double stranded RNA (dsRNA) substrate may be formed upon hybridization of an engineered polynucleotide of the present disclosure to a target RNA. As disclosed herein, a bulge may refer to the structure formed upon formation of the dsRNA substrate, where nucleotides in either the engineered polynucleotide or the target RNA are not complementary to their positional counterparts on the opposite strand. A bulge may change the secondary or tertiary structure of the dsRNA substrate. A bulge may have from 1 to 4 nucleotides on the engineered polynucleotide side of the dsRNA substrate or the target RNA side of the dsRNA substrate. In some embodiments, the engineered polynucleotides of the present disclosure have 2 bulges. In some embodiments, the engineered polynucleotides of the present disclosure have 3 bulges. In some embodiments, the engineered polynucleotides of the present disclosure have 4 bulges. In some embodiments, the presence of a bulge in a dsRNA substrate may position ADAR to selectively edit the target A in the target RNA and reduce off-target editing of non-targets. In some embodiments, the presence of a bulge in a dsRNA substrate may recruit additional ADAR. Bulges in dsRNA substrates disclosed herein may recruit other proteins, such as other RNA editing entities. In some embodiments, a bulge positioned 5′ of the edit site may facilitate base-flipping of the target A to be edited. A bulge may also help confer sequence specificity. A bulge may help direct ADAR editing by constraining it in an orientation that yield selective editing of the target A. In some embodiments, selective editing of the target A is achieved by positioning the target A between two bulges (e.g., positioned between a 5′ end bulge and a 3′ end bulge, based on the engineered polynucleotide). In some embodiments, the two bulges are both symmetrical bulges. In some embodiments, the two bulges each are formed by 2 nucleotides on the engineered polynucleotide side of the dsRNA target and 2 nucleotides on the target RNA side of the dsRNA substrate. In some embodiments, the two bulges each are formed by 3 nucleotides on the engineered polynucleotide side of the dsRNA target and 3 nucleotides on the target RNA side of the dsRNA substrate. In some embodiments, the two bulges each are formed by 4 nucleotides on the engineered polynucleotide side of the dsRNA target and 4 nucleotides on the target RNA side of the dsRNA substrate. In some embodiments, the target A is position between the two bulges, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a bulge (e.g., from a 5′end bulge or a 3′ end bulge). In some embodiments, additional structural features are located between the bulges (e.g., between the 5′end bulge and the 3′end bulge). In some embodiments, a mismatch in a bulge comprises a nucleotide base for editing in the target RNA (e.g., an A/C mismatch in the bulge, wherein part of the bulge in the engineered polynucleotide comprises a C mismatched to an A in the part of the bulge in the target RNA, and the A is edited).

In an aspect, a double stranded RNA (dsRNA) substrate may be formed upon hybridization of an engineered polynucleotide of the present disclosure to a target RNA. A bulge may be a symmetrical bulge or an asymmetrical bulge. A bulge may be formed by 1 to 4 participating nucleotides on either the guide RNA side or the target RNA side of the dsRNA substrate. A symmetrical bulge may be formed when the same number of nucleotides may be present on each side of the bulge. A symmetrical bulge may have from 2 to 4 nucleotides on the engineered polynucleotide side of the dsRNA substrate or the target RNA side of the dsRNA substrate. For example, a symmetrical bulge in a dsRNA substrate of the present disclosure may have the same number of nucleotides on the engineered polynucleotide side and the target RNA side of the dsRNA substrate. A symmetrical bulge of the present disclosure may be formed by 2 nucleotides on the engineered polynucleotide side of the dsRNA target and 2 nucleotides on the target RNA side of the dsRNA substrate. A symmetrical bulge of the present disclosure may be formed by 3 nucleotides on the engineered polynucleotide side of the dsRNA target and 3 nucleotides on the target RNA side of the dsRNA substrate. A symmetrical bulge of the present disclosure may be formed by 4 nucleotides on the engineered polynucleotide side of the dsRNA target and 4 nucleotides on the target RNA side of the dsRNA substrate.

A double stranded RNA (dsRNA) substrate may be formed upon hybridization of an engineered guide RNA of the present disclosure to a target RNA. A bulge may be a symmetrical bulge or an asymmetrical bulge. An asymmetrical bulge may be formed when a different number of nucleotides may be present on each side of the bulge. An asymmetrical bulge may have from 1 to 4 participating nucleotides on either the guide RNA side or the target RNA side of the dsRNA substrate. For example, an asymmetrical bulge in a dsRNA substrate of the present disclosure may have different numbers of nucleotides on the engineered guide RNA side and the target RNA side of the dsRNA substrate. An asymmetrical bulge of the present disclosure may be formed by 0 nucleotides on the engineered guide RNA side of the dsRNA substrate and 1 nucleotide on the target RNA side of the dsRNA substrate. An asymmetrical bulge of the present disclosure may be formed by 0 nucleotides on the target RNA side of the dsRNA substrate and 1 nucleotide on the engineered guide RNA side of the dsRNA substrate. An asymmetrical bulge of the present disclosure may be formed by 0 nucleotides on the engineered guide RNA side of the dsRNA substrate and 2 nucleotides on the target RNA side of the dsRNA substrate. An asymmetrical bulge of the present disclosure may be formed by 0 nucleotides on the target RNA side of the dsRNA substrate and 2 nucleotides on the engineered guide RNA side of the dsRNA substrate. An asymmetrical bulge of the present disclosure may be formed by 0 nucleotides on the engineered guide RNA side of the dsRNA substrate and 3 nucleotides on the target RNA side of the dsRNA substrate. An asymmetrical bulge of the present disclosure may be formed by 0 nucleotides on the target RNA side of the dsRNA substrate and 3 nucleotides on the engineered guide RNA side of the dsRNA substrate. An asymmetrical bulge of the present disclosure may be formed by 0 nucleotides on the engineered guide RNA side of the dsRNA substrate and 4 nucleotides on the target RNA side of the dsRNA substrate. An asymmetrical bulge of the present disclosure may be formed by 0 nucleotides on the target RNA side of the dsRNA substrate and 4 nucleotides on the engineered guide RNA side of the dsRNA substrate. An asymmetrical bulge of the present disclosure may be formed by 1 nucleotide on the engineered guide RNA side of the dsRNA substrate and 2 nucleotides on the target RNA side of the dsRNA substrate. An asymmetrical bulge of the present disclosure may be formed by 1 nucleotide on the target RNA side of the dsRNA substrate and 2 nucleotides on the engineered guide RNA side of the dsRNA substrate. An asymmetrical bulge of the present disclosure may be formed by 1 nucleotide on the engineered guide RNA side of the dsRNA substrate and 3 nucleotides on the target RNA side of the dsRNA substrate. An asymmetrical bulge of the present disclosure may be formed by 1 nucleotide on the target RNA side of the dsRNA substrate and 3 nucleotides on the engineered guide RNA side of the dsRNA substrate. An asymmetrical bulge of the present disclosure may be formed by 1 nucleotide on the engineered guide RNA side of the dsRNA substrate and 4 nucleotides on the target RNA side of the dsRNA substrate. An asymmetrical bulge of the present disclosure may be formed by 1 nucleotide on the target RNA side of the dsRNA substrate and 4 nucleotides on the engineered guide RNA side of the dsRNA substrate. An asymmetrical bulge of the present disclosure may be formed by 2 nucleotides on the engineered guide RNA side of the dsRNA substrate and 3 nucleotides on the target RNA side of the dsRNA substrate. An asymmetrical bulge of the present disclosure may be formed by 2 nucleotides on the target RNA side of the dsRNA substrate and 3 nucleotides on the engineered guide RNA side of the dsRNA substrate. An asymmetrical bulge of the present disclosure may be formed by 2 nucleotides on the engineered guide RNA side of the dsRNA substrate and 4 nucleotides on the target RNA side of the dsRNA substrate. An asymmetrical bulge of the present disclosure may be formed by 2 nucleotides on the target RNA side of the dsRNA substrate and 4 nucleotides on the engineered guide RNA side of the dsRNA substrate. An asymmetrical bulge of the present disclosure may be formed by 3 nucleotides on the engineered guide RNA side of the dsRNA substrate and 4 nucleotides on the target RNA side of the dsRNA substrate. An asymmetrical bulge of the present disclosure may be formed by 3 nucleotides on the target RNA side of the dsRNA substrate and 4 nucleotides on the engineered guide RNA side of the dsRNA substrate. In some embodiments, an asymmetrical bulge increases efficiency of editing a target A. In some embodiments, an asymmetrical bulge that increases efficiency of editing a target A is an asymmetrical bulge that is formed to reduce the number of adenosines in the sequence of the engineered polynucleotide. Non-limiting examples of an asymmetrical bulge that increases efficiency of editing a target A are an asymmetrical bulge formed by 0 nucleotides on the engineered polynucleotide side of the dsRNA substrate and 1 nucleotide on the target RNA side of the dsRNA substrate; an asymmetrical bulge of formed by 0 nucleotides on the engineered polynucleotide side of the dsRNA substrate and 2 nucleotides on the target RNA side of the dsRNA substrate; an asymmetrical bulge of formed by 0 nucleotides on the engineered polynucleotide side of the dsRNA substrate and 3 nucleotides on the target RNA side of the dsRNA substrate; an asymmetrical bulge of formed by 0 nucleotides on the engineered polynucleotide side of the dsRNA substrate and 4 nucleotides on the target RNA side of the dsRNA substrate; an asymmetrical bulge of formed by 1 nucleotide on the engineered polynucleotide side of the dsRNA substrate and 2 nucleotides on the target RNA side of the dsRNA substrate; an asymmetrical bulge of formed by 1 nucleotide on the engineered polynucleotide side of the dsRNA substrate and 3 nucleotides on the target RNA side of the dsRNA substrate; an asymmetrical bulge of formed by 1 nucleotide on the engineered polynucleotide side of the dsRNA substrate and 4 nucleotides on the target RNA side of the dsRNA substrate; an asymmetrical bulge of formed by 2 nucleotides on the engineered polynucleotide side of the dsRNA substrate and 3 nucleotides on the target RNA side of the dsRNA substrate; an asymmetrical bulge of formed by 2 nucleotides on the engineered polynucleotide side of the dsRNA substrate and 4 nucleotides on the target RNA side of the dsRNA substrate; and an asymmetrical bulge of formed by 3 nucleotides on the engineered polynucleotide side of the dsRNA substrate and 4 nucleotides on the target RNA side of the dsRNA substrate.

In some cases, a structural feature may be a loop. A loop may be comprised of 2 or more nucleotides (nts) that do not hybridize, but instead, loops may be flanked on both sides by hybridized RNA, causing a distended “loop” of non-hybridized nucleotides to be pushed out. In a case of an engineered guide RNA hybridized to target mRNA, a loop may be formed by one of the two strands, altering the editing landscape. Loops may be also formed at the ends of hairpins, in which the loop may be flanked on both sides with hybridized RNA from a single strand of RNA. Such loop structures can arise from: artificial RNA sequences capable of forming a loop structure; artificial sequences capable of forming a loop structure; or RNA sequences taken from known substrate RNAs for editing entities resident in the cell.

In an aspect, a double stranded RNA (dsRNA) substrate may be formed upon hybridization of an engineered polynucleotide of the present disclosure to a target RNA. As disclosed herein, an internal loop may refer to the structure formed upon formation of the dsRNA substrate, where nucleotides in either the engineered polynucleotide or the target RNA are not complementary to their positional counterparts on the opposite strand and where one side of the internal loop, either on the target RNA side or the engineered polynucleotide side of the dsRNA substrate, has greater than 5 nucleotides. Internal loops present in the vicinity of the edit site may help with base flipping of the target A in the target RNA to be edited. A double stranded RNA (dsRNA) substrate is formed upon hybridization of an engineered polynucleotide of the present disclosure to a target RNA. An internal loop may be a symmetrical internal loop or an asymmetrical internal loop. In some embodiments, selective editing of the target A is achieved by positioning the target A between two loops (e.g., positioned between a 5′ end loop and a 3′ end loop, based on the engineered polynucleotide). In some embodiments, the two loops are both symmetrical loops. In some embodiments, the two loops each are formed by 5 nucleotides on the engineered polynucleotide side of the dsRNA target and 5 nucleotides on the target RNA side of the dsRNA substrate. In some embodiments, the two loops each are formed by 6 nucleotides on the engineered polynucleotide side of the dsRNA target and 6 nucleotides on the target RNA side of the dsRNA substrate. In some embodiments, the two loops each are formed by 7 nucleotides on the engineered polynucleotide side of the dsRNA target and 7 nucleotides on the target RNA side of the dsRNA substrate. In some embodiments, the two loops each are formed by 8 nucleotides on the engineered polynucleotide side of the dsRNA target and 8 nucleotides on the target RNA side of the dsRNA substrate. In some embodiments, the two loops each are formed by 9 nucleotides on the engineered polynucleotide side of the dsRNA target and 9 nucleotides on the target RNA side of the dsRNA substrate. In some embodiments, the two loops each are formed by 10 nucleotides on the engineered polynucleotide side of the dsRNA target and 10 nucleotides on the target RNA side of the dsRNA substrate. In some embodiments, the target A is position between the two loops, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a loop (e.g., from a 5′end loop or a 3′ end loop). In some embodiments, additional structural features are located between the loops (e.g., between the 5′end loop and the 3′end loop). In some embodiments, a mismatch in a loop comprises a nucleotide base for editing in the target RNA (e.g., an A/C mismatch in the loop, wherein part of the bulge in the engineered polynucleotide comprises a C mismatched to an A in the part of the loop in the target RNA, and the A is edited).

A symmetrical internal loop is formed when the same number of nucleotides is present on each side of the internal loop. For example, a symmetrical internal loop in a dsRNA substrate of the present disclosure may have the same number of nucleotides on the engineered polynucleotide side and the target RNA side of the dsRNA substrate. A symmetrical internal loop of the present disclosure may be formed by 5 nucleotides on the engineered polynucleotide side of the dsRNA target and 5 nucleotides on the target RNA side of the dsRNA substrate. A symmetrical internal loop of the present disclosure may be formed by 6 nucleotides on the engineered polynucleotide side of the dsRNA target and 6 nucleotides on the target RNA side of the dsRNA substrate. A symmetrical internal loop of the present disclosure may be formed by 7 nucleotides on the engineered polynucleotide side of the dsRNA target and 7 nucleotides on the target RNA side of the dsRNA substrate. A symmetrical internal loop of the present disclosure may be formed by 8 nucleotides on the engineered polynucleotide side of the dsRNA target and 8 nucleotides on the target RNA side of the dsRNA substrate. A symmetrical internal loop of the present disclosure may be formed by 9 nucleotides on the engineered polynucleotide side of the dsRNA target and 9 nucleotides on the target RNA side of the dsRNA substrate. A symmetrical internal loop of the present disclosure may be formed by 10 nucleotides on the engineered polynucleotide side of the dsRNA target and 10 nucleotides on the target RNA side of the dsRNA substrate. One side of the internal loop, either on the target RNA side or the engineered polynucleotide side of the dsRNA substrate, may be formed by from 5 to 150 nucleotides. One side of the internal loop may be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop may be formed by 5 nucleotides. One side of the internal loop may be formed by 10 nucleotides. One side of the internal loop may be formed by 15 nucleotides. One side of the internal loop may be formed by 20 nucleotides. One side of the internal loop may be formed by 25 nucleotides. One side of the internal loop may be formed by 30 nucleotides. One side of the internal loop may be formed by 35 nucleotides. One side of the internal loop may be formed by 40 nucleotides. One side of the internal loop may be formed by 45 nucleotides. One side of the internal loop may be formed by 50 nucleotides. One side of the internal loop may be formed by 55 nucleotides. One side of the internal loop may be formed by 60 nucleotides. One side of the internal loop may be formed by 65 nucleotides. One side of the internal loop may be formed by 70 nucleotides. One side of the internal loop may be formed by 75 nucleotides. One side of the internal loop may be formed by 80 nucleotides. One side of the internal loop may be formed by 85 nucleotides. One side of the internal loop may be formed by 90 nucleotides. One side of the internal loop may be formed by 95 nucleotides. One side of the internal loop may be formed by 100 nucleotides. One side of the internal loop may be formed by 110 nucleotides. One side of the internal loop may be formed by 120 nucleotides. One side of the internal loop may be formed by 130 nucleotides. One side of the internal loop may be formed by 140 nucleotides. One side of the internal loop may be formed by 150 nucleotides. One side of the internal loop may be formed by 200 nucleotides. One side of the internal loop may be formed by 250 nucleotides. One side of the internal loop may be formed by 300 nucleotides. One side of the internal loop may be formed by 350 nucleotides. One side of the internal loop may be formed by 400 nucleotides. One side of the internal loop may be formed by 450 nucleotides. One side of the internal loop may be formed by 500 nucleotides. One side of the internal loop may be formed by 600 nucleotides. One side of the internal loop may be formed by 700 nucleotides. One side of the internal loop may be formed by 800 nucleotides. One side of the internal loop may be formed by 900 nucleotides. One side of the internal loop may be formed by 1000 nucleotides. An internal loop may be a symmetrical internal loop or an asymmetrical internal loop. Internal loops present in the vicinity of the edit site may help with base flipping of the target A in the target RNA to be edited. A double stranded RNA (dsRNA) substrate is formed upon hybridization of an engineered polynucleotide of the present disclosure to a target RNA. An internal loop may be a symmetrical internal loop or an asymmetrical internal loop. A symmetrical internal loop is formed when the same number of nucleotides is present on each side of the internal loop. For example, a symmetrical internal loop in a dsRNA substrate of the present disclosure may have the same number of nucleotides on the engineered polynucleotide side and the target RNA side of the dsRNA substrate. A symmetrical internal loop of the present disclosure may be formed by from 5 to 150 nucleotides on the engineered polynucleotide side of the dsRNA target and from 5 to 150 nucleotides on the target RNA side of the dsRNA substrate, wherein the number of nucleotides is the same on the engineered side of the dsRNA target and the target RNA side of the dsRNA substrate. A symmetrical internal loop of the present disclosure may be formed by from 5 to 1000 nucleotides on the engineered polynucleotide side of the dsRNA target and from 5 to 1000 nucleotides on the target RNA side of the dsRNA substrate, wherein the number of nucleotides is the same on the engineered side of the dsRNA target and the target RNA side of the dsRNA substrate. A symmetrical internal loop of the present disclosure may be formed by 5 nucleotides on the engineered polynucleotide side of the dsRNA target and 5 nucleotides on the target RNA side of the dsRNA substrate. A symmetrical internal loop of the present disclosure may be formed by 6 nucleotides on the engineered polynucleotide side of the dsRNA target and 6 nucleotides on the target RNA side of the dsRNA substrate. A symmetrical internal loop of the present disclosure may be formed by 7 nucleotides on the engineered polynucleotide side of the dsRNA target and 7 nucleotides on the target RNA side of the dsRNA substrate. A symmetrical internal loop of the present disclosure may be formed by 8 nucleotides on the engineered polynucleotide side of the dsRNA target and 8 nucleotides on the target RNA side of the dsRNA substrate. A symmetrical internal loop of the present disclosure may be formed by 9 nucleotides on the engineered polynucleotide side of the dsRNA target and 9 nucleotides on the target RNA side of the dsRNA substrate. A symmetrical internal loop of the present disclosure may be formed by 10 nucleotides on the engineered polynucleotide side of the dsRNA target and 10 nucleotides on the target RNA side of the dsRNA substrate. A symmetrical internal loop of the present disclosure may be formed by 15 nucleotides on the engineered polynucleotide side of the dsRNA target and 15 nucleotides on the target RNA side of the dsRNA substrate. A symmetrical internal loop of the present disclosure may be formed by 20 nucleotides on the engineered polynucleotide side of the dsRNA target and 20 nucleotides on the target RNA side of the dsRNA substrate. A symmetrical internal loop of the present disclosure may be formed by 30 nucleotides on the engineered polynucleotide side of the dsRNA target and 30 nucleotides on the target RNA side of the dsRNA substrate. A symmetrical internal loop of the present disclosure may be formed by 40 nucleotides on the engineered polynucleotide side of the dsRNA target and 40 nucleotides on the target RNA side of the dsRNA substrate. A symmetrical internal loop of the present disclosure may be formed by 50 nucleotides on the engineered polynucleotide side of the dsRNA target and 50 nucleotides on the target RNA side of the dsRNA substrate. A symmetrical internal loop of the present disclosure may be formed by 60 nucleotides on the engineered polynucleotide side of the dsRNA target and 60 nucleotides on the target RNA side of the dsRNA substrate. A symmetrical internal loop of the present disclosure may be formed by 70 nucleotides on the engineered polynucleotide side of the dsRNA target and 70 nucleotides on the target RNA side of the dsRNA substrate. A symmetrical internal loop of the present disclosure may be formed by 80 nucleotides on the engineered polynucleotide side of the dsRNA target and 80 nucleotides on the target RNA side of the dsRNA substrate. A symmetrical internal loop of the present disclosure may be formed by 90 nucleotides on the engineered polynucleotide side of the dsRNA target and 90 nucleotides on the target RNA side of the dsRNA substrate. A symmetrical internal loop of the present disclosure may be formed by 100 nucleotides on the engineered polynucleotide side of the dsRNA target and 100 nucleotides on the target RNA side of the dsRNA substrate. A symmetrical internal loop of the present disclosure may be formed by 110 nucleotides on the engineered polynucleotide side of the dsRNA target and 110 nucleotides on the target RNA side of the dsRNA substrate. A symmetrical internal loop of the present disclosure may be formed by 120 nucleotides on the engineered polynucleotide side of the dsRNA target and 120 nucleotides on the target RNA side of the dsRNA substrate. A symmetrical internal loop of the present disclosure may be formed by 130 nucleotides on the engineered polynucleotide side of the dsRNA target and 130 nucleotides on the target RNA side of the dsRNA substrate. A symmetrical internal loop of the present disclosure may be formed by 140 nucleotides on the engineered polynucleotide side of the dsRNA target and 140 nucleotides on the target RNA side of the dsRNA substrate. A symmetrical internal loop of the present disclosure may be formed by 150 nucleotides on the engineered polynucleotide side of the dsRNA target and 150 nucleotides on the target RNA side of the dsRNA substrate. A symmetrical internal loop of the present disclosure may be formed by 200 nucleotides on the engineered polynucleotide side of the dsRNA target and 200 nucleotides on the target RNA side of the dsRNA substrate. A symmetrical internal loop of the present disclosure may be formed by 250 nucleotides on the engineered polynucleotide side of the dsRNA target and 250 nucleotides on the target RNA side of the dsRNA substrate. A symmetrical internal loop of the present disclosure may be formed by 300 nucleotides on the engineered polynucleotide side of the dsRNA target and 300 nucleotides on the target RNA side of the dsRNA substrate. A symmetrical internal loop of the present disclosure may be formed by 350 nucleotides on the engineered polynucleotide side of the dsRNA target and 350 nucleotides on the target RNA side of the dsRNA substrate. A symmetrical internal loop of the present disclosure may be formed by 400 nucleotides on the engineered polynucleotide side of the dsRNA target and 400 nucleotides on the target RNA side of the dsRNA substrate. A symmetrical internal loop of the present disclosure may be formed by 450 nucleotides on the engineered polynucleotide side of the dsRNA target and 450 nucleotides on the target RNA side of the dsRNA substrate. A symmetrical internal loop of the present disclosure may be formed by 500 nucleotides on the engineered polynucleotide side of the dsRNA target and 500 nucleotides on the target RNA side of the dsRNA substrate. A symmetrical internal loop of the present disclosure may be formed by 600 nucleotides on the engineered polynucleotide side of the dsRNA target and 600 nucleotides on the target RNA side of the dsRNA substrate. A symmetrical internal loop of the present disclosure may be formed by 700 nucleotides on the engineered polynucleotide side of the dsRNA target and 700 nucleotides on the target RNA side of the dsRNA substrate. A symmetrical internal loop of the present disclosure may be formed by 800 nucleotides on the engineered polynucleotide side of the dsRNA target and 800 nucleotides on the target RNA side of the dsRNA substrate. A symmetrical internal loop of the present disclosure may be formed by 900 nucleotides on the engineered polynucleotide side of the dsRNA target and 900 nucleotides on the target RNA side of the dsRNA substrate. A symmetrical internal loop of the present disclosure may be formed by 1000 nucleotides on the engineered polynucleotide side of the dsRNA target and 1000 nucleotides on the target RNA side of the dsRNA substrate.

In an aspect, a double stranded RNA (dsRNA) substrate is formed upon hybridization of an engineered polynucleotide of the present disclosure to a target RNA. An internal loop may be a symmetrical internal loop or an asymmetrical internal loop. An asymmetrical internal loop is formed when a different number of nucleotides is present on each side of the internal loop. For example, an asymmetrical internal loop in a dsRNA substrate of the present disclosure may have different numbers of nucleotides on the engineered polynucleotide side and the target RNA side of the dsRNA substrate. An asymmetrical internal loop of the present disclosure may be formed by from 5 to 150 nucleotides on the engineered polynucleotide side of the dsRNA substrate and from 5 to 150 nucleotides on the target RNA side of the dsRNA substrate, wherein the number of nucleotides is the different on the engineered side of the dsRNA target than the number of nucleotides on the target RNA side of the dsRNA substrate. An asymmetrical internal loop of the present disclosure may be formed by from 5 to 1000 nucleotides on the engineered polynucleotide side of the dsRNA substrate and from 5 to 1000 nucleotides on the target RNA side of the dsRNA substrate, wherein the number of nucleotides is the different on the engineered side of the dsRNA target than the number of nucleotides on the target RNA side of the dsRNA substrate. An asymmetrical internal loop of the present disclosure may be formed by 5 nucleotides on the engineered polynucleotide side of the dsRNA substrate and 6 nucleotides on the target RNA side of the dsRNA substrate. An asymmetrical internal loop of the present disclosure may be formed by 5 nucleotides on the target RNA side of the dsRNA substrate and 6 nucleotides on the engineered polynucleotide side of the dsRNA substrate. An asymmetrical internal loop of the present disclosure may be formed by 5 nucleotides on the engineered polynucleotide side of the dsRNA substrate and 7 nucleotides on the target RNA side of the dsRNA substrate. An asymmetrical internal loop of the present disclosure may be formed by 5 nucleotides on the target RNA side of the dsRNA substrate and 7 nucleotides on the engineered polynucleotide side of the dsRNA substrate. An asymmetrical internal loop of the present disclosure may be formed by 5 nucleotides on the engineered polynucleotide side of the dsRNA substrate and 8 nucleotides internal loop the target RNA side of the dsRNA substrate. An asymmetrical internal loop of the present disclosure may be formed by 5 nucleotides on the target RNA side of the dsRNA substrate and 8 nucleotides on the engineered polynucleotide side of the dsRNA substrate. An asymmetrical internal loop of the present disclosure may be formed by 5 nucleotides on the engineered polynucleotide side of the dsRNA substrate and 9 nucleotides internal loop the target RNA side of the dsRNA substrate. An asymmetrical internal loop of the present disclosure may be formed by 5 nucleotides on the target RNA side of the dsRNA substrate and 9 nucleotides on the engineered polynucleotide side of the dsRNA substrate. An asymmetrical internal loop of the present disclosure may be formed by 5 nucleotides on the engineered polynucleotide side of the dsRNA substrate and 10 nucleotides internal loop the target RNA side of the dsRNA substrate. An asymmetrical internal loop of the present disclosure may be formed by 5 nucleotides on the target RNA side of the dsRNA substrate and 10 nucleotides on the engineered polynucleotide side of the dsRNA substrate. An asymmetrical internal loop of the present disclosure may be formed by 6 nucleotides on the engineered polynucleotide side of the dsRNA substrate and 7 nucleotides internal loop the target RNA side of the dsRNA substrate. An asymmetrical internal loop of the present disclosure may be formed by 6 nucleotides on the target RNA side of the dsRNA substrate and 7 nucleotides on the engineered polynucleotide side of the dsRNA substrate. An asymmetrical internal loop of the present disclosure may be formed by 6 nucleotides on the engineered polynucleotide side of the dsRNA substrate and 8 nucleotides internal loop the target RNA side of the dsRNA substrate. An asymmetrical internal loop of the present disclosure may be formed by 6 nucleotides on the target RNA side of the dsRNA substrate and 8 nucleotides on the engineered polynucleotide side of the dsRNA substrate. An asymmetrical internal loop of the present disclosure may be formed by 6 nucleotides on the engineered polynucleotide side of the dsRNA substrate and 9 nucleotides internal loop the target RNA side of the dsRNA substrate. An asymmetrical internal loop of the present disclosure may be formed by 6 nucleotides on the target RNA side of the dsRNA substrate and 9 nucleotides on the engineered polynucleotide side of the dsRNA substrate. An asymmetrical internal loop of the present disclosure may be formed by 6 nucleotides on the engineered polynucleotide side of the dsRNA substrate and 10 nucleotides internal loop the target RNA side of the dsRNA substrate. An asymmetrical internal loop of the present disclosure may be formed by 6 nucleotides on the target RNA side of the dsRNA substrate and 10 nucleotides on the engineered polynucleotide side of the dsRNA substrate. An asymmetrical internal loop of the present disclosure may be formed by 7 nucleotides on the engineered polynucleotide side of the dsRNA substrate and 8 nucleotides internal loop the target RNA side of the dsRNA substrate. An asymmetrical internal loop of the present disclosure may be formed by 7 nucleotides on the target RNA side of the dsRNA substrate and 8 nucleotides on the engineered polynucleotide side of the dsRNA substrate. An asymmetrical internal loop of the present disclosure may be formed by 7 nucleotides on the engineered polynucleotide side of the dsRNA substrate and 9 nucleotides internal loop the target RNA side of the dsRNA substrate. An asymmetrical internal loop of the present disclosure may be formed by 7 nucleotides on the target RNA side of the dsRNA substrate and 9 nucleotides on the engineered polynucleotide side of the dsRNA substrate. An asymmetrical internal loop of the present disclosure may be formed by 7 nucleotides on the engineered polynucleotide side of the dsRNA substrate and 10 nucleotides internal loop the target RNA side of the dsRNA substrate. An asymmetrical internal loop of the present disclosure may be formed by 7 nucleotides on the target RNA side of the dsRNA substrate and 10 nucleotides on the engineered polynucleotide side of the dsRNA substrate. An asymmetrical internal loop of the present disclosure may be formed by 8 nucleotides on the engineered polynucleotide side of the dsRNA substrate and 9 nucleotides internal loop the target RNA side of the dsRNA substrate. An asymmetrical internal loop of the present disclosure may be formed by 8 nucleotides on the target RNA side of the dsRNA substrate and 9 nucleotides on the engineered polynucleotide side of the dsRNA substrate. An asymmetrical internal loop of the present disclosure may be formed by 8 nucleotides on the engineered polynucleotide side of the dsRNA substrate and 10 nucleotides internal loop the target RNA side of the dsRNA substrate. An asymmetrical internal loop of the present disclosure may be formed by 8 nucleotides on the target RNA side of the dsRNA substrate and 10 nucleotides on the engineered polynucleotide side of the dsRNA substrate. An asymmetrical internal loop of the present disclosure may be formed by 9 nucleotides on the engineered polynucleotide side of the dsRNA substrate and 10 nucleotides internal loop the target RNA side of the dsRNA substrate. An asymmetrical internal loop of the present disclosure may be formed by 9 nucleotides on the target RNA side of the dsRNA substrate and 10 nucleotides on the engineered polynucleotide side of the dsRNA substrate. An asymmetrical internal loop of the present disclosure may be formed by 5 nucleotides on the target RNA side of the dsRNA substrate and 50 nucleotides on the engineered polynucleotide side of the dsRNA substrate. An asymmetrical internal loop of the present disclosure may be formed by 5 nucleotides on the target RNA side of the dsRNA substrate and 100 nucleotides on the engineered polynucleotide side of the dsRNA substrate. An asymmetrical internal loop of the present disclosure may be formed by 5 nucleotides on the target RNA side of the dsRNA substrate and 150 nucleotides on the engineered polynucleotide side of the dsRNA substrate. An asymmetrical internal loop of the present disclosure may be formed by 5 nucleotides on the target RNA side of the dsRNA substrate and 200 nucleotides on the engineered polynucleotide side of the dsRNA substrate. An asymmetrical internal loop of the present disclosure may be formed by 5 nucleotides on the target RNA side of the dsRNA substrate and 300 nucleotides on the engineered polynucleotide side of the dsRNA substrate. An asymmetrical internal loop of the present disclosure may be formed by 5 nucleotides on the target RNA side of the dsRNA substrate and 400 nucleotides on the engineered polynucleotide side of the dsRNA substrate. An asymmetrical internal loop of the present disclosure may be formed by 5 nucleotides on the target RNA side of the dsRNA substrate and 500 nucleotides on the engineered polynucleotide side of the dsRNA substrate. An asymmetrical internal loop of the present disclosure may be formed by 5 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 5 nucleotides on the engineered polynucleotide side of the dsRNA substrate. An asymmetrical internal loop of the present disclosure may be formed by 500 nucleotides on the target RNA side of the dsRNA substrate and 5 nucleotides on the engineered polynucleotide side of the dsRNA substrate. An asymmetrical internal loop of the present disclosure may be formed by 400 nucleotides on the target RNA side of the dsRNA substrate and 5 nucleotides on the engineered polynucleotide side of the dsRNA substrate. An asymmetrical internal loop of the present disclosure may be formed by 300 nucleotides on the target RNA side of the dsRNA substrate and 5 nucleotides on the engineered polynucleotide side of the dsRNA substrate. An asymmetrical internal loop of the present disclosure may be formed by 200 nucleotides on the target RNA side of the dsRNA substrate and 5 nucleotides on the engineered polynucleotide side of the dsRNA substrate. An asymmetrical internal loop of the present disclosure may be formed by 150 nucleotides on the target RNA side of the dsRNA substrate and 5 nucleotides on the engineered polynucleotide side of the dsRNA substrate. An asymmetrical internal loop of the present disclosure may be formed by 100 nucleotides on the target RNA side of the dsRNA substrate and 5 nucleotides on the engineered polynucleotide side of the dsRNA substrate. An asymmetrical internal loop of the present disclosure may be formed by 50 nucleotides on the target RNA side of the dsRNA substrate and 5 nucleotides on the engineered polynucleotide side of the dsRNA substrate. An asymmetrical internal loop of the present disclosure may be formed by 50 nucleotides on the target RNA side of the dsRNA substrate and 100 nucleotides on the engineered polynucleotide side of the dsRNA substrate. An asymmetrical internal loop of the present disclosure may be formed by 50 nucleotides on the target RNA side of the dsRNA substrate and 150 nucleotides on the engineered polynucleotide side of the dsRNA substrate. An asymmetrical internal loop of the present disclosure may be formed by 50 nucleotides on the target RNA side of the dsRNA substrate and 200 nucleotides on the engineered polynucleotide side of the dsRNA substrate. An asymmetrical internal loop of the present disclosure may be formed by 50 nucleotides on the target RNA side of the dsRNA substrate and 300 nucleotides on the engineered polynucleotide side of the dsRNA substrate. An asymmetrical internal loop of the present disclosure may be formed by 50 nucleotides on the target RNA side of the dsRNA substrate and 400 nucleotides on the engineered polynucleotide side of the dsRNA substrate. An asymmetrical internal loop of the present disclosure may be formed by 50 nucleotides on the target RNA side of the dsRNA substrate and 500 nucleotides on the engineered polynucleotide side of the dsRNA substrate. An asymmetrical internal loop of the present disclosure may be formed by 50 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 50 nucleotides on the engineered polynucleotide side of the dsRNA substrate. An asymmetrical internal loop of the present disclosure may be formed by 500 nucleotides on the target RNA side of the dsRNA substrate and 50 nucleotides on the engineered polynucleotide side of the dsRNA substrate. An asymmetrical internal loop of the present disclosure may be formed by 400 nucleotides on the target RNA side of the dsRNA substrate and 50 nucleotides on the engineered polynucleotide side of the dsRNA substrate. An asymmetrical internal loop of the present disclosure may be formed by 300 nucleotides on the target RNA side of the dsRNA substrate and 50 nucleotides on the engineered polynucleotide side of the dsRNA substrate. An asymmetrical internal loop of the present disclosure may be formed by 200 nucleotides on the target RNA side of the dsRNA substrate and 50 nucleotides on the engineered polynucleotide side of the dsRNA substrate. An asymmetrical internal loop of the present disclosure may be formed by 150 nucleotides on the target RNA side of the dsRNA substrate and 50 nucleotides on the engineered polynucleotide side of the dsRNA substrate. An asymmetrical internal loop of the present disclosure may be formed by 100 nucleotides on the target RNA side of the dsRNA substrate and 50 nucleotides on the engineered polynucleotide side of the dsRNA substrate. An asymmetrical internal loop of the present disclosure may be formed by 100 nucleotides on the target RNA side of the dsRNA substrate and 150 nucleotides on the engineered polynucleotide side of the dsRNA substrate. An asymmetrical internal loop of the present disclosure may be formed by 100 nucleotides on the target RNA side of the dsRNA substrate and 200 nucleotides on the engineered polynucleotide side of the dsRNA substrate. An asymmetrical internal loop of the present disclosure may be formed by 100 nucleotides on the target RNA side of the dsRNA substrate and 300 nucleotides on the engineered polynucleotide side of the dsRNA substrate. An asymmetrical internal loop of the present disclosure may be formed by 100 nucleotides on the target RNA side of the dsRNA substrate and 400 nucleotides on the engineered polynucleotide side of the dsRNA substrate. An asymmetrical internal loop of the present disclosure may be formed by 100 nucleotides on the target RNA side of the dsRNA substrate and 500 nucleotides on the engineered polynucleotide side of the dsRNA substrate. An asymmetrical internal loop of the present disclosure may be formed by 100 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 100 nucleotides on the engineered polynucleotide side of the dsRNA substrate. An asymmetrical internal loop of the present disclosure may be formed by 500 nucleotides on the target RNA side of the dsRNA substrate and 100 nucleotides on the engineered polynucleotide side of the dsRNA substrate. An asymmetrical internal loop of the present disclosure may be formed by 400 nucleotides on the target RNA side of the dsRNA substrate and 100 nucleotides on the engineered polynucleotide side of the dsRNA substrate. An asymmetrical internal loop of the present disclosure may be formed by 300 nucleotides on the target RNA side of the dsRNA substrate and 100 nucleotides on the engineered polynucleotide side of the dsRNA substrate. An asymmetrical internal loop of the present disclosure may be formed by 200 nucleotides on the target RNA side of the dsRNA substrate and 100 nucleotides on the engineered polynucleotide side of the dsRNA substrate. An asymmetrical internal loop of the present disclosure may be formed by 150 nucleotides on the target RNA side of the dsRNA substrate and 100 nucleotides on the engineered polynucleotide side of the dsRNA substrate. An asymmetrical internal loop of the present disclosure may be formed by 150 nucleotides on the target RNA side of the dsRNA substrate and 200 nucleotides on the engineered polynucleotide side of the dsRNA substrate. An asymmetrical internal loop of the present disclosure may be formed by 150 nucleotides on the target RNA side of the dsRNA substrate and 300 nucleotides on the engineered polynucleotide side of the dsRNA substrate. An asymmetrical internal loop of the present disclosure may be formed by 150 nucleotides on the target RNA side of the dsRNA substrate and 400 nucleotides on the engineered polynucleotide side of the dsRNA substrate. An asymmetrical internal loop of the present disclosure may be formed by 150 nucleotides on the target RNA side of the dsRNA substrate and 500 nucleotides on the engineered polynucleotide side of the dsRNA substrate. An asymmetrical internal loop of the present disclosure may be formed by 150 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 150 nucleotides on the engineered polynucleotide side of the dsRNA substrate. An asymmetrical internal loop of the present disclosure may be formed by 500 nucleotides on the target RNA side of the dsRNA substrate and 5 nucleotides on the engineered polynucleotide side of the dsRNA substrate. An asymmetrical internal loop of the present disclosure may be formed by 400 nucleotides on the target RNA side of the dsRNA substrate and 150 nucleotides on the engineered polynucleotide side of the dsRNA substrate. An asymmetrical internal loop of the present disclosure may be formed by 300 nucleotides on the target RNA side of the dsRNA substrate and 150 nucleotides on the engineered polynucleotide side of the dsRNA substrate. An asymmetrical internal loop of the present disclosure may be formed by 200 nucleotides on the target RNA side of the dsRNA substrate and 300 nucleotides on the engineered polynucleotide side of the dsRNA substrate. An asymmetrical internal loop of the present disclosure may be formed by 200 nucleotides on the target RNA side of the dsRNA substrate and 400 nucleotides on the engineered polynucleotide side of the dsRNA substrate. An asymmetrical internal loop of the present disclosure may be formed by 200 nucleotides on the target RNA side of the dsRNA substrate and 500 nucleotides on the engineered polynucleotide side of the dsRNA substrate. An asymmetrical internal loop of the present disclosure may be formed by 200 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 200 nucleotides on the engineered polynucleotide side of the dsRNA substrate. An asymmetrical internal loop of the present disclosure may be formed by 500 nucleotides on the target RNA side of the dsRNA substrate and 200 nucleotides on the engineered polynucleotide side of the dsRNA substrate. An asymmetrical internal loop of the present disclosure may be formed by 400 nucleotides on the target RNA side of the dsRNA substrate and 200 nucleotides on the engineered polynucleotide side of the dsRNA substrate. An asymmetrical internal loop of the present disclosure may be formed by 300 nucleotides on the target RNA side of the dsRNA substrate and 200 nucleotides on the engineered polynucleotide side of the dsRNA substrate. An asymmetrical internal loop of the present disclosure may be formed by 300 nucleotides on the target RNA side of the dsRNA substrate and 400 nucleotides on the engineered polynucleotide side of the dsRNA substrate. An asymmetrical internal loop of the present disclosure may be formed by 300 nucleotides on the target RNA side of the dsRNA substrate and 500 nucleotides on the engineered polynucleotide side of the dsRNA substrate. An asymmetrical internal loop of the present disclosure may be formed by 300 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 300 nucleotides on the engineered polynucleotide side of the dsRNA substrate. An asymmetrical internal loop of the present disclosure may be formed by 500 nucleotides on the target RNA side of the dsRNA substrate and 300 nucleotides on the engineered polynucleotide side of the dsRNA substrate. An asymmetrical internal loop of the present disclosure may be formed by 400 nucleotides on the target RNA side of the dsRNA substrate and 300 nucleotides on the engineered polynucleotide side of the dsRNA substrate. An asymmetrical internal loop of the present disclosure may be formed by 400 nucleotides on the target RNA side of the dsRNA substrate and 500 nucleotides on the engineered polynucleotide side of the dsRNA substrate. An asymmetrical internal loop of the present disclosure may be formed by 400 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 400 nucleotides on the engineered polynucleotide side of the dsRNA substrate. An asymmetrical internal loop of the present disclosure may be formed by 500 nucleotides on the target RNA side of the dsRNA substrate and 400 nucleotides on the engineered polynucleotide side of the dsRNA substrate. An asymmetrical internal loop of the present disclosure may be formed by 500 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 500 nucleotides on the engineered polynucleotide side of the dsRNA substrate. In some embodiments, an asymmetrical loop increases efficiency of editing a target A. In some embodiments, an asymmetrical loop that increases efficiency of editing a target A is an asymmetrical bulge that is formed to reduce the number of adenosines in the sequence of the engineered polynucleotide. Non-limiting examples of an asymmetrical loop that increases efficiency of editing a target A are an asymmetrical loop formed by 5 nucleotides on the engineered polynucleotide side of the dsRNA substrate and 20 nucleotide on the target RNA side of the dsRNA substrate; an asymmetrical bulge of formed by 10 nucleotides on the engineered polynucleotide side of the dsRNA substrate and 50 nucleotides on the target RNA side of the dsRNA substrate; an asymmetrical bulge of formed by 60 nucleotides on the engineered polynucleotide side of the dsRNA substrate and 80 nucleotides on the target RNA side of the dsRNA substrate; an asymmetrical bulge of formed by 18 nucleotides on the engineered polynucleotide side of the dsRNA substrate and 24 nucleotides on the target RNA side of the dsRNA substrate; an asymmetrical bulge of formed by 100 nucleotide on the engineered polynucleotide side of the dsRNA substrate and 150 nucleotides on the target RNA side of the dsRNA substrate; an asymmetrical bulge of formed by 70 nucleotide on the engineered polynucleotide side of the dsRNA substrate and 75 nucleotides on the target RNA side of the dsRNA substrate; an asymmetrical bulge of formed by 8 nucleotide on the engineered polynucleotide side of the dsRNA substrate and 15 nucleotides on the target RNA side of the dsRNA substrate; an asymmetrical bulge of formed by 45 nucleotides on the engineered polynucleotide side of the dsRNA substrate and 46 nucleotides on the target RNA side of the dsRNA substrate; an asymmetrical bulge of formed by 45 nucleotides on the engineered polynucleotide side of the dsRNA substrate and 50 nucleotides on the target RNA side of the dsRNA substrate; and an asymmetrical bulge of formed by 7 nucleotides on the engineered polynucleotide side of the dsRNA substrate and 15 nucleotides on the target RNA side of the dsRNA substrate.

Structural features that comprise a bulge or loop can be of any size. In some cases, a bulge or loop comprise at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 600, 700, 800, 900, or 1000 bases. In some cases, a bulge or loop comprise at least about 1-10, 5-15, 10-20, 15-25, 20-30, 1-30, 1-40, 1-50, 1-60, 1-70, 1-80, 1-90, 1-100, 1-110, 1-120, 1-130, 1-140, 1-150, 1-200, 1-250, 1-300, 1-350, 1-400, 1-450, 1-500, 1-600, 1-700, 1-800, 1-900, 1-1000, 20-50, 20-60, 20-70, 20-80, 20-90, 20-100, 20-110, 20-120, 20-130, 20-140, 20-150, 1-200, 1-250, 1-300, 1-350, 1-400, 1-450, 1-500, 1-600, 1-700, 1-800, 1-900, 1-1000, 30-40, 30-50, 30-60, 30-70, 30-80, 30-90, 30-100, 30-110, 30-120, 30-130, 30-140, 30-150, 30-200, 30-250, 30-300, 30-350, 30-400, 30-450, 30-500, 30-600, 30-700, 30-800, 30-900, 30-1000, 40-50, 40-60, 40-70, 40-80, 40-90, 40-100, 40-110, 40-120, 40-130, 40-140, 40-150, 40-200, 40-250, 40-300, 40-350, 40-400, 40-450, 40-500, 40-600, 40-700, 40-800, 40-900, 40-1000, 50-60, 50-70, 50-80, 50-90, 50-100, 50-110, 50-120, 50-130, 50-140, 50-150, 50-200, 50-250, 50-300, 50-350, 50-400, 50-450, 50-500, 50-600, 50-700, 50-800, 50-900, 50-1000, 60-70, 60-80, 60-90, 60-100, 60-110, 60-120, 60-130, 60-140, 60-150, 60-200, 60-250, 60-300, 60-350, 60-400, 60-450, 60-500, 60-600, 60-700, 60-800, 60-900, 60-1000, 70-80, 70-90, 70-100, 70-110, 70-120, 70-130, 70-140, 70-150, 70-200, 70-250, 70-300, 70-350, 70-400, 70-450, 70-500, 70-600, 70-700, 70-800, 70-900, 70-1000, 80-90, 80-100, 80-110, 80-120, 80-130, 80-140, 80-150, 80-200, 80-250, 80-300, 80-350, 80-400, 80-450, 80-500, 80-600, 80-700, 80-800, 80-900, 80-1000, 90-100, 90-110, 90-120, 90-130, 90-140, 90-150, 90-200, 90-250, 90-300, 90-350, 90-400, 90-450, 90-500, 90-600, 90-700, 90-800, 90-900, 90-1000, 100-110, 100-120, 100-130, 100-140, 100-150, 100-200, 100-250, 100-300, 100-350, 100-400, 100-450, 100-500, 100-600, 100-700, 100-800, 100-900, 100-1000, 110-120, 110-130, 110-140, 110-150, 110-200, 110-250, 110-300, 110-350, 110-400, 110-450, 110-500, 110-600, 110-700, 110-800, 110-900, 110-1000, 120-130, 120-140, 120-150, 120-200, 120-250, 120-300, 120-350, 120-400, 120-450, 120-500, 120-600, 120-700, 120-800, 120-900, 120-1000, 130-140, 130-150, 130-200, 130-250, 130-300, 130-350, 130-400, 130-450, 130-500, 130-600, 130-700, 130-800, 130-900, 130-1000, 140-150, 140-200, 140-250, 140-300, 140-350, 140-400, 140-450, 140-500, 140-600, 140-700, 140-800, 140-900, 140-1000, 150-200, 150-250, 150-300, 150-350, 150-400, 150-450, 150-500, 150-600, 150-700, 150-800, 150-900, 150-1000, 200-250, 200-300, 200-350, 200-400, 200-450, 200-500, 200-600, 200-700, 200-800, 200-900, 200-1000, 250-300, 250-350, 250-400, 250-450, 250-500, 250-600, 250-700, 250-800, 250-900, 250-1000, 300-350, 300-400, 300-450, 300-500, 300-600, 300-700, 300-800, 300-900, 300-1000, 350-400, 350-450, 350-500, 350-600, 350-700, 350-800, 350-900, 350-1000, 400-450, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, or 900-1000 bases in total.

In some cases, a structural feature may be a structured motif. As disclosed herein, a structured motif comprises two or more structural features in a dsRNA substrate. A structured motif can comprise of any combination of structural features, such as in the above claims, to generate an ideal substrate for ADAR editing at a precise location(s). These structural motifs could be artificially engineered to maximized ADAR editing, and/or these structural motifs can be modeled to recapitulate known ADAR substrates.

In some cases, a polynucleotide provided herein can be circularized or in a circular configuration. In some aspects, an at least partially circular polynucleotide lacks a 5′ hydroxyl or a 3′ hydroxyl.

In some embodiments, an engineered guide RNA can comprise a backbone comprising a plurality of sugar and phosphate moieties covalently linked together. In some cases, a backbone of an engineered guide RNA can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered guide RNA can lack a 5′ reducing hydroxyl, a 3′ reducing hydroxyl, or both, capable of being exposed to a solvent. In some embodiments, a backbone of an engineered guide RNA can lack a 5′ reducing hydroxyl, a 3′ reducing hydroxyl, or both, capable of being exposed to nucleases. In some embodiments, a backbone of an engineered guide RNA can lack a 5′ reducing hydroxyl, a 3′ reducing hydroxyl, or both, capable of being exposed to hydrolytic enzymes. In some instances, a backbone of an engineered guide RNA can be represented as a polynucleotide sequence in a circular 2-dimensional format with one nucleotide after the other. In some instances, a backbone of an engineered guide RNA can be represented as a polynucleotide sequence in a looped 2-dimensional format with one nucleotide after the other. In some cases, a 5′ hydroxyl, a 3′ hydroxyl, or both, may be joined through a phosphorus-oxygen bond. In some cases, a 5′ hydroxyl, a 3′ hydroxyl, or both, may be modified into a phosphoester with a phosphorus-containing moiety.

The engineered guide RNA described herein can have any frequency of bases. For example, a polynucleotide can have a percent adenine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent cytosine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent thymine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%1, 1-5%, 3-8%, 5-12%0, 10-15%, 8-20%, 15-25%, 20-30%, 25-35%, or up to about 30-40%. A polynucleotide can have a percent guanine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent uracil of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%.

In some cases, the engineered guide RNA can undergo quality control after a modification. In some cases, quality control may include PAGE, HPLC, MS, or any combination thereof. In some cases, a mass of a polynucleotide can be determined. A mass can be determined by LC-MS assay. A mass can be 30,000 amu, 50,000 amu, 70,000 amu, 90,000 amu, 100,000 amu, 120,000 amu, 150,000 amu, 175,000 amu, 200,000 amu, 250,000 amu, 300,000 amu, 350,000 amu, 400,000 amu, to about 500,000 amu. A mass can be of a sodium salt of a polynucleotide.

In some cases, an endotoxin level of a polynucleotide can be determined. A clinically/therapeutically acceptable level of an endotoxin can be less than 3 EU/mL. A clinically/therapeutically acceptable level of an endotoxin can be less than 10 EU/mL. A clinically/therapeutically acceptable level of an endotoxin can be less than 8 EU/mL. A clinically/therapeutically acceptable level of an endotoxin can be less than 5 EU/mL. A clinically/therapeutically acceptable level of an endotoxin can be less than 4 EU/mL. A clinically/therapeutically acceptable level of an endotoxin can be less than 3 EU/mL. A clinically/therapeutically acceptable level of an endotoxin can be less than 2 EU/mL. A clinically/therapeutically acceptable level of an endotoxin can be less than 1 EU/mL. A clinically/therapeutically acceptable level of an endotoxin can be less than 0.5 EU/mL.

In some cases, the engineered guide RNA can undergo sterility testing. A clinically/therapeutically acceptable level of a sterility testing can be 0 or denoted by no growth on a culture. A clinically/therapeutically acceptable level of a sterility testing can be less than 0.5% growth. A clinically/therapeutically acceptable level of a sterility testing can be less than 1% growth.

In some cases, any one of the engineered guide RNAs that comprise recruiting sequences may also comprise structural features described herein.

Also provided are linear engineered guide RNAs. The linear engineered guide RNAs can substantially lack structural features provided herein. For example, a linear polynucleotide can lack a structural feature or can have less than about 2 structural features or partial structures. A partial structure can comprise a portion of the bases required to achieve a structural feature as described herein. In some instances, the linear engineered guide RNA, upon binding to the target nucleic acid (e.g., target RNA) forms at least one structure feature described herein. In some cases, the forming of the at least one structure feature recruits the nucleic acid editing entity (e.g., RNA editing entity) to the target nucleic acid for editing of the target nucleic acid. In other cases, a linear engineered guide RNA can comprise any one of: 5′ hydroxyl, a 3′ hydroxyl, or both. Any one of these can be capable of being exposed to solvent and maintain linearization.

In some embodiments, the engineered guide RNA can comprise an engineered scaffold. In some embodiments, the engineered scaffold can be a structural loop stabilized scaffold. In some instances, the structural loop stabilized scaffold comprises nucleic acid structures such as RNA structures. In some embodiments, the structural loop stabilized scaffold comprises secondary RNA structure, tertiary RNA structure, quaternary structure, or a combination thereof. In some cases, the structural loop stabilized scaffold comprises structures of an RNA aptamer. In some instances, the structural loop stabilized scaffold comprises structures of any RNA species (e.g. ribosomal RNA, regulatory RNA, or tRNA). In some instances, the structural loop stabilized scaffold comprises a secondary structure comprising: an acceptor stem composed of a plurality of ribonucleotides of the 5′ end of the ribonucleotide chain and the plurality of ribonucleotides preceding the last 4 ribonucleotides of the 3′ end of the ribonucleotide chain, thus forming a double-stranded structure comprising a plurality of pairs of ribonucleotides. In some cases, it the ribonucleotides constituted by the ribonucleotide of the 5′ end of the ribonucleotide chain and the ribonucleotide that precedes the last 4 ribonucleotides of the 3′ end of the ribonucleotide chain can be unpaired. The structural loop stabilized scaffold can further comprise a secondary structure comprising a D arm comprising a plurality of pairs of ribonucleotides and a D loop comprising 1 to 100 ribonucleotides, formed by folding of a part of the ribonucleotide chain that follows the plurality of ribonucleotides of the 5′ end of the ribonucleotide chain. In some embodiments, the structural loop stabilized scaffold comprises a secondary structure comprising a stem that can be an equivalent of an anticodon region of a tRNA and a loop of the anticodon region of the tRNA (stem-loop of the anticodon), formed by the folding of a part of the ribonucleotide chain that follows the D arm and the D loop. In some embodiments, the structural loop stabilized scaffold comprises a secondary structure comprising a variable loop constituted by from 1 to 100 ribonucleotides and formed by a part of the ribonucleotide chain that follows the stem of the anticodon and the loop of the anticodon. In some embodiments, the structural loop stabilized scaffold comprises a secondary structure comprising) a T arm comprising a plurality of pairs of ribonucleotides, and a T loop comprising 1 to 100 ribonucleotides, formed by the folding of a part of the ribonucleotide chain that follows the variable loop and precedes the ribonucleotides of the 3′ end of the ribonucleotide chain of the acceptor stem. In some embodiments, the scaffold described herein comprises a tRNA scaffold, where the structures of the tRNA can be incorporated into the engineered guide RNA described herein.

In some embodiments, an engineered guide RNA comprising the engineered scaffold can be circular or looped scaffold. In some cases, an engineered guide RNA comprising the scaffold can be a loop. In some instances, a circular shape can comprise a loop shape. In some instances, a loop shape can comprise a circular shape. Loop formation or circularization can prevent exposed ends of a polynucleotide from being degraded and can significantly increase the half-life of a polynucleotide, in vivo, ex vivo, or in vitro. In some embodiments, a circular or looped engineered guide RNA can prevent one or more exposed ends from hydrolytic degradation. In some instances, a circular or looped engineered guide RNA can significantly increase a half-life of the polynucleotide as compared to a comparable polynucleotide that may not be circular or may not be a loop. In some embodiments, forming a circular or looped engineered guide RNA can significantly increase a half-life of an engineered guide RNA (e.g., guide RNA) when delivered in vivo, as compared to a comparable engineered guide RNA (e.g. guide RNA) that is in some cases not circular or not a loop. In some cases, forming a circular or looped engineered guide RNA can significantly reduce an amount (such as a therapeutically effective amount) of the engineered guide RNA dosed to a subject, as compared to a comparable engineered guide RNA that can be not circular or not a loop. In some embodiments, forming a circular or looped engineered guide RNA can significantly enhance efficiency of editing, can significantly reduce off-target editing, enhance efficiency of recruiting an RNA editing entity, or a combination thereof, as compared to a comparable engineered guide RNA that may not be circular or may not be a loop. In some cases, a circular or looped engineered guide RNA can significantly increase the synthesis of the engineered guide RNA as compared to a comparable engineered guide RNA that can be not circular or not a loop. In some embodiments, a circular or looped engineered guide RNA can significantly increase the transport of the engineered guide RNA into a cell, as compared to a comparable engineered guide RNA that can be not circular or not a loop. In some cases, a circular or looped engineered guide RNA can significantly increase the intracellular retention of the engineered guide RNA, as compared to a comparable engineered guide RNA that can be not circular or not a loop.

In one aspect, the scaffold comprises a tRNA scaffold. In one aspect, the SLS scaffold comprises a tRNA comprising an engineered guide RNA (including a targeting sequence) inserted into the anticodon region, wherein the engineered targeting sequence binds to at least a part of the target RNA. In one aspect, the tRNA may be an endogenous tRNA with a modified anticodon stem region recognizing the codon in the target RNA comprising a mutation. In one aspect, the SLS may be a tRNA scaffold that cannot be charged with an amino acid. In some embodiments, the tRNA may be an orthogonal tRNA charged with a non-canonical amino acid. In one aspect, the engineered polynucleotide may be administered along with a corresponding tRNA synthetase. In some embodiments, the corresponding synthetase may be E. coli Glutaminyl-tRNA synthetase. In some embodiments involving an orthogonal tRNA, the non-canonical amino acid may be pyrrolysine.

In one aspect, the engineered scaffold comprises at least one sequence that has at least 50%, 55%, 60%, 65%, 70%, 75%, 80%, 85%, 90%, 92%, 95% or 99% similarity to a tRNA sequence that forms a T loop secondary structure. In one aspect, the SLS scaffold comprises at least one sequence that has no more than 50%, 55%, 60%, 65%, 70%, 75%, 80%, 85%, 90%, 92%, 95% or 99% similarity to a tRNA sequence that forms a T loop secondary structure. In one aspect, the SLS scaffold comprises at least one sequence that has at least 50%, 55%, 60%, 65%, 70%, 75%, 80%, 85%, 90%, 92%, 95% or 99% similarity to a tRNA sequence that forms a D loop secondary structure. In one aspect, the SLS scaffold comprises at least one sequence that has no more than 50%, 55%, 60%, 65%, 70%, 75%, 80%, 85%, 90%, 92%, 95% or 99% similarity to a tRNA sequence that forms a D loop secondary structure. In one aspect, the SLS scaffold comprises at least one sequence that has at least 50%, 55%, 60%, 65%, 70%, 75%, 80%, 85%, 90%, 92%, 95% or 99% similarity to a tRNA sequence that forms an anticodon loop secondary structure. In one aspect, the SLS scaffold comprises at least one sequence that has no more than 50%, 55%, 60%, 65%, 70%, 75%, 80%, 85%, 90%, 92%, 95% or 99% similarity to a tRNA sequence that forms an anticodon loop secondary structure. In one aspect, the SLS scaffold comprises at least one sequence that has at least 50%, 55%, 60%, 65%, 70%, 75%, 80%, 85%, 90%, 92%, 95% or 99% similarity to a tRNA sequence that forms a tRNA variable arm secondary structure. In one aspect, the SLS scaffold comprises at least one sequence that has no more than 50%, 55%, 60%, 65%, 70%, 75%, 80%, 85%, 90%, 92%, 95% or 99% similarity to a tRNA sequence that forms a tRNA variable arm secondary structure. In one aspect, the SLS scaffold comprises at least one sequence that has at least 50%, 55%, 60%, 65%, 70%, 75%, 80%, 85%, 90%, 92%, 95% or 99% similarity to a tRNA sequence that forms a tRNA acceptor stem secondary structure. In one aspect, the SLS scaffold comprises at least one sequence that has no more than 50%, 55%, 60%, 65%, 70%, 75%, 80%, 85%, 90%, 92%, 95% or 99% similarity to a tRNA sequence that forms a tRNA acceptor stem secondary structure. In one aspect, the SLS scaffold comprises at least one sequence that has at least 50%, 55%, 60%, 65%, 70%, 75%, 80%, 85%, 90%, 92%, 95% or 99% similarity to a tRNA sequence that forms a tRNA pseudoknot secondary structure in the acceptor arm. In one aspect, the SLS scaffold comprises at least one sequence that has no more than 50%, 55%, 60%, 65%, 70%, 75%, 80%, 85%, 90%, 92%, 95% or 99% similarity to a tRNA sequence that forms a tRNA pseudoknot secondary structure in the acceptor arm.

In one aspect, the SLS scaffold comprises a secondary structure. In one aspect, the secondary structure comprises an RNA editing entity recruiting domain. In one aspect, the SLS scaffold comprises a recruiting sequence for an RNA editing entity. In one aspect, the SLS scaffold comprises at least one stem-loop structure. In one aspect, the SLS scaffold stabilizes the binding of the targeting sequence to the target RNA.

A circular or looped engineered guide polynucleotide, such as an engineered guide RNA can be formed directly or indirectly by forming a linkage (such as a covalent linkage) between more than one end of a RNA sequence, such as a 5′ end and a 3′ end. An RNA sequence can comprise an engineered guide RNA (such as a recruiting domain, targeting domain, or both). A linkage can be formed by employing an enzyme, such as a ligase. A suitable ligase (or synthetase) can include a ligase that forms a covalent bond. A covalent bond can include a carbon-oxygen bond, a carbon-sulfur bond, a carbon-nitrogen bond, a carbon-carbon bond, a phosphoric ester bond, or any combination thereof. A linkage can also be formed by employing a recombinase. An enzyme can be recruited to an RNA sequence to form a linkage. A circular or looped RNA can be formed by ligating more than one end of an RNA sequence using a linkage element. In some embodiments, a linkage can be formed by a ligation reaction. In some instances, a linkage can be formed by a homologous recombination reaction. A linkage element can employ click chemistry to form a circular or looped RNA. A linkage element can be an azide-based linkage. A circular or looped RNA can be formed by genetically encoding or chemically synthesizing the circular or looped RNA. A circular or looped RNA can be formed by employing a self-cleaving enzyme, such as a ribozyme, tRNA, aptamer, catalytically active fragment of any of these, or any combination thereof. For example, a ribozyme, a tRNA, an aptamer, a catalytically active fragment of any of these, or any combination thereof can be added to a 3′ end, a 5′ end, or both of a precursor of the engineered RNA. In another example, a ribozyme, a tRNA, an aptamer, a catalytically active fragment of any of these, or any combination thereof can be added to a 3′ terminal end, a 5′ terminal end, or both of a precursor of the engineered RNA. A self-cleaving ribozyme can comprise, for example, an RNase P RNA a Hammerhead ribozyme (e.g., a Schistosoma mansoni ribozyme), a glmS ribozyme, an HDV-like ribozyme, an R2 element, or a group I intron. In some cases, the self-cleaving ribozyme can be a trans-acting ribozyme that joins one RNA end on which it may be present to a separate RNA end. In some cases, a self-cleaving element or an aptamer can be configured to facilitate self-circularization of an engineered guide RNA described herein.

A looped or circular engineered guide polynucleotide, such as an engineered guide RNA can provide various benefits as compared to a non-circular or non-looped guide polynucleotide. A looped or circular engineered guide RNA can provide greater stability, improved recruitment of RNA-editing entities (such as endogenous RNA editing entitys), longer half-lives, improved RNA-editing efficiency, or any combination thereof, as compared to a comparable engineered guide RNA that can be not circular or not a loop. A looped or circular engineered guide polynucleotide can provide one or more of these improved qualities and can retain genetic encodability as compared guide polynucleotides comprising other types of modifications designed to improve guide stability—such as chemical modifications or sugar additions. A looped or circular engineered guide polynucleotide lacking chemical modifications can be capable of being genetically encoded, capable of being delivered by a vector, and retain improved stability.

In some embodiments, RNA editing can be evaluated by determining by the percent RNA editing of a target RNA. In some cases, RNA editing can be determined by changes in a level of protein. In some cases, a level of a protein can be measured by a Western blot. In some cases, a level of a protein can be measured by densitometry with a quantitative protein gel. In some cases, the percent RNA editing of a target RNA can be determined at different time points (e.g. 24 hours, 48 hours, 96 hours) after transfection with an engineered guide RNA by reverse transcribing the target RNA to cDNA then using Sanger sequencing to determine the percent RNA editing of a target RNA. In some cases, the cDNA can be amplified prior to sequencing by polymerase chain reaction. Sanger traces from Sanger sequencing can be analyzed to assess the editing efficiency of guide RNAs. In some cases, next generation sequencing technologies (e.g. sequencing by synthesis) can be used to determine percent RNA editing of a target RNA. For example, RNA sequencing can be used to determine the percent RNA editing of a target RNA after transfection with a guide RNA or guide polynucleotide. In some instances, the individual sequencing reads can be analyzed to determine the percent RNA editing.

In some instances, chemical modifications to enhance guide stability, synthesis, localization, intracellular retention, or lengthen half-lives may not be genetically encodable. An engineered guide RNA can be circular, substantially circular, or otherwise linked in a contiguous fashion (e.g. can be arranged as a loop) and can also retain a substantially similar secondary structure as a substantially similar engineered guide RNA that may not be circular or may not be a loop. A circular or looped engineered guide RNA can be pre-strained.

Compositions and methods provided herein can be utilized to modulate expression of a target. Modulation can refer to altering the expression of a gene or portion thereof at one of various stages, with a view to alleviate a disease or condition associated with the gene or a mutation in the gene. Modulation can be mediated at the level of transcription or post-transcriptionally. Modulating transcription can correct aberrant expression of splice variants generated by a mutation in a gene. In some cases, compositions and methods provided herein can be utilized to regulate gene translation of a target. Modulation can refer to decreasing or knocking down the expression of a gene or portion thereof by decreasing the abundance of a transcript. The decreasing the abundance of a transcript can be mediated by decreasing the processing, splicing, turnover or stability of the transcript; or by decreasing the accessibility of the transcript by translational machinery such as ribosome. In some cases, an engineered guide RNA described herein can facilitate a knockdown. A knockdown can reduce the expression of a target RNA. In some cases, a knockdown can be accompanied by editing of an mRNA. In some cases, a knockdown can occur with substantially little to no editing of an mRNA. In some instances, a knockdown can occur by targeting an untranslated region of the target RNA, such as a 3′ UTR, a 5′ UTR or both. In some cases, a knockdown can occur by targeting a coding region of the target RNA. In some instances, a knockdown can be mediated by an RNA editing entity (e.g., ADAR). In some instances, an RNA editing entity can cause a knockdown by hydrolytic deamination of multiple adenosines in an RNA. Hydrolytic deamination of multiple adenosines in an RNA can be referred to as hyper-editing. In some cases, hyper-editing can occur in cis (e.g., in an Alu element) or in trans (e.g. in a target RNA by an engineered guide RNA).

In some embodiments, the engineered guide RNA, upon binding to the target RNA, can recruit RNA editing entity to edit the target RNA. In some embodiments, the RNA editing entity is: ADAR or APOBEC; a catalytically active fragment of ADAR or APOBEC; fusion polypeptide comprising ADAR or APOBEC or a catalytically active fragment of ADAR or APOBEC; or a combination thereof. In some embodiments, the RNA editing entity can comprise ADAR1, ADAR2, ADAR3 or a combination thereof. In some embodiments the RNA editing entity can be endogenous to a cell. In some embodiments the RNA editing entity can be exogenous (e.g., expressed from an exogenous vector).

In some embodiments, the engineered guide RNA can be fused to a targeting moiety for directing the engineered guide RNA to a specific cell type. For example, the engineered guide RNA can be fused to a targeting moiety comprising an antibody to direct the engineered guide RNA to a specific cell type. In some embodiments, the engineered guide RNA can be encapsulated in particles such as nanoparticles and liposomes. In some cases, the particles encapsulating the engineered guide RNA can comprise the targeting moiety. Exemplary types of cells that can be targeted by the targeting moiety includes a skin cell, a lung cell, a heart cell, an epithelial cell, a reproductive cell, an eye cell, a kidney cell, a liver cell, a pancreas cell, an intestinal cell, a muscle cell, a gland cell, an eye cell, a brain cell, or a blood cell. In some cases, a cell can comprise a neuron, a photoreceptor cell, a retinal pigment epithelium cell, a glia cell, a myoblast cell, a myotube cell, a hepatocyte, a lung epithelial cell, or a fibroblast cell. In some cases, a cell can be a stem cell, such as embryonic stem cells, pluripotent stem cells, or totipotent stem cells. In some instances, a cell can comprise a human cell. In some cases, a cell can comprise a leukocyte. In some embodiments, a cell can comprise a lymphocyte. In some instances, a cell can comprise a T-cell. In some case, a cell can comprise a helper CD4+ T-cell, a cytotoxic CD8+ T-cell, a memory T-cell, a regulatory CD4+ T-cell, a natural killer T-cell, a mucosal associated T-cell, a gamma delta T-cell, or any combination thereof. In some embodiments, a cell can comprise a B-cell. In some cases, a cell can comprise a plasmablast, a plasma cell, a lymphoplasmacytoid cell, a memory B-cell, a follicular B-cell, a marginal zone B-cell, a B-1 cell, a regulatory B cell, or any combination thereof. In some embodiments, the cell targeted by the targeting moiety can be neuronal cell, liver cell, or macular cell.

### Chemical Modification

An engineered guide RNA as described herein for use in treating a disease or condition in a subject comprises at least one chemical modification. In some embodiments, the engineered guide RNA comprises at least one, two, three, four, five, six, seven, eight, nine, ten, 11, 12, 13, 14, 15, 16, 17, 18, 19, 20, 30, 50, 100, or more chemical modifications.

Exemplary chemical modifications comprise any one of: 5′ adenylate, 5′ guanosine-triphosphate cap, 5′ N7-Methylguanosine-triphosphate cap, 5′ triphosphate cap, 3′ phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3phosphorothioate or any combinations thereof.

A chemical modification can be made at any location of the engineered guide RNA. In some cases, a modification may be located in a 5′ or 3′ end. In some cases, a polynucleotide comprises a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150. More than one modification can be made to the engineered guide RNA. In some cases, a modification can be permanent. In other cases, a modification can be transient. In some cases, multiple modifications may be made to the engineered guide RNA. the engineered guide RNA modification can alter physio-chemical properties of a nucleotide, such as their conformation, polarity, hydrophobicity, chemical reactivity, base-pairing interactions, or any combination thereof.

A chemical modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases may be of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In some embodiments, chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some embodiments, chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the engineered guide RNA comprises at least one chemically modified base. In some instances, the engineered guide RNA comprises 2, 3, 4, 5, 6, 7, 8, 9, 10, 15, 20, or more modified bases. In some cases, chemical modifications to the base moiety include natural and synthetic modifications of adenine, guanine, cytosine, thymine, or uracil, and purine or pyrimidine bases.

In some embodiments, the at least one chemical modification of the engineered guide RNA comprises a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; Replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Chemical modifications to the engineered guide RNA include any modification contained herein, while some exemplary modifications are recited in Table 2.

TABLE 2
Exemplary Chemical Modification

| Modification of | |
|---|---|
| engineered guide RNA | Examples |
| Modification of one or both | sulfur (S), selenium (Se), BR3 (wherein R can be, e.g., hydrogen, |
| of the non-linking | alkyl, or aryl), C (e.g., an alkyl group, an aryl group, and the like), |
| phosphate oxygens in the | H, NR2, wherein R can be, e.g., hydrogen, alkyl, or aryl, or |
| phosphodiester backbone | wherein R can be, e.g., alkyl or aryl |
| linkage | |
| Modification of one or more | sulfur (S), selenium (Se), BR3 (wherein R can be, e.g., hydrogen, |
| of the linking phosphate | alkyl, or aryl), C (e.g., an alkyl group, an aryl group, and the like), |
| oxygens in the | H, NR2, wherein R can be, e.g., hydrogen, alkyl, or aryl, or |
| phosphodiester backbone | wherein R can be, e.g., alkyl or aryl |
| linkage | |
| Replacement of the | methyl phosphonate, hydroxylamino, siloxane, carbonate, |
| phosphate moiety with | carboxymethyl, carbamate, amide, thioether, ethylene oxide linker, |
| “dephospho” linkers | sulfonate, sulfonamide, thioformacetal, formacetal, oxime, |
| | methyleneimino, methylenemethylimino, methylenehydrazo, |
| | methylenedimethylhydrazo, or methyleneoxymethylimino |
| Modification or | Nucleic acid analog (examples of nucleotide analogs can be found |
| replacement of a naturally | in PCT/US2015/025175, PCT/US2014/050423, |
| occurring nucleobase | PCT/US2016/067353, PCT/US2018/041503, PCT/US18/041509, |
| | PCT/US2004/011786, or PCT/US2004/011833, all of which are |
| | expressly incorporated by reference in their entireties |
| Modification of the ribose- | phosphorothioate, phosphonothioacetate, phosphoroselenates, |
| phosphate backbone | boranophosphates, borano phosphate esters, hydrogen |
| | phosphonates, phosphonocarboxylate, phosphoroamidates, alkyl or |
| | aryl phosphonates, phosphonoacetate, or phosphotriesters |
| Modification of 5′ end of | 5′ cap or modification of 5′ cap —OH |
| polynucleotide | |
| Modification of 3′ end of | 3′ tail or modification of 3′ end —OH |
| polynucleotide | |
| Modification of the | phosphorothioate, phosphonothioacetate, phosphoroselenates, |
| deoxyribose phosphate | borano phosphates, borano phosphate esters, hydrogen |
| backbone | phosphonates, phosphoroamidates, alkyl or aryl phosphonates, or |
| | phosphotriesters |
| Substitution of the | methyl phosphonate, hydroxylamino, siloxane, carbonate, |
| phosphate group | carboxymethyl, carbamate, amide, thioether, ethylene oxide linker, |
| | sulfonate, sulfonamide, thioformacetal, formacetal, oxime, |
| | methyleneimino, methylenemethylimino, methylenehydrazo, |
| | methylenedimethylhydrazo, or methyleneoxymethylimino. |
| Modification of the | morpholino, cyclobutyl, pyrrolidine, or peptide nucleic acid (PNA) |
| ribophosphate backbone | nucleoside surrogates |
| Modifications to the sugar | Locked nucleic acid (LNA), unlocked nucleic acid (UNA), or |
| of a nucleotide | bridged nucleic acid (BNA) |
| Modification of a | 2′-O-methyl, 2′-O-methoxy-ethyl (2′-MOE), 2′-fluoro, 2′- |
| constituent of the ribose | aminoethyl, 2′-deoxy-2′-fuloarabinou-cleic acid, 2′-deoxy, 2′-O- |
| sugar | methyl, 3′-phosphorothioate, 3′-phosphonoacetate (PACE), or 3′- |
| | phosphonothioacetate (thioPACE) |
| Modifications to the base of | Modification of A, T, C, G, or U |
| a nucleotide | |
| Stereopure of nucleotide | S conformation of phosphorothioate or R conformation of |
| | phosphorothioate |

### Modification of Phosphate Backbone

In some embodiments, the chemical modification comprises modification of one or both of the non-linking phosphate oxygens in the phosphodiester backbone linkage or modification of one or more of the linking phosphate oxygens in the phosphodiester backbone linkage. As used herein, “alkyl” may be meant to refer to a saturated hydrocarbon group which may be straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” may refer to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” may refer to an aliphatic group containing at least one double bond. As used herein, “alkynyl” may refer to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” may refer to an alkyl moiety in which an alkyl hydrogen atom may be replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” may refer to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” may refer to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” may refer to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way may be a stereogenic center. The stereogenic phosphorous atom can possess either the “R” configuration (herein Rp) or the “S” configuration (herein Sp). In some cases, the engineered guide RNA can comprise stereopure nucleotides comprising S conformation of phosphorothioate or R conformation of phosphorothioate. In some embodiments, the chiral phosphate product may be present in a diastereomeric excess of 50%, 60%, 70%, 80%, 90%, or more. In some embodiments, the chiral phosphate product may be present in a diastereomeric excess of 95%. In some embodiments, the chiral phosphate product may be present in a diastereomeric excess of 96%. In some embodiments, the chiral phosphate product may be present in a diastereomeric excess of 97%. In some embodiments, the chiral phosphate product may be present in a diastereomeric excess of 98%. In some embodiments, the chiral phosphate product may be present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) may be attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It may be also understood in a nucleotide substitute that both the sugar and the phosphate moieties of the nucleotide can be replaced, by for example an amide type linkage (aminoethylglycine) (PNA). It may be also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some embodiments, the chemical modification described herein comprises modification of a phosphate backbone. In some embodiments, the engineered guide RNA described herein comprises at least one chemically modified phosphate backbone. Exemplary chemically modification of the phosphate group or backbone can include replacing one or more of the oxygens with a different substituent. Furthermore, the modified nucleotide present in the engineered guide RNA can include the replacement of an unmodified phosphate moiety with a modified phosphate as described herein. In some embodiments, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group may be achiral. However, replacement of one of the non-bridging oxygens with one of the above atoms or groups of atoms can render the phosphorous atom chiral; that may be to say that a phosphorous atom in a phosphate group modified in this way may be a stereogenic center. The stereogenic phosphorous atom can possess either the “R” configuration (herein Rp) or the “S” configuration (herein Sp). In such case, the chemically modified engineered guide RNA can be stereopure (e.g. S or R confirmation). In some cases, the chemically modified engineered guide RNA comprises stereopure phosphate modification. For example, the chemically modified engineered guide RNA can comprise S conformation of phosphorothioate or R conformation of phosphorothioate.

Phosphorodithioates have both non-bridging oxygens replaced by sulfur. The phosphorus center in the phosphorodithioates may be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

he phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some embodiments, at least one phosphate group of the engineered guide RNA can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g. 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some embodiments, the chemical modification described herein comprises modification by replacement of a phosphate group. In some embodiments, the engineered guide RNA described herein comprises at least one chemically modification comprising a phosphate group substitution or replacement. Exemplary phosphate group replacement can include non-phosphorus containing connectors. In some embodiments, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some embodiments, the chemical modification described herein comprises modifying ribophosphate backbone of the engineered guide RNA. In some embodiments, the engineered guide RNA described herein comprises at least one chemically modified ribophosphate backbone. Exemplary chemically modified ribophosphate backbone can include scaffolds that can mimic nucleic acids can also be constructed wherein the phosphate linker and ribose sugar may be replaced by nuclease resistant nucleoside or nucleotide surrogates. In some embodiments, the nucleobases can be tethered by a surrogate backbone. Examples can include morpholino, cyclobutyl, pyrrolidine and peptide nucleic acid (PNA) nucleoside surrogates.

### Modification of Sugar

In some embodiments, the chemical modification described herein comprises modifying of sugar. In some embodiments, the engineered guide RNA described herein comprises at least one chemically modified sugar. Exemplary chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative). In some cases, the deoxy modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the Γ position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that may be in the L form, e.g. L-nucleosides. In some aspects, the engineered guide RNA described herein includes the sugar group ribose, which may be a 5-membered ring having an oxygen. Exemplary modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose may be replaced by glycol units attached to phosphodiester bonds), threose nucleic acid. In some embodiments, the modifications to the sugar of the engineered guide RNA comprises modifying the engineered guide RNA to include locked nucleic acid (LNA), unlocked nucleic acid (UNA), or bridged nucleic acid (BNA).

### Modification of a Constituent of the Ribose Sugar

In some embodiments, the engineered guide RNA described herein comprises at least one chemical modification of a constituent of the ribose sugar. In some embodiments, the chemical modification of the constituent of the ribose sugar can include 2′-O-methyl, 2′-O-methoxy-ethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position may be unsubstituted or substituted with a thio-tert-butyl group (SC(CH₃)₃) (and analogs thereof); a methyleneamino group (CH₂NH2) (and analogs thereof) or a cyano group (CN) (and analogs thereof).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R═H, C₁-C₁₂ alkyl or a protecting group); and combinations thereof.

In some instances, the engineered guide RNA described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O, S-, or N-alkenyl; O-, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙCH₃)]₂, where n and m may be from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₐ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)2-O-2′ (ENA); 4′-CH(CH₃)—O-2′ and 4′-CH(CH₂OCH₃)—O-2′, and analogs thereof, 4′-C(CH₃)(CH₃)—O-2′ and analogs thereof.

### Modifications on the Base of Nucleotide

In some embodiments, the chemical modification described herein comprises modification of the base of nucleotide (e.g. the nucleobase). Exemplary nucleobases can include adenine (A), thymine (T), guanine (G), cytosine (C), and uracil (U). These nucleobases can be modified or replaced to in the engineered guide RNA described herein. The nucleobase of the nucleotide can be independently selected from a purine, a pyrimidine, a purine or pyrimidine analog. In some embodiments, the nucleobase can be naturally-occurring or synthetic derivatives of a base.

In some embodiments, the chemical modification described herein comprises modifying an uracil. In some embodiments, the engineered guide RNA described herein comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, l-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethy])-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, l-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some embodiments, the chemical modification described herein comprises modifying a cytosine. In some embodiments, the engineered guide RNA described herein comprises at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N₄-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein comprises modifying a adenine. In some embodiments, the engineered guide RNA described herein comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′-O-dimethyl-adenosine, N6-Methyl-2′-deoxyadenosine, N6, N6, 2′-O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the chemical modification described herein comprises modifying a guanine. In some embodiments, the engineered guide RNA described herein comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undemriodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 6-O-phenyl-2′-deoxyinosine, 2′-O-ribosylguanosine, 1-thio-guanosine, 6-O-methyguanosine, O⁶-Methyl-2′-deoxyguanosine, 2′-F-ara-guanosine, and 2′-F-guanosine.

In some cases, the chemical modification of the engineered guide RNA can include introducing or substituting a nucleic acid analog or an unnatural nucleic acid into the engineered guide RNA. In some embodiments, nucleic acid analog can be any one of the chemically modified nucleic acid described herein. Exemplary nucleic acid analog can be found in PCT/US2015/025175, PCT/US2014/050423, PCT/US2016/067353, PCT/US2018/041503, PCT/US18/041509, PCT/US2004/011786, or PCT/US2004/011833, all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2,-O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl] carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some embodiments, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-tauri nomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

A modified base of a unnatural nucleic acid includes, but may be not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base may be replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification can comprise chemically modifying the 5′ or 3′ end such as 5′ cap or 3′ tail of the engineered guide RNA. In some embodiments, the engineered guide RNA can comprise a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyladenosine, can be incorporated into the gRNA. In some embodiments, sugar-modified ribonucleotides can be incorporated, e.g., wherein the 2′ OH-group may be replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

### Method of Delivery

Described herein, in some embodiments, are methods of delivering the engineered guide RNAs described herein to a cell. In some embodiments, the method comprises delivering directly or indirectly an engineered guide RNA to the cell. In some cases, the engineered guide RNA comprises at least one chemical modification, where a double stranded polynucleotide forms between at least a portion of the engineered guide RNA and a target RNA. In some instances, the double stranded polynucleotide comprises at least one structural feature for recruiting a RNA editing entity to the double stranded polynucleotide. The recruitment of the RNA editing entity results in the editing of the target RNA by the RNA editing entity. In some embodiments, the method comprises contacting the cell with the composition or the engineered guide RNA described herein. In some embodiments, the method comprises expressing the composition or the engineered guide RNA described herein in the cell. In some embodiments, the engineered guide RNA or vector encoding a non-chemically modified engineered guide RNA or the RNA editing entity can be delivered into the cell via any of the transfection methods described herein. In some embodiments, a non-chemically modified engineered guide RNA or RNA editing entity can be delivered into the cell via the use of expression vectors. In the context of an expression vector, the vector can be readily introduced into the cell described herein by any method in the art. For example, the expression vector can be transferred into the cell by physical, chemical, or biological means.

Physical methods for introducing the engineered guide RNA or vector encoding the non-chemically modified engineered guide RNA or the RNA editing entity into the cell can include calcium phosphate precipitation, lipofection, particle bombardment, microinjection, gene gun, electroporation, and the like. Methods for producing cells comprising vectors and/or exogenous nucleic acids may be suitable for methods herein. One method for the introduction of engineered guide RNA or vector encoding the non-chemically modified engineered guide RNA or the RNA editing entity into a host cell may be calcium phosphate transfection.

Chemical means for introducing the engineered guide RNA or vector encoding the non-chemically modified engineered guide RNA or the RNA editing entity into the cell can include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo may be a liposome (e.g., an artificial membrane vesicle). Other methods of state-of-the-art targeted delivery of nucleic acids may be available, such as delivery of engineered guide RNA or vector encoding the engineered guide RNA or the RNA editing entity with targeted nanoparticles or other suitable sub-micron sized delivery system.

In the case where a non-viral delivery system may be utilized, an exemplary delivery vehicle may be a liposome. The use of lipid formulations may be contemplated for the introduction of the engineered guide RNA or vector encoding the non-chemically modified engineered guide RNA or the RNA editing entity into a cell (in vitro, ex vivo or in vivo). In another aspect, the engineered guide RNA or vector encoding the non-chemically modified engineered guide RNA or the RNA editing entity can be associated with a lipid. The engineered guide RNA or vector encoding the non-chemically modified engineered guide RNA or the RNA editing entity associated with a lipid, in some embodiments, may be encapsulated in the aqueous interior of a liposome, interspersed within the lipid bilayer of a liposome, attached to a liposome via a linking molecule that may be associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they may be present in a bilayer structure, as micelles, or with a “collapsed” structure. Alternately, they may be simply be interspersed in a solution, possibly forming aggregates that are not uniform in size or shape. Lipids may be fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use may be obtained from commercial sources. For example, in some embodiments, dimyristyl phosphatidylcholine (“DMPC”) may be obtained from Sigma, St. Louis, Mo.; in some embodiments, dicetyl phosphate (“DCP”) may be obtained from K & K Laboratories (Plainview, N.Y.); cholesterol (“Choi”), in some embodiments, may be obtained from Calbiochem-Behring; dimyristyl phosphatidylglycerol (“DMPG”). Stock solutions of lipids in chloroform or chloroform/methanol may be often stored at about −20° C. Chloroform may be used as the only solvent since it may be more readily evaporated than methanol. “Liposome” may be a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They may form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure may be also be encompassed. For example, the lipids, in some embodiments, assume a micellar structure or merely exist as nonuniform aggregates of lipid molecules. Also contemplated are lipofectamine-nucleic acid complexes.

In some cases, the engineered guide RNA or vector encoding the non-chemically modified engineered guide RNA or the RNA editing entity described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some cases, the engineered guide RNA or vector encoding the non-chemically modified engineered guide RNA or the RNA editing entity described herein can be administered to the subject in need thereof via the use of the transgenic cells generated by introduction of the engineered guide RNA or vector encoding the non-chemically modified engineered guide RNA or the RNA editing entity first into allogeneic or autologous cells. In some cases, the cell can be isolated. In some embodiments, the cell can be isolated from the subject. In some embodiments, the cell can be an immune cell such as T cell.

In some embodiments, the engineered guide RNA, upon binding to the target RNA, may be more efficient in recruiting the RNA editing entity for editing the target RNA relative to an otherwise identical reference polynucleotide (e.g. reference polynucleotide without a chemical modification). In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more efficient in recruiting the RNA editing entity for editing the target RNA relative to an otherwise identical reference polynucleotide. In some embodiments, the efficiency of editing the target RNA can be measured to amplifying and sequencing the edited target RNA by methods such as Sanger sequencing or sequencing of ddPCR product.

In some embodiments, the engineered guide RNA, upon binding to the target RNA, may be more specific in recruiting the RNA editing entity for editing the target RNA relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more specific in recruiting the RNA editing entity for editing the target RNA relative to an otherwise identical reference polynucleotide. In some embodiments, the specificity of editing of the target RNA can be determined to amplifying and sequencing the edited target RNA by methods such as Sanger sequencing or sequencing of ddPCR product.

In some embodiments, the engineered guide RNA comprises an increased resistance towards degradation by hydrolysis compared to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by hydrolysis relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by hydrolysis relative to an otherwise identical reference polynucleotide, when the engineered guide RNA may be contacted with a cell. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by hydrolysis relative to an otherwise identical reference polynucleotide, when the engineered guide RNA may be administered to a subject in need thereof. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by hydrolysis relative to an otherwise identical reference polynucleotide, when the engineered guide RNA may be in circulation in the subject. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by hydrolysis relative to an otherwise identical reference polynucleotide, when the engineered guide RNA may be contacted with the target RNA.

In some embodiments, the engineered guide RNA comprises an increased resistance towards degradation by nuclease digestion compared to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by nuclease digestion relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by nuclease digestion relative to an otherwise identical reference polynucleotide, when the engineered guide RNA may be contacted with a cell. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by nuclease digestion relative to an otherwise identical reference polynucleotide, when the engineered guide RNA may be administered to a subject in need thereof. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by nuclease digestion relative to an otherwise identical reference polynucleotide, when the engineered guide RNA may be in circulation in the subject. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more resistant towards degradation by nuclease digestion relative to an otherwise identical reference polynucleotide, when the engineered guide RNA may be contacted with the target RNA.

In some embodiments, the engineered guide RNA induces less immunogenicity relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce immunogenicity relative to immunogenicity induced by an otherwise identical reference polynucleotide. In some embodiments, the engineered guide RNA induces less immunogenicity relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce immunogenicity relative to immunogenicity induced by an otherwise identical reference polynucleotide, when the engineered guide RNA may be in a cell. In some embodiments, the engineered guide RNA induces less immunogenicity relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce immunogenicity relative to immunogenicity induced by an otherwise identical reference polynucleotide, when the engineered guide RNA may be administered to a subject in need thereof. In some embodiments, the engineered guide RNA induces less immunogenicity relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce immunogenicity relative to immunogenicity induced by an otherwise identical reference polynucleotide, when the engineered guide RNA may be in circulation in the subject. In some embodiments, the engineered guide RNA induces less immunogenicity relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce immunogenicity relative to immunogenicity induced by an otherwise identical reference polynucleotide, when the engineered guide RNA may be contacted with the target RNA.

In some embodiments, the engineered guide RNA induces less innate immune response relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce innate immune response relative to innate immune response induced by an otherwise identical reference polynucleotide. In some embodiments, the engineered guide RNA induces less innate immune response relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce innate immune response relative to innate immune response induced by an otherwise identical reference polynucleotide, when the engineered guide RNA may be in a cell. In some embodiments, the engineered guide RNA induces less innate immune response relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce innate immune response relative to innate immune response induced by an otherwise identical reference polynucleotide, when the engineered guide RNA may be administered to a subject in need thereof. In some embodiments, the engineered guide RNA induces less innate immune response relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce innate immune response relative to innate immune response induced by an otherwise identical reference polynucleotide, when the engineered guide RNA may be in circulation in the subject. In some embodiments, the engineered guide RNA induces less innate immune response relative to an otherwise identical reference polynucleotide. In some embodiments, the engineered guide RNA may be at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce innate immune response relative to innate immune response induced by an otherwise identical reference polynucleotide, when the engineered guide RNA may be contacted with the target RNA.

In some embodiments, the engineered guide RNA, when contacted with the target RNA, may be less likely to induce off-target editing of the target RNA by the RNA editing entity relative to the off-target editing of the target RNA by the same RNA editing entity induced by an otherwise identical reference polynucleotide. In some embodiments, the engineered guide RNA may be at least at least 10%, 20%, 30%, 40%, 50%, 60%, 70%, 80%, 90%, two fold, three fold, four ford, five fold, six fold, seven fold, eight fold, nine fold, 10 fold, 20 fold, 30 fold, 40 fold, 50 fold, 100 fold, 500 fold, 1000 fold, or more less likely to induce off-target editing relative to the otherwise identical reference polynucleotide. In some embodiments, the prevalence of the off-targeting can be measured to amplifying and sequencing the edited target RNA by methods such as Sanger sequencing or sequencing of ddPCR product.

### Methods of Treatment

Disclosed herein, in some embodiments, are methods of treating a subject by administrating of a therapeutic effective amount of the pharmaceutical composition comprising the engineered guide RNA or the composition described herein to the subject. In some embodiment, the method of treating or preventing a disease or a condition in a subject in need thereof comprises administering to the subject having the disease or the condition an engineered guide RNA, thereby treating or preventing the disease or the condition in the subject, wherein the engineered guide RNA comprises at least one chemical modification relative to an otherwise identical reference polynucleotide, and wherein the engineered guide RNA: associates with at least a portion of a target RNA; forms at least one structural feature in association with the target RNA, the at least one structural feature recruits an RNA editing entity; and facilitates a chemical modification of a base of a nucleotide in the target RNA by the RNA editing entity.

In some cases, the disease or condition being treated by the method described herein may be caused by mutations in RAB7A, ABCA4, SERPINA1, SERPINA1 E342K, HEXA, LRRK2, SNCA, APP, CFTR, ALAS1, ATP7B, ATP7B G1226R, HFE C282Y, LIPA c.894 G>A, PCSK9 start site, or SCNN1A start site, a fragment any of these, or any combination thereof. In some embodiments, the disease or the condition comprises a neurological disease or condition, where the neurological or neurodevelopmental disease or condition comprises Parkinson's disease, Alzheimer's disease, or dementia. In some embodiments, the disease or the condition comprises a liver disease or condition such as liver cirrhosis. In some embodiments, the liver disease or condition may be alpha-1 antitrypsin deficiency (AAT deficiency). In some embodiments the disease or the condition comprises macular degeneration. In some embodiments, the macular degeneration may be Stargardt's disease.

In some embodiments, the pharmaceutical composition can be administered to the subject alone (e.g., standalone treatment). In some embodiments, the pharmaceutical composition may be administered in combination with an additional agent. In some embodiments, the pharmaceutical composition may be a first-line treatment for the disease or condition. In some embodiments, the pharmaceutical composition may be a second-line, third-line, or fourth-line treatment. In some embodiments, the pharmaceutical composition can comprise at least one, two, three, four, five, six, seven, eight, nine, ten, 20, 30 or more engineered guide RNAs. In general, method disclosed herein comprises administering the pharmaceutical composition by oral administration. However, in some instances, method can comprise administering the pharmaceutical composition by intraperitoneal injection. In some instances, method can comprise administering the pharmaceutical composition in the form of an anal suppository. In some instances, method can comprise administering the pharmaceutical composition by intravenous (“i.v.”) administration. It may be conceivable that one can also administer the pharmaceutical composition disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation may be preferred over systemic routes. Routes, dosage, time points, and duration of administrating therapeutics can be adjusted. In some embodiments, administration of therapeutics may be prior to, or after, onset of either, or both, acute and chronic symptoms of the disease or condition.

Suitable dose and dosage administrated to a subject may be determined by factors including, but no limited to, the particular the pharmaceutical composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

In some embodiments, the administration of the pharmaceutical composition may be hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges can be adjusted based on subject's response to the treatment. Some routes of administration will require higher concentrations of effective amount of therapeutics than other routes.

In certain embodiments, where the subject's condition does not improve, upon the doctor's discretion the administration of the pharmaceutical composition may be administered chronically, that is, for an extended period of time, including throughout the duration of the subject's life in order to ameliorate or otherwise control or limit the symptoms of the subject's disease or condition. In certain embodiments wherein a subject's status does improve, the dose of the pharmaceutical composition being administered can be temporarily reduced or temporarily suspended for a certain length of time (i.e., a “drug holiday”). In specific embodiments, the length of the drug holiday may be between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of the pharmaceutical composition being administered can be temporarily reduced or temporarily suspended for a certain length of time (i.e., a “drug diversion”). In specific embodiments, the length of the pharmaceutical composition diversion may be between 2 days and 1 year, including by way of example only, 2 days, 3 days, 4 days, 5 days, 6 days, 7 days, 10 days, 12 days, 15 days, 20 days, 28 days, or more than 28 days. The dose reduction during the pharmaceutical composition diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule may be optionally reinstated.

In some embodiments, once improvement of the subject's conditions has occurred, a maintenance dose may be administered if necessary. Subsequently, in specific embodiments, the dosage or the frequency of administration, or both, may be reduced, as a function of the symptoms, to a level at which the improved disease, disorder or condition may be retained. In certain embodiments, however, the subject requires intermittent treatment on a long-term basis upon any recurrence of symptoms.

Toxicity and therapeutic efficacy of such therapeutic regimens may be determined by standard pharmaceutical procedures in cell cultures or experimental animals, including, but not limited to, the determination of the LD50 and the ED50. The dose ratio between the toxic and therapeutic effects may be the therapeutic index and it may be expressed as the ratio between LD50 and ED50. In certain embodiments, the data obtained from cell culture assays and animal studies may be used in formulating the therapeutically effective daily dosage range and/or the therapeutically effective unit dosage amount for use in mammals, including humans. In some embodiments, the daily dosage amount of the composition described herein lies within a range of circulating concentrations that include the ED50 with minimal toxicity. In certain embodiments, the daily dosage range and/or the unit dosage amount varies within this range depending upon the dosage form employed and the route of administration utilized.

The pharmaceutical composition can be used alone or in combination with an additional agent. In some cases, an “additional agent” as used herein may be administered alone. The pharmaceutical composition and the additional agent can be administered together or sequentially. The combination therapies can be administered within the same day, or can be administered one or more days, weeks, months, or years apart.

### Disease Applications and Targets

An engineered polynucleotide as described herein (i.e. a chemically modified engineered polynucleotide) can be used to treat a disease or condition in a subject. A disease or condition can comprise a neurodegenerative disease, a muscular disorder, a metabolic disorder, an ocular disorder (e.g. an ocular disease), a cancer, a liver disease (Alpha-1 antitrypsin (AAT) deficiency), or any combination thereof. The disease or condition can comprise cystic fibrosis, albinism, alpha-1-antitrypsin deficiency, Alzheimer disease, Amyotrophic lateral sclerosis, Asthma, (3-thalassemia, Cadasil syndrome, Charcot-Marie-Tooth disease, Chronic Obstructive Pulmonary Disease (COPD), dementia, Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Parkinson's disease, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, a tauopathy, a synucleinopathy, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, various forms of cancer (e.g., BRCA1 and 2 linked breast cancer and ovarian cancer). In some cases, a treatment of a disease or condition such as a neurodegenerative disease (e.g. Alzheimer's, Parkinson's) can comprise producing an edit, a knockdown or both of amyloid precursor protein (APP), tau, alpha-synuclein, or any combination thereof. In some cases, APP, tau, and alpha-synuclein can comprise a pathogenic variant. In some instances, APP can comprise a pathogenic variant such as A673V mutation or A673T mutation. In some cases, a treatment of a disease or condition such as a neurodegenerative disease (Parkinson's) can comprise producing an edit, a knockdown or both of a pathogenic variant of LRRK2. In some cases, a pathogenic variant of LRRK can comprise a G2019S mutation. The disease or condition can comprise a muscular dystrophy, an ornithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, a tauopathy, a synucleinopathy, or any combination thereof. In some cases, an engineered polynucleotide can correct a missense mutation in a patient with Rett (e.g. mutate a stop codon to encode for a Trp). In some cases, an engineered polynucleotide can correct a missense mutation or induce a knockdown in a patient with Parkinson's. In some cases, an engineered polynucleotide can induce a mutation in a patient with Alzheimer's, which can reduce cleavage by a protein at a cleavage site in APP. In some cases, an engineered polynucleotide can generate exon skipping in a patient with muscular dystrophy. In some cases, an engineered polynucleotide can correct a mutation in HexA in a patient with Tay-Sachs disease. In some cases, an engineered polynucleotide can correct a mutation in HexA in a patient with Tay-Sachs disease. In some cases, an engineered polynucleotide can correct a mutation in a patient with AAT deficiency (e.g. edit SERPINA1). In some cases, Administration of a composition can be sufficient to: (a) decrease expression of a gene relative to an expression of the gene prior to administration; (b) edit at least one point mutation in a subject, such as a subject in need thereof; (c) edit at least one stop codon in the subject to produce a readthrough of a stop codon; (d) produce an exon skip in the subject, or (e) any combination thereof. A disease or condition can comprise a muscular dystrophy. A muscular dystrophy can include myotonic, Duchenne, Becker, Limb-girdle, facioscapulohumeral, congenital, oculopharyngeal, distal, Emery-Dreifuss, or any combination thereof. A disease or condition can comprise pain, such as chronic pain. Pain can include neuropathic pain, nociceptive pain, or a combination thereof. Nociceptive pain can include visceral pain, somatic pain, or a combination thereof. Certain specification diseases or conditions and associated targets are referenced below.

APP. In some embodiments, the present disclosure provides compositions and methods of use thereof of chemically modified engineered polynucleotides that are capable of facilitating RNA editing of an amyloid precursor protein (APP). For example, chemically modified engineered polynucleotides can facilitate editing of the cleavage site in APP, so that beta/gamma secretases exhibit reduced cleavage of APP or can no longer cut APP and, therefore, reduced levels of Abeta 40/42 or no Abetas can be produced. In some embodiments, a chemically modified engineered polynucleotide of the present disclosure can target any one of or any combination of the following sites in APP for RNA editing: K670E, K670R, K670G, M671V, A673V, A673T, D672G, E682G, H684R, K687R, K687E, or K687G, I712X, or T714X. Sequences of chemically modified engineered polynucleotides that target the APP gene or transcript may comprise a targeting domain with at least about: 70%, 75%, 80%, 85%, 90%, 95%, or 100% complementarity to the APP gene or transcript. Said chemically modified engineered polynucleotides may be administered via any route of administration disclosed herein to a subject in need thereof. The subject may be human and may be at risk of developing or has developed Alzheimer's disease. The subject may be human and may be at risk of developing or has developed a neurological disease in which APP impacts disease pathology. Thus, the chemically modified engineered polynucleotides may be used in a method of treatment of neurological diseases (e.g., Alzheimer's disease).

ABCA4. In some embodiments, the present disclosure provides compositions and methods of use thereof of engineered polynucleotides (i.e. chemically modified engineered polynucleotides) that are capable of facilitating RNA editing of an ATP Binding Cassette Subfamily A Member 4 (ABCA4). For example, chemically modified engineered polynucleotides can facilitate correction of a G with an A at nucleotide position 6320 in an ABCA4 gene; G with an A at nucleotide position 5714 in a ABCA4 gene; and/or a G with an A at nucleotide position 5714 in a ABCA4 gene. Sequences of chemically modified engineered polynucleotides that target the ABCA4 gene or transcript may comprise a targeting domain with at least about: 70%, 75%, 80%, 85%, 90%, 95%, or 100% complementarity to the ABCA4 gene or transcript. Said chemically modified engineered polynucleotides may be administered via any route of administration disclosed herein to a subject in need thereof. The subject may be human and may be at risk of developing or has developed Stargardt macular degeneration. Such Stargardt macular degeneration may be at least partially caused by a mutation of ABCA4, for which an engineered polynucleotide sequence described herein can facilitate editing in, thus correcting the mutation in ABCA4 and reducing the incidence of Stargardt macular degeneration in the subject. Thus, the chemically modified engineered polynucleotides may be used in a method of treatment of Stargardt macular degeneration.

Alpha-synuclein (SNCA). The Alpha-synuclein gene is made up of 5 exons and encodes a 140 amino-acid protein with a predicted molecular mass of ˜14.5 kDa. The encoded product is an intrinsically disordered protein with unknown functions. Usually, Alpha-synuclein is a monomer. Under certain stress conditions or other unknown causes, α-synuclein self-aggregates into oligomers. Lewy-related pathology (LRP), primarily comprised of Alpha-synuclein in more than 50% of autopsy-confirmed Alzheimer's disease patients' brains. While the molecular mechanism of how Alpha-synuclein affects the development of Alzheimer's disease is unclear, experimental evidence has shown that Alpha-synuclein interacts with Tau-p and may seed the intracellular aggregation of Tau-p. Moreover, Alpha-synuclein could regulate the activity of GSK3β, which can mediate Tau-hyperphosphorylation. Alpha-synuclein can also self-assemble into pathogenic aggregates (Lewy bodies). Both Tau and α-synuclein can be released into the extracellular space and spread to other cells. Vascular abnormalities impair the supply of nutrients and removal of metabolic byproducts, cause microinfarcts, and promote the activation of glial cells. Therefore, a multiplex strategy to substantially reduce Tau formation, alpha-synuclein formation, or a combination thereof can be important in effectively treating neurodegenerative diseases.

The domain structure of Alpha-synuclein comprises an N-terminal A2 lipid-binding alpha-helix domain, a Non-amyloid R component (NAC) domain, and a C-terminal acidic domain. The lipid-binding domain consists of five KXKEGV imperfect repeats. The NAC domain consists of a GAV motif with a VGGAVVTGV consensus sequence and three GXXX sub-motifs—where X is any of Gly, Ala, Val, Ile, Leu, Phe, Tyr, Trp, Thr, Ser or Met. The C-terminal acidic domain contains a copper-binding motif with a DPDNEA consensus sequence. Molecularly, Alpha-synuclein is suggested to play a role in neuronal transmission and DNA repair.

In some cases, a region of Alpha-synuclein can be targeted utilizing compositions provided herein. In some cases, a region of the Alpha-synuclein mRNA can be targeted with the engineered polynucleotides disclosed herein for knockdown. In some cases, a region of the exon or intron of the Alpha-synuclein mRNA can be targeted. In some embodiments, a region of the non-coding sequence of the Alpha-synuclein mRNA, such as the 5′UTR and 3′UTR, can be targeted. In other cases, a region of the coding sequence of the Alpha-synuclein mRNA can be targeted. Suitable regions include but are not limited to a N-terminal A2 lipid-binding alpha-helix domain, a Non-amyloid R component (NAC) domain, or a C-terminal acidic domain.

In some aspects, an alpha-synuclein mRNA sequence is targeted. In some cases, any one of the 3,177 residues of the sequence may be targeted utilizing the compositions and methods provided herein. In some cases, a target residue may be located among residues 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, 2601-2700, 2701-2800, 2801-2900, 2901-3000, 3001-3100, and/or 3101-3177.

In some embodiments, the present disclosure provides compositions and methods of use thereof of engineered polynucleotides (i.e. chemically modified engineered polynucleotides) that are capable of facilitating RNA editing of SNCA. In some embodiments, a chemically modified engineered polynucleotide can knock down expression of SNCA, for example, by facilitating editing at a 3′ UTR of an SNCA gene. Sequences of chemically modified engineered polynucleotides that target the SNCA gene or transcript may comprise a targeting domain with at least about: 70%, 75%, 80%, 85%, 90%, 95%, or 100% complementarity to the SNCA gene or transcript. Said chemically modified engineered polynucleotides may be administered via any route of administration disclosed herein to a subject in need thereof. The subject may be human and may be at risk of developing or has developed Alzheimer's disease or Parkinson's disease. The subject may be human and may be at risk of developing or has developed a neurological disease in which overexpression of SNCA impacts disease pathology. Thus, the chemically modified engineered polynucleotides may be used in a method of treatment of neurological diseases (e.g., Alzheimer's disease).

SERPINA1. In some embodiments, the present disclosure provides compositions and methods of use thereof of engineered polynucleotides (i.e. chemically modified engineered polynucleotides) that are capable of facilitating RNA editing of serpin family A member 1 (SERPINA1). For example, chemically modified engineered polynucleotides can facilitate correction of a G to A mutation at nucleotide position 9989 of a SERPINA1 gene. In some embodiments, a chemically modified engineered polynucleotide can target, for example, E342 of SERPINA1. Sequences of chemically modified engineered polynucleotides configured to target a SERPINA1 gene or transcript may comprise a targeting domain with at least about: 70%, 75%, 80%, 85%, 90%, 95%, or 100% sequence identity to the SEPRINA1 gene or transcript. Said chemically modified engineered polynucleotides may be administered via any route of administration disclosed herein to a subject in need thereof. The subject may be human and may be at risk of developing or has developed alpha-1 antitrypsin deficiency. Such alpha-1 antitrypsin deficiency may be at least partially caused by a mutation of SERPINA1, for which an engineered polynucleotide sequence described herein can facilitate editing in, thus correcting the mutation in SERPINA1 and reducing the incidence of alpha-1 antitrypsin deficiency in the subject. Thus, the chemically modified engineered polynucleotides may be used in a method of treatment of alpha-1 antitrypsin deficiency.

LRRK2. Leucine-rich repeat kinase 2 (LRRK2) has been associated with familial and sporadic cases of Parkinson's Disease and immune-related disorders like Crohn's disease. Its aliases include LRRK2, AURA17, DARDARIN, PARK8, RIPK7, ROCO2, or leucine-rich repeat kinase 2. The LRRK2 gene is made up of 51 exons and encodes a 2527 amino-acid protein with a predicted molecular mass of about 286 kDa. The encoded product is a multi-domain protein with kinase and GTPase activities. LRRK2 can be found in various tissues and organs including but not limited to adrenal, appendix, bone marrow, brain, colon, duodenum, endometrium, esophagus, fat, gall bladder, heart, kidney, liver, lung, lymph node, ovary, pancreas, placenta, prostate, salivary gland, skin, small intestine, spleen, stomach, testis, thyroid, and urinary bladder. LRRK2 can be ubiquitously expressed but is generally more abundant in the brain, kidney, and lung tissue. Cellularly, LRRK2 has been found in astrocytes, endothelial cells, microglia, neurons, and peripheral immune cells.

Over 100 mutations have been identified in LRRK2; six of them-G2019S, R1441C/G/H, Y1699C, and 12020T—have been shown to cause Parkinson's Disease through segregation analysis. G2019S and R1441C are the most common disease-causing mutations in inherited cases. In sporadic cases, these mutations have shown age-dependent penetrance: The percentage of individuals carrying the G2019S mutation that develops the disease jumps from 17% to 85% when the age increases from 50 to 70 years old. In some cases, mutation-carrying individuals never develop the disease.

At its catalytic core, LRRK2 contains the Ras of complex proteins (Roc), C-terminal of ROC (COR), and kinase domains. Multiple protein-protein interaction domains flank this core: an armadillo repeats (ARM) region, an ankyrin repeat (ANK) region, a leucine-rich repeat (LRR) domain are found in the N-terminus joined by a C-terminal WD40 domain. The G2019S mutation is located within the kinase domain. It has been shown to increase the kinase activity; for R1441C/G/H and Y1699C, these mutations can decrease the GTPase activity of the Roc domain. Genome-wide association study has found that common variations in LRRK2 increase the risk of developing sporadic Parkinson's Disease. While some of these variations are nonconservative mutations that affect the protein's binding or catalytic activities, others modulate its expression. These results suggest that specific alleles or haplotypes can regulate LRRK2 expression.

Pro-inflammatory signals upregulate LRRK2 expression in various immune cell types, suggesting that LRRK2 is a critical regulator in the immune response. Studies have found that both systemic and central nervous system (CNS) inflammation are involved in Parkinson's Disease's symptoms. Moreover, LRRK2 mutations associated with Parkinson's Disease modulate its expression levels in response to inflammatory stimuli. Many mutations in LRRK2 are associated with immune-related disorders such as inflammatory bowel disease such as Crohn's Disease. For example, both G2019S and N2081D increase LRRK2's kinase activity and are over-represented in Crohn's Disease patients in specific populations. Because of its critical role in these disorders, LRRK2 is an important therapeutic target for Parkinson's Disease and Crohn's Disease. In particular, many mutations, such as point mutations including G2019S, play roles in developing these diseases, making LRRK2 an attractive for therapeutic strategy such as RNA editing.

In some embodiments, the present disclosure provides compositions and methods of use thereof of engineered polynucleotides (i.e. chemically modified engineered polynucleotides) that are capable of facilitating RNA editing of LRRK2. In some embodiments, chemically modified engineered polynucleotides can target the following mutations in LRRK2: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, 51096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, 51647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, or Q2490NfsX3. Sequences of chemically modified engineered polynucleotides configured to target a LRRK2 gene or transcript may comprise a targeting domain with at least about: 70%, 75%, 80%, 85%, 90%, 95%, or 100% complementarity to the LRRK2 gene or transcript. Said chemically modified engineered polynucleotides may be administered via any route of administration disclosed herein to a subject in need thereof. The subject may be human and may be at risk of developing or has developed a disease or condition associated with mutations in LRRK2 (e.g. diseases of the central nervous system (CNS) or gastrointestinal (GI) tract). For example, such diseases of conditions can include Crohn's disease or Parkinson's disease. Such CNS or GI tract diseases (e.g. Crohn's disease or Parkinson's disease) may be at least partially caused by a mutation of LRRK2, for which an engineered polynucleotide sequence described herein can facilitate editing in, thus correcting the mutation in LRRK2 and reducing the incidence of the CNS or GI tract disease in the subject. Thus, the chemically modified engineered polynucleotides may be used in a method of treatment of diseases such as Crohn's disease or Parkinson's disease.

DMD. In some embodiments, the present disclosure provides compositions and methods of use thereof of engineered polynucleotides (i.e. chemically modified engineered polynucleotides) that are capable of facilitating RNA editing of a Duchenne muscular dystrophy (DMD) gene. In some embodiments, chemically modified engineered polynucleotides can target an exon of a DMD gene, such as exon 51, 45, 53, 44, 46, 52, 50, 43, 6, 7, 8, 55, 2, 11, 17, 19, 21, 57, 59, 62, 63, 65, 66, 69, 74 and/or 75 in the DMD gene pre-mRNA that at least in part encodes a dystrophin protein. Sequences of chemically modified engineered polynucleotides configured to target a DMD gene or transcript may comprise a targeting domain with at least about: 70%, 75%, 80%, 85%, 90%, 95%, or 100% complementarity to the DMD gene or transcript. Said chemically modified engineered polynucleotides may be administered via any route of administration disclosed herein to a subject in need thereof. The subject may be human and may be at risk of developing or has developed a disease or condition associated with mutations in a DMD gene such as DMD. DMD may be at least partially caused by a mutation of a DMD gene, for which an engineered polynucleotide sequence described herein can facilitate editing in, thus correcting the mutation in DMD gene and reducing the incidence of the DMD in the subject. Thus, the chemically modified engineered polynucleotides may be used in a method of treatment of diseases such as DMD.

TUBB4A. In some embodiments, the present disclosure provides compositions and methods of use thereof of engineered polynucleotides (i.e. chemically modified engineered polynucleotides) that are capable of facilitating RNA editing of the TUBB4A gene. In some embodiments, chemically modified engineered polynucleotides can target the D249N mutation in TUBB4A, which can be caused by a 745G>A nucleotide mutation. Sequences of chemically modified engineered polynucleotides configured to target a TUBB4A gene or transcript may comprise a targeting domain with at least about: 70%, 75%, 80%, 85%, 90%, 95%, or 100% complementarity to the TUBB4A gene or transcript. Said chemically modified engineered polynucleotides may be administered via any route of administration disclosed herein to a subject in need thereof. The subject may be human and may be at risk of developing or has developed a disease or condition associated with mutations in a TUBB4A gene such as Hypomyelination with Atrophy of Basal Ganglia and cerebellum (H-ABC). H-ABC may be at least partially caused by a mutation of a TUBB4A gene, for which an engineered polynucleotide sequence described herein can facilitate editing in, thus correcting the mutation in TUBB4A gene and reducing the incidence of the H-ABC in the subject. Thus, the chemically modified engineered polynucleotides may be used in a method of treatment of diseases such as H-ABC.

### Pharmaceutical Compositions

A pharmaceutical composition, as used herein, may refer to a mixture of a pharmaceutical composition, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more pharmaceutical composition as discussed herein. In practicing the methods of treatment or use provided herein, therapeutically effective amounts of pharmaceutical compositions described herein may be administered in a pharmaceutical composition to a mammal having a disease, disorder, or condition to be treated, e.g., an inflammatory disease, fibrostenotic disease, and/or fibrotic disease. In some embodiments, the mammal may be a human. A therapeutically effective amount can vary widely depending on the severity of the disease, the age and relative health of the subject, the potency of the pharmaceutical composition used and other factors. The pharmaceutical compositions can be used singly or in combination with one or more pharmaceutical compositions as components of mixtures. The pharmaceutical commotions described herein comprise the engineered guide RNA, the compositions, the cells contacted with the engineered guide RNA or contacted with the composition comprising the engineered guide RNA, or a combination thereof.

The pharmaceutical formulations described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a pharmaceutical composition may be manufactured in a conventional manner, such as, by way of example only, by means of conventional mixing, dissolving, granulating, dragee-making, levigating, emulsifying, encapsulating, entrapping or compression processes.

The pharmaceutical compositions may include at least a pharmaceutical composition as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, pharmaceutical compositions exist in unsolvated form or in solvated forms with pharmaceutically acceptable solvents such as water, ethanol, and the like. The solvated forms of the pharmaceutical compositions are also considered to be disclosed herein.

In some embodiments, a pharmaceutical composition exists as a tautomer. Tautomers may be included within the scope of the agents presented herein. As such, it may be understood that a pharmaceutical composition or a salt thereof may exhibit the phenomenon of tautomerism whereby two chemical compounds that may be capable of facile interconversion by exchanging a hydrogen atom between two atoms, to either of which it forms a covalent bond. Since the tautomeric compounds exist in mobile equilibrium with each other they can be regarded as different isomeric forms of the same compound.

In some embodiments, a pharmaceutical composition exists as an enantiomer, diastereomer, or other steroisomeric form. The agents disclosed herein include all enantiomeric, diastereomeric, and epimeric forms as well as mixtures thereof.

In some embodiments, pharmaceutical compositions described herein can be prepared as prodrugs. A “prodrug” may refer to an agent that may be converted into the parent drug in vivo. Prodrugs may be useful because, in some situations, they can be easier to administer than the parent drug. They may, for instance, be bioavailable by oral administration whereas the parent may not. The prodrug may also have improved solubility in pharmaceutical compositions over the parent drug. An example, without limitation, of a prodrug would be a pharmaceutical composition described herein, which may be administered as an ester (the “prodrug”) to facilitate transmittal across a cell membrane where water solubility may be detrimental to mobility but which then may be metabolically hydrolyzed to the carboxylic acid, the active enzyme, once inside the cell where water-solubility may be beneficial. A further example of a prodrug might be a short peptide (polyaminoacid) bonded to an acid group where the peptide may be metabolized to reveal the active moiety. In certain embodiments, upon in vivo administration, a prodrug may be chemically converted to the biologically, pharmaceutically or therapeutically active form of the pharmaceutical composition. In certain embodiments, a prodrug may be enzymatically metabolized by one or more steps or processes to the biologically, pharmaceutically or therapeutically active form of the pharmaceutical composition.

Prodrug forms of the pharmaceutical compositions, wherein the prodrug may be metabolized in vivo to produce an agent as set forth herein may be included within the scope of the claims. Prodrug forms of the herein described pharmaceutical compositions, wherein the prodrug may be metabolized in vivo to produce an agent as set forth herein may be included within the scope of the claims. In some cases, some of the pharmaceutical compositions described herein can be a prodrug for another derivative or active compound. In some embodiments described herein, hydrazones may be metabolized in vivo to produce a pharmaceutical composition.

### Kits and Article of Manufacture

Disclosed herein, in certain embodiments, are kits and articles of manufacture for use with one or more methods described herein. In some cases, the kits or articles of manufacture comprise the engineered guide RNA, the composition, the cell, or the pharmaceutical compositions described herein. Such kits include a carrier, package, or container that may be compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers may be formed from a variety of materials such as glass or plastic.

In some embodiments, a kit includes a suitable packaging material to house the contents of the kit. In some cases, the packaging material may be constructed by well-known methods, preferably to provide a sterile, contaminant-free environment. The packaging materials employed herein can include, for example, those customarily utilized in commercial kits sold for use with nucleic acid sequencing systems. Exemplary packaging materials include, without limitation, glass, plastic, paper, foil, and the like, capable of holding within fixed limits a component set forth herein.

The packaging material can include a label which indicates a particular use for the components. The use for the kit that may be indicated by the label can be one or more of the methods set forth herein as appropriate for the particular combination of components present in the kit.

Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like.

It will be understood that not all components necessary for a particular reaction need be present in a particular kit. Rather one or more additional components can be provided from other sources. The instructions provided with a kit can identify the additional component(s) that may be provided and where they can be obtained.

Use of absolute or sequential terms, for example, “will,” “will not,” “shall,” “shall not,” “must,” “must not,” “first,” “initially,” “next,” “subsequently,” “before,” “after,” “lastly,” and “finally,” are in some events not meant to limit scope of the present embodiments disclosed herein but as exemplary.

As used herein, the singular forms “a”, “an” and “the” may be intended to include the plural forms as well, unless the context clearly indicates otherwise. Furthermore, to the extent that the terms “including”, “includes”, “having”, “has”, “with”, or variants thereof may be used in either the detailed description and/or the claims, such terms may be intended to be inclusive in a manner similar to the term “comprising.”

As used herein, the phrases “at least one”, “one or more”, and “and/or” may be open-ended expressions that may be both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and can be used both exclusively and inclusively. For example, the term “A or B” may refer to “A or B”, “A but not B”, “B but not A”, and “A and B”. In some cases, context may dictate a particular meaning.

Any systems, methods, software, and platforms described herein may be modular. Accordingly, terms such as “first” and “second” do not necessarily imply priority, order of importance, or order of acts.

The term “about” or “approximately” as used herein when referring to a measurable value such as an amount or concentration and the like, may be meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values can be described in the application and claims, unless otherwise stated the term “about” meaning within an acceptable error range for the particular value should be assumed. Also, where ranges, subranges, or both, of values can be provided, the ranges or subranges can include the endpoints of the ranges or subranges. The terms “substantially”, “substantially no”, “substantially not”, “substantially free”, and “approximately” can be used when describing a magnitude, a position or both to indicate that the value described can be within a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

The term “and/or” as used in a phrase such as “A and/or B” herein may be intended to include both A and B; A or B; A (alone); and B (alone). Likewise, the term “and/or” as used in a phrase such as “A, B, and/or C” may be intended to encompass each of the following embodiments: A, B, and C; A, B, or C; A or C; A or B; B or C; A and C; A and B; B and C; A (alone); B (alone); and C (alone).

As used herein, the term “comprising” may be intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms may be within the scope of this disclosure.

The term “effective amount” or “therapeutically effective amount” may refer to the amount of an agent that may be sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it may be administered in combination with other compounds, timing of administration, the tissue to be imaged, and the physical delivery system in which it may be carried.

The terms “polypeptide”, “peptide”, and “protein” may be used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” may refer to either natural and/or unnatural or synthetic amino acids, including glycine and both the D or L optical isomers, and amino acid analogs and peptidomimetics.

The term “subject,” “host,” “individual,” and “patient” may be as used interchangeably herein to refer to animals, typically mammalian animals. Any suitable mammal can be treated by a method, cell or composition described herein. A mammal can be administered a vector, an engineered guide RNA, a precursor guide RNA, a nucleic acid, a polynucleotide, an engineered polynucleotide, or a pharmaceutical composition, as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal may be a human. A mammal can be any age or at any stage of development (e.g., an adult, teen, child, infant, or a mammal in utero). A mammal can be male or female. A mammal can be a pregnant female. In some embodiments a subject may be a human. In some embodiments, a subject has or may be suspected of having a disease such as a neurodegenerative disease. In some embodiments, a subject has or can be suspected of having a cancer or neoplastic disorder. In other embodiments, a subject has or can be suspected of having a disease or disorder associated with aberrant protein expression. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

The term “sample” as used herein, generally may refer to any sample of a subject (such as a blood sample or a tissue sample). A sample or portion thereof may comprise cell, such as a stem cell. A portion of a sample may be enriched for the stem cell. The stem cell may be isolated from the sample. A sample may comprise a tissue, a cell, serum, plasma, exosomes, a bodily fluid, or any combination thereof. A bodily fluid may comprise urine, blood, serum, plasma, saliva, mucus, spinal fluid, tears, semen, bile, amniotic fluid, cerebrospinal fluid, or any combination thereof. A sample or portion thereof may comprise an extracellular fluid obtained from a subject. A sample or portion thereof may comprise cell-free nucleic acid, DNA or RNA. A sample or portion thereof may be analyzed for a presence or absence or one or more mutations. Genomic data may be obtained from the sample or portion thereof. A sample may be a sample suspected or confirmed of having a disease or condition. A sample may be a sample removed from a subject via a non-invasive technique, a minimally invasive technique, or an invasive technique. A sample or portion thereof may be obtained by a tissue brushing, a swabbing, a tissue biopsy, an excised tissue, a fine needle aspirate, a tissue washing, a cytology specimen, a surgical excision, or any combination thereof. A sample or portion thereof may comprise tissues or cells from a tissue type. For example, a sample may comprise a nasal tissue, a trachea tissue, a lung tissue, a pharynx tissue, a larynx tissue, a bronchus tissue, a pleura tissue, an alveoli tissue, breast tissue, bladder tissue, kidney tissue, liver tissue, colon tissue, thyroid tissue, cervical tissue, prostate tissue, heart tissue, muscle tissue, pancreas tissue, anal tissue, bile duct tissue, a bone tissue, brain tissue, spinal tissue, kidney tissue, uterine tissue, ovarian tissue, endometrial tissue, vaginal tissue, vulvar tissue, uterine tissue, stomach tissue, ocular tissue, sinus tissue, penile tissue, salivary gland tissue, gut tissue, gallbladder tissue, gastrointestinal tissue, bladder tissue, brain tissue, spinal tissue, a blood sample, or any combination thereof.

“Eukaryotic cells” comprise all life kingdoms except monera. They can be easily distinguished through a membrane-bound nucleus. Animals, plants, fungi, and protists may be eukaryotes or organisms whose cells may be organized into complex structures by internal membranes and a cytoskeleton. The most characteristic membrane-bound structure may be the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

The term “protein”, “peptide”, and “polypeptide” may be used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” may refer to either natural and/or unnatural or synthetic amino acids, including glycine and both the D and L optical isomers, amino acid analogs and peptidomimetics. As used herein, the term “fusion protein” may refer to a protein comprised of domains from more than one naturally occurring or recombinantly produced protein, where generally each domain serves a different function. In this regard, the term “linker” may refer to a protein fragment that may be used to link these domains together—optionally to preserve the conformation of the fused protein domains and/or prevent unfavorable interactions between the fused protein domains which may compromise their respective functions.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed.

In some cases, the identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

The terms “polynucleotide” and “oligonucleotide” may be used interchangeably and refer to a polymeric form of nucleotides of any length, either deoxyribonucleotides or ribonucleotides or analogs thereof. Polynucleotides can have any three-dimensional structure and may perform any function, known or unknown. The following may be non-limiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA). A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also may refer to both double and single stranded molecules. Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction. Unless otherwise specified or required, any embodiment of this disclosure that may be a polynucleotide encompasses both the double stranded form and each of two complementary single stranded forms known or predicted to make up the double stranded form. Some embodiments refer to a DNA sequence. In some embodiments, the DNA sequence may be interchangeable with a similar RNA sequence. Some embodiments refer to an RNA sequence. In some embodiments, the RNA sequence may be interchangeable with a similar DNA sequence. In some embodiments, Us and Ts may be interchanged in a sequence provided herein.

Polynucleotides useful in the methods of the disclosure can comprise natural nucleic acid sequences and variants thereof, artificial nucleic acid sequences, or a combination of such sequences.

A polynucleotide may be composed of a specific sequence of four nucleotide bases: adenine (A); cytosine (C); guanine (G); thymine (T); and uracil (U) for thymine when the polynucleotide may be RNA. In some embodiments, the polynucleotide may comprise one or more other nucleotide bases, such as inosine (I), a nucleoside formed when hypoxanthine may be attached to ribofuranose via a β-N9-glycosidic bond, resulting in the chemical structure:

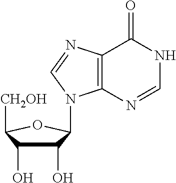

Inosine may be read by the translation machinery as guanine (G).

The term “polynucleotide sequence” may be the alphabetical representation of a polynucleotide molecule. This alphabetical representation can be input into databases in a computer having a central processing unit and used for bioinformatics applications such as functional genomics and homology searching.

As used herein, “expression” may refer to the process by which polynucleotides may be transcribed into mRNA and/or the process by which the transcribed mRNA may be subsequently being translated into peptides, polypeptides, or proteins. If the polynucleotide may be derived from genomic DNA, expression may include splicing of the mRNA in a eukaryotic cell.

The terms “equivalent” or “biological equivalent” may be used interchangeably when referring to a particular molecule, biological, or cellular material and intend those having minimal homology while still maintaining desired structure or functionality.

The term “encode” as it may be applied to polynucleotides may refer to a polynucleotide which may be said to “encode” a polypeptide if, in its native state or when manipulated by methods well known to those skilled in the art, it can be transcribed and/or translated to produce the mRNA for the polypeptide and/or a fragment thereof. The antisense strand may be the complement of such a nucleic acid, and the encoding sequence can be deduced therefrom.

As used herein, the term “functional” may be used to modify any molecule, biological, or cellular material to intend that it accomplishes a particular, specified effect.

The term “mutation” as used herein, may refer to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations may be those which have no effect on the resulting protein. As used herein the term “point mutation” may refer to a mutation affecting only one nucleotide in a gene sequence. “Splice site mutations” may be those mutations present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. The reference DNA sequence can be obtained from a reference database. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (e.g., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation may indicate that a tissue or sample may be benign. As an alternative, absence of a mutation may not indicate that a tissue or sample may be benign. Methods as described herein can comprise identifying a presence of a mutation in a sample.

“Messenger RNA” or “mRNA” may be a nucleic acid molecule that may be transcribed from DNA and then processed to remove non-coding sections known as introns. The resulting mRNA may be exported from the nucleus (or another locus where the DNA may be present) and translated into a protein. The term “pre-mRNA” may refer to the strand prior to processing to remove non-coding sections.

“Canonical amino acids” refer to those 20 amino acids found naturally in the human body shown in the table below with each of their three letter abbreviations, one letter abbreviations, structures, and corresponding codons as shown in Table 3.

TABLE 3
Canonical amino acids and abbreviations
non-polar, aliphatic residues

| | | | | |
|---|---|---|---|---|
| Glycine | Gly | G | 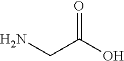 | GGU GGC GGA GGG |
| Alanine | Ala | A | 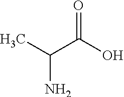 | GCU GCC GCA GCG |
| Valine | Val | V | 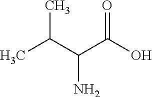 | GUU GUC GUA GUG |
| Leucine | Leu | L | 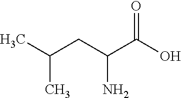 | UUA UUG CUU CUC CUA CUG |
| Isoleucine | Ile | I | 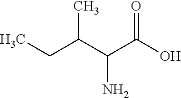 | AUU AUC AUA |
| Proline | Pro | P | 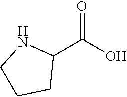 | CCU CCC CCA CCG |

aromatic residues

| | | | | |
|---|---|---|---|---|
| Phenylalanine | Phe | F | 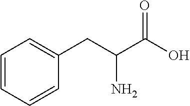 [SMILES: NC(Cc1ccccc1)C(=O)O] | UUUUUC |
| Tyrosine | Tyr | Y | 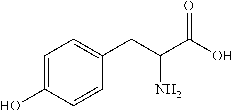 [SMILES: NC(Cc1ccc(O)cc1)C(=O)O] | UAU UAC |
| Tryptophan | Trp | W | 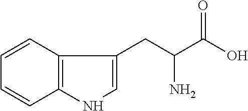 [SMILES: NC(Cc1c[nH]c2ccccc12)C(=O)O] | UGG |

polar, non-charged residues

| | | | | |
|---|---|---|---|---|
| Serine | Ser | S | 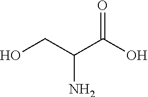 | UCU UCC UCA UCG AGU AGC |
| Threonine | Thr | T | 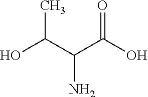 | ACU ACC ACA ACG |
| Cysteine | Cys | C | 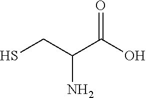 | UGU UGC |
| Methionine | Met | M | 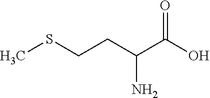 | AUG |
| Asparagine | Asn | N | 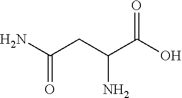 | AAU AAC |
| Glutamine | Gin | Q | 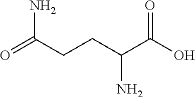 | CAA CAG |

positively charged residues

| | | | | |
|---|---|---|---|---|
| Lysine | Lys | K | 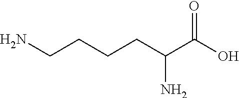 | AAA AAG |
| Arginine | Arg | R | 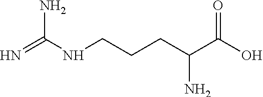 [SMILES: N=C(N)NCCCC(N)C(=O)O] | CGU CGC CGA CGG AGA AGG |
| Histidine | His | H | 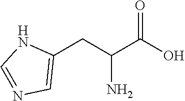 [SMILES: NC(Cc1cnc[nH]1)C(=O)O] | CAU CAC |

negatively charged residues

| | | | | |
|---|---|---|---|---|
| Aspartate | Asp | D | 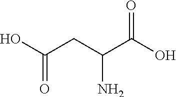 | GAU GAC |
| Glutamate | Glu | E | 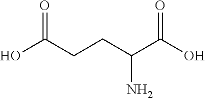 | GAA GAG |

The term “non-canonical amino acids” may refer to those synthetic or otherwise modified amino acids that fall outside this group, typically generated by chemical synthesis or modification of canonical amino acids (e.g. amino acid analogs). The present disclosure employs proteinogenic non-canonical amino acids in some of the methods and vectors disclosed herein. A non-limiting example of a non-canonical amino acid may be pyrrolysine (Pyl or O), the chemical structure provided below:

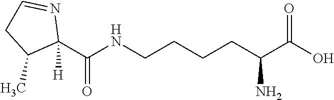 [SMILES: C[C@@H]1CC=N[C@H]1C(=O)NCCCC[C@H](N)C(=O)O]

Inosine (I) may be another exemplary non-canonical amino acid, which may be commonly found in tRNA and may be essential for proper translation according to “wobble base pairing.” A non-limiting example of a structure of inosine is provided above.

The term “ADAR” as used herein may refer to an Adenosine Deaminase Acting on RNA that can convert adenosines (A) to inosines (I) in an RNA sequence. ADAR1 and ADAR2 may be two exemplary species of ADAR that may be involved in RNA editing in vivo. Non-limiting exemplary sequences for ADAR1 may be found under the following reference numbers: HGNC: 225; Entrez Gene: 103; Ensembl: ENSG 00000160710; OMIM: 146920; UniProtKB: P55265; and GeneCards: GC01M154554, as well as biological equivalents thereof. Non-limiting exemplary sequences for ADAR2 may be found under the following reference numbers: HGNC: 226; Entrez Gene: 104; Ensembl: ENSG00000197381; OMIM: 601218; UniProtKB: P78563; and GeneCards: GC21P045073, as well as biological equivalents thereof. Biologically active fragments of ADAR may also be provided herein and can be included when referring to an ADAR.

The term “deficiency” as used herein may refer to lower than normal (physiologically acceptable) levels of a particular agent. In context of a protein, a deficiency may refer to lower than normal levels of the full-length protein.

The term “complementary” or “complementarity” may refer to the ability of a nucleic acid to form hydrogen bond(s) with another nucleic acid sequence by either traditional Watson-Crick or other non-traditional types. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary”, “partially complementary”, “at least partially complementary”, or as used herein may refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or may refer to two nucleic acids that hybridize under stringent conditions (e.g., stringent hybridization conditions). Nucleic acids can include nonspecific sequences. As used herein, the term “nonspecific sequence” or “not specific” may refer to a nucleic acid sequence that contains a series of residues that can be not designed to be complementary to or can be only partially complementary to any other nucleic acid sequence.

As used herein, the term “domain” may refer to a particular region of a protein or polypeptide and can be associated with a particular function. For example, “a domain which associates with an RNA hairpin motif” may refer to the domain of a protein that binds one or more RNA hairpin. This binding may optionally be specific to a particular hairpin.

It may be to be inferred without explicit recitation and unless otherwise intended, that when the present disclosure relates to a polypeptide, protein, polynucleotide or antibody, an equivalent or a biological equivalent of such may be intended within the scope of this disclosure. As used herein, the term “biological equivalent thereof” may be intended to be synonymous with “equivalent thereof” when referring to a reference protein, antibody, polypeptide or nucleic acid, intends those having minimal homology while still maintaining desired structure or functionality. Unless specifically recited herein, it may be contemplated that any polynucleotide, polypeptide or protein mentioned herein also includes equivalents thereof. For example, an equivalent can have at least about 70% homology or identity, at least 80% homology or identity, at least about 85%, at least about 90%, at least about 95%, or at least about 98% percent homology or identity and exhibits substantially equivalent biological activity to the reference protein, polypeptide, or nucleic acid. Alternatively, when referring to polynucleotides, an equivalent thereof may be a polynucleotide that hybridizes under stringent conditions to the reference polynucleotide or its complement.

The terms “increased”, “increasing”, or “increase” may be used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” may be used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms may be meant a statistically significant decrease in such level. The decrease can be, for example, at least 10%, at least 20%, at least 30%, at least 40% or more, and may be preferably down to a level accepted as within the range of normal for an individual without a given disease.

A peptide nucleic acid (PNA) may be a synthetic DNA/RNA analog wherein a peptide-like backbone replaces the sugar-phosphate backbone of DNA or RNA. PNA oligomers show higher binding strength and greater specificity in binding to complementary DNAs, with a PNA/DNA base mismatch being more destabilizing than a similar mismatch in a DNA/DNA duplex. This binding strength and specificity also applies to PNA/RNA duplexes. PNAs may in some cases not be easily recognized by either nucleases or proteases, making them resistant to enzyme degradation. PNAs may be stable over a wide pH range.

A locked nucleic acid (LNA) may be a modified RNA nucleotide, wherein the ribose moiety of an LNA nucleotide may be modified with an extra bridge connecting the 2′ oxygen and 4′ carbon. The bridge “locks” the ribose in the 3′-endo (North) conformation, which may be often found in the A-form duplexes. LNA nucleotides can be mixed with DNA or RNA residues in the oligonucleotide whenever desired. Such oligomers can be synthesized chemically and may be commercially available. The locked ribose conformation enhances base stacking and backbone pre-organization.

The section headings used herein may be for organizational purposes and may in some instances not be construed as limiting the subject matter described.

While preferred embodiments of the present invention have been shown and described herein, it will be obvious to those skilled in the art that such embodiments may be provided by way of example only. It is in some cases not intended that the invention be limited by the specific examples provided within the specification. While the invention has been described with reference to the aforementioned specification, the descriptions and illustrations of the embodiments herein are not necessarily meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein can be employed in practicing the invention. It may be therefore contemplated that the invention shall also cover any such alternatives, modifications, variations or equivalents. It is in some cases intended that the claims define the scope of the invention and that methods and structures within the scope of these claims and their equivalents be covered thereby.

### NUMBERED EMBODIMENTS

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. An engineered guide RNA comprising that comprises:
(a) at least one chemical modification, and
(b) a targeting domain, wherein the targeting domain hybridizes to a target RNA when administered to a subject, thereby forming a complex that recruits an RNA editing entity present in a cell of the subject;
wherein the RNA editing entity, when associated with the engineered guide RNA and the target RNA, performs a targeted editing of a base of a nucleotide of the target RNA.
   Embodiment 2. The engineered guide RNA of Embodiment 1, wherein the engineered guide RNA is single-stranded.
   Embodiment 3. The engineered guide RNA of Embodiment 1 or Embodiment 2, wherein the engineered guide RNA comprises a mismatch relative to the target RNA.
   Embodiment 4. The engineered guide RNA of Embodiment 2, wherein the mismatch comprises a base in the engineered guide RNA opposite to and unpaired with a base in the target RNA molecule.
   Embodiment 5. The engineered guide RNA of Embodiment 2, wherein the mismatch comprises an A/C mismatch and wherein the A is in the target RNA molecule and the C is in the engineered guide RNA.
   Embodiment 6. The engineered guide RNA of Embodiment 5, wherein the A in the A/C mismatch comprises the base of the nucleotide in the target RNA molecule chemically modified by the RNA editing entity.
   Embodiment 7. The engineered guide RNA of Embodiment 6, wherein the engineered guide RNA comprises a C opposite the base of the nucleotide in the target RNA chemically modified by the RNA editing entity.
   Embodiment 8. The engineered guide RNA of Embodiment 6, wherein the target RNA molecule comprises a G adjacent to and 5′ of the base of the nucleotide in the target RNA chemically modified by the RNA editing entity.
   Embodiment 9. The engineered guide RNA of Embodiment 7, wherein the engineered guide further comprises a G adjacent to and 5′ of the C opposite to and unpaired with the A in the target RNA molecule chemically modified by the RNA editing entity.
   Embodiment 10. The engineered guide RNA of any of Embodiment 6-Embodiment 9, wherein the engineered guide RNA comprises an unmodified nucleotide on either side of the mismatch.
   Embodiment 11. The engineered guide RNA of any one of Embodiment 6-Embodiment 10, wherein the engineered guide RNA does not comprise a second mismatch within 2 nucleotides of the mismatch.
   Embodiment 12. The engineered guide RNA of any one of the preceding Embodiments, wherein the at least one chemical modification is positioned:
(a) proximal to a 5′ end of the engineered guide RNA;
(b) proximal to a 5′ end of a region of the engineered guide RNA; or
(c) both (a) and (b).
   Embodiment 13. The engineered guide RNA of any one of the preceding Embodiments, wherein the at least one chemical modification is positioned:
(a) proximal to a 3′ end of the engineered guide RNA;
(b) proximal to a 3′ end of a region of the engineered guide RNA; or
(c) both (a) and (b).
   Embodiment 14. The engineered guide RNA of any one of the preceding Embodiments, wherein the at least one chemical modification is positioned proximal to a 5′ end of the engineered guide RNA, proximal to a 5′ end of a region of the engineered guide RNA, proximal to a 3′ end of the engineered guide RNA, proximal to a 3′ end of a region of the engineered guide RNA, or any combination thereof.
   Embodiment 15. The engineered guide RNA of any one of the preceding Embodiments, wherein the engineered guide RNA, when present in an aqueous solution and not bound to the target RNA, does not bind to the RNA editing entity with a dissociation constant less than about 100 nM.
   Embodiment 16. The engineered guide RNA of any one of the preceding Embodiments, wherein the engineered guide RNA, when present in an aqueous solution and not bound to the target RNA, lacks at least one of a hairpin, a bulge, a polynucleotide loop, or a structural domain, or any combination thereof.
   Embodiment 17. The engineered guide RNA of any one of the preceding Embodiments, wherein the complex comprises a structural feature that comprises a bulge, an internal loop, a hairpin, a mismatch, a wobble base pair, or any combination thereof.
   Embodiment 18. The engineered guide RNA of Embodiment 17, wherein the structural feature comprises a bulge.
   Embodiment 19. The engineered guide RNA of Embodiment 18, wherein the bulge comprises an asymmetric bulge.
   Embodiment 20. The engineered guide RNA of Embodiment 18, wherein the bulge comprises a symmetric bulge.
   Embodiment 21. The engineered guide RNA of Embodiment 17, wherein the structural feature comprises an internal loop.
   Embodiment 22. The engineered guide RNA of Embodiment 21, wherein the internal loop comprises an asymmetric internal loop.
   Embodiment 23. The engineered guide RNA of Embodiment 21, wherein the internal loop comprises a symmetric internal loop.
   Embodiment 24. The engineered guide RNA of Embodiment 17, wherein the structural feature comprises the hairpin, wherein the hairpin comprises a double stranded RNA non-targeting domain.
   Embodiment 25. The engineered guide RNA of any one of the preceding Embodiments, wherein the at least one chemical modification comprises a substitution of one or both of non-linking phosphate oxygen atoms in a phosphodiester backbone linkage of the engineered guide RNA as provided in Table 2.
   Embodiment 26. The engineered guide RNA of any one of the preceding Embodiments, wherein the at least one chemical modification comprises a substitution of one or more of linking phosphate oxygen atoms in a phosphodiester backbone linkage of the engineered guide RNA as provided in Table 2.
   Embodiment 27. The engineered guide RNA of any one of the preceding Embodiments, wherein the at least one chemical modification comprises a modification to a sugar of a nucleotide of the engineered guide RNA as provided in Table 2.
   Embodiment 28. The engineered guide RNA of Embodiment 27, wherein the modification to the sugar of the nucleotide of the engineered guide RNA comprises at least one locked nucleic acid (LNA).
   Embodiment 29. The engineered guide RNA of Embodiment 27, wherein the modification to the sugar of the nucleotide of the engineered guide RNA comprises at least one unlocked nucleic acid (UNA).
   Embodiment 30. The engineered guide RNA of Embodiment 27, wherein the modification to the sugar comprises a modification of a constituent of the sugar, wherein the sugar comprises a ribose sugar.
   Embodiment 31. The engineered guide RNA of Embodiment 30, wherein the modification to the constituent of the ribose sugar of the nucleotide of the engineered guide RNA comprises a 2′-O-methyl group.
   Embodiment 32. The engineered guide RNA of any one of the preceding Embodiments, wherein the at least one chemical modification comprises replacement of a phosphate moiety of the engineered guide RNA with a dephospho linker as provided in Table 2.
   Embodiment 33. The engineered guide RNA of any one of the preceding Embodiments, wherein the at least one chemical modification comprises a modification of a phosphate backbone of the engineered guide RNA as provided in Table 2.
   Embodiment 34. The engineered guide RNA of Embodiment 33, wherein the engineered guide RNA comprises a phosphothioate group.
   Embodiment 35. The engineered guide RNA of any one of the preceding Embodiments, wherein the at least one chemical modification comprises a modification to a base of a nucleotide of the engineered guide RNA.
   Embodiment 36. The engineered guide RNA of Embodiment 35, wherein the at least one chemical modification comprises an unnatural base of a nucleotide as provided in Table 2.
   Embodiment 37. The engineered guide RNA of any one of the preceding Embodiments, wherein the at least one chemical modification comprises a morpholino group, a cyclobutyl group, pyrrolidine group, or peptide nucleic acid (PNA) nucleoside surrogate.
   Embodiment 38. The engineered guide RNA of any one of the preceding Embodiments, wherein the at least one chemical modification comprises at least one stereopure nucleic acid as provided in Table 2.
   Embodiment 39. The engineered guide RNA of any one of the preceding Embodiments, wherein the engineered guide RNA comprises from 1 to 100 chemical modifications, each of which can be independently the same or different.
   Embodiment 40. The engineered guide RNA of any one of any one of the preceding Embodiments, wherein the at least one chemical modification does not comprise a naturally occurring chemical modification to a nucleic acid in a eukaryotic cell.
   Embodiment 41. The engineered guide RNA of any one of the preceding Embodiments, wherein the at least one chemical modification increases specificity of the engineered guide RNA binding to the target RNA compared to a specificity of an otherwise identical reference polynucleotide without the at least one chemical modification.
   Embodiment 42. The engineered guide RNA of any one of the preceding Embodiments, wherein the at least one chemical modification increases resistance to nuclease digestion of the engineered guide RNA compared to resistance to nuclease digestion of an otherwise identical reference polynucleotide without the at least one chemical modification as measured in an in vitro assay.
   Embodiment 43. The engineered guide RNA of any one of the preceding Embodiments, wherein the at least one chemical modification decreases immunogenicity of the engineered guide RNA compared to immunogenicity of an otherwise identical reference polynucleotide without the at least one chemical modification as measured in an in vitro assay.
   Embodiment 44. The engineered guide RNA of any one of the preceding Embodiments, wherein the engineered guide RNA comprises DNA.
   Embodiment 45. The engineered guide RNA of any one of preceding Embodiments, wherein the target RNA comprises RAB7A, ABCA4, SERPINA1, SERPINA1 E342K, HEXA, LRRK2, SNCA, APP, Tau, CFTR, ALAS1, ATP7B, ATP7B G1226R, HFE C282Y, LIPA c.894 G>A, PCSK9 start site, or SCNN1A start site, a fragment any of these, or any combination thereof.
   Embodiment 46. The engineered guide RNA of Embodiment 45, wherein the target RNA comprises SERPINA1 E342K.
   Embodiment 47. The engineered guide RNA of Embodiment 45, wherein the engineered guide RNA has at least 70%, at least 80%, at least 85%, at least 90%, at least 92%, at least 95%, at least 97%, or at least 99% sequence identity with any one of SEQ ID NOs: 1-2.
   Embodiment 48. The engineered guide RNA of Embodiment 45, wherein the target RNA encodes ABCA4.
   Embodiment 49. The engineered guide RNA of Embodiment 45, wherein the engineered guide RNA has at least 70%, at least 80%, at least 85%, at least 90%, at least 92%, at least 95%, at least 97%, or at least 99% sequence identity with SEQ ID NO: 1.
   Embodiment 50. The engineered guide RNA of Embodiment 45, wherein the engineered guide RNA has at least 70%, at least 80%, at least 85%, at least 90%, at least 92%, at least 95%, at least 97%, or at least 99% sequence identity with SEQ ID NO: 2.
   Embodiment 51. The engineered guide RNA of any one of the preceding Embodiments, wherein the engineered guide RNA is isolated, or purified, or both.
   Embodiment 52. The engineered guide RNA of any one of the preceding Embodiments, wherein the RNA editing entity is:
(a) ADAR or APOBEC;
(b) a catalytically active fragment of (a);
(c) fusion polypeptide comprising (a) or (b); or
(d) any combination of (a)-(c).
   Embodiment 53. The engineered guide RNA of Embodiment 52, wherein the RNA editing entity comprises ADAR, and wherein the ADAR comprises ADAR1, ADAR2, ADAR3, or a combination thereof.
   Embodiment 54. The engineered guide RNA of any one of the preceding Embodiments, wherein the RNA editing entity is endogenous to the cell of the subject.
   Embodiment 55. The engineered guide RNA of any one of the preceding Embodiments, wherein the RNA editing entity is exogenously provided.
   Embodiment 56. The engineered guide RNA of any one of the preceding Embodiments, further comprising a structural loop stabilized scaffold.
   Embodiment 57. The engineered guide RNA of Embodiment 56, wherein the structural loop stabilized scaffold comprises a stem loop, a junction, a T junction, a clover leaf, a pseudoknot, or any combination thereof.
   Embodiment 58. The engineered guide RNA of Embodiment 56 or Embodiment 57, wherein the structural loop stabilized scaffold comprises at least 1, least 2, at least 3, at least 4, at least 5, at least 6, at least 7, at least 8, at least 9, or at least 10 stem loop structures.
   Embodiment 59. The engineered guide RNA of any one of Embodiment 56-Embodiment 58, wherein the structural loop stabilized scaffold comprises a tRNA scaffold.
   Embodiment 60. The engineered guide RNA of any one of the preceding Embodiments, further comprising an RNA editing entity recruiting domain.
   Embodiment 61. The engineered guide RNA of any one of the preceding Embodiments, wherein the engineered guide RNA is conjugated to a targeting moiety.
   Embodiment 62. The engineered guide RNA of Embodiment 61, wherein the targeting moiety targets a neuronal cell.
   Embodiment 63. The engineered guide RNA of Embodiment 61, wherein the targeting moiety targets a liver cell.
   Embodiment 64. The engineered guide RNA of Embodiment 61, wherein the targeting moiety targets a macular cell.
   Embodiment 65. The engineered guide RNA of any one of the preceding Embodiments, wherein the engineered guide RNA is encapsulated in particles.
   Embodiment 66. The engineered guide RNA of Embodiment 65, wherein the particles comprise nanoparticles.
   Embodiment 67. The engineered guide RNA of Embodiment 65, wherein the particles comprise liposomes.
   Embodiment 68. An isolated cell comprising the engineered guide RNA of any one of the preceding Embodiments.
   Embodiment 69. The isolated cell of Embodiment 68, wherein the isolated cell comprises an immune cell.
   Embodiment 70. The isolated cell of Embodiment 69, wherein the immune cell comprises a T cell.
   Embodiment 71. An isolated plurality of cells comprising the engineered guide RNA of any one of the preceding Embodiments.
   Embodiment 72. The isolated plurality of cells of Embodiment 71, wherein the isolated plurality of cells comprises immune cells.
   Embodiment 73. The isolated plurality of cells of Embodiment 72, wherein the immune cells are T cells.
   Embodiment 74. A pharmaceutical composition in unit dose form comprising:
(a) the engineered guide RNA of any one of Embodiment 1-Embodiment 67; the isolated cell of any one of Embodiment 68-Embodiment 70, or the isolated plurality of cells of any one of Embodiment 71-Embodiment 73; and
(b) a pharmaceutically acceptable: excipient, carrier, or diluent.
   Embodiment 75. A kit comprising:
(a) the engineered guide RNA of any one of Embodiment 1-Embodiment 67; the isolated cell of any one of Embodiment 68-Embodiment 70, or the isolated plurality of cells of any one of Embodiment 71-Embodiment 73, or the pharmaceutical composition of Embodiment 74; and
(b) a container.
   Embodiment 76. A method of delivering the engineered guide RNA of any one of Embodiment 1-Embodiment 67 or the pharmaceutical composition of Embodiment 74 to a cell, the method comprising: delivering directly or indirectly an engineered guide RNA to the cell.
   Embodiment 77. A method of treating or preventing a disease or a condition in a subject in need thereof, the method comprising: administering to the subject the engineered guide RNA of any one of Embodiment 1-Embodiment 67 or the pharmaceutical composition of Embodiment 74.
   Embodiment 78. The method of Embodiment 75, wherein the pharmaceutical composition is administered to the subject intrathecally, intraocularly, intravitreally, retinally, intravenously, intramuscularly, intraventricularly, intracerebrally, intracerebellarly, intracerebroventricularly, intraperenchymally, subcutaneously, or a combination thereof.
   Embodiment 79. The method of Embodiment 78, wherein the administering comprises a therapeutically effective amount sufficient to treat the subject.
   Embodiment 80. The method of Embodiment 77 or Embodiment 78, wherein the disease or the condition comprises a neurological disease or condition.
   Embodiment 81. The method of Embodiment 80, wherein the neurological or neurodevelopmental disease or condition comprises Parkinson's disease, Alzheimer's disease, or dementia.
   Embodiment 82. The method of Embodiment 77 or Embodiment 78, wherein the disease or the condition comprises a liver disease or condition.
   Embodiment 83. The method of Embodiment 82, wherein the liver disease or condition comprises liver cirrhosis.
   Embodiment 84. The method of Embodiment 82, wherein the liver disease or condition comprises alpha-1 antitrypsin deficiency (AAT deficiency).
   Embodiment 85. The method of Embodiment 77 or Embodiment 78, wherein the disease or the condition comprises macular degeneration.
   Embodiment 86. The method of Embodiment 85, wherein the macular degeneration comprises Stargardt's disease.

### EXAMPLES

The following illustrative examples are representative of embodiments of the stimulation, systems, and methods described herein and are not meant to be limiting in any way.

### Example 1. Treating a Neurological Disease by Editing RNA

A subject will be diagnosed with Parkinson's disease stemmed from a G2019S mutation in the LRRK2 gene. The subject will be prescribed a dosing regimen of a pharmaceutical composition comprising an chemically modified engineered guide RNA, as disclosed herein, for recruiting nucleic acid editing entity to the target LRRK2 transcript comprising the G2019S mutation. The chemically modified engineered guide RNA, upon binding to the LRRK2 transcript (e.g. LRRK2 pre-mRNA or LRRK2 mRNA) forms a structural feature in association with the target LRRK2 transcript, which at least will then recruit the nucleic acid editing entity (e.g. an RNA editing entity) to edit the mutation of the LRRK2 transcript. In some cases, the chemically modified engineered guide RNA increases the efficiency of recruiting the RNA editing entity as compared to an unmodified engineered guide RNA. In some cases, the chemically modified engineered guide RNA increases the specificity of the RNA editing entity for editing the LRRK2 transcript. In some embodiments, the chemically modified engineered guide RNA increases resistance towards nuclease digestion of hydrolysis when the engineered guide RNA may be administered to the subject. The pharmaceutical composition can be formulated for administering to the subject by parenchymal injection in an effective amount to treat the subject for Parkinson's disease.

### Example 2. RNA Editing by Engineered Guide RNA

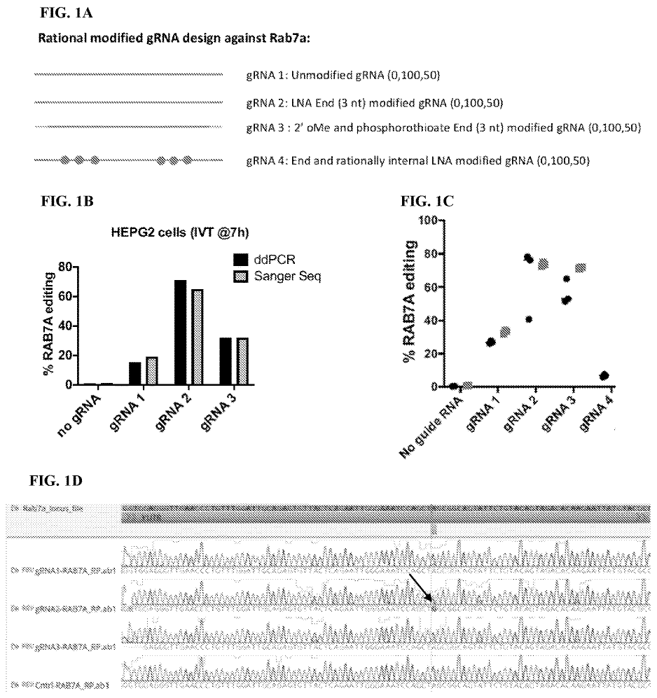

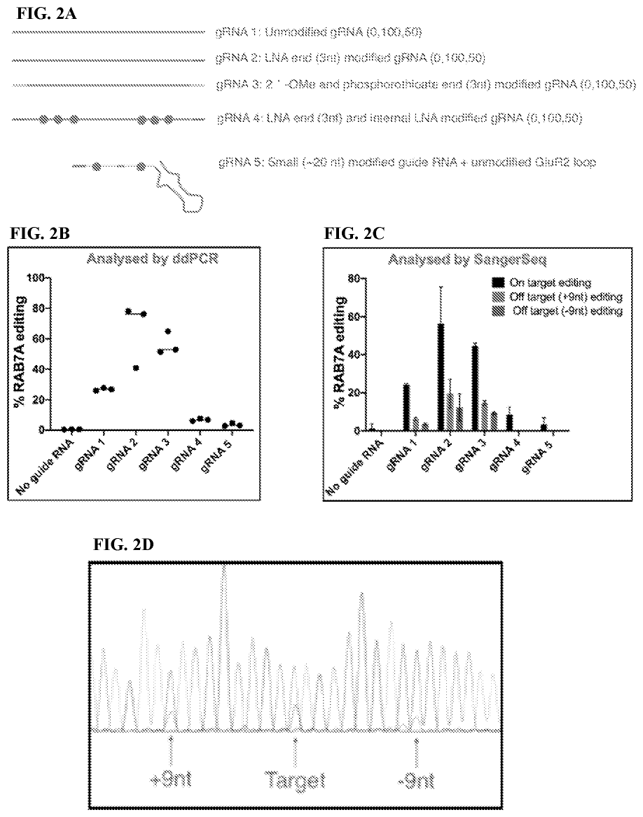

Engineered guide RNA comprising unmodified or chemically modified gRNA were resuspended in nuclease-free water at a final concentration of 40 uM. Chemical modification of the engineered guide RNA can be at any position of the engineered guide RNA. In some cases, the chemical modification can be within the stem regions. FIG. 1A and FIG. 2A illustrate the exemplary engineered guide RNAs comprising the chemically modified gRNA for editing RAB7A.

K562 cells were maintained in RPMI media, supplemented with 10% FBS and 1% Penicillin/Streptomycin solution at 37° C. and 5% CO2. 40 pmol of gRNAs were electroporated in 2×10\hat{ }5 cells using 4D-Nucleofector™ X Kit using 16-well Nucleocuvette Strips (Lonza) following recommended protocol. Post-electroporation, the cells were immediately placed in 500 ml of pre-warmed culture media and cultured for 3 h (for the end point experiment) or for designated times (for the temporal study). Cells were harvested and cellular RNA were isolated using RNeasy Mini Kit (Qiagen). 60 ng of total RNA were used make cDNA using First-Strand cDNA Synthesis kit, using a random hexamer primer approach (Thermofisher Scientific). cDNAs were then used in both sanger sequencing and droplet digital PCR (ddPCR) to measure editing efficiency.

For Sanger sequencing approach, the target region of RAB7A was amplified via PCR with the following primers:

| | |
|---|---|
| RAB7AFP: | AGGCCTGTAAGGTGGAGGG |
| RAB7ARP: | TGAAATAACGGCAATTTATCCATTGCACATAC |

The column purified PCR products were sequenced using sanger sequencing (Genewiz) and editing was analyzed using EditR program.

For droplet digital PCR (ddPCR), A 20 μl ddPCR reaction was prepared with 10 μl of Bio-Rad ddPCR 2× Supermix for Probes (no dUTP), 1 μl of 9 μM forward primer (FP), 1 μl of 9 μM reverse primer (RP), 1 μl of 5 μM target internal probe (FAM), 1 μl of 5 μM reference internal probe (HEX), 5 μl of 0.5 ng/μl cDNA, and 1 μl nuclease free water in a 96 well PCR plate. 20 μl of the PCR reaction was used for droplet generation on the Bio-Rad QX200 droplet generator following manufacturer's instructions. The droplets were then transferred to a Bio-Rad 96-well ddPCR plate and heat sealed with a foil seal at 180° C. for 5 seconds. Thermal cycling was performed on the Bio-Rad C1000 thermal cycler using the following conditions: 95° C. for 10 minutes, followed by 40 cycles of 94° C. for 30 sec and 60° C. for 1 minute; 98° C. for 10 minutes and hold at 4° C. Temperature ramp rates were set at 2° C./s. Following amplification, the droplets were read on the Bio-Rad QX200 ddPCR system. The droplets may then be analyzed using Bio-Rad Quantasoft Analysis Pro software with wild-type and drop-off populations determined through manual gating. The following primers and probes were used:

RAB7A_ddPCR_FP:
AGGCCTGTAAGGTGGAGGG
RAB7A_ddPCR_RP:
AATTTATCCATTGCACATAC
Target internal probe:
/56-FAM/CAGAATTGGGAAATCCAGCTAG/3IABkFQ/
Reference internal probe:
/5HEX/ACTGTCTAGTTCCCTTCTGTG/3IABkFQ/

Some gRNA sequences targeting RAb7A are summarized below in Table 4.

TABLE 4
Guide Sequences against RAB7A

| gRNA | Sequence |
|---|---|
| gRNA1 | TGATAAAAGGCGTACATAATTCTTGTGTCTACTGTACAGAATAC |
| | TGCCGCCAGCTGGATTTCCCAATTCTGAGTAACACTCTGCAATC |
| | CAAACAGGGTTC |
| gRNA2 | + T + G + ATAAAAGGCGTACATAATTCTTGTGTCTACTGTA |
| | CAGAATACTGCCGCCAGCTGGATTTCCCAATTCTGAGTAACACT |
| | CTGCAATCCAAACAGGG + T + T + C |
| gRNA3 | mT*mG*mA*TAAAAGGCGTACATAATTCTTGTGTCTACTGTACA |
| | GAATACTGCCGCCAGCTGGATTTCCCAATTCTGAGTAACACTCT |
| | GCAATCCAAACAGGG*mT*mT*mC |
| gRNA4 | + T + GA + TAAAAGGCG + TA + CA + TAATTCTTGTG |
| | T + CTA + C + TGTACAGAATAC + TG + C + CGCCAG |
| | CTGGAT + TT + CCCAA + TT + C + TGAG + TAA + |
| | CACTC + TG + CAATCCAAA + CAGGG + T + T + C |

+ indicates an LNA
m indicates a 2′OMe base modification
*indicates a phosphorothioate backbone modification

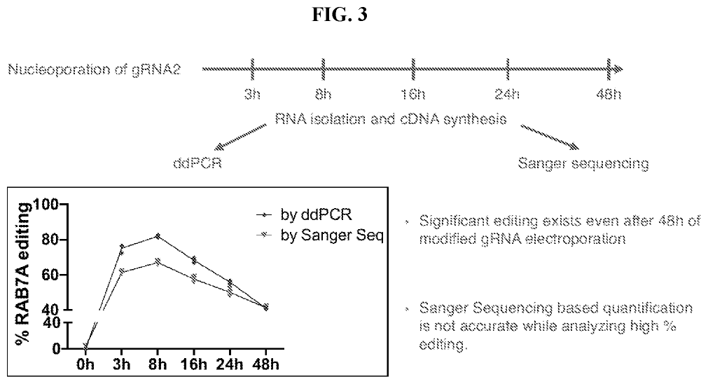

As seen in FIG. 1B-D and FIG. 2B-D, the engineered guide RNA comprising the gRNA comprising the at least one chemical modification described herein modified RAB7A with increased efficiency compared to editing with unmodified gRNA. In some instances, the engineered guide RNA comprising the chemical modification exhibited decreased off-target editing (FIG. 2D). In some embodiments, the editing of the RAB7A exhibited a duration of editing that corresponded to the presence of the engineered guide RNA comprising the gRNA (FIG. 3) introduced into the cell. As shown in FIG. 3, the chemical modification of introducing LNA into the engineered guide RNA extended the half-life of the engineered guide RNA comprising the gRNA for editing RAB7A to at least 48 hours after introducing the engineered guide RNA to the cell.

Varying the length of chemically modified guide RNAs was tested in K562 cells. The above described methods for cell transfection and sequencing were carried out with the chemically modified guides described below in Table 5.

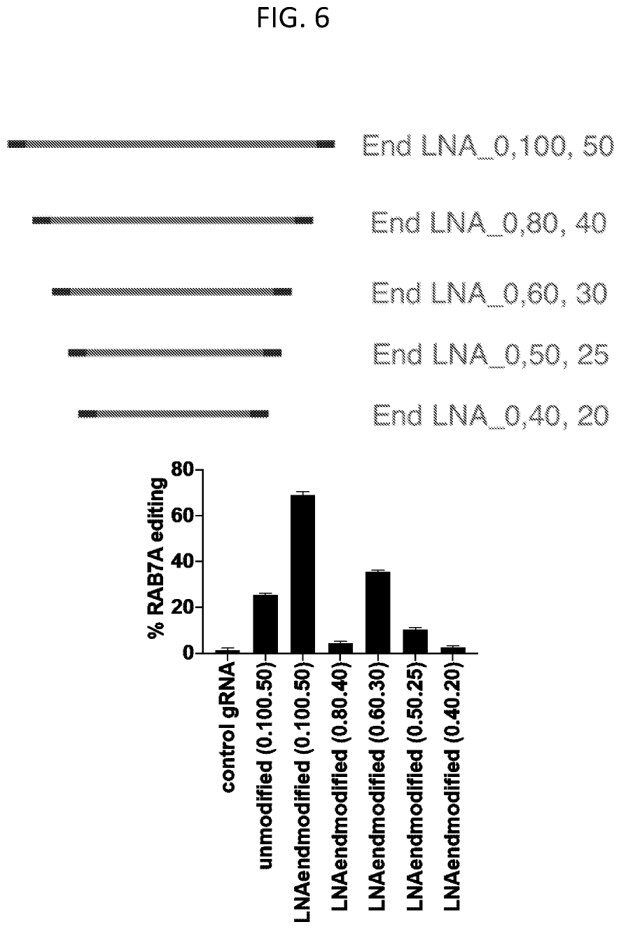

TABLE 5
Guide Sequences in FIG. 6

| gRNA | Sequence |
|---|---|
| control gRNA | AACATCGUAGAUUGAAGCCACAAAAUCCACAGCACAC |
| (to SNCA) | AAAGACCCUGCCACCAUGUAUUCACUUCAGUGAAAGG |
| | GAAGCACCGAAAUGCUGAGUGGGGGC |
| unmodified | TGATAAAAGGCGTACATAAGTCTTGTGTCTACTGTAC |
| (0, 100, 50) | AGAAGACTGCCGCCAGCTGGATTTCCCAATTCTGAGT |
| | AACACTCTGCAATCCAAACAGGGTTC |
| End LNA_ | + T + G + ATAAAAGGCGTACATAAGTCTTGTGTC |
| 0, 100, 50 | TACTGTACAGAAGACTGCCGCCAGCTGGATTTCCCAA |
| | TTCTGAGTAACACTCTGCAATCCAAACAGGG + T + |
| | T + C |
| End LNA_ | + C + G + TACATAAGTCTTGTGTCTACTGTACAG |
| 0, 80, 40 | AAGACTGCCGCCAGCTGGATTTCCCAATTCTGAGTAA |
| | CACTCTGCAAT + C + C + A |
| End LNA_ | + T + C + TTGTGTCTACTGTACAGAAGACTGCCG |
| 0, 60, 30 | CCAGCTGGATTTCCCAATTCTGAGTAAC + A + |
| | C + T |
| End LNA_ | + T + G + TCTACTGTACAGAAGACTGCCGCCAGC |
| 0, 50, 25 | TGGATTTCCCAATTCTGA + G + T + A |
| End LNA_ | + A + C + TGTACAGAAGACTGCCGCCAGCTGGAT |
| 0, 40, 20 | TTCCCAAT + T + C + T |

+ indicates an LNA

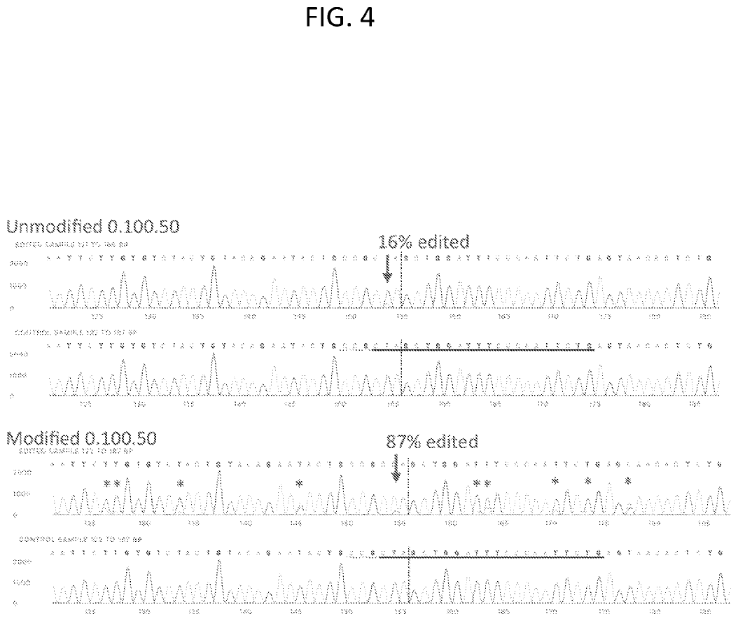

Chemically modified guide RNAs also displayed higher levels of RNA editing of RAB7A in ARPE-19 retinal epithelial cells. Cells were transfected with 60 pmol of unmodified gRNA and chemically modified gRNA. RNA was isolated at 7 hours and the cDNA was PCR amplified any Sanger sequenced. The EditR software was used to quantify percent RNA editing. As seen in FIG. 4, a chemically modified engineered gRNA (gRNA 3 of FIG. 1A), yielded 87% editing of the target A in RAB7A as compared to an unmodified gRNA (gRNA1 of FIG. 1A), which only yielded 16% editing of the target A in RAB7A. The target A may be indicated by an arrow.

### Example 3. Chemically Modified Engineered Guide RNAs for Editing of SNCA

This example describes chemically modified engineered guide RNAs for editing of the 3′UTR of SNCA. Transfection and sequencing were carried out as described in EXAMPLE 2. Chemically modified engineered guide RNAs comprising LNA end modifications at the 3 nucleotides at the 5′ and 3′ end of the guide yielded higher percent editing (˜30-40% editing) of the SNCA 3′UTR as compared to unmodified guide RNAs (˜20% editing) to the same region. Some sequences of guides are shown below in Table 6.

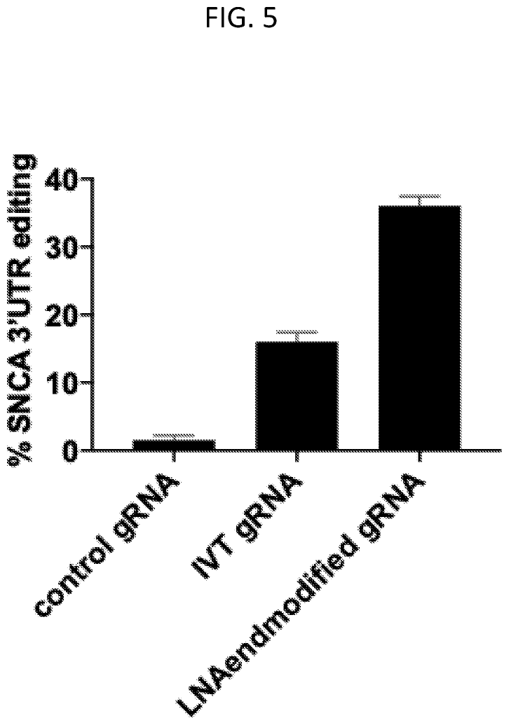

TABLE 6
Guide Sequences in FIG. 5

| gRNA | Sequence |
|---|---|
| control gRNA | TGATAAAAGGCGTACATAAGTCTTGTGTCTACTGTAC |
| (to Rab7a) | AGAAGACTGCCGCCAGCTGGATTTCCCAATTCTGAGT |
| | AACACTCTGCAATCCAAACAGGGTTC |
| IVT gRNA | AACATCGUAGAUUGAAGCCACAAAAUCCACAGCACAC |
| | AAAGACCCUGCCACCAUGUAUUCACUUCAGUGAAAGG |
| | GAAGCACCGAAAUGCUGAGUGGGGGC |
| LNA end | + A + A + CATCGUAGAUUGAAGCCACAAAAUCCA |
| modified | CAGCACACAAAGACCCUGCCACCAUGUAUUCACUUCA |
| gRNA | GUGAAAGGGAAGCACCGAAAUGCUGAGUGGG + G + |
| | G + C |

+ indicates an LNA

### Example 4. Chemically Modified Engineered Guide RNAs for Editing of RAB7A

The extent of LNA and 2′OMe chemical modification on RNA editing and serum stability was also tested. K562 cells (2×10\hat{ }5 cells) were electroporated with 40 pmol of gRNAs using 4D-Nucleofector™ X Kit and 16-well Nucleocuvette Strips (Lonza). Post-electroporation, the cells were immediately placed in 500 ml of pre-warmed culture media and cultured for 3 h. Cells were harvested and RNA was isolated for Sanger sequencing. For stability testing, gRNAs were exposed to phosphate buffered saline (“PBS”) or fetal bovine serum (“FBS”; PBS having 10% FBS). After 18 hours of incubation, an agarose gel to evaluate serum stability.

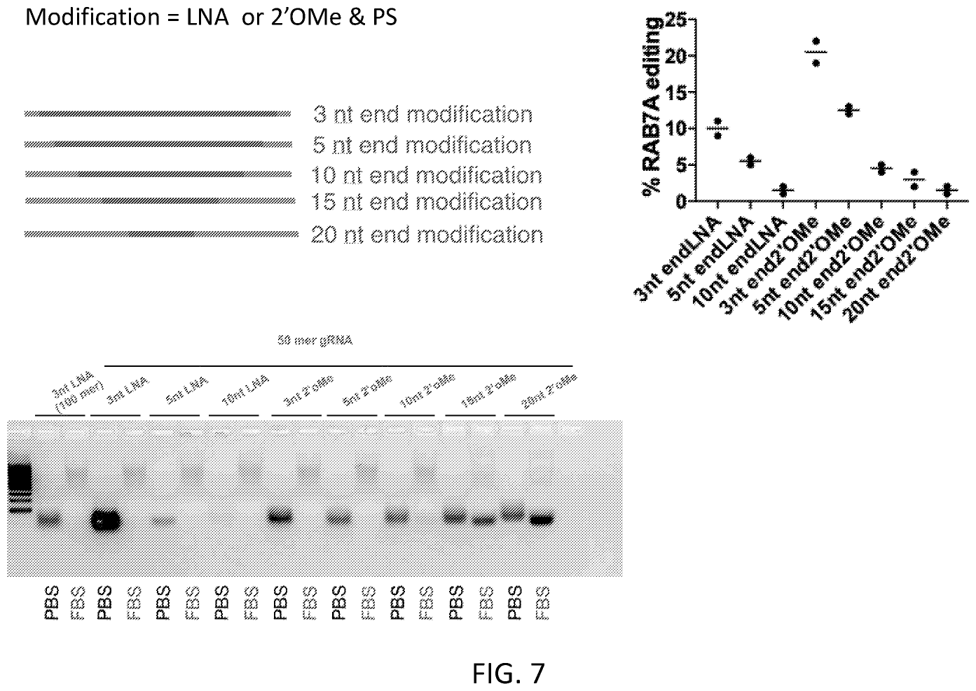

FIG. 7 shows schematics of the various gRNAs tested (top left), results of RNA editing (top right), and gRNA stability (bottom). As show in FIG. 7, levels of RNA editing of RAB7A decreased with an increased number of chemical modifications. As also shown in FIG. 7, serum stability increased with an increased number of chemical modifications. An agarose gel was run under non-denaturing conditions and multiple bands were observed. This may be indicate that the various gRNAs adopt different structural conformations.

gRNA sequences are shown in TABLE 7 below. + indicates an LNA and italics indicates a 2′OMe.

TABLE 7

| gRNA | Sequence |
|---|---|
| 3 nt end LNA | + T + G + TCTACTGTACAGAAGACTGCCGCC |
| | AGCTGGATTTCCCAATTCTGA + G + T + A |
| 5 nt end LNA | + T + G + T + C + TACTGTACAGAAGACT |
| | GCCGCCAGCTGGATTTCCCAATTCT + G + |
| | A + G + T + A |
| 10 nt end LNA | + T + G + T + C + T + A + C + T + |
| | G + TACAGAAGACTGCCGCCAGCTGGATTTCCC |
| | A + A + T + T + C + T + G + A + |
| | G + T + A |
| 3 nt end 2′OMe | TGTCTACTGTACAGAAGACTGCCGCCAGCTGGAT |
| | TTCCCAATTCTGAGTA |
| 5 nt end 2′OMe | TGTCTACTGTACAGAAGACTGCCGCCAGCTGGAT |
| | TTCCCAATTCTGAGTA |
| 10 nt end 2′OMe | TGTCTACTGTACAGAAGACTGCCGCCAGCTGGAT |
| | TTCCCAATTCTGAGTA |
| 15 nt end 2′OMe | TGTCTACTGTACAGAAGACTGCCGCCAGCTGGAT |
| | TTCCCAATTCTGAGTA |
| 20 nt end 2′OMe | TGTCTACTGTACAGAAGACTGCCGCCAGCTGGAT |
| | TTCCCAATTCTGAGTA |

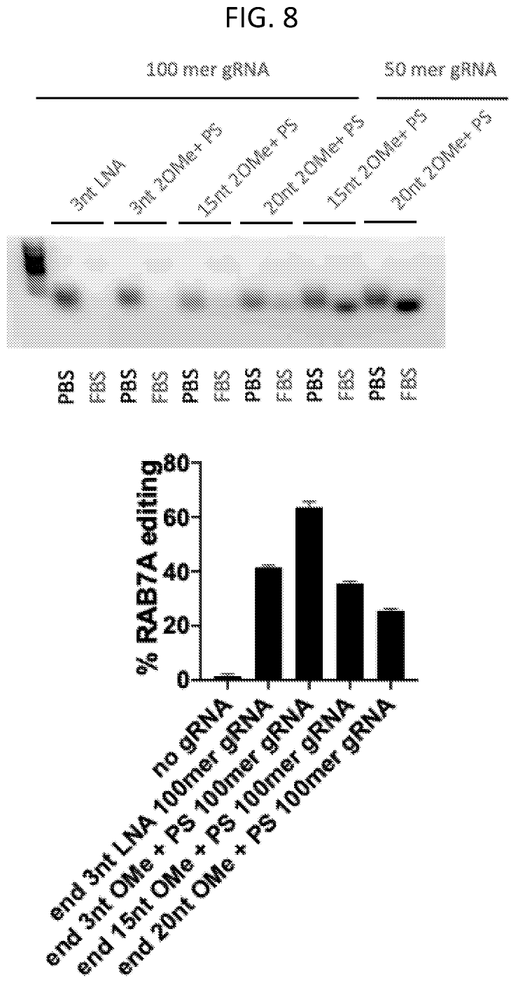

FIG. 8 shows an RNA agarose gel and a graph of RNA editing for guides of a length of 100 and 50. As shown, an increased number of chemical modifications improved serum stability. Further, longer lengths of guides (100 mers) exhibited with varying levels of chemical modifications exhibited a high percent RNA editing of ˜20 to 60%.

gRNA sequences are shown in TABLE 8 below. + indicates an LNA, italics indicates a 2′OMe, and * indicates phosphorothioate (PS) modifications to the phosphate backbone.

TABLE 8

| gRNA | Sequence |
|---|---|
| 100 mer gRNA | + T + G + ATAAAAGGCGTACATAAGTCT |
| 3 nt end LNA | TGTGTCTACTGTACAGAAGACTGCCGCCAGC |
| | TGGATTTCCCAATTCTGAGTAACACTCTGCA |
| | ATCCAAACAGGG + T + T + C |
| 100 mer gRNA | T*G*A*TAAAAGGCGTACATAAGTCTTGTGT |
| 3 nt 2′OMe + PS | CTACTGTACAGAAGACTGCCGCCAGCTGGAT |
| | TTCCCAATTCTGAGTAACACTCTGCAATCCA |
| | AACAGGGT*T*C* |
| 100 mer gRNA | T*G*A*T*A*A*A*A*G*G*C*G*T*A*C*A |
| 15 nt 2′OMe + PS | TAAGTCTTGTGTCTACTGTACAGAAGACTGC |
| | CGCCAGCTGGATTTCCCAATTCTGAGTAACA |
| | CTCTGCAA*T*C*C*A*A*A*C*A*G*G*G* |
| | T*T*C* |
| 100 mer gRNA | T*G*A*T*A*A*A*A*G*G*C*G*T*A*C*A |
| 20 nt 2′OMe + PS | *T*A*A*G*TCTTGTGTCTACTGTACAGAAG |
| | ACTGCCGCCAGCTGGATTTCCCAATTCTGAG |
| | TAACACTC*T*G*C*A*A*T*C*C*A*A*A* |
| | C*A*G*G*G*T*T*C* |
| 50 mer gRNA | T*G*T*C*T*A*C*T*G*T*A*C*A*G*A*G |
| 15 nt 2′OMe + PS | ACTGCCGCCAGCTGATT*T*C*C*C*A*A*T |
| | *T*C*T*G*A*G*T*A |
| 50 mer gRNA | T*G*T*C*T*A*C*T*G*T*A*C*A*G*A*A |
| 20 nt 2′OMe + PS | *G*A*C*T*GCCGCCAGCT*G*G*A*T*T*T |
| | *C*C*C*A*A*T*T*C*T*G*A*G*T*A |

### Example 5. Chemically Modified Engineered Guide RNAs for Editing of ACTB, SNCA, and ABCA4

RNA editing of three targets (ACTB, SNCA, and ABCA4) was assessed with chemically modified guides of the present disclosure, where the gRNAs were 100 nucleotides in length. In all cases, gRNAs against the target were modified to include 3 locked nucleic acids (LNAs) at the 5′ and 3′ end of the gRNA.

For ACTB experiments were carried out as follows. The extent of LNA and 2′OMe chemical modification on RNA editing and serum stability was also tested. K562 cells or HepG2 cells (2×10\hat{ }5 cells) were electroporated with 40 pmol of gRNAs using 4D-Nucleofector™ X Kit and 16-well Nucleocuvette Strips (Lonza). Post-electroporation, the cells were immediately placed in 500 ml of pre-warmed culture media and cultured for 7 h, 24 h, or 48 h. Cells were harvested and RNA was isolated for Sanger sequencing.

For SNCA experiments were carried out as follows. The extent of LNA and 2′OMe chemical modification on RNA editing and serum stability was also tested. HEK293 cells (2×10\hat{ }5 cells) were electroporated with 40 pmol of gRNAs using 4D-Nucleofector™ X Kit and 16-well Nucleocuvette Strips (Lonza). Post-electroporation, the cells were immediately placed in 500 ml of pre-warmed culture media and cultured for 7 h. Cells were harvested and RNA was isolated for Sanger sequencing.

For ABCA4 experiments were carried out as follows. The extent of LNA and 2′OMe chemical modification on RNA editing and serum stability was also tested. HEK293 cells (2×10\hat{ }5 cells) carrying an ABCA4 minigene with the ABCA4 1961E mutation were electroporated with 40 pmol of gRNAs using 4D-Nucleofector™ X Kit and 16-well Nucleocuvette Strips (Lonza). Post-electroporation, the cells were immediately placed in 500 ml of pre-warmed culture media and cultured for 7 h. Cells were harvested and RNA was isolated for Sanger sequencing.

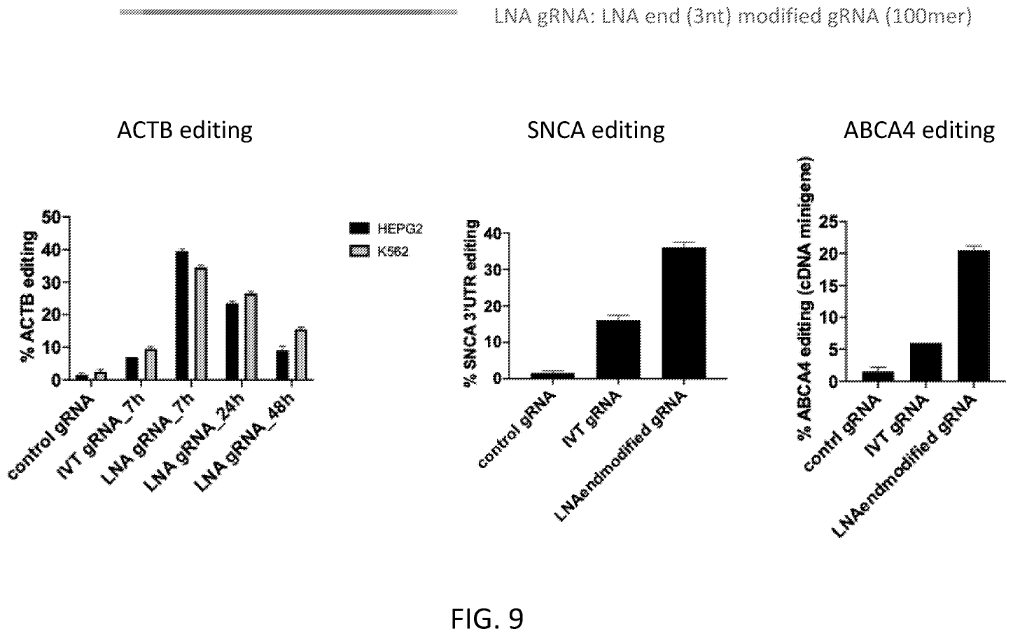

As shown in FIG. 9, chemically modified gRNAs successfully mediated editing of all 3 targets at higher levels than the IVT guides without modifications.

gRNA sequences are shown in TABLE 9 below. + indicates an LNA.

TABLE 9

| gRNA | Sequence |
|---|---|
| SNCA LNA | + A + A + C + A + TCGTAGATTGAAGCCACAAAAT |
| gRNA | CCACAGCACACAAAGACCCTGCCACCATGTATTCACTTCA |
| | GTGAAAGGGAAGCACCGAAATGCTGAGTGGG + G + |
| | G + C |
| ACTB LNA | + G + C + GAAGATTAAAAAAATTTTGCATTACATAAT |
| gRNA | TTACACGAAAGCAATGCCATCACCTCCCCTGTGTGGACTT |
| | GGGAGAGGACTGGGCCATTCTCC + T + T + A + |
| | G + A |
| ABCA4 LNA | + C + C + CCAGTGAGCATCTTGAATGTGGTTGTATTG |
| gRNA | CCGGCACCATTCACTCCCAGGAGGCCAAAGCACTCTCCAG |
| | TGAGAACTCGGACCACAGCCTCCCG + C + T + G |

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It may be intended that the following claims define the scope of the disclosure and that methods and structures within the scope of these claims and their equivalents be covered thereby.

What is claimed is:

1. An engineered guide RNA that comprises:
(a) at least one chemical modification, and
(b) a targeting domain, wherein the targeting domain hybridizes to a target RNA when administered to a subject, thereby forming a complex that recruits an RNA editing entity present in a cell of the subject;
wherein the RNA editing entity, when associated with the engineered guide RNA and the target RNA, performs a targeted editing of a base of a nucleotide of the target RNA.

2. The engineered guide RNA of claim 1, wherein the engineered guide RNA comprises a mismatch relative to the target RNA.

3. The engineered guide RNA of claim 2, wherein the mismatch comprises a base in the engineered guide RNA opposite to and unpaired with a base in the target RNA molecule.

4. The engineered guide RNA of claim 2, wherein the mismatch comprises an A/C mismatch and wherein the A is in the target RNA molecule and the C is in the engineered guide RNA.

5. The engineered guide RNA of claim 4, wherein the A in the A/C mismatch comprises the base of the nucleotide in the target RNA molecule chemically modified by the RNA editing entity.

6. The engineered guide RNA of claim 5, wherein the engineered guide RNA comprises a C opposite the base of the nucleotide in the target RNA chemically modified by the RNA editing entity.

7. The engineered guide RNA of claim 5, wherein the target RNA molecule comprises a G adjacent to and 5′ of the base of the nucleotide in the target RNA chemically modified by the RNA editing entity.

8. The engineered guide RNA of claim 6, wherein the engineered guide further comprises a G adjacent to and 5′ of the C opposite to and unpaired with the A in the target RNA molecule chemically modified by the RNA editing entity.

9. The engineered guide RNA of any of claims 5-8, wherein the engineered guide RNA comprises an unmodified nucleotide on either side of the mismatch.

10. The engineered guide RNA of any one of claims 5-9, wherein the engineered guide RNA does not comprise a second mismatch within 2 nucleotides of the mismatch.

11. The engineered guide RNA of any one of the preceding claims, wherein the at least one chemical modification is positioned:
(a) proximal to a 5′ end of the engineered guide RNA;
(b) proximal to a 5′ end of a region of the engineered guide RNA; or
(c) both (a) and (b).

12. The engineered guide RNA of any one of the preceding claims, wherein the at least one chemical modification is positioned:
(a) proximal to a 3′ end of the engineered guide RNA;
(b) proximal to a 3′ end of a region of the engineered guide RNA; or
(c) both (a) and (b).

13. The engineered guide RNA of any one of the preceding claims, wherein the at least one chemical modification is positioned proximal to a 5′ end of the engineered guide RNA, proximal to a 5′ end of a region of the engineered guide RNA, proximal to a 3′ end of the engineered guide RNA, proximal to a 3′ end of a region of the engineered guide RNA, or any combination thereof.

14. The engineered guide RNA of any one of the preceding claims, wherein the engineered guide RNA, when present in an aqueous solution and not bound to the target RNA, does not bind to the RNA editing entity with a dissociation constant less than about 100 nM.

15. The engineered guide RNA of any one of the preceding claims, wherein the at least one chemical modification comprises a substitution of one or both of non-linking phosphate oxygen atoms in a phosphodiester backbone linkage of the engineered guide RNA as provided in Table 2.

16. The engineered guide RNA of any one of the preceding claims, wherein the at least one chemical modification comprises a substitution of one or more of linking phosphate oxygen atoms in a phosphodiester backbone linkage of the engineered guide RNA as provided in Table 2.

17. The engineered guide RNA of any one of the preceding claims, wherein the at least one chemical modification comprises a modification to a sugar of a nucleotide of the engineered guide RNA as provided in Table 2.

18. The engineered guide RNA of claim 17, wherein the modification to the sugar of the nucleotide of the engineered guide RNA comprises at least one locked nucleic acid (LNA).

19. The engineered guide RNA of claim 17, wherein the modification to the sugar of the nucleotide of the engineered guide RNA comprises at least one unlocked nucleic acid (UNA).

20. The engineered guide RNA of claim 17, wherein the modification to the sugar comprises a modification of a constituent of the sugar, wherein the sugar comprises a ribose sugar.

21. The engineered guide RNA of claim 20, wherein the modification to the constituent of the ribose sugar of the nucleotide of the engineered guide RNA comprises a 2′-O-methyl group.

22. The engineered guide RNA of any one of the preceding claims, wherein the at least one chemical modification comprises replacement of a phosphate moiety of the engineered guide RNA with a dephospho linker as provided in Table 2.

23. The engineered guide RNA of any one of the preceding claims, wherein the at least one chemical modification comprises a modification of a phosphate backbone of the engineered guide RNA as provided in Table 2.

24. The engineered guide RNA of claim 23, wherein the engineered guide RNA comprises a phosphothioate group.

25. The engineered guide RNA of any one of the preceding claims, wherein the at least one chemical modification comprises a modification to a base of a nucleotide of the engineered guide RNA.

26. The engineered guide RNA of claim 25, wherein the at least one chemical modification comprises an unnatural base of a nucleotide as provided in Table 2.

27. The engineered guide RNA of any one of the preceding claims, wherein the at least one chemical modification comprises a morpholino group, a cyclobutyl group, pyrrolidine group, or peptide nucleic acid (PNA) nucleoside surrogate.

28. The engineered guide RNA of any one of the preceding claims, wherein the at least one chemical modification comprises at least one stereopure nucleic acid as provided in Table 2.

29. The engineered guide RNA of any one of the preceding claims, wherein the engineered guide RNA comprises from 1 to 100 chemical modifications, each of which can be independently the same or different.

30. The engineered guide RNA of any one of any one of the preceding claims, wherein the at least one chemical modification does not comprise a naturally occurring chemical modification to a nucleic acid in a eukaryotic cell.

31. The engineered guide RNA of any one of the preceding claims, wherein the at least one chemical modification increases specificity of the engineered guide RNA binding to the target RNA compared to a specificity of an otherwise identical reference polynucleotide without the at least one chemical modification.

32. The engineered guide RNA of any one of the preceding claims, wherein the at least one chemical modification increases resistance to nuclease digestion of the engineered guide RNA compared to resistance to nuclease digestion of an otherwise identical reference polynucleotide without the at least one chemical modification as measured in an in vitro assay.

33. The engineered guide RNA of any one of the preceding claims, wherein the at least one chemical modification decreases immunogenicity of the engineered guide RNA compared to immunogenicity of an otherwise identical reference polynucleotide without the at least one chemical modification as measured in an in vitro assay.

34. The engineered guide RNA of any one of preceding claims, wherein the target RNA comprises RAB7A, ABCA4, SERPINA1, SERPINA1 E342K, HEXA, LRRK2, SNCA, APP, Tau, CFTR, ALAS1, ATP7B, ATP7B G1226R, HFE C282Y, LIPA c.894 G>A, PCSK9 start site, or SCNN1A start site, a fragment any of these, or any combination thereof.

35. The engineered guide RNA of claim 34, wherein the target RNA comprises SERPINA1 E342K.

36. The engineered guide RNA of claim 34, wherein the engineered guide RNA has at least 70%, at least 80%, at least 85%, at least 90%, at least 92%, at least 95%, at least 97%, or at least 99% sequence identity with any one of SEQ ID NOs: 1-2.

37. The engineered guide RNA of claim 34, wherein the target RNA encodes ABCA4.

38. The engineered guide RNA of any one of the preceding claims, wherein the RNA editing entity is:
(a) ADAR or APOBEC;
(b) a catalytically active fragment of (a);
(c) fusion polypeptide comprising (a) or (b); or
(d) any combination of (a)-(c).

39. The engineered guide RNA of claim 38, wherein the RNA editing entity comprises ADAR, and wherein the ADAR comprises ADAR1, ADAR2, ADAR3, or a combination thereof.

40. The engineered guide RNA of any one of the preceding claims, wherein the RNA editing entity is endogenous to the cell of the subject.

41. The engineered guide RNA of any one of the preceding claims, wherein the RNA editing entity is exogenously provided.

42. The engineered guide RNA of any one of the preceding claims, further comprising a structural loop stabilized scaffold.

43. The engineered guide RNA of claim 42, wherein the structural loop stabilized scaffold comprises a stem loop, a junction, a T junction, a clover leaf, a pseudoknot, or any combination thereof.

44. The engineered guide RNA of claim 42 or 43, wherein the structural loop stabilized scaffold comprises at least 1, least 2, at least 3, at least 4, at least 5, at least 6, at least 7, at least 8, at least 9, or at least 10 stem loop structures.

45. The engineered guide RNA of any one of claims 42-44, wherein the structural loop stabilized scaffold comprises a tRNA scaffold.

46. The engineered guide RNA of any one of the preceding claims, further comprising an RNA editing entity recruiting domain.

47. The engineered guide RNA of any one of the preceding claims, wherein the engineered guide RNA is conjugated to a targeting moiety.

48. The engineered guide RNA of claim 47, wherein the targeting moiety targets a neuronal cell.

49. The engineered guide RNA of claim 47, wherein the targeting moiety targets a liver cell.

50. The engineered guide RNA of claim 47, wherein the targeting moiety targets a macular cell.

51. The engineered guide RNA of any one of the preceding claims, wherein the engineered guide RNA is encapsulated in particles.

52. The engineered guide RNA of claim 51, wherein the particles comprise nanoparticles.

53. The engineered guide RNA of claim 51, wherein the particles comprise liposomes.

54. A pharmaceutical composition in unit dose form comprising:
(a) the engineered guide RNA of any one of claims 1-53; and
(b) a pharmaceutically acceptable: excipient, carrier, or diluent.

55. A method of treating or preventing a disease or a condition in a subject in need thereof, the method comprising: administering to the subject the engineered guide RNA of any one of claims 1-53.

56. The method of claim 55, wherein the administering is intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

57. The method of claim 55 or 56, wherein the disease or the condition comprises a neurological disease or condition.

58. The method of claim 57, wherein the neurological or neurodevelopmental disease or condition comprises Parkinson's disease, Alzheimer's disease, or dementia.

59. The method of claim 55 or 56, wherein the disease or the condition comprises a liver disease or condition.

60. The method of claim 59, wherein the liver disease or condition comprises liver cirrhosis.

61. The method of claim 59, wherein the liver disease or condition comprises alpha-1 antitrypsin deficiency (AAT deficiency).

62. The method of claim 55 or 56, wherein the disease or the condition comprises macular degeneration.

63. The method of claim 62, wherein the macular degeneration comprises Stargardt's disease.


SEQUENCE LISTING (44 sequences)

SEQ ID NO: 1 - DNA, 13946 bp, Homo sapiens
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacaggtgggagggacagcagggcttagagtgggggtcattgtgcagatgggaaaacaaaggcccagagaggggaagaaatgcccaggagctaccgagggcaggcgacctcaaccacagcccagtgctggagctgtgagtggatgtagagcagcggaatatccattcagccagctcaggggaaggacaggggccctgaagccaggggatggagctgcagggaagggagctcagagagaaggggaggggagtctgagctcagtttcccgctgcctgaaaggagggtggtacctactcccttcacagggtaactgaatgagagactgcctggaggaaagctcttcaagtgtggcccaccccaccccagtgacaccagcccctgacacgggggagggagggcagcatcaggaggggctttctgggcacacccagtacccgtctctgagctttccttgaactgttgcattttaatcctcacagcagctcaacaaggtacataccgtcaccatccccattttacagatagggaaattgaggctcggagcggttaaacaactcacctgaggcctcacagccagtaagtgggttccctggtctgaatgtgtgtgctggaggatcctgtgggtcactcgcctggtagagccccaaggtggaggcataaatgggactggtgaatgacagaaggggcaaaaatgcactcatccattcactctgcaagtatctacggcacgtacgccagctcccaagcaggtttgcgggttgcacagcgggcgatgcaatctgatttaggcttttaaagggattgcaatcaagtggggccccactagcctcaaccctgtacctcccctcccctccacccccagcagtctccaaaggcctccaacaaccccagagtgggggccatgtatccaaagaaactccaagctgtatacggatcacactggttttccaggagcaaaaacagaaacaggcctgaggctggtcaaaattgaacctcctcctgctctgagcagcctggggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgagaggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctgtgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctgttcactgctgagcaggagccccttcctgctgaaatggtcctggggagtgcagcaggctctccgggaagaaatctaccatctctcgggcaggagctcaacctgtgtgcaggtacagggagggcttcctcacctggtgcccactcatgcattacgtcagttattcctcatccctgtccaaaggattcttttctccattgtacagctatgaagctagtgctcaaagaagtgaagtcatttaccccaggccccctgccagtaagtgacagggcctggtcacacttgggtttatttattgcccagttcaacaggttgtttgaccataggcgagattctcttccctgcaccctgccgggttgctcttggtcccttattttatgctcccgggtagaaatggtgtgagattaggcagggagtggctcgcttccctgtccctggccccgcaaagagtgctcccacctgccccgatcccagaaatgtcaccatgaagccttcattcttttggtttaaagcttggcctcagtgtccgtacaccatggggtacttggccagatggcgactttctcctctccagtcgccctcccaggcactagcttttaggagtgcagggtgctgcctctgatagaagggccaggagagagcaggttttggagtcctgatgttataaggaacagcttgggaggcataatgaacccaacatgatgcttgagaccaatgtcacagcccaattctgacattcatcatctgagatctgaggacacagctgtctcagttcatgatctgagtgctgggaaagccaagacttgttccagctttgtcactgacttgctgtatagcctcaacaaggccctgaccctctctgggcttcaaactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcatgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggccaacacggtgaaacctcagctctactaaaaatacaaaaattagccaggcgtggtggcacacgcctgtaatcccagctatttgggaagctgagacagaagaatttcttgaacccgggaggtggaggtttcagtgagccgagatcacgccactgcactccaccctggcagataaagcgagactctgtctcaaaaaaaacccaaaaacctatgttagtgtacagagggccccagtgaagtcttctcccagccccactttgcacaactggggagagtgaggccccaggaccagaggattcttgctaaaggccaagtggatagtgatggccctgccagggctagaagccacaacctctggccctgaggccactcagcatatttagtgtccccaccctgcagaggcccaactccctcctgaccactgagccctgtaatgatgggggaatttccataagccatgaaggactgcacaaagttcagttgggaagtgaaagagaaattaaagggagatggaaatatacagcactaattttagcaccgtctttagttctaacaacactagctagctgaagaaaaatacaaacatgtattatgtaatgtgtggtctgttccatttggattacttagaggcacgagggccaggagaaaggtggtggagagaaaccagctttgcacttcatttgttgctttattggaaggaaacttttaaaagtccaagggggttgaagaatctcaatatttgttatttccagctttttttctccagtttttcatttcccaaattcaaggacacctttttctttgtattttgttaagatgatggttttggttttgtgactagtagttaacaatgtggctgccgggcatattctcctcagctaggacctcagttttcccatctgtgaagacggcaggttctacctagggggctgcaggctggtggtccgaagcctgggcatatctggagtagaaggatcactgtggggcagggcaggttctgtgttgctgtggatgacgttgactttgaccattgctcggcagagcctgctctcgctggttcagccacaggccccaccactccctattgtctcagccccgggtatgaaacatgtattcctcactggcctatcacctgaagcctttgaatttgcaacacctgccaacccctccctcaaaagagttgccctctcagatccttttgatgtaaggtttggtgttgagacttatttcactaaattctcatacataaacatcactttatgtatgaggcaaaatgaggaccagggagatgaatgacttgtcctggctcatacacctggaaagtgacagagtcagattagatcccaggtctatctgaagttaaaagaggtgtcttttcacttcccacctcctccatctactttaaagcagcacaaacccctgctttcaaggagagatgagcgtctctaaagcccctgacagcaagagcccagaactgggacaccattagtgacccagacggcaggtaagctgactgcaggagcatcagcctattcttgtgtctgggaccacagagcattgtggggacagccccgtctcttgggaaaaaaaccctaagggctgaggatccttgtgagtgttgggtgggaacagctcccaggaggtttaatcacagcccctccatgctctctagctgttgccattgtgcaagatgcatttcccttctgtgcagcagtttccctggccactaaatagtgggattagatagaagccctccaagggcttccagcttgacatgattcttgattctgatctggcccgattcctggataatcgtgggcaggcccattcctcttcttgtgcctcattttcttcttttgtaaaacaatggctgtaccatttgcatcttagggtcattgcagatgtaagtgttgctgtccagagcctgggtgcaggacctagatgtaggattctggttctgctacttcctcagtgacattgaatagctgacctaatctctctggctttggtttcttcatctgtaaaagaaggatattagcattagcacctcacgggattgttacaagaaagcaatgaattaacacatgtgagcacggagaacagtgcttggcatatggtaagcactacgtacattttgctattcttctgattctttcagtgttactgatgtcggcaagtacttggcacaggctggtttaataatccctaggcacttccacgtggtgtcaatccctgatcactgggagtcatcatgtgccttgactcggggcctggcccccccatctctgtcttgcaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggtaaggttgctcaaccagcctgagctgttcccatagaaacaagcaaaaatattctcaaaccatcagttcttgaactctccttggcaatgcattatgggccatagcaatgcttttcagcgtggattcttcagttttctacacacaaacactaaaatgttttccatcattgagtaatttgaggaaataatagattaaactgtcaaaactactgacagctctgcagaacttttcagagcctttaatgtccttgtgtatactgtatatgtagaatatataatgcttagaactatagaacaaattgtaatacactgcataaagggatagtttcatggaacatactttacacgactctagtgtcccagaatcagtatcagttttgcaatctgaaagacctgggttcaaatcctgcctctaacacaattagcttttgacaaaaacaatgcattctacctctttgaggtgctaatttctcatcttagcatggacaaaataccattcttgctgtcaggtttttttaggattaaacaaatgacaaagactgtggggatggtgtgtggcatacagcaggtgatggactcttctgtatctcaggctgccttcctgcccctgaggggttaaaatgccagggtcctgggggccccagggcattctaagccagctcccactgtcccaggaaaacagcataggggaggggaggtgggaggcaaggccaggggctgcttcctccactctgaggctcccttgctcttgaggcaaaggagggcagtggagagcagccaggctgcagtcagcacagctaaagtcctggctctgctgtggccttagtgggggcccaggtccctctccagccccagtctcctccttctgtccaatgagaaagctgggatcaggggtccctgaggcccctgtccactctgcatgcctcgatggtgaagctctgttggtatggcagaggggaggctgctcaggcatctgcatttcccctgccaatctagaggatgaggaaagctctcaggaatagtaagcagaatgtttgccctggatgaataactgagctgccaattaacaaggggcagggagccttagacagaaggtaccaaatatgcctgatgctccaacattttatttgtaatatccaagacaccctcaaataaacatatgattccaataaaaatgcacagccacgatggcatctcttagcctgacatcgccacgatgtagaaattctgcatcttcctctagttttgaattatccccacacaatctttttcggcagcttggatggtcagtttcagcaccttttacagatgatgaagctgagcctcgagggatgtgtgtcgtcaagggggctcagggcttctcagggaggggactcatggtttctttattctgctacactcttccaaaccttcactcacccctggtgatgcccaccttcccctctctccaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtgattccccaacctgagggtgaccaagaagctgcccacacctcttagccatgttgggactgaggcccatcaggactggccagagggctgaggagggtgaaccccacatccctgggtcactgctactctgtataaacttggcttccagaatgaggccaccactgagttcaggcagcgccatccatgctccatgaggaggacagtacccaggggtgaggaggtaaaggtctcgtccctggggacttcccactccagtgtggacactgtcccttcccaatatccagtgcccagggcagggacagcagcaccaccacacgttctggcagaaccaaaaaggaacagatgggcttcctggcaaaggcagcagtggagtgtggagttcaagggtagaatgtccctggggggacgggggaagagcctgtgtggcaaggcccagaaaagcaaggttcggaattggaacagccaggccatgttcgcagaaggcttgcgtttctctgtcactttatcggtgctgttagattgggtgtcctgtagtaagtgatacttaaacatgagccacacattagtgtatgtgtgtgcattcgtgattatgcccatgccctgctgatctagttcgttttgtacactgtaaaaccaagatgaaaatacaaaaggtgtcgggttcataataggaatcgaggctggaatttctctgttccatgccagcacctcctgaggtctctgctccaggggttgagaaagaacaaagaggctgagagggtaacggatcagagagcccagagccaagctgcccgctcacaccagaccctgctcagggtggcattgtctccccatggaaaaccagagaggagcactcagcctggtgtggtcactcttctcttatccactaaacggttgtcactgggcactgccaccagccccgtgtttctctgggtgtagggccctggggatgttacaggctgggggccaggtgacccaacactacagggcaagatgagacaggcttccaggacacctagaatatcagaggaggtggcatttcaagcttttgtgattcattcgatgttaacattctttgactcaatgtagaagagctaaaagtagaacaaaccaaagccgagttcccatcttagtgtgggtggaggacacaggagtaagtggcagaaataatcagaaaagaaaacacttgcactgtggtgggtcccagaagaacaagaggaatgctgtgccatgccttgaatttcttttctgcacgacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggtgagatcaccctgacgaccttgttgcaccctggtatctgtagggaagaatgtgtgggggctgcagctctgtcctgaggctgaggaaggggccgagggaaacaaatgaagacccaggctgagctcctgaagatgcccgtgattcactgacacgggacgtggtcaaacagcaaagccaggcaggggactgctgtgcagctggcactttcggggcctcccttgaggttgtgtcactgaccctgaatttcaactttgcccaagaccttctagacattgggccttgatttatccatactgacacagaaaggtttgggctaagttgtttcaaaggaatttctgactccttcgatctgtgagatttggtgtctgaattaatgaatgatttcagctaaagatgacacttattttggaaaactaaaggcgaccaatgaacaactgcagttccatgaatggctgcattatcttggggtctgggcactgtgaaggtcactgccagggtccgtgtcctcaaggagcttcaagccgtgtactagaaaggagagagccctggaggcagacgtggagtgacgatgctcttccctgttctgagttgtgggtgcacctgagcagggggagaggcgcttgtcaggaagatggacagaggggagccagccccatcagccaaagccttgaggaggagcaaggcctatgtgacagggagggagaggatgtgcagggccagggccgtccagggggagtgagcgcttcctgggaggtgtccacgtgagccttgctcgaggcctgggatcagccttacaacgtgtctctgcttctctcccctccaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 2 - DNA, 32767 bp, Homo sapiens
ggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaaggtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgaagagcaggccaaaggcaagcaagaaccccctcgaacagcttcccagcttagtgaggccttatttcattgattctctgaggcacattgttttttcacatgttagcatttctgaaattgggatgcagctcacgatcaagtcacagtttaactggacacattatttttctttcttagtggtgcagaaaagtaacagtgtgtcttacaattgactgcgtcctagattctgtgagatgcaatacgttattaaccatcacgcacatttcctgaactctttcaatgagcagacaccagcctgggttagactggagccctaaaagcacgacacagattccaccctggactggcttctgttctgcctgggaaaacccaaagtacgtttggagaccaagagcaacataaagtagcataggtggaatagtccatgagaagtgcgagcaaaaggtgccggagatcagagaacaccaagactgtacttgtaaatgacaactggctttgtgcaattttttctgggaaaggataaggagtgactatagaactgtaaagaaagaatgcactttgctacagccttgcagagttgtgcaaatgccgatgactaaaggagctgaaagaggaaggaggggataagggatgggggctgggtaggggtgagattaggaccctgggagctgcaagccactggagagatcaggaggaaagggagggagacctgctttaggcgagaagagaacagtatttgttccaaatctcggttcagaataagttcatgtaggtgatggggccaactggaacaggtgaaggcctatgaatgagtgtctcagttagggtctccttagagtttaatatgaaaaggtgttagctaagtacagagctggtacctgagagagtaaaaggaaactctaaggtatcatggaggtagcaattgcaggacacagctcccacccctagggctgagagaaccaagggaagagacaggaattattaagacttggagcatagatgagaggtctgtggagctgacattaggacttgggaggaaggcgtgcatggaggctgctgctggatctctgaacctgacctcgggtctggacccctgaggagaaagccctggcaggttggtgcatgtggggccgagggacaatagcttaacaaccagcataaaagagagcagcatgggacacgcttcaaccatgcgcatggatggctccaaaacctgtgtgtggctggcccaggacgcagggaggctgcagggggaagagacaagttaaacctgacttgtctgggaagcaccattgtcctcaggtcactttcctctgtcaagcctggtgctgaagttatctgttgtctccaggggccaagtattaagagtaatcagaaactcagtcctttcttctaggagcttcccttcttgcatgaaaatcctgataaaactggaaaaaaaaacctcatgattaaattttttcatgtattcattctttccttctatcaaaaaataatctccaggcaccgtgctaggttcattggtatacaatggcaacaagacctcccagcccctgcctatgtgaggcatctgtggactgcggaggaaaatccaatatgccattgttctctctttcccataagaaattacaattctcagttcattttattctcactgtgctctttgtgaccctcaaagggggtcacatgataacaggactgtagctgctggcctaaaatgagcccattcctgtggcgctcatgtcgctgtgacagagaataaccctgttttcagaatgctctggtgccctccctctcaatctggcctttcgctggcatgggtgggcgactcctgctcagggactctgccttctccacagtgtgctcccagggagatggagccactcgggctgagggccttggccagggcacctcccagggctgggcctggtctgggctggcgttcactggatgccatcctgatggcctggaaattgagatttctgtctggcacgcctcccgatggctccccacctgctaccacattccaggagcttccaggatgtctgggtaagacagaggcacccccaacagattcagtagctctgagagggatctgtggctccttcctaagcttgcggttcttctggaaacttctgcctctagaagatggtccctctaagaaaagtacaaccacccagcccataattcagctcccaggttttccctcaaacctccatgtctcctgtaagcagagcaagagtaaaatcagataccaaatttcctcattcctcagctcccaatccctaagggcataagatgaaaatcttcagatctctgctttcctccctctttttttcttcctctgttaacatttgtcaagtgttactaagtgtctggcactgtactaagtgcatcacctccctgaactctccgaacagttccacgagagaggcctctctgtgatccccccggtactgatgaggtcactgaggctccagagaaggattagtaactggtggggttggacctgggattcacacccatgctgcgtgacccaggacaggcaggcatggccgttacaccacactgacccccgtggatcgagatctatccaatagtctggtcactgatatcactaagatagagtggccatataatttatcatccaatcagggcagttttgcaagtgaaagggagcactattaataattgcactgggacaataaatgtaaaccaacactggacctggaaaactgggacgtgtgtttgccctataccaaggtaagctagacacagccactgccttcatggagttcagaaccaggcaggggcggctcccacgtataattactgtgcagcacaacgtggagaccgtggagtagaaggaaacacggatgggaggtgaggaggaggtctgtgagctcagaggaggcaccggggctggagagggtgagagaagacttcccaaggagttcatcctgataacgtgcattcccaatgacgagcgctctctccactgcacaagacaagtatacatctgcccgtgttggctgtggacctggcgctgtgtcagggagggtttatgaagatcactaggtgggtctcttggtgtcatcccttcatcccagcttctgggttaggatggatatctgtgggggggcctgaggactcatgaaagtggggcgctaatcatgttttggacaccacaccctggagcacctgggacagctgtggcctttgtcctgggttcagcatcaagccgaggatgtggcaagtaaagagaggctgggcaccaactccagtgtacccaggctccgggtcatgtttgtccaggctaagaattctgtcctggttctcagtgcagaaggaagaatcatggggctcattttaggccttggctgccttctgttaaattgaaaacagagcaggaaggaagaaaatttaacaggctcagttctaaaacaacaagcacaactgtgcccttgccagaaacccctcctccccatgttgattgaatggtaaagagaggaggggaggtgagagggagagagagagagaggaagagagagagaaaggaaagaaaggaaagaagaagaaagaaagaaaaggaaagaaagaaagaaagaaagaaagaaagaaagaaagaaagaaagaaagaaagaaagagaaagaaagaaaggagggagggagggaaggggaaaagaaaagaaaagaaaaagaaaaaaagaaggaaataccagtttgggaaaaaagaattttccaccagcccttctgagccttggctgggcttaattaaagttacagacatgtgtaaagggcagggtagggggagtctgagctgctgagaaaacatgtttttaattatactgtggaatttctccctggggtatgcctgtacgcagttaagcgtcaaggacagggatgccgctctggggaggggaagctgagcatgattttggaagccggcagaagaggctattgtgaaaaccagacctgtcaggctaggaaaagaatggctggtggtctttgaccagggagtgacgcgtgaaatgcagcaaccgcccccgccccccgccaaaaacaaacacactctcacagagttagaacaacagtgacctctcaacaaatatttttcaaagattaccaaccaaccattacctagagcagcggttctcaaccttggctgcacggtggaactacctgagacgtgttaaaaagaagaaccctgatgtcccatgccccaagattctgatgtagttgatctggggtatgatctgagaccccggcatgttttcagcctgcagccacatgagaagtgctgacctaatcaacaggggtgatgatttgaggggcggggactataggcaaaaaaaaacagcctaattcaaggatgagaagagggcacaggtgaggtgggaacagtcctagggccagacaaagaaggaagggagaaaggaggtgctgatccctcccctactcctgagaggaggcctttaagtcaccgtgccttgtggagaccagattcttcaaaaatacaagaatgagtgagtgagggagtgggtggatgccaggagagtgcgtgacaagccttgcaagggaggatgacaatgcactagcttggtttggaaattttacccctggaacaggcaggccaagctggctggtcccctccctgatacacagccctccctctttatatatggagcaggggacggtgtgtggctggtttcttagcaagcaccatggttccaagttggcaactggggagttctgaatccaaaaaggagggagatgaacgtaagtggagggcaggcctacaaggttgcagataagcttaattctgtctccttactcttctgcctttgcaacaaccctgtgatcttgcgacaaccctgtaaggcaataacaaatggctcatgtttattgagtgttacctcatgccatattgtgctttcgtgtttaacacaattgtctcatttcaccctcacgactgctctgggaggtaggtcctggtatcacatccatttcacagatgagaccatttggcacggaagagttgagtgggctgcccaaggtcacatagctaagatggaacaggctggataggaaccccagtaacttgacctcagagtaaccttctcttaaccctgagtgtacactgtaggaaaaatgagcagtcccatttcagagaggacaaaactgagactcagaggttaagcaagccccaaagtggttgttaacccagatcttcccactaactcccaaatcagcatcagtgtttaacgtaccagacctctcccagatagatgttgccgcatggaagacagccgatctacgtgatagaaagccaatattgcaagcagtcgtctaaaggagtcaaatgtgttggatttgaactggatgtctcatttctttggtgaagacactggaaacaacttccaggtttcatcaattgctcctatcactcaacgttgctatcttactgaacttgttccccagccttacccactgatggaatgatccagaatggaagacaagacaccaatgtacatgaccctgggggaggctgtttcttaaatctacagactgttggtgacctgagccccatgtcaccaaaggctttcctggagaagcctcctagaccagtcttgacaaaggctcactcattccgtggatatttattgggcacctattatgagttctgccccatgtggggtgctggaatcacagtagtgacaacgacagatgaggttcctgtcctcaggaagcttactgcccttgagggcttcacttacttggaggagtgatgaacctgaagtgcggtgtgtgttaagaagcggaagtccagggccaggcgcggtggctcacgcctgtaatcccagcactttgggaggctgaggcaggcggatcaccaggtcaggagatcgagaccatcctggctaacatggtgaaaccccgtctctactaaaaatacaaaaaaattagccgggcatggtggtgggcacctgcagtcccagctactcaggaggctgaggcaggagagtggcgtgaacctgggaggcagagcttgcagtgagccaagatcgtgccactgcactccagcctgggcaacagagtgagactccgtctcaaaaagaaaaaaaaaagtgcctcacggagagtctattcttttcttcccatattgtgtgtgtgtgtgcgcgcttcctccaacacatcctccctatatatattttgagtaaaacatcttgtaaaaagttacagctacataatcaccacctgtccctaaatagtttttgctttttctttcttcaatgcacgatcattttcccccatcaatttattttttagtttcttataatcttgttgccagtaggctgttttttaaaaagcagaacatggtttgttcttactagcaggaaaggagcatttattgagcctctgctatggtgtcttttattttgctgagagcctatttacatttctttgagaggaaaacaacaaagggttacatgaaagaccatgtgaatagcccctagctgatctattaaacttgctattccccggccagctgcttcagatctccttcagatcttatgtgtttccttcctaaggtccctggagtaagggttgcatagacctattctactctccaactcacatgtccctctccctcttcctctccataattccacatctccaacccccacccctatgtgcaatgccacagggtgtggactgccacagccactggatctgcttttggaatcaagagtccttaagctccaaatggaaccgaaatttaaataccaactttcaaccatatgttaacatcagcagcctcttccaatgtaaaaacccatggcagtgtgccctgctttgtttctttaagcaatagaaacttgaaggaagcatgttggtaggccagatttttgttggctttgcaatggatcacagtcatttattcactcattcattcactgattcattaaatgaccacatttgcaagggcaaggtaatggggagggccagaaaggacactggccccagaaacaggaggctggattttggttctgatgctgccactgctgatgtgacactgcacaggtcacctgcctcctctgagcctctttccttaactgcagagtgagtggctacagagaaatctttactacctgttagatcagcattacctgggagcttgttagaaatgcaagctctggtggggccatactgaacccaaatctgcattcatgtgcatagtgacagctaaaatgcactgaagcagatgatcttgatgatcctttatgaaagtctcatgctaatgcagttttctaaaatagaggcagagtggaacccagatggacacaaaatctggttgatataataaaacaaggtagagggtgtatggtggggagggggtaaaggaaggaaactgtttaggtaaagataccacaaccaaagtcctactgcacacatgggatctgaggagggctgtgtctgctctggttacgttttctataatctcttagcaccactgaactttctctctttttgttttgtttttccagattcgctttgtggtggaactcgtgtggcctttatctttatttctggtcttgatctggttaaggaatgccaacccactctacagccatcatgaatgtaagcatagcagggtagcttgggcaagccctgaagagactttggtctgggccttttgtctagaaagatcttggggtgggagtgtggggatcagatctgcttatcatcatttcatgtctatgatgcatgtaacagatttatcaatgttacacaaattataatttttaaaaagtctttagagacagggtctcactctgttgccgaggctggagtacagtgttaggaccatggcacactgcagcttctatctcttgggctcaagtgatcctcctgcctgggcttccaaagtgctggaattataggcatgagccactgctcccagctaatttttttgttttttgtggagacagagtcactacattgcccgggctggtcttgaactcctggcctcaagtgatcctcccacctcagcgttctaaagcactgggattacaagcatgagccaccttgtccagcccaaattttcatgttttaatcctacacattctaagcaaatacttgtgtgtagttactaagggactgtgcacttatttttgtttgctttgttgttgctagtttttatttttttatacctaaactctctcgttttaaagagaacagatttgtagatgagttctcgaaaatatttcaggaatcaatatagagaatatgttatacatggtgccagagaaaaatgaggacaagagatgctatacaatcgtactgaagaaaaattttatttcttggacccctgaggtgtctgcagacctgaaaggaacctagtgagagcctcttttacactctgcccctgtgggaaagccttcacctggtttccggccctctatgtggtgaatgtggaagcctcaagcgttatgcaaatctgcccagtcctctattcttgatcttcaccttctcgttcatgagtttcaggccccagttctgaatcagcctcctgtccatcagactcttctttacctctccccgaggagcccataacctgcagccctactgcatgcttggggtaggtgctcagttcaccgtggttgaaggaatagacgagcgtctgctcaagcagcagcagcaactgcgtggagtcttcttgaactaacactcctatgcccctctcggcacaaaatgacgtgtccccccttgcttccccttcacatttccacccatgcctattacaacatccgtctgtctccccactacaccgggagcttgagagaagaggccatgtctctagcacccagcacagggactggcacacatgagatgctcctgcttcttaaatgctgagaatgaaggaggacatcagaggggcccgggccccttcccaaaaaggccaactcctaggtctgcatcctgcttggtctccatgactaatcccgtcttgtcctcattttctgttttaaaggccatttccccaacaaggcgatgccctcagcaggaatgctgccgtggctccaggggatcttctgcaatgtgaacaatccctgttttcaaagccccaccccaggagaatctcctggaattgtgtcaaactataacaactccatgtaagtgttgagatccctaccatgcaggggaggaagttgcacaccccttcacgtgctgaaatgcacacgtgcgtgcacggagcatggagcactgagtgttcttgtggctttgctgagcccctaacctcttaggagcagagcaggtttcctctctggaacattctgttaactgtcagggcacttggggagaaatctccaagctaaggccacgtgcacaaaatttcttggtccttatatccccagaatgtgacctggagtctgatggcagcccgctgcagagatgtgtccactgccttctggtcattgacctgcttgggtggagtgaatcattgtaggagaaaaactcagttccctcaccctgatcaacctggacagatctctcttcctttaaaagctttcttggacatctaagggctaggaaaaatgtcagggagcattgggaaggtaaatgaagtcaggtttacaaagtcaagtttacttcttgggagaaaaatacaatttccaaatcctctgttataattgccatcggccccctggagtggtgagatctcggaatatggctcgggtgcagtggctcttcactgtgggcctgcaggctattctgaaaagctgatgaaaaccaatgacccctcttccaagaaaaatggccacataccaaacattacactgtacatctgatttcagggaattgtagatgccaggttagtagcctcaggtctagggtcaaaattcaagtcgaatcccacaggaagagggtctgccttcggaattccctttcagagcattgggagaacatcatgggagcatattctagagacagaggcttagggtgtggacagggccatccctcacccactgtgctgaccttaagcagcaccttgtgcagcccatacctgaaggccaccagcaaaggcctgttggggagcaggctttacccgacctgtataaacaccaggctaggtgaaaactgagatacctggttactttagttttttccttgggggagctcagtatgattcttccaggagaagcctgcttttagactaaaaagaaaaaaagtttgataggtcaacctaatgattggaggtggccttccccactgtgaacaaactatggctgcatgtgccctacaatggcagagttgagtagttgtgatagagactgtatgatctgtaagcctgtaatttttatgtttgctgacccctggattaccagatgatagaagaggaaacatctgtcttcctagcaaagtcaaggaagtggcatttagcaggactcatattgctgcaagcactgccttgcagttttagtttacaactgcactttcagcttaagaaacacctgcccatccagagagatcgtgtggggtcacatggtgggatcagggaggcctgaagacagctcagtggaggctgcatggagctttggtgggaacggccctggcagtgtctatagatgttattgcggaaaactgaggggtgggagttggagaagggggctccagactctagctgtacttggcatttgaacccggaaagttgggtttcatgttttgcactcacattatgagtgaaatattggcttattcaaggttcttttgcttgcaaggcacggaaacccattcaagcaatcttaaaccccagaaggaaatctatgatttggatactagacattctcacagagccaagggcagcaaggcggggctcaggagaggcaggccaagacctggagagctgtcaggagctgcttcctcaactctcttccatctgggcctgccagccctggcctctgtatctactccattcacctctctccatggaccagtctcccctgctcctcaatgcctgggctgccattgttcatgcaattcacaatacctcggcctgggcaatcagaagctcatctctgaacaccatccaaattcctgggaacaaatcgggttgacccagctttattctccctgtcccatcagccttggcagaggcgtgcatgtgcatgcgtgccaatgtgtgtgtgcagggaggtccttgtggatgaagcatggctgtcagagcctacctgcgtgaatgggtggaagggcaggtctcagagaattgggtaaaaactggataaaccctccagtgatatccaccaatgtcaccctgtttaaggcttctctgggcaagagacacacagagcatgggaccgagaggcgagcagaccctgccaaaactgggagactgaatagatcgctcaccatccttgtcagttagcctatatgtacaaggaagtaaaattatctctttctcctgccttggcagtattgtaaggatactcaatgtagtagctaggccagacacatagtatctttaaatatagcatgagatggccaagcacggtggctcatgcctgtaatcccagcactttgggaggctgaggcgggtggatcacgaggtcaggagatcgagaccatcctggctaacacgatgaagccccgtctctactaaaaatataaaaaattagctgggtgtggtggcgggcgcctgtagtcccagctactcgggaggctgaggcaggagaatagcgtgaacccggggggcagagcttgcagtgagccgagatcacgccactgcactccagcctgggtgacagagcgagataaaaaaaaaaaatagcatgagatattattactgttataaaaataacagctatttccttattaatgaggctttgtccttacagcttggcaagggtatatcgagattttcaagaactcctcatgaatgcaccagagagccagcaccttggccgtatttggacagagctacacatcttgtcccaattcatggacaccctccggactcacccggagagaattgcaggtaagcatgactgcagtgctctcaagcatcatttccctcacctatggagagactgaagatataggaaagaacagggagagttggtgaaaaatatactagcggaggcaggaagggatggggtctggaggcggcttgaacatcaccttggtgaagatgcctcttcctccacagaagcctggaaggtaggaagttgggaaggaaggcaggaaaggtctcatccacgttaagtctagagacagaaagaatgctaagagagatggcactatgggaagtatgaggctaggtcaagggctagaagcaggggagacgagtttacagagtttcgtaaagatatagagcaactctcacagagttctagagcgagagctaaccaggaacatgaagcagcaaggccaactatcattaaggagccagggaggtcagagatcatgtattatcatgacataaatatgcataattgtactatttctcccagtaatatttagcacccaggccccgaggcagagcaagtggagagtgggtgatgcagggctgggggtgtgtatggaggcaccacagaaggtcaacaggcagcgggctgaaggcagggactggactacatgcatcaagtccaggctgcacgaggaaggatgagaaggcagatgagcacggaaatggactgggggaaatgaagaggcaagggaatagaagtctcagtgggtgccatgaccctgtttaagtgattgagaaaatgaacaagatgaaaaggttaatggctgtggtcagaaagtgaaatatgtgaattcaggatttcgaaggtagggtgggtgatgactggcccccagatgcggccatggtgaagtggggcaaaggtgcaggtgcatggtgaggggaaggaggaaatgggaggtgatgatgttggccccacacggacaccacggttgtgcaggaagatggcaggagctgggcaccagggtgggagccacctggagtcaggaagagtgaagagaaaggatgaagaggctccctctcctgtgtctctcctccccaggagaagaacaagaaacaatccgaaagtaataacaccaatgtgcctttacaaagtgtgagtgggtgttgtgtgctgtcacgtgtgtagtaggctcctctgtggatggctagagggactggacatggccactggatcccacttgcaagagcagaggaaaagagtggtcgtgaggaagtaaagccccccaaaatccaggggttgctgcagctttgggtgtggagcgtgccctctgaggaaaggctgctctgggggagattgcccaggaaacggggctcagaggccacgaaagcagctgttaggggcttctgggagatgtgtgctcctaggattagggagttgactctaaggatgaccttagaggttaacagggatgagaaaggggtcaccaaggggtctaccaggggaatgggagaggctgtattgatagaacagcttctgctgcaggttccaaacaagaaatgtgggagaatggttgaaatcagccccgggggcaccttcccgtgcatgcgtgcagctccttcaacattcagtcgaccttcagtgcctcctgtgagccaggcactgggctagtctctgggggtggagagatgagtcaggcaaatgccagccctcagagggctcacagggcagaaggtgagagatgagtgagcagaaaatgaccacagcgcgtgtggggcccagtggagggaaggaggggattcaggagcacaggagagtcaacaggggaaacttctccgaggagaatctgatcctcctcccatctggccaccttctgaagccctctctccccatccaagtgagaaaggacaggcgtatgaccagattggtgtatgaagatgctgaattacgttctcattgtttcaaactagtaaaccatagattttatgtagtaacttctacaaactgcattacaaacactccattctttgttgccctgggtagaagtttattttagtgagcccaagtttgaggaaccttatatggtatgagtacaattaccattttaatagtaagaaatcccccttcccctgtgtaccaaccagaaggtgtttttttcctaatttaaacaaacagatgcagacgtgggctgtccagctcctggcgggatgacatacctcatgcatccagtgggtttgatgatgaggcagacatttcacttaagtgcctgatcatcagattgagtcctgctgggaggaagtgtgaaggaagtaatttcaaaccacagtttctctgtggcttttacaatgtggatatgagaaccaaaatcactacttcttaaccccagagcaggactgattttgaattggtatgcaggcggttccttctgcaggcttcgggctgtgagaagtccctaacagagcaaatctggggacaagggctcaggaaaggttggccacggccccctaggaatgggggctctgcaagatccctggccttagaggctgtgagagggaacaggggtccatccccaagtaagggacacggtctttgaggaaatcccaggccagggcctgaagggcactgtcaggaacacaggctgtttcagtctgttgagattcaccggggcgctgctcactgtgagcacggactcctcaggccaatgtggcagaagagcccacctttgaaagcgagcgggtgggggtggcggggctggtgctggtgcgtgcttctgcacagccacctgggaaggtatgccgctggttgacccaggcagaggttttctttcatggcaaacctgcagtactgcattctcagcagggaggattaatggtaaaagaccaggcatggagcccccttccctctccctcgaagcaagctctgtggtctctcaatcatctttaaaacaccttcttcccgggagcctcctacattctcctggcttccctcccacccccaccctcagctcctggggcctcagcagccccacccccaagcctctaatcttcccagggaagggaacaagaagaaccacattttaaacgaaatttatttttctttcctcaggctcccagttcacatttctccctcaggagtctagggaagcttctgtctggtatcggcctcctcttcacctgggcccccgccctcctcaggtgtaccagaagccagcacactcccccttcccccccagagccacagcagccctgtctcctgggtggtcttgtgtgccaagcctgggcaacatcactcccagcttttcttgttttgccccttctccccagcaagatatttgtatgtaaggtcaggtgagtgagttaaagaataacgaagagataaacagtcaaatggagtcctgactgtcaggtcaagacaacagttatttactgaatgcctcatgtcattcaacagacatttattgagactctgattggatgtcagtctttaatgctgggtgtcagagagaggtgacttcaagggcttgcatctgtgcacccagcattgctaggtacaatgaggagtataataaaagcaggagccatagcccccaactctcaagagatctcccatgtgtgtatgtctgcatatgcgtgcgtgtgcatgtgtgcgcatgtgtgcatgtgtgtgtgcatgtgtgtgcatgcgtgtgtgtgtgcgtgtgttggggatggtgttggtggagtgagagtgtacaaggctgtgtatgaaggggtaattgggaaaagaacaatggagctggcacccagggacaggaggaaaagcaggagggctgggtttggaagacagccggatttatgtttttgaagagggaagactagaatataagggagcagcccttctcagagccctcctcctcccttcgggccctgtgtccagctttccccaaagtccttggatctttcctatgcaaaggggagtgacagtgggcaccactctcagggaacccattactgtgagagaagccactgtgccactgtgtggtcgaacttcaagaccggcttcccctgccccagctgcatggacaggcctgtggggttggcgcaagacccttccagaggaaactagctgcaacataaatccggatatggtgctgttcagggaaaggcacaacctggggatgagaagggtggctgtccagcacacaggggcaggcctcttggccactgggggaggggagaatttggagaggaagaggatgggatgccgtggaattgggaccaggaaagaatggggacatgtgatggttaaagctagttagagaagaactgggagataaacagtcacccatgcccctgaagcactcggggtgaagagattggcattttcacgcaccccagtgctttccctttgtgttgaagtcccttcgtagacatccaggcccataaggctcttctctggccagagcctcatgaactatagcactagcagggttgaggccaagcattggccctggaagccagccgaggaggagggtgcttgtgtgaatctcccaggaggggtaagaattatattaattcgatcataataagcatttattgagtgctgttttgaggcctgggagctaagcacttcacattccttaccccgcatcaacaatcctatgaggtagatgtggaaaatgcagacacggggacaggctcaatcacttgccccaaggtcaccttaactgttaggtgttctttatgcctccttataaagaaaccctgcttcccacaggtgttgagaggagctggagggagcttgactagggctcatcaggcaagccccggcatgtgcctggctctcctctttctacctggagcttttcctgcccttaatggccccaactcatttctcttagtccatgtcagtgccctgagcatctcagcccaagctgagatgatagaaacacccagaggggtcctctaccctgtgacagctgcggtgtgggaagagcacgtgtctcctccaatcctagaccagagtttctcagcctcagcatcactgacacttggggctagataatcctttgtgtgggggagggaggagtgtcttgggccttgcaggatgtttagcagcatctctggcctctacccaccagcacctccccagttgtgacacccagaaatgtctttagatcttgccaaatatttccaggaggatgaaattcccctgtttcagttccccagccccacctcaatgagaagcactgtcctagaccaaccccacaaagcatctgacacccccatccagccctggctaactttttccaccttcttactaaattgggcccagctgcttcagcagtcaatgtgttgggggcagcccactggcaagagcctcacctctaggggctcccagagaccccaagaacagaaccttcctctgagagttgagttacaagtgtttccaatcgactctggctgttttcctttttttgacccatttccccttcaacaccctgttctttctcttattcatatgtaggaagaggaatacgaataagggatatcttgaaagatgaagaaacactgacactatttctcattaaaaacatcggcctgtctgactcagtggtctaccttctgatcaactctcaagtccgtccagagcaggtagggggatgtcactggccagtggtccctggaggggagggaagcacccagcctgagaaaggcaagaaatatattggcttttttcttctttcttccttgtgttcacattcagaatccatcacttaatgccttgtatttagaaaaaaaccgggggatcacttgagatcgtgatcattttcaacataggattcgaagctgtacacatcctggtgaccttaaaacatctcaggtttttataactggaaggaaccttagagatcatggggcacaaccttctctttatagatgaggaaacagaaatctattcatttattactcaaatatttagggacagttgtaggtactagaacacagtgtgaaccagacaggcaaaaccccaggccagggagcttccattccagtggggccacaggcgatgctcaggtaagcagagactccgctgtgtgacttctggctgtgatgggtgctgcaaggaaaatccggtagagtcgagggttagagagggacggaggggcaggtttaagggggatgctcaggaaggccttcctgaggaggtggtatttgagcagagttgtctgtcagccacacagtaagtgagaggggagttccgggcttggaagctgccagcacagtgctggcaagtgctggggtggcgtcccgaggctacagaacctgagatgctgcagaagagcccacttctgctttcctggaccacttccttctcagcaccaggcaaactccttcttctatcccctggcacatttctgacctgtgtatacgcccccaatttatctaacccctttaaataatctcctctatttatgcagagcattcttaccactaactcacgacttgcacatcccttagctcccttactcctcacaacaatcctgagatgggtcagagaaggaggcttgcgcgtctggtgatggggtgatttgtgcacagttacagggctagaaattgtcagagccagatggaatccaggtcctctcaatcctaatccagtgtttcttacttcagtcctgtggctctcaaagcccagagaccagcagcatcagcgatgcctgggagcttgttaggaatgcaaattatcagggcccactccaggtgaactgggtccaaagccctgggataaggcctagcaatctgtgcttcacaagccctccaggtgattccgcaggctcaggtgtgagagctgcagctgtcctctgggccttctgggctccccgcccagcttcttcagtgtgatgaacacagcgagaatgctagatctgcagcagctgatatcccagacaccctcccgactccctcctggctgggtctgatcctcctccagactccaggagagaacgagacataaacagaacttcagagcctgtgttaaccctgagatcaaggtctgcacagggtgctgtctgagtccagaggagtgagggaccccaccccacctggtcagcaccagctcctggaagcaggttctcacactggttccctgcacaatgaaggagctcatacctgcttttctggcttctcagaccctgaggttttcaccgaaactagacaaggggaacctagggtcagcctggaggcagggtgagcttggcgcctgcagtgcccaggccctgggtggtgcggctccggccaggccctgtttagcttcctctcccacccccacagagggggtgctgtcggcaccgattgctcattttcccctttgctttctcttcagctcgtaaaactcaagtcctgacaatgccttgatgacttccagttggtaataaaagggagatgaagataaggacaggaatttcggggaaatttctttccagttccttactaatgtgacatttagatctctagtactgtgcttctggcatcagtgccaaggcctttcatgttggagaatggaggccggggtcaccaggttgtgcctttatttcatgttgctggctctgatgagctgatgctctgctgattagcaaacgctgagccatctgcgcttcgcagaggcacgttccagccaacccggccctccctgcccacttcccaggatgctttgccttgtgggctcacctgtcttctagctcctgatctgtatctccacctccatccagttccggggctccttatcagcactgttcccagaactgtccatcacgatggcaacgttctctctgggcgctgtccaacatgggagctcgcctctgtgttgtcactcatgctcattgaacatggatttgtgtcctttaccatcaggactggatacccctcctggtcctttctgcctggggtcttagcacagctcagaaggaacctcaccattccctctctccatctagggaattagaagatgacaggggcacagttctctggctcacccccagcccagtaaactcctggacatgcttcaaggcccagctcagatgttgcctcctcagtgaaataatttataaacccacccttctttgtcctgccttctccctcttccctactcactggagagttaacaggtgatggttaagctctgggttcaaatctcacaaggccacacacttagctatgtgacttcaggcaagttaattaaccactctgtgcctctcgtttcctcatttgtaaaatggaaatagtaaaagtgcctaccagcatggcagttgaagttaaaagaaataatatatgtgaacacttggaagggcgcctgacacatagtaaactctcagtaaatactagctgcttttagtggctattcttaacacaccctcttcagtgctctggtttcactatgttttatgggtccctgagatcgaaagtgtccacaccgactcatggtcagctgtaacctgtgcctcgtgtggggaccaggctgccatgtgtagtctggacagtgtaggaggtggcagagctcaggcctgttctgccctccagcccagagagccacgtcgttagatgtcatgggagactgtggtgccccgggaatctcacgaatttgcccacggtactcagtgtctgtccaatgctatgggagtccaggactctaggagccagttaaggtgctgggtggccacaggtccctggccaaggtccaggcctctcccctgccacctgatcctcgagaggccatcacgagggttgtacttcaagaaccactatccttgagctacctaggagctgcagaatgtgcactctgcagggcttagggcctgcagacaagatagatgcagggtgtctagttaaattcgaacttcagataaacaacaaataattttttcaaataattgtgttctattcggtccctatttgggacatatttgtactaaaaagtattcatttatctgaaattcagattcgactgggcatctggtgcttttgtttgctaaatccaagagcaaatttgttctagctacttctcaaccccaccttcagagaggaagccttgatggtactgtaacatcatgctgtaagaaggggatcccttgaattgtaaatggcactctgataagatgaggtatggggattgtattggtttcctgttgctgctgtcataaattaccacaaacttagtggcttcaaacaacacagatgcattatcttacagttctggaggtcacaagtctgaaagttagggcatcagcaggactgcattccttactgcggagttctagagaaaaatccattttcctgcctccttcagcctccagagacacgccacattctttggctagtggtctgcttccatctccaaggccagtgggggcttatcaagtctttctcacatcacatgactctgtttcttctgcctccctcttctacatttaagggacccttgtgattacacaggggcccacctagaaaagccaaaataatctccttattttaaaatcagctaatcagtggctttaatcccatctgcgatcttaattcctgtcgccatgtaacacaaggtattcccaggttctgtgggttaggacgtgggtgtctttcctaccacagggcagtttctagtgttgcctcttctccctgcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtggtaagggaggggtttggctgctcgccaattgcaaggtgattcctggggtagcagagcctcacgaattgaccttggggagggcgtgagcctggtgttctggacaatccttgcaaaagctccaggctcccagggctcaaaaaatcacaactgatagtatttctagaacagtggcccagggacccagaagtcactatgaggttcaccattaggtatgtggctgtggcatgtttgtgtccactctaaatgtggggataatcccctttacctcctctaacagagtggtaaaggaaggaggaggcctggtttgactccctgacctgctatttcctagccaggtgatcatggtaagatattgaaccttttctggtcccagtactcatctataaaacaaatataatactttacagagtggtaggaattatacaagaaaagtatacgcaaaacatttcataaattttaataaatgatggccccatgcttcttcctctggaaatggtctcaacctcaatggttggtgtttctagagagaaaaaacgacagagaaagtttcatagtctcaaaaatttggaaagccctgatctagctcaaccctttgttctagaactgcatcccagacagactgcttgggacctgaaaatatctcctcctttgctagaaggataagatgagaaggaattagataaaggaggtgtagagcagaggttttcacactgcaaagtgcataaaaaccatcagagggccgggcgcagtggctcacgcctgtaatcccagcactttgggaggccgaggcgggcggatcatgaggtcaggagatagagaccatcctggctaacacggtgaaaccccgtctctactaaaaaacacacacacacaaaaattagccaggtgtggtggcgggcgcctgtaatcccagctactgaggaggctgaggccggagaatggcgtgaacccgggaggcggagcttgcagtgagccgagattgcgccactgcactccagcctgggtgacagagcaagactccgtctcaataaaaacaaacaaacaaacaaaccaaaaaaacccatcagagaagttggtaaaagatgcaagtgctaaatccccacccccaatcactgtgattcagaagaaccaggccaggcccagaatctatcctgttaccttaggcgattctgatgaagaccattgtaggccacactttcagaaacactcaaaattagaatccttcagagaaggtggcatatataatatttctagcatggaattatgttttttttcttttgcctacattttaatttctagaactgtgttgtagggaatgtcagtcactaagaacttgattgaggaactgtgttttgtctgtttcatgactgctctctcaagtcccaggaaactcactttcagcttgtcttaaaaagcaagctgaaggcttttaaaaatgaagcaacatgaaataagacaccgcagtttctggcacggtccacgcttaatccccttcaatgtgtgactttccgtggaaagttactctacgattttcccagctcgtcagggtggggccccagagtgagtatgaagggtcagagcctagggatgccaccatcagtgagagcccaggaccccagaaaaggtctcttggctcaccacactgtaggaaaaataaaaagcaatgtagtccaaatgtctctatccaaagtttcaaaaagaacttgattttagacacgctccttgacttgttttcagaatcagacagaagagtgaggcaacaaaggtcccttattccaggcagctgaataccagcacagccaggagtccagtgctggtgtttgcagagccaccagaggctccctctcaggtgtccagggcccgcatgctttgtagaatgggcagaatgagcaatgtctgtgcacctgggctttgcaggcagggcctgggtacccaggttcgtgcaatcctctcgtcaccatgaagggagcagcatcattcttcccttcttgaagcaccttggccaccagtataggtaaatttacctcccaggacatgaccattgattctgggatgtcaatgccagagatagtagggtaaatcggcacctgggtaaaactttccattggagactagaaccaaaactcaggacactggcttccaaatgtttctttatcagacaagaaagaccaagtctttccttacgtcttcacatgctgccttggcaaatgctagcattcacaaaccctgggctaccttgacctgtcacccttgcagacctcagacgggtcctgggggcttgctttctcggtttctgtatgcaggcactcaaacctgcatcaggcacctgtgaagggccgggcactgtgctgaggccaaggctccaaatgtgaaccttccaccctcactgaactcacagccagaccagagacaagcaaacaggacatttcacagcagtgcagcctagaaagggccaacaccagcagcatttgtccccccgagcggtagcttttagaagcttccccagtgattcaatgtgtcctacaaatgcctggcccccactcccagagattctgagtcagctggcctagggtgcagccttgacttcactgtgttaaaaagcttcccagataagtccaatgtccggccaagattgagaatcactgacctagagtttaatttaccacctcagtctctatagaccacgcataataatagtaccccacacacctctgagggtccaaagaactttcatttgatcacccatgagaccaccgtggtgtggagatgctttctctctcctgttctcttaacaaagctggtgagcgacagagcctgcagtggaccgggagatggcccagaggagaaagctctgccgtagtcggcctcagttaaccacggagcaccacccctacctgctctcctctcactcctgcttccgtctcggtggagaaagatccaaccgaagcaggacacatctagtcttctggtgcctttaaaatgtacttttccatttgacaaatggattacactaaaaacaaaaatttacaaaaaaaaaaaaaaaacctgaaagaaattgcaggcattaaaatgggactttgcctttattgctcctgggcccatcctatttgggtttttagaaaaacaagcctgaggcaggcccagaaaggctcagggcagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgagaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggaaacaaaactaaatcaggaaggtttttgttgttgtttttggggggttttatgaaaatattcaagccacagcaaatatatttgatttatagcattagtattttttctgcctgcatctacaaaaatctttacctattaccatcaaaatatcctctgggtgaatggatttcaacaaagaagaaataaaaatgaaatagaagagaggccccttcgtgcacattgagcctactggctggattgtcacttgcctgccttgatgtcttttcagctccaggcaggcagtaggccagggcttattttcatgacagatcagatgttcttttatggatttacaaagaaagaaatactgagaagtcaaaactgaagtcacttaagacaagagcaggcccctgggaaggctgccattgaggataatgagtcctggggtcctggcctttgttcagtaaatacgcactaggcgcctacaatgtgtgcaccaatgtgtgaggcgtcaggttctctccagggtcagttggttttaagaaaggttttggcttctgatatgttttatctctacagaacagtagctcttaacctttcttatgggttaggattaccttcgagaatctgactacagctctagacctgttccctaaagaaaactaagttcacagggacacacaggatggggctcatggagcagctgaagccagaccccaggttaatagcctttacattaaaatgtttttctacctaccactaatatgcattctttagtaagcggtctcaatatacaccgattcttccttaactctgtttatgaagtattcagcatcctccctgcccccttcagcatcctccctgcccctgagcacaggatccaatggcgtgaggaccacaggcctgggcagctgctggggcatacaggcatctcttagtggctgagagactgggccctggctctatgttggctcctaacttgctgccatttaaaggaaatcttagcctcccatccgtaaaatcgagaaaataagacttgtcctacacagctcatgaaatagtaatgaaattcacattagagaagagatggaaaaacactttgaacaaaaagcattttgctcttataaaagcacagcctcttttgagaggccctttgctccccatttctccttcttcagacccccccagactaggagaaggtctgtctcatggagtgaccttttggctgcctctagattccaagctcagttttgctttcattaaccacagatactgggacggacagaaaaagacctagtttctgttgagccaaagagtctcataacttgtctgttcacatacccaagagcccaccctctagttgagacactcagttccctctcattctgggagactgcatgtctctgtgacctcctggtagagaccgtttgacatgtcccccaaccccccagtgattgagtctgaattctccactgatgacgcatttcctagcactcagggtgtcccctcctggttgccccctcaccactgaagcccgcttcctcccttttcatttgatgcttaacaactgtcagtttgcaagaaacatgcttcaaatccacattctcccagttgcctagcaacaacttccctcccggataaatgtgggtttcctgtagctcagcccaggactgaacacagcagcacacacttctgtccactgcttcaactgcttttcacctctggtctgcatgccttcaagactgcagctcatccctcccttcagaaccttccatagcctgcagaggccatgtctgccccaaaaagacacattgaacctgaggctacttatttacccttgtgttaggtatatcctcaacttagaaattaatactgtttccagattgtcttctttgaatcacagaaagtaaaacaacaaaacattcaatgcttaagacatttcatgtgcggttgggtgacatctgtttgatgaacacatttgatccaaagcatcagaaatactatgccaacaagactttttaggaggtgataaacatgtctgttctaccttaagaaaaaaatattacacagtcccaagggagagacatggttttgatcccagacaacccaagcagagacctcttagggccggaatcatcttggctgctgcctaggaccttatatcaatttcttaagcacaggatcaaggcctaaaggccccttagactgacctcagttagtagaggcagatcccttcacagccttatcttccttagaggtctagtctgaccttgaacttcggctggcagtgctgtcagttgtgatgtgtgacatggaagagttatttgttacttggaaaattaagagaacttatttggcataggaaattgtgtgtgtgtgtgtgtgtgtgtgtgtgtgtgtgtgtgtgtgtgtgtgagatgatgtttgccattttgatctgtgacttttttttccagaaatagtttctcagttccattccaactaaacttacagtctcttccggttctttgacagaaacaattcatgtgaatttgaacagataatagggaagggggaaccaaaagaagaggagagccctgggaaagttattttataatttatggcaacctcagtcaggcaactgtgaacaggtacatatggagggctccctcgggactaggcagtattcagagatgtaaggtgtgaggaccggaccctcatcatttaccattcccactaaaaagagctgggaaggaaattgtagctgtagcaccaggcacgtaactggagcttagtaactatttggtgaaggaatattattaaattattaacaagatggaaaaaagggtattaaccacacaaaaatacatctcaagctattgtttctctgttccctttcccccaaattcctagtcttgctcttatctggctgtctctctagtcactctttcttgctgactctcttcacgttcctttctccacctggaattcctgggccctccccttttactgacagacactgtcctcactctcacagtcatcagtttgtctctttacaaacctcagctcaagtgtcacttccccgtccccaggtgaaactgactgctccctccctgtaagtcaccatgatgactgctatatatagccctcatggaacctaaaacctcaacagacacagtctctttcctactctgttatagtttatttactcattaattaccacaacacgtattattgagcacctactgtgtaccatgcccagaagataaaagacaaacaaaataaaacctattcctatgcttaatgagtttacagtctagtggagagatagatacattaaaaaataacagcaaaccaaaataaaagtggtaaataaatgcactgagaaagacaggaatagctaggaggggcacctaatccctagggaaggaaagctggaagagcatggtgatgggggaagaaggctttctggagaaggtgaggtagtttgaaatgagttgactctggccagtaggggtagagtgagaatggggtgagacagggtgggttggtcattttgatccattagtcctcaaagtgataggactagtggctaaggactgcaggctttacagaagcctacaaaactatttgagatttgaagtttttttttttttttaattggctccaaaagaaaatgaaaaaactttagaattataatgaatgaatattaaatgaatatttaaggaaggtaattttattcaacttcattgttaaatttagttaaaacaagcccttgagtttcattcaacactgttttatcataccgttgatgagagaaaacaaaactgattcctggccagggccactgtcagcgtggggtttgcacatctttcccatgtctgcttgggttagctccaggtactcctgtttcccccacatccccaagatgtgcccattagtggaaacggtgtgtctgcatgattccaacgtgagtgagtgtgggtgtgggagtgagtgcccctgccatgggagggcatcctgtccaggttagattcctaccttgtgccctgagctgctgggatggaatccagccacccatgactctgaactgaaataattgggtgaataattatcttactttttaattaatctttgaaaatgtatgtatagttcacatgtatttcaatatttaatattagaagtattttagtctttattttgaagtttggtgatttattgtaaccagaaacaagctatagaaacttaattttgggccaagtgcagtggctcacacctataatcccagcattttgggaggccgaggcagacgcatcacttgaggtccggagttcaagatcagcctggccaacatggtaaaaccctgtctctactaaaaaatacaaaaattagccagatgtggtgggcacctgtagtcccagctacttgggtggctgaagcaggagaatcacttgaacccgggaggcggagcagtgagcagagatcgtgccactgcactcccacctaggcgacagtgtgacactccatctcaaaaaaaaaaaaaaatagaaaagaaagaaacttaattctggtttatatcaattagcctgtggtaaaattggtttcattatagccatttcacttagttgaagtttccaataacctgtggatgaattaagtgaggatttactatattcataaaatcttaaattccaaagcctgtttgcagttcaggtttttccactttacaaacacttctaagtattcacaatgattgcttaaaattcataccagataaatcattaaataagttgttcaaagtcaaataatttcataagtaaaaattaggagcttttagaaaactatacctacatagacctagacctatagatagacagagatctgaatagatatggacacagatgctttccaaagtgttcatgtgatgtgtggtggagtttcaagaccagagtgtgcctggggcctgcagaagtaaaggagaggggatggagagaagattgtccacatggccatgggcaatctcccacccacactcaagtgaggaagacaggaaacaaattcagaaagaagagaaaataatcaaaactgatgggagcttgtgactgatttacttatgcgcagcctccctggagacatgagtgtggctgttccttaggttgtgcctctgggctcctaccccctcttagatgccttcctattatctaggacctggttgctttttgtctgcatagcttctttggattccagtctttgatgccagcttcctcctaaagtagcctttcagatgtcccttggttaccctctgctatctaagggctcatcctaccccacactcattcccagcaccaatttctggatctccaggctggagatttagacaatgggatgggaagaacccatgatgggtcccagacagaaagtggtgccagccacagaaagggcacacaggcacagaagttggtttggggtaagacgatgtggtcagttcagaacacgctggatctaggcagatgcccagcagacagttggatatgtaagtctgaagctctggggagaggtctaggttggaggtacagatttagaagtcatcaacaaaaaggtagcagattaaatgataaaggaaatgagactatc

SEQ ID NO: 3 - RNA, 45 nt, Unknown
  source: /note=“Description of Unknown GluR2 sequence”
guggaauaguauaacaauaugcuaaauguuguuauaguaucccac

SEQ ID NO: 4 - protein, 6 aa, Unknown
  source: /note=“Description of Unknown Lipid-binding domain sequence”
  MOD_RES (2)..(2): Any amino acid
KXKEGV

SEQ ID NO: 5 - protein, 9 aa, Unknown
  source: /note=“Description of Unknown GAV motif sequence”
VGGAVVTGV

SEQ ID NO: 6 - protein, 6 aa, Unknown
  source: /note=“Description of Unknown Copper-binding motif sequence”
DPDNEA

SEQ ID NO: 7 - DNA, 19 bp, Artificial Sequence
  source: /note=“Description of Artificial Sequence Synthetic primer”
aggcctgtaaggtggaggg

SEQ ID NO: 8 - DNA, 32 bp, Artificial Sequence
  source: /note=“Description of Artificial Sequence Synthetic primer”
tgaaataacggcaatttatccattgcacatac

SEQ ID NO: 9 - DNA, 20 bp, Artificial Sequence
  source: /note=“Description of Artificial Sequence Synthetic primer”
aatttatccattgcacatac

SEQ ID NO: 10 - DNA, 22 bp, Artificial Sequence
  source: /note=“Description of Artificial Sequence Synthetic probe”
cagaattgggaaatccagctag

SEQ ID NO: 11 - DNA, 21 bp, Artificial Sequence
  source: /note=“Description of Artificial Sequence Synthetic probe”
actgtctagttcccttctgtg

SEQ ID NO: 12 - DNA, 100 bp, Artificial Sequence
  source: /note=“Description of Artificial Sequence Synthetic polynucleotide”
tgataaaaggcgtacataattcttgtgtctactgtacagaatactgccgccagctggatttcccaattctgagtaacactctgcaatccaaacagggttc

SEQ ID NO: 13 - DNA, 100 bp, Artificial Sequence; identical to SEQ ID NO: 12
  source: /note=“Description of Artificial Sequence Synthetic polynucleotide”

SEQ ID NO: 14 - DNA, 100 bp, Artificial Sequence; identical to SEQ ID NO: 12
  source: /note=“Description of Artificial Sequence Synthetic oligonucleotide”
  source: /note=“Description of Combined DNA/RNA Molecule Synthetic oligonucleotide”

SEQ ID NO: 15 - DNA, 100 bp, Artificial Sequence; identical to SEQ ID NO: 12
  source: /note=“Description of Artificial Sequence Synthetic polynucleotide”

SEQ ID NO: 16 - DNA, 100 bp, Artificial Sequence
  source: /note=“Description of Artificial Sequence Synthetic oligonucleotide”
  source: /note=“Description of Combined DNA/RNA Molecule Synthetic oligonucleotide”
aacatcguagauugaagccacaaaauccacagcacacaaagacccugccaccauguauucacuucagugaaagggaagcaccgaaaugcugagugggggc

SEQ ID NO: 17 - DNA, 100 bp, Artificial Sequence
  source: /note=“Description of Artificial Sequence Synthetic polynucleotide”
tgataaaaggcgtacataagtcttgtgtctactgtacagaagactgccgccagctggatttcccaattctgagtaacactctgcaatccaaacagggttc

SEQ ID NO: 18 - DNA, 100 bp, Artificial Sequence; identical to SEQ ID NO: 17
  source: /note=“Description of Artificial Sequence Synthetic polynucleotide”

SEQ ID NO: 19 - DNA, 80 bp, Artificial Sequence
  source: /note=“Description of Artificial Sequence Synthetic oligonucleotide”
cgtacataagtcttgtgtctactgtacagaagactgccgccagctggatttcccaattctgagtaacactctgcaatcca

SEQ ID NO: 20 - DNA, 60 bp, Artificial Sequence
  source: /note=“Description of Artificial Sequence Synthetic oligonucleotide”
tcttgtgtctactgtacagaagactgccgccagctggatttcccaattctgagtaacact

SEQ ID NO: 21 - DNA, 50 bp, Artificial Sequence
  source: /note=“Description of Artificial Sequence Synthetic oligonucleotide”
tgtctactgtacagaagactgccgccagctggatttcccaattctgagta

SEQ ID NO: 22 - DNA, 40 bp, Artificial Sequence
  source: /note=“Description of Artificial Sequence Synthetic oligonucleotide”
actgtacagaagactgccgccagctggatttcccaattct

SEQ ID NO: 23 - DNA, 100 bp, Artificial Sequence; identical to SEQ ID NO: 16
  source: /note=“Description of Artificial Sequence Synthetic oligonucleotide”
  source: /note=“Description of Combined DNA/RNA Molecule Synthetic oligonucleotide”

SEQ ID NO: 24 - DNA, 50 bp, Artificial Sequence; identical to SEQ ID NO: 21
  source: /note=“Description of Artificial Sequence Synthetic oligonucleotide”

SEQ ID NO: 25 - DNA, 50 bp, Artificial Sequence; identical to SEQ ID NO: 21
  source: /note=“Description of Artificial Sequence Synthetic oligonucleotide”

SEQ ID NO: 26 - DNA, 50 bp, Artificial Sequence; identical to SEQ ID NO: 21
  source: /note=“Description of Artificial Sequence Synthetic oligonucleotide”
  source: /note=“Description of Combined DNA/RNA Molecule Synthetic oligonucleotide”

SEQ ID NO: 27 - DNA, 100 bp, Artificial Sequence; identical to SEQ ID NO: 17
  source: /note=“Description of Artificial Sequence Synthetic polynucleotide”

SEQ ID NO: 28 - DNA, 100 bp, Artificial Sequence; identical to SEQ ID NO: 17
  source: /note=“Description of Artificial Sequence Synthetic polynucleotide”

SEQ ID NO: 29 - DNA, 100 bp, Artificial Sequence; identical to SEQ ID NO: 17
  source: /note=“Description of Artificial Sequence Synthetic polynucleotide”

SEQ ID NO: 30 - DNA, 50 bp, Artificial Sequence; identical to SEQ ID NO: 21
  source: /note=“Description of Artificial Sequence Synthetic oligonucleotide”

SEQ ID NO: 31 - DNA, 50 bp, Artificial Sequence; identical to SEQ ID NO: 21
  source: /note=“Description of Artificial Sequence Synthetic oligonucleotide”

SEQ ID NO: 32 - DNA, 100 bp, Artificial Sequence
  source: /note=“Description of Artificial Sequence Synthetic polynucleotide”
aacatcgtagattgaagccacaaaatccacagcacacaaagaccctgccaccatgtattcacttcagtgaaagggaagcaccgaaatgctgagtgggggc

SEQ ID NO: 33 - DNA, 100 bp, Artificial Sequence
  source: /note=“Description of Artificial Sequence Synthetic polynucleotide”
gcgaagattaaaaaaattttgcattacataatttacacgaaagcaatgccatcacctcccctgtgtggacttgggagaggactgggccattctccttaga

SEQ ID NO: 34 - DNA, 100 bp, Artificial Sequence
  source: /note=“Description of Artificial Sequence Synthetic polynucleotide”
ccccagtgagcatcttgaatgtggttgtattgccggcaccattcactcccaggaggccaaagcactctccagtgagaactcggaccacagcctcccgctg

SEQ ID NO: 35 - DNA, 102 bp, Unknown
  source: /note=“Description of Unknown RAB7A sequence”
ggtggagggttgaaccctgtttggattgcagagtgttactcagaattgggaaatccagctagcggcagtattctgtacagtagacacaagaattatgtacgc

SEQ ID NO: 36 - DNA, 101 bp, Artificial Sequence
  source: /note=“Description of Artificial Sequence Synthetic polynucleotide”
gtggagggttgaaccctgtttggattgcagagtgttactcagaattgggaaatccagctggcggcagtattctgtacagtagacacaagaattatgtacgc

SEQ ID NO: 37 - DNA, 98 bp, Artificial Sequence
  source: /note=“Description of Artificial Sequence Synthetic oligonucleotide”
gagggttgaaccctgtttggattgcagagtgttactcagaattgggaaatccagctagcggcagtattctgtacagtagacacaagaattatgtacgc

SEQ ID NO: 38 - DNA, 66 bp, Unknown
  source: /note=“Description of Unknown RAB7A sequence”
aattcttgtgtctactgtacagaatactgccgctagctggatttcccaattctgagtaacactctg

SEQ ID NO: 39 - DNA, 66 bp, Artificial Sequence
  source: /note=“Description of Artificial Sequence Synthetic oligonucleotide”
aattcttgtgtctactgtacagaatactgccgccagctggatttcccaattctgagcaacactctg

SEQ ID NO: 40 - DNA, 50 bp, Artificial Sequence; identical to SEQ ID NO: 21
  source: /note=“Description of Artificial Sequence Synthetic oligonucleotide”
  source: /note=“Description of Combined DNA/RNA Molecule Synthetic oligonucleotide”

SEQ ID NO: 41 - DNA, 50 bp, Artificial Sequence; identical to SEQ ID NO: 21
  source: /note=“Description of Artificial Sequence Synthetic oligonucleotide”
  source: /note=“Description of Combined DNA/RNA Molecule Synthetic oligonucleotide”

SEQ ID NO: 42 - DNA, 50 bp, Artificial Sequence; identical to SEQ ID NO: 21
  source: /note=“Description of Artificial Sequence Synthetic oligonucleotide”
  source: /note=“Description of Combined DNA/RNA Molecule Synthetic oligonucleotide”

SEQ ID NO: 43 - DNA, 50 bp, Artificial Sequence; identical to SEQ ID NO: 21
  source: /note=“Description of Artificial Sequence Synthetic oligonucleotide”
  source: /note=“Description of Combined DNA/RNA Molecule Synthetic oligonucleotide”

SEQ ID NO: 44 - DNA, 102 bp, Artificial Sequence; identical to SEQ ID NO: 35
  source: /note=“Description of Artificial Sequence Synthetic polynucleotide”